(19) United States
(12) Patent Application Publication
(10) Pub. No.: US 2024/0361216 A1
(43) Pub. Date: Oct. 31, 2024

(54) FILTRATION SAMPLING AND TESTING DEVICES

(71) Applicant: HERO SCIENTIFIC LTD., Jerusalem (IL)

(72) Inventors: Robert Eric LEVITZ, Beit Shemesh (IL); Zvi Feldman, Sde Zvi (IL); Michael Librus, Netanya (IL); Rinat Adi, Holon (IL)

(73) Assignee: HERO SCIENTIFIC LTD., Jerusalem (IL)

(21) Appl. No.: 18/763,483

(22) Filed: Jul. 3, 2024

Related U.S. Application Data
(63) Continuation of application No. PCT/IL2023/050014, filed on Jan. 5, 2023.
Application No. PCT/IL2023/050014 is a continuation-in-part of application No. PCT/IL2022/050024, filed on Jan. 6, 2022.
(60) Provisional application No. 63/134,282, filed on Jan. 6, 2021.
(60) Provisional application No. 63/432,231, filed on Dec. 13, 2022.
(60) Provisional application No. 63/388,851, filed on Jul. 13, 2022.

(51) Int. Cl. G01N 1/40; B01D 33/01; G01N 33/543

(52) U.S. Cl. CPC G01N 1/4077; B01D 33/0183; G01N 33/54388; G01N 2001/4088

(57) ABSTRACT

A kit is provided that includes an extraction tube or a transport tube; a filter, a liquid for bathing the filter within the tube; and a filter shaft. The filter shaft includes a distal portion that is coupled to a central portion of the filter. The filter shaft is configured to insert the filter into the tube for bathing the filter in the liquid. Other embodiments are also described.

### CROSS-REFERENCE TO RELATED APPLICATIONS

The present application is a continuation of International Application PCT/IL2023/050014, filed Jan. 5, 2023, which published as PCT Publication WO 2023/131948 to Levitz et al., and which:
(a) is a continuation-in-part of International Application PCT/IL2022/050024, filed Jan. 6, 2022, which published as PCT Publication WO 2022/149135 to Feldman et al., which claims the benefit of U.S. Provisional Application 63/134,282, filed Jan. 6, 2021; and
(b) claims the benefit of U.S. Provisional Application 63/432,231, filed Dec. 13, 2022, and U.S. Provisional Application 63/388,851, filed Jul. 13, 2022.

All of the above-referenced applications are assigned to the assignee of the present application and incorporated herein by reference.

### FIELD OF THE APPLICATION

Applications of the present invention relate to sampling biological liquids.

### BACKGROUND OF THE APPLICATION

Many techniques exist for testing for the presence of bacteria and viruses for aiding in disease diagnosis. For example, testing for the influenza virus includes molecular-based detection methods, viral culture methods, and immunoassay methods. Influenza virus testing includes the testing of nasal swabs, nasopharyngeal swabs, nasal aspirates, nasopharyngeal aspirates, nasal washes, nasopharyngeal washes, throat swabs, and a combination of samples.

PCT Publication WO 2018/158768 to Fruchter et al. describes inter alia a method for testing for presence of a particulate selected from the group consisting of: a microorganism, a fungus, a bacteria, a spore, a virus, a mite, a biological cell, a biological antigen, a protein, a protein antigen, and a carbohydrate antigen. The method includes (a) collecting, in a tube, fluid that potentially contains the particulate, (b) using a plunger to push the fluid through a filter removably disposed at a distal portion of the tube or at a distal end of the plunger, and subsequently, (c) while the filter is inside the tube, ascertaining if any of the particulate was trapped by the filter by applying a particulate-presence-testing-facilitation solution to the filter.

PCT Publication WO 2020/049569 to Fruchter et al. describes inter alia a testing device for testing for the presence of particulate in a liquid. The testing device includes a liquid container for containing the liquid; a filter, disposed in or downstream of the liquid container; a liquid-pressure source, which is arranged to apply pressure to drive the liquid contained in the liquid container through the filter; and a filter chamber that is (a) disposed downstream of the liquid container, (b) shaped so as to define an inlet, and (c) in fluid communication with the filter.

US Patent Application Publication 2011/0318814 to Kshirsagar et al. describes inter alia a method for isolating microorganisms from a sample, the sample including sample matrix and microorganisms, the method including the steps of providing a receptacle, the receptacle configured to allow filtering of the sample and to reversibly contain the sample and a concentration agent; adding the sample to the receptacle, wherein a microorganism-bound composition will be formed in the receptacle, the microorganism-bound composition including concentration agent-bound microorganisms and sample matrix; and filtering the microorganism-bound composition through a filter to collect the concentration agent-bound microorganisms on the filter. The filter has an average pore size that is greater than the average size of the microorganisms. Kits and systems are also described.

U.S. Pat. No. 5,077,012 to Guirguis describes an apparatus for collecting biological fluids and holding samples taken from a biological fluid for qualitative and quantitative testing. The apparatus comprises a tubular container open at both ends with a quantitative test storage unit removably secured to one of said tubular container ends. The quantitative test storage unit has an open end, a cytology membrane mounted in the storage unit and a retaining rib. A shuttle assembly is slidably mounted in the tubular container comprising a cylindrical hollow piston defining a chamber, a thumb cover covering one end of the piston and a fluid flow aperture formed in the piston and a qualitative sample container assembly removable secured to the piston. The qualitative sample container assembly comprises a clip on membrane assembly including a membrane containing immobilized antibodies and a filter housing mounted to the clip on membrane assembly. The filter housing is adapted to be seated in the quantitative test storage unit after being slidably transported along the tubular container by the piston.

U.S. Pat. No. 8,322,539 to Ellis et al. describes a filter vial for separating biological and chemical fluids having a cylindrical sidewall with an open top and a closed bottom. A protrusion extends upwards from the middle of the bottom to form an annular recess. A tubular plunger has an open bottom end to which is fastened an annular cup having an outer sidewall sized to fit into and seal against the vial's sidewall. An inner sidewall of the annular cup holds a filter over an opening in the bottom of the plunger and forms a shaped cavity leading to that filter. The annular cup on the plunger fits into the annular recess in the vial to force fluid from the vial through the filter and into the plunger.

US Patent Application Publication 2015/0076069 to Ellis et al. describes a filter vial and piston where the vial has a cylindrical wall with a closed bottom and open top and with the hollow, tubular piston therein. The piston has a position stop located and configured to abut a mating position stop on the vial to limit relative movement of the piston and vial. The position stops are located to stop the distal end of the piston away from the bottom of the vial a distance of about 10-30% the height of the vial. That a material to be dissolved in a liquid in the vial and to remove unwanted molecules from the liquid with the piston forcing the liquid through the filter but not squeezing the slurry of material so as to force it into the filter.

### SUMMARY OF THE APPLICATION

Some applications of the present invention provide a kit comprising an extraction or transport tube, a filter, and a liquid for bathing the filter within the tube, such as one or more reagents. A filter shaft includes a distal portion that is coupled to a central portion of the filter. The filter shaft is configured to insert the filter into the tube for bathing the filter in the liquid. Typically, the filter is circular when flat.

For some applications, the kit further comprises a diagnostic test for testing a portion of the one or more reagents for the presence of a target analyte released into the one or more reagents from a biological particulate trapped by the filter. For example, the diagnostic test may comprise a lateral flow test strip. For example, the target analyte may be an antigen.

For some applications, the diagnostic test comprises a housing, such as a cartridge or a card, and the lateral flow test strip is contained at least partially within the housing. The housing is shaped so as to define a channel in fluid communication with a sample pad of the lateral flow test trip. A bunched-up portion of the filter is squeezed to squeeze a portion of the liquid from the filter by inserting the bunched-up portion of the filter into the channel. This squeezing is typically performed by longitudinally compacting the bunched-up portion of the filter against a distal blocking surface intercepted by a straight central longitudinal axis of the channel, such as by distally pushing the bunched-up portion of the filter by the filter shaft. Generally, an internal wall of the channel constrains horizontal expansion of the bunched-up portion as the bunched-up portion is longitudinally compacted, thereby preventing the bunched-up portion of the filter from horizontally spreading out rather than becoming compacted.

For some applications, the kit further comprises a collection vial, which is shaped so as to define a vial opening and a shaft-passage hole at an end of the collection vial opposite the vial opening. The filter shaft is disposed partially within the collection vial, and includes a proximal portion that is slidably disposed passing through the shaft-passage hole such that the distal portion of the filter shaft passes through the vial opening. The kit is configured such that proximal movement of the filter shaft with respect to the collection vial pulls the filter at least partially into the collection vial via the vial opening, and optionally bunches up at least a portion of the filter.

In some applications of the present invention, a method is provided that comprises passing at least a portion of a liquid specimen sample through a filter so as to trap a biological particulate present in the liquid specimen, bunching up at least a portion of the filter, and placing a liquid in an extraction or transport tube; for example, the liquid may comprise one or more reagents. Before or after placing the liquid in the tube, the bunched-up portion of the filter is inserted into the tube and the filter is bathed in the liquid in the tube. Thereafter, a portion of the liquid is tested for the presence of a target analyte released into the liquid from the biological particulate trapped by the filter.

Some applications of the present invention provide sampling devices for concentrating a liquid specimen sample. The sampling devices may be used in combination with the techniques described above, or may be used separately from these techniques. Some of the sampling devices comprise a filtration assembly, which comprises a tubular container for receiving the liquid specimen sample, a plunger, and a filter disposed in the tubular container. The filtration assembly is configured such that movement of a plunger head within the tubular container, when the liquid specimen sample is contained in the tubular container and the filter is disposed in the tubular container, pushes at least a portion of the liquid specimen sample through the filter. The sampling device is configured such that the filter is removable from the tubular container while the plunger head remains within the tubular container. Once the filter has been removed, a diagnostic test may be performed for the presence of particulate trapped by the filter.

In some applications, the plunger comprises a plunger head and a plunger rod. The plunger rod has a distal end portion to which the plunger head is coupled, and is shaped so as to define an internal plunger space having a plunger-space proximal opening through a proximal end of the plunger rod. These sampling devices are configured such that the filter is removable from the tubular container via the plunger-space proximal opening while the plunger head remains within the tubular container.

In some configurations, the plunger head is shaped so as to define a plunger-head opening through the plunger head and into the internal plunger space. The sampling device comprises a filter-withdrawal shaft, which includes a distal portion that is coupled to a central portion of the filter, and which is disposed passing through the internal plunger space. The sampling device is configured such that proximal withdrawal of the filter-withdrawal shaft out of the internal plunger space, while the plunger head remains within the tubular container, pulls the filter into the internal plunger space via the plunger-head opening and out of the internal plunger space via the plunger-space proximal opening, and removes the filter-withdrawal shaft and the filter from the filtration assembly.

There are therefore provided, in accordance with respective applications of the present invention, the following Inventive Concepts:

Inventive Concept 1. A kit comprising:
a tube selected from the group of tubes consisting of: an extraction tube and a transport tube;
a filter;
a liquid for bathing the filter within the tube; and
a filter shaft, (a) which includes a distal portion that is coupled to a central portion of the filter, and (b) which is configured to insert the filter into the tube for bathing the filter in the liquid.

Inventive Concept 2. The kit according to Inventive Concept 1, wherein the filter is circular when flat.

Inventive Concept 3. The kit according to Inventive Concept 1, further comprising a filter reinforcement, which is coupled to a surface of the filter so as to cover 1%-50% of a surface area of the surface.

Inventive Concept 4. The kit according to Inventive Concept 1, wherein the filter shaft extends away from a first side of the filter and does not extend away from a second side of the filter opposite the first side, or extends away from the second side of the filter by less than 2 mm.

Inventive Concept 5. The kit according to Inventive Concept 1, wherein the tube comprises a flexible material.

Inventive Concept 6. The kit according to Inventive Concept 1, wherein a proximal end opening of the tube is shaped as a funnel.

Inventive Concept 7. The kit according to Inventive Concept 1, wherein the liquid is selected from the group consisting of: a lysis buffer, an extraction buffer, saline solution, and a transport medium.

Inventive Concept 8. The kit according to Inventive Concept 1, wherein the distal portion of the filter shaft is indirectly coupled to the central portion of the filter.

Inventive Concept 9. The kit according to Inventive Concept 1, wherein the distal portion of the filter shaft is directly coupled to the central portion of the filter.

Inventive Concept 10. The kit according to Inventive Concept 9, further comprising a distal plate that is fixed to a distal end of the distal portion of the filter shaft such that the central portion of the filter is between the distal end and the distal plate, so as to directly couple the filter to the filter shaft.

Inventive Concept 11. The kit according to any one of Inventive Concepts 1-10, wherein the kit is configured to limit maximum distal advancement of the filter within the tube.

Inventive Concept 12. The kit according to Inventive Concept 11, wherein the kit is configured to set an extent of the distal advancement of the filter within the tube to a predetermined distance of the distal advancement, so as to set an axial location of the filter within the tube.

Inventive Concept 13. The kit according to any one of Inventive Concepts 1-10, wherein the liquid comprises reagents and the tube is the extraction tube.

Inventive Concept 14. The kit according to any one of Inventive Concepts 1-10, wherein the liquid comprises one or more reagents and the tube is the extraction tube.

Inventive Concept 15. The kit according to any one of Inventive Concepts 13-14, further comprising a diagnostic test for testing for the presence of a biological particulate trapped by the filter.

Inventive Concept 16. The kit according to Inventive Concept 15, wherein the diagnostic test is configured to test a portion of the reagents for the presence of a target analyte released into the reagents from the biological particulate trapped by the filter.

Inventive Concept 17. The kit according to Inventive Concept 15, wherein the diagnostic test comprises a lateral flow test strip.

Inventive Concept 18. The kit according to Inventive Concept 17, wherein the diagnostic test comprises a housing.

Inventive Concept 19. The kit according to Inventive Concept 18, wherein the housing comprises a housing selected from the group consisting of: a cartridge and a card, and wherein the lateral flow test strip is contained at least partially within the housing.

Inventive Concept 20. The kit according to Inventive Concept 18,
wherein the lateral flow test strip comprises a sample pad,
wherein the housing is shaped so as to define a channel in fluid communication with the sample pad,
wherein the kit comprises a distal blocking surface that is intercepted by a straight longitudinal axis of the channel, and
wherein the kit is configured such that upon insertion and distal advancement of a bunched-up portion of the filter in the channel, the bunched-up portion of the filter is compacted against the distal blocking surface.

Inventive Concept 21. The kit according to Inventive Concept 20, wherein the straight longitudinal axis of the channel forms an angle of 30-90 degrees with the distal blocking surface.

Inventive Concept 22. The kit according to Inventive Concept 20, wherein the channel is shaped such that the insertion and the distal advancement of the bunched-up portion of the filter through the channel, while the at least a portion of the filter is bunched up, brings the bunched-up portion of the filter into direct contact with the sample pad of the lateral flow test strip, the sample pad defining the distal blocking surface.

Inventive Concept 23. The kit according to Inventive Concept 22, wherein the channel is shaped such that the insertion and the distal advancement of the bunched-up portion of the filter through the channel, while the at least a portion of the filter is bunched up such that an entirety of a perimeter of the filter points distally, brings at least a portion of the perimeter into direct contact with the sample pad of the lateral flow test strip.

Inventive Concept 24. The kit according to Inventive Concept 20, wherein the sample pad is disposed at an upstream end portion of the lateral flow test strip, and the lateral flow test strip further comprises (a) an absorbent pad disposed at a downstream end portion of the lateral flow test strip, and (b) a membrane disposed longitudinally between the sample pad and the absorbent pad, the membrane comprising a test area, which comprises a test line and a control line, and
wherein the housing is configured such that when the housing is placed on a flat surface, the test area of the membrane is farther from flat surface than the sample pad is from the flat surface.

Inventive Concept 25. The kit according to Inventive Concept 20, wherein the channel has an internal length of 1-5 cm.

Inventive Concept 26. The kit according to Inventive Concept 18, wherein the housing is shaped so as to define an elongate test-strip receptacle for placement of the lateral flow test strip therein.

Inventive Concept 27. The kit according to Inventive Concept 26, wherein the kit is configured to allow insertion of the lateral flow test strip into the elongate test-strip receptacle during a test procedure, without disassembling the housing.

Inventive Concept 28. The kit according to Inventive Concept 26,
wherein the lateral flow test strip comprises a sample pad,
wherein the housing is shaped so as to define a channel in fluid communication with the sample pad when the lateral flow test strip is placed within the elongate test-strip receptacle,
wherein the kit comprises a distal blocking surface that is intercepted by a straight longitudinal axis of the channel,
wherein the kit is configured such that upon insertion and distal advancement of a bunched-up portion of the filter in the channel, the bunched-up portion of the filter is compacted against the distal blocking surface, and
wherein a central longitudinal axis of the elongate test-strip receptacle (a) defines an angle of less than less than 45 degrees with a straight central longitudinal axis of the channel, or (b) is parallel with the straight central longitudinal axis of the channel.

Inventive Concept 29. The kit according to Inventive Concept 28, wherein the housing is configured to be used while the straight central longitudinal axis of the channel defines an angle of 60-90 degrees with a surface that is horizontal with respect to the Earth.

Inventive Concept 30. The kit according to Inventive Concept 17, wherein the lateral flow test strip is implemented as a dipstick.

Inventive Concept 31. The kit according to Inventive Concept 15, wherein the diagnostic test comprises a rapid molecular test.

Inventive Concept 32. The kit according to Inventive Concept 15, further comprising a testing tube separate and distinct from the extraction tube.

Inventive Concept 33. The kit according to any one of Inventive Concepts 1-10,
wherein the kit further comprises a collection vial, which is shaped so as to define a vial opening and a shaft-passage hole at an end of the collection vial opposite the vial opening,
wherein the filter shaft is disposed partially within the collection vial, and includes a proximal portion that is slidably disposed passing through the shaft-passage hole such that the distal portion of the filter shaft passes through the vial opening, and wherein the kit is configured such that proximal movement of the filter shaft with respect to the collection vial pulls the filter at least partially into the collection vial via the vial opening.

Inventive Concept 34. The kit according to Inventive Concept 33, wherein the kit is configured such that the proximal movement of the filter shaft with respect to the collection vial pulls the filter at least partially into the collection vial via the vial opening and bunches up at least a portion of the filter.

Inventive Concept 35. The kit according to Inventive Concept 33, further comprising a seal that inhibits fluid leakage between the proximal portion of the filter shaft and the shaft-passage hole.

Inventive Concept 36. The kit according to Inventive Concept 33, wherein the extraction tube and the collection vial are configured such that upon insertion of the collection vial at least partially into the extraction tube and upon distal advancement of the collection vial within the extraction tube, the tube prevents the collection vial from reaching a distal end of the extraction tube, such that the collection vial slides up a portion of the filter shaft as the filter is exposed from the collection vial and is positioned near the distal end of the extraction tube.

Inventive Concept 37. The kit according to Inventive Concept 33,
further comprising a diagnostic test comprising (a) a lateral flow test strip, which comprises a sample pad; and (b) a housing, wherein the lateral flow test strip is contained at least partially within the housing, and wherein the housing is shaped so as to define a channel in fluid communication with the sample pad, and
wherein the channel and the collection vial are configured such that upon insertion of the collection vial at least partially into the channel and upon distal advancement of the collection vial within the channel, the channel prevents the collection vial from reaching a distal end of the channel, such that the collection vial slides up a portion of the filter shaft as the filter is exposed from the collection vial and is positioned near the distal end of the channel.

Inventive Concept 38. The kit according to Inventive Concept 37, wherein the housing comprises a housing selected from the group consisting of: a cartridge and a card.

Inventive Concept 39. The kit according to Inventive Concept 33, further comprising a testing tube separate and distinct from the extraction tube, wherein the testing tube and the collection vial are configured such that upon insertion of the collection vial at least partially into the testing tube and upon distal advancement of the collection vial within the testing tube, the testing tube prevents the collection vial from reaching a distal end of the testing tube, such that the collection vial slides up a portion of the filter shaft as the filter is exposed from the collection vial and is positioned near the distal end of the testing tube.

Inventive Concept 40. The kit according to any one of Inventive Concepts 1-10, further comprising a sampling device for concentrating a liquid specimen sample, the sampling device comprising a filtration assembly, which comprises:
a container, which is shaped so as to define an inner wall and a proximal container opening for receiving the liquid specimen sample; and
a filter support, which is shaped so as to define (a) a support surface on which the filter is removably disposed, and (b) a plurality of filtrate-passage openings through the filter support,
wherein the filter shaft is removably disposed partially within the filtration assembly,
wherein the filtration assembly is configured to, when the liquid specimen sample is contained in the container and the filter is disposed on the support surface, push at least a portion of the liquid specimen sample through the filter, and
wherein the sampling device is configured such that withdrawal of the filter shaft out of the filtration assembly removes the filter shaft and the filter from the filtration assembly and bunches up at least a portion of the filter, thereby facilitating insertion of the bunched-up filter into the tube.

Inventive Concept 41. The kit according to Inventive Concept 40,
wherein the container is tubular,
wherein the filtration assembly further comprises a plunger, which (i) comprises a plunger head and (ii) is insertable into the tubular container via the proximal container opening, such that a lateral surface of the plunger head forms a fluid-tight movable seal with the inner wall, and
wherein the filtration assembly is configured such that movement of the plunger head within the tubular container, when the liquid specimen sample is contained in the tubular container and the filter is disposed on the support surface, pushes at least a portion of the liquid specimen sample through the filter.

Inventive Concept 42. The kit according to Inventive Concept 41, wherein the sampling device is configured such that the filter is removable from the tubular container while the plunger head, including the filter support thereof, remains within the tubular container.

Inventive Concept 43. The kit according to Inventive Concept 40, wherein the sampling device is configured such that the withdrawal of the filter shaft out of the filtration assembly removes the filter shaft and the filter from the filtration assembly and bunches up the at least a portion of the filter into a flower-like arrangement.

Inventive Concept 44. The kit according to Inventive Concept 40, wherein the sampling device is configured such that the withdrawal of the filter shaft out of the filtration assembly removes the filter shaft and the filter from the filtration assembly and bunches up the at least a portion of the filter such that an entirety of a perimeter of the filter extends distally away from the distal portion of the filter shaft.

Inventive Concept 45. The kit according to Inventive Concept 40, wherein the sampling device is configured such that the withdrawal of the filter shaft out of the filtration assembly removes the filter shaft and the filter from the filtration assembly and bunches up the at least a portion of the filter such that an entirety of a perimeter of the filter points distally.

Inventive Concept 46. The kit according to Inventive Concept 40, wherein the sampling device is configured such that the withdrawal of the filter shaft out of the filtration assembly removes the filter shaft and the filter from the filtration assembly and bunches up the at least a portion of the filter such that the bunched-up portion of the filter defines an internal space open distally.

Inventive Concept 47. The kit according to Inventive Concept 40, wherein the filter is removably disposed in a flat shape on the support surface of the filter support.

Inventive Concept 48. The kit according to Inventive Concept 40,
wherein the filtration assembly is configured to, when the liquid specimen sample is contained in the container and the filter is disposed on the support surface, push the at least a portion of the liquid specimen sample through the filter in an upstream-to-downstream direction, thereby trapping, on an upstream surface of the filter, a portion of biological particulate present in the liquid specimen, and
wherein the sampling device is configured such that the withdrawal of the filter shaft out of the filtration assembly removes the filter shaft and the filter from the filtration assembly and bunches up the at least a portion of the filter such that the upstream surface of the filter is inside the bunched-up filter.

Inventive Concept 49. The kit according to Inventive Concept 40,
wherein the filtration assembly to, when the liquid specimen sample is contained in the container and the filter is disposed on the support surface, push the at least a portion of the liquid specimen sample through the filter in an upstream-to-downstream direction, thereby trapping, on an upstream surface of the filter, a portion of biological particulate present in the liquid specimen, and
wherein the sampling device is configured such that the withdrawal of the filter shaft out of the filtration assembly removes the filter shaft and the filter from the filtration assembly and bunches up the at least a portion of the filter such that a downstream surface of the filter is inside the bunched-up filter.

Inventive Concept 50. The kit according to Inventive Concept 40,
wherein the kit further comprises a filter receptacle that is coupled to a distal portion of the filter shaft and shaped so as to define a distal receptacle opening, and
wherein the sampling device is configured such that the withdrawal of the filter shaft out of the filtration assembly pulls the central portion of the filter into the filter receptacle via the distal receptacle opening, thereby causing the remainder of the filter to become bunched up and be disposed at least partially outside the filter receptacle.

Inventive Concept 51. The kit according to Inventive Concept 50, wherein the filter receptacle is slidably coupled to the distal portion of the filter shaft.

Inventive Concept 52. The kit according to Inventive Concept 40,
wherein the sampling device further comprises a collection vial, which is disengageably coupled to the filtration assembly, and
wherein the sampling device is configured such that the filter is advanceable into the collection vial while the collection vial is disengageably coupled to the filtration assembly.

Inventive Concept 53. The kit according to Inventive Concept 52,
wherein the container is tubular,
wherein the filtration assembly further comprises a plunger, which (i) comprises a plunger head and (ii) is insertable into the tubular container via the proximal container opening, such that a lateral surface of the plunger head forms a fluid-tight movable seal with the inner wall,
wherein the filtration assembly is configured such that movement of the plunger head within the tubular container, when the liquid specimen sample is contained in the tubular container and the filter is disposed on the support surface, pushes at least a portion of the liquid specimen sample through the filter, and
wherein the sampling device is configured such that the collection vial is decouplable from the filtration assembly while the plunger head remains within the tubular container.

Inventive Concept 54. The kit according to Inventive Concept 53,
wherein the collection vial is shaped so as to define a vial opening and a shaft-passage hole at an end of the collection vial opposite the vial opening,
wherein the sampling device is configured such that the filter is advanceable into the collection vial via the vial opening while the collection vial is disengageably coupled to the filtration assembly,
wherein the filter shaft is disposed partially within the collection vial within the filtration assembly, and includes a proximal portion that is slidably disposed passing through the shaft-passage hole such that the distal portion of the filter shaft passes through the vial opening, and
wherein the sampling device is configured such that proximal withdrawal of the filter shaft, while the distal portion of the filter shaft is coupled to the filter and while the plunger head remains within the tubular container, pulls the filter into the collection vial via the vial opening.

Inventive Concept 55. The kit according to Inventive Concept 54, further comprising a seal that inhibits fluid leakage between the proximal portion of the filter shaft and the shaft-passage hole.

Inventive Concept 56. The kit according to Inventive Concept 54, wherein the sampling device is configured such that further proximal withdrawal of the filter shaft out of the filtration assembly, while the plunger head remains within the tubular container, pulls the collection vial out of the filtration assembly.

Inventive Concept 57. The kit according to Inventive Concept 56, wherein the tube and the collection vial are configured such that upon insertion of the collection vial at least partially into the tube and upon distal advancement of the collection vial within the tube, the tube prevents the collection vial from reaching a distal end of the tube, such that the collection vial slides up a portion of the filter shaft as the filter is exposed from the collection vial and positioned near the distal end of the tube.

Inventive Concept 58. The kit according to any one of Inventive Concepts 1-57, wherein the filter comprises a polyethersulfone (PES) membrane filter.

Inventive Concept 59. A method comprising:
passing at least a portion of a liquid specimen sample through a filter;
bunching up at least a portion of the filter;
bathing the bunched-up portion of the filter in a liquid in a tube selected from the group of tubes consisting of: an extraction tube and a transport tube; and
thereafter, testing a portion of the liquid for the presence of a biological particulate released from the filter into the liquid.

Inventive Concept 60. The method according to Inventive Concept 59, wherein bathing the bunched-up portion of the filter in the liquid in the tube comprises inserting the bunched-up portion of the filter into the tube.

Inventive Concept 61. The method according to Inventive Concept 60,
wherein bunching up the at least a portion of the filter comprises bunching up the at least a portion of the filter such that an entirety of a perimeter of the filter points distally, and
wherein inserting the bunched-up portion of the filter into the tube comprises inserting, in a distal direction, the bunched-up portion of the filter into the tube while the entirety of the perimeter of the filter points distally.

Inventive Concept 62. The method according to Inventive Concept 60,
wherein bunching up the at least a portion of the filter comprises bunching up the at least a portion of the filter such that the bunched-up portion of the filter defines an internal space open distally, and
wherein inserting the bunched-up portion of the filter into the tube comprises inserting, in a distal direction, the bunched-up portion of the filter into the tube while the bunched-up portion of the filter defines the internal space open distally.

Inventive Concept 63. The method according to Inventive Concept 60, wherein a proximal end opening of the tube is shaped as a funnel, and wherein inserting the bunched-up portion of the filter into the tube comprises inserting the bunched-up portion of the filter into the tube via the proximal end opening of the tube.

Inventive Concept 64. The method according to Inventive Concept 59, wherein bunching up the at least a portion of the filter comprises bunching up the at least a portion of the filter while inserting the filter into the tube.

Inventive Concept 65. A method comprising:
passing at least a portion of a liquid specimen sample through a filter;
bunching up at least a portion of the filter;
placing a liquid in a tube selected from the group of tubes consisting of: an extraction tube and a transport tube;
before or after placing the liquid in the tube, inserting the bunched-up portion of the filter into the tube;
bathing the filter in the liquid in the tube; and
thereafter, testing a portion of the liquid for the presence of a biological particulate released from the filter into the liquid.

Inventive Concept 66. The method according to any one of Inventive Concepts 59 and 65, wherein the liquid is selected from the group consisting of: a lysis buffer, an extraction buffer, saline solution, and a transport medium.

Inventive Concept 67. The method according to any one of Inventive Concepts 59 and 65, wherein the filter is circular when flat.

Inventive Concept 68. The method according to any one of Inventive Concepts 59 and 65, wherein a filter reinforcement is coupled to a surface of the filter so as to cover 1%-50% of a surface area of the surface.

Inventive Concept 69. The method according to any one of Inventive Concepts 59 and 65, wherein bunching up the at least a portion of the filter comprises bunching up the at least a portion of the filter into a flower-like arrangement.

Inventive Concept 70. The method according to Inventive Concept 65,
wherein bunching up the at least a portion of the filter comprises bunching up the at least a portion of the filter such that an entirety of a perimeter of the filter points distally, and
wherein inserting the bunched-up portion of the filter into the tube comprises inserting, in a distal direction, the bunched-up portion of the filter into the tube while the entirety of the perimeter of the filter points distally.

Inventive Concept 71. The method according to Inventive Concept 65,
wherein bunching up the at least a portion of the filter comprises bunching up the at least a portion of the filter such that the bunched-up portion of the filter defines an internal space open distally, and
wherein inserting the bunched-up portion of the filter into the tube comprises inserting, in a distal direction, the bunched-up portion of the filter into the tube while the bunched-up portion of the filter defines the internal space open distally.

Inventive Concept 72. The method according to Inventive Concept 65, wherein a proximal end opening of the tube is shaped as a funnel, and wherein inserting the bunched-up portion of the filter into the tube comprises inserting the bunched-up portion of the filter into the tube via the proximal end opening of the tube.

Inventive Concept 73. The method according to any one of Inventive Concepts 59 and 65, wherein passing the at least a portion of the liquid specimen sample through the filter comprises passing the at least a portion of the liquid specimen sample through the filter while the filter is in a flat shape.

Inventive Concept 74. The method according to any one of Inventive Concepts 59 and 65,
wherein passing the at least a portion of the liquid specimen sample through the filter comprises passing the at least a portion of the liquid specimen sample through the filter in an upstream-to-downstream direction, thereby trapping, on an upstream surface of the filter, a portion of biological particulate present in the liquid specimen, and
wherein bunching up the at least a portion of the filter comprises bunching up the at least a portion of the filter such that the upstream surface of the filter is inside the bunched-up portion of the filter.

Inventive Concept 75. The method according to any one of Inventive Concepts 59 and 65,
wherein passing the at least a portion of the liquid specimen sample through the filter comprises passing the at least a portion of the liquid specimen sample through the filter in an upstream-to-downstream direction, thereby trapping, on an upstream surface of the filter, a portion of biological particulate present in the liquid specimen, and
wherein bunching up the at least a portion of the filter comprises bunching up the at least a portion of the filter such that a downstream surface of the filter is inside the bunched-up portion of the filter.

Inventive Concept 76. The method according to any one of Inventive Concepts 59 and 65, wherein testing the portion of the liquid comprises performing a rapid molecular test.

Inventive Concept 77. The method according to any one of Inventive Concepts 59 and 65, wherein the liquid specimen sample includes gargled fluid.

Inventive Concept 78. The method according to any one of Inventive Concepts 59 and 65, wherein the filter comprises a polyethersulfone (PES) membrane filter.

Inventive Concept 79. The method according to any one of Inventive Concepts 59-78,
wherein the liquid includes reagents and the tube is the extraction tube,
wherein bathing the filter in the liquid in the tube comprises bathing the filter in the reagents in the extraction tube, and
wherein testing the portion of the liquid comprises testing the portion of the reagents for the presence of the biological particulate released by the filter into the reagents.

Inventive Concept 80. The method according to Inventive Concept 79, wherein testing comprises discarding a portion of the reagents after bathing the filter in the reagents in the extraction tube.

Inventive Concept 81. The method according to Inventive Concept 79, wherein testing comprises draining excess reagents from the extraction tube and thereafter squeezing the bunched-up portion of the filter to squeeze the portion of the reagents from the filter. Inventive Concept 82. The method according to Inventive Concept 79, wherein testing comprises removing the bunched-up portion of the filter from the extraction tube and thereafter expelling the portion of the reagents from the bunched-up portion of the filter.

Inventive Concept 83. The method according to Inventive Concept 82, wherein expelling the portion of the reagents from the bunched-up portion of the filter comprises squeezing the bunched-up portion of the filter to squeeze the portion of the reagents from the bunched-up portion of the filter after the bunched-up portion of the filter is removed from the extraction tube.

Inventive Concept 84. The method according to Inventive Concept 83, wherein expelling the portion of the reagents from the bunched-up portion of the filter comprises inserting the filter into a testing tube separate and distinct from the extraction tube, and squeezing the bunched-up portion of the filter while the filter is within the testing tube.

Inventive Concept 85. The method according to Inventive Concept 84, wherein testing the portion of the reagents comprises:
removing the filter from the testing tube; and
thereafter, inserting a lateral flow test strip into the reagents within the testing tube.

Inventive Concept 86. The method according to Inventive Concept 83, wherein testing for the presence of the biological particulate comprises bringing the bunched-up portion of the filter into direct contact with a sample pad of a lateral flow test strip.

Inventive Concept 87. The method according to Inventive Concept 86, wherein bringing the bunched-up portion of the filter into the direct contact with the sample pad comprises orienting a filter shaft so that a straight central longitudinal axis of the filter shaft forms an angle of 30-90 degrees with the sample pad, wherein the filter shaft includes a distal portion coupled to the filter.

Inventive Concept 88. The method according to Inventive Concept 86, wherein bunching up the at least a portion of the filter comprises bunching up the at least a portion of the filter such that the bunched-up portion of the filter defines an internal space open distally.

Inventive Concept 89. The method according to Inventive Concept 86, wherein bunching up the at least a portion of the filter comprises bunching up the at least a portion of the filter into a flower-like arrangement.

Inventive Concept 90. The method according to Inventive Concept 86, wherein bringing the filter into the direct contact with the sample pad comprises squeezing the bunched-up portion of the filter against the sample pad of the lateral flow test strip.

Inventive Concept 91. The method according to Inventive Concept 90, wherein squeezing the bunched-up portion of the filter against the sample pad comprises squeezing the bunched-up portion of the filter by longitudinally compacting the bunched-up portion of the filter against the sample pad.

Inventive Concept 92. The method according to Inventive Concept 90, wherein squeezing the bunched-up portion of the filter against the sample pad delivers to the sample pad both (a) some of the reagents contained in or on the filter at locations of the filter that directly contact the sample pad, and (b) some of the reagents contained in or on the filter at locations other than the locations of the filter that directly contact the sample pad.

Inventive Concept 93. The method according to Inventive Concept 83,
wherein the filter is coupled to a distal portion of a filter shaft that is disposed partially within a flexible collection vial that is shaped so as to define a vial opening and a shaft-passage hole at an end of the flexible collection vial opposite the vial opening,
wherein the filter shaft includes a proximal portion that is slidably disposed passing through the shaft-passage hole such that the distal portion of the filter shaft passes through the vial opening, and
wherein expelling the portion of the reagents from the filter comprises:
   pulling the filter at least partially into the flexible collection vial via the vial opening, by proximally moving the filter shaft with respect to the flexible collection vial; and
   squeezing the bunched-up portion of the filter by squeezing the flexible collection vial.

Inventive Concept 94. The method according to Inventive Concept 82, wherein removing the filter from the extraction tube comprises removing the filter from the extraction tube without first squeezing the bunched-up portion of the filter within the extraction tube.

Inventive Concept 95. The method according to Inventive Concept 79, wherein testing comprises testing the portion of the reagents by applying the portion of the reagents to a lateral flow test strip.

Inventive Concept 96. The method according to Inventive Concept 95, wherein the lateral flow test strip is contained at least partially within a housing.

Inventive Concept 97. The method according to Inventive Concept 96, wherein the housing is selected from the group consisting of: a cartridge and a card.

Inventive Concept 98. The method according to Inventive Concept 95, wherein the lateral flow test strip is implemented as a dipstick.

Inventive Concept 99. The method according to Inventive Concept 98, wherein applying the portion of the reagents to the lateral flow test strip comprises inserting at least a portion of the dipstick into the extraction tube.

Inventive Concept 100. The method according to Inventive Concept 99, wherein applying the portion of the reagents to the lateral flow test strip comprises removing the filter from the extraction tube before inserting the at least a portion of the dipstick into the extraction tube.

Inventive Concept 101. The method according to Inventive Concept 100, wherein testing the portion of the reagents comprises, before removing the filter from the extraction tube, squeezing the bunched-up portion of the filter to squeeze the portion of the reagents from the filter while the bunched-up portion of the filter is within the extraction tube.

Inventive Concept 102. The method according to any one of Inventive Concepts 59-78,
wherein the liquid includes one or more reagents and the tube is the extraction tube,
wherein bathing the filter in the liquid in the tube comprises bathing the filter in the one or more reagents in the extraction tube, and
wherein testing the portion of the liquid comprises testing the portion of the one or more reagents for the presence of the biological particulate released by the filter into the one or more reagents.

Inventive Concept 103. The method according to Inventive Concept 102, wherein testing comprises discarding a portion of the one or more reagents after bathing the filter in the one or more reagents in the extraction tube.

Inventive Concept 104. The method according to Inventive Concept 102, wherein testing comprises draining excess reagent from the extraction tube and thereafter squeezing the bunched-up portion of the filter to squeeze the portion of the one or more reagents from the filter.

Inventive Concept 105. The method according to Inventive Concept 102, wherein testing comprises removing the bunched-up portion of the filter from the extraction tube and thereafter expelling the portion of the one or more reagents from the bunched-up portion of the filter.

Inventive Concept 106. The method according to Inventive Concept 105, wherein expelling the portion of the one or more reagents from the bunched-up portion of the filter comprises squeezing the bunched-up portion of the filter to squeeze the portion of the one or more reagents from the bunched-up portion of the filter after the filter is removed from the extraction tube.

Inventive Concept 107. The method according to Inventive Concept 106, wherein expelling the portion of the one or more reagents from the bunched-up portion of the filter comprises inserting the filter into a testing tube separate and distinct from the extraction tube, and squeezing the bunched-up portion of the filter while the filter is within the testing tube.

Inventive Concept 108. The method according to Inventive Concept 107, wherein testing the portion of the one or more reagents comprises:
removing the filter from the testing tube; and
thereafter, inserting a lateral flow test strip into the one or more reagents within the testing tube.

Inventive Concept 109. The method according to Inventive Concept 106, wherein testing for the presence of the biological particulate comprises bringing the bunched-up portion of the filter into direct contact with a sample pad of a lateral flow test strip. Inventive Concept 110. The method according to Inventive Concept 109, wherein bringing the bunched-up portion of the filter into the direct contact with the sample pad comprises orienting a filter shaft so that a straight central longitudinal axis of the filter shaft forms an angle of 30-90 degrees with the sample pad, wherein the filter shaft includes a distal portion coupled to the filter.

Inventive Concept 111. The method according to Inventive Concept 109, wherein bunching up the at least a portion of the filter comprises bunching up the at least a portion of the filter such that the bunched-up portion of the filter defines an internal space open distally.

Inventive Concept 112. The method according to Inventive Concept 109, wherein bunching up the at least a portion of the filter comprises bunching up the at least a portion of the filter into a flower-like arrangement.

Inventive Concept 113. The method according to Inventive Concept 109, wherein bringing the filter into the direct contact with the sample pad comprises squeezing the bunched-up portion of the filter against the sample pad of the lateral flow test strip.

Inventive Concept 114. The method according to Inventive Concept 113, wherein squeezing the bunched-up portion of the filter against the sample pad comprises squeezing the bunched-up portion of the filter by longitudinally compacting the bunched-up portion of the filter against the sample pad.

Inventive Concept 115. The method according to Inventive Concept 113, wherein squeezing the bunched-up portion of the filter against the sample pad delivers to the sample pad both (a) some of the one or more reagents contained in or on the filter at locations of the filter that directly contact the sample pad, and (b) some of the one or more reagents contained in or on the filter at locations other than the locations of the filter that directly contact the sample pad.

Inventive Concept 116. The method according to Inventive Concept 106,
wherein the filter is coupled to a distal portion of a filter shaft that is disposed partially within a flexible collection vial that is shaped so as to define a vial opening and a shaft-passage hole at an end of the flexible collection vial opposite the vial opening,
wherein the filter shaft includes a proximal portion that is slidably disposed passing through the shaft-passage hole such that the distal portion of the filter shaft passes through the vial opening, and
wherein expelling the portion of the one or more reagents from the filter comprises:
   pulling the filter at least partially into the flexible collection vial via the vial opening, by proximally moving the filter shaft with respect to the flexible collection vial; and
   squeezing the bunched-up portion of the filter by squeezing the flexible collection vial.

Inventive Concept 117. The method according to Inventive Concept 105, wherein removing the filter from the extraction tube comprises removing the filter from the extraction tube without first squeezing the bunched-up portion of the filter within the extraction tube.

Inventive Concept 118. The method according to Inventive Concept 102, wherein testing comprises testing the portion of the one or more reagents by applying the portion of the one or more reagents to a lateral flow test strip.

Inventive Concept 119. The method according to Inventive Concept 118, wherein the lateral flow test strip is contained at least partially within a housing.

Inventive Concept 120. The method according to Inventive Concept 119, wherein the housing is selected from the group consisting of: a cartridge and a card.

Inventive Concept 121. The method according to Inventive Concept 118, wherein the lateral flow test strip is implemented as a dipstick.

Inventive Concept 122. The method according to Inventive Concept 121, wherein applying the portion of the one or more reagents to the lateral flow test strip comprises inserting at least a portion of the dipstick into the extraction tube.

Inventive Concept 123. The method according to Inventive Concept 122, wherein applying the portion of the one or more reagents to the lateral flow test strip comprises removing the filter from the extraction tube before inserting the at least a portion of the dipstick into the extraction tube.

Inventive Concept 124. The method according to Inventive Concept 123, wherein testing the portion of the one or more reagents comprises, before removing the filter from the extraction tube, squeezing the bunched-up portion of the filter to squeeze the portion of the one or more reagents from the filter while the bunched-up portion of the filter is within the extraction tube.

Inventive Concept 125. The method according to any one of Inventive Concepts 59-78, wherein testing the portion of the liquid comprises squeezing the bunched-up portion of the filter to squeeze the portion of the liquid from the filter.

Inventive Concept 126. The method according to Inventive Concept 125, wherein squeezing the bunched-up portion of the filter comprises squeezing the bunched-up portion of the filter while the bunched-up portion of the filter is within the tube.

Inventive Concept 127. The method according to Inventive Concept 126, wherein squeezing the bunched-up portion of the filter while the bunched-up portion of the filter is within the tube comprises squeezing the bunched-up portion of the filter at least one time without expelling any of the liquid from the tube.

Inventive Concept 128. The method according to Inventive Concept 126, wherein squeezing the bunched-up portion of the filter comprises squeezing the bunched-up portion of the filter at least one time to both squeeze the portion of the liquid from the filter and to expel some of the liquid from the tube.

Inventive Concept 129. The method according to Inventive Concept 126, wherein squeezing the bunched-up portion of the filter while the bunched-up portion of the filter is within the tube comprises:
squeezing the bunched-up portion of the filter at least a first time without expelling any of the liquid from the tube; and
thereafter, squeezing the bunched-up portion of the filter at least a second time to both squeeze an additional portion of the liquid from the filter and to expel some of the liquid from the tube.

Inventive Concept 130. The method according to Inventive Concept 126, wherein the tube includes a flexible material, and wherein squeezing the bunched-up portion of the filter comprises squeezing a longitudinal portion of the tube along which longitudinal portion the bunched-up portion of the filter is disposed.

Inventive Concept 131. The method according to Inventive Concept 126, further comprising, after bathing the filter in the liquid in the tube and before squeezing the bunched-up portion of the filter, removing excess liquid from the tube.

Inventive Concept 132. The method according to Inventive Concept 125, wherein squeezing the bunched-up portion of the filter comprises removing the filter from the tube and squeezing the bunched-up portion of the filter while the bunched-up portion of the filter is outside the tube.

Inventive Concept 133. The method according to Inventive Concept 132, wherein testing for the presence of the biological particulate comprises bringing the bunched-up portion of the filter into direct contact with a sample pad of a lateral flow test strip.

Inventive Concept 134. The method according to Inventive Concept 132, wherein squeezing the bunched-up portion of the filter to squeeze the portion of the liquid from the filter comprises, after removing the filter from the tube, inserting the bunched-up portion of the filter into a channel and longitudinally compacting the bunched-up portion of the filter against a distal blocking surface intercepted by a straight central longitudinal axis of the channel.

Inventive Concept 135. The method according to Inventive Concept 134, wherein the channel is in fluid communication with a sample pad of a lateral flow test strip.

Inventive Concept 136. The method according to Inventive Concept 135,
wherein the lateral flow test strip is disposed at least partially within a housing such that a test area of the lateral flow test strip is visible through one or more result windows defined by the housing, and
wherein the housing further defines the channel.

Inventive Concept 137. The method according to Inventive Concept 136,
wherein bunching up the at least a portion of the filter comprises bunching up the at least a portion of the filter such that an entirety of a perimeter of the filter points distally, and
wherein inserting the bunched-up portion of the filter into the channel comprises inserting, in a distal direction, the bunched-up portion of the filter into the channel while the entirety of the perimeter of the filter points distally.

Inventive Concept 138. The method according to Inventive Concept 136, wherein inserting the bunched-up portion of the filter into the channel comprises advancing a distal portion of the bunched-up portion of the filter through the channel and into direct contact with the sample pad of the lateral flow test strip, the sample pad defining the distal blocking surface.

Inventive Concept 139. The method according to Inventive Concept 138,
wherein bunching up the at least a portion of the filter comprises bunching up the at least a portion of the filter such that an entirety of a perimeter of the filter points distally,
wherein inserting the bunched-up portion of the filter into the channel comprises inserting, in a distal direction, the bunched-up portion of the filter into the channel while the entirety of the perimeter of the filter points distally, and
wherein advancing the distal portion of the bunched-up portion of the filter comprises advancing the distal portion of the bunched-up portion of the filter through the channel so that at least a portion of the perimeter makes direct contact with the sample pad of the lateral flow test strip.

Inventive Concept 140. The method according to Inventive Concept 136,
wherein the sample pad is disposed at an upstream end portion of the lateral flow test strip, and the lateral flow test strip further includes (a) an absorbent pad disposed at a downstream end portion of the lateral flow test strip, and (b) a membrane disposed longitudinally between the sample pad and the absorbent pad, the membrane comprising a test area, which comprises a test line and a control line, and
wherein the housing is configured such that when the housing is placed on a flat surface, the test area of the membrane is farther from flat surface than the sample pad is from the flat surface.

Inventive Concept 141. The method according to Inventive Concept 132, wherein removing the filter from the tube comprises removing the filter from the tube without first squeezing the bunched-up portion of the filter within the tube.

Inventive Concept 142. The method according to Inventive Concept 125, wherein squeezing the bunched-up portion of the filter comprises inserting an object into the tube and using the object to squeeze the bunched-up portion of the filter.

Inventive Concept 143. The method according to Inventive Concept 125, wherein testing the portion of the liquid for the presence of the biological particulate comprises using a diagnostic test to test the portion of the liquid for the presence of the biological particulate, and wherein squeezing the bunched-up portion of the filter comprises inserting a portion of the diagnostic test into the tube and using the portion of the diagnostic test to squeeze the bunched-up portion of the filter.

Inventive Concept 144. The method according to any one of Inventive Concepts 59-78, wherein bunching up the at least a portion of the filter comprises bunching up the at least a portion of the filter while a central portion of the filter is coupled to a distal portion of a filter shaft.

Inventive Concept 145. The method according to Inventive Concept 144, wherein the filter shaft extends away from a first side of the filter and does not extend away from a second side of the filter opposite the first side, or extends away from the second side of the filter by less than 2 mm.

Inventive Concept 146. The method according to Inventive Concept 144,
wherein bunching up the at least a portion of the filter comprises bunching up the at least a portion of the filter such that an entirety of a perimeter of the filter extends distally away from the distal portion of the filter shaft, and
wherein inserting the bunched-up portion of the filter into the tube comprises inserting, in a distal direction, the bunched-up portion of the filter into the tube while the entirety of the perimeter of the filter extends distally away from the distal portion of the filter shaft.

Inventive Concept 147. The method according to Inventive Concept 144,
wherein bunching up the at least a portion of the filter comprises bunching up the at least a portion of the filter such that an entirety of a perimeter of the filter extends distally away from the distal portion of the filter shaft, and
wherein bathing the bunched-up portion of the filter in the liquid in the tube comprises inserting, in a distal direction, the bunched-up portion of the filter into the tube while the entirety of the perimeter of the filter extends distally away from the distal portion of the filter shaft.

Inventive Concept 148. The method according to Inventive Concept 144, wherein the distal portion of the filter shaft is indirectly coupled to the central portion of the filter. Inventive Concept 149. The method according to Inventive Concept 144, wherein the distal portion of the filter shaft is directly coupled to the central portion of the filter.

Inventive Concept 150. The method according to Inventive Concept 149, wherein a distal plate is fixed to a distal end of the distal portion of the filter shaft such that the central portion of the filter is between the distal end and the distal plate, so as to directly couple the filter to the filter shaft.

Inventive Concept 151. The method according to Inventive Concept 144,
wherein the filter shaft is disposed partially within a collection vial that is shaped so as to define a vial opening and a shaft-passage hole at an end of the collection vial opposite the vial opening,
wherein the filter shaft includes a proximal portion that is slidably disposed passing through the shaft-passage hole such that the distal portion of the filter shaft passes through the vial opening, and
wherein bunching up the at least a portion of the filter comprises pulling the filter at least partially into the collection vial via the vial opening, by proximally moving the filter shaft with respect to the collection vial.

Inventive Concept 152. The method according to Inventive Concept 151, wherein the collection vial includes a seal that inhibits fluid leakage between the proximal portion of the filter shaft and the shaft-passage hole.

Inventive Concept 153. The method according to Inventive Concept 151, wherein inserting the bunched-up portion of the filter into the tube comprises inserting the collection vial at least partially into the tube and distally advancing the collection vial within the tube, such that the tube prevents the collection vial from reaching a distal end of the tube, such that the collection vial slides up a portion of the filter shaft as the filter is exposed from the collection vial and is positioned near the distal end of the tube.

Inventive Concept 154. The method according to Inventive Concept 151, wherein bathing the bunched-up portion of the filter in the liquid in the tube comprises inserting the bunched-up portion of the filter into the tube by inserting the collection vial at least partially into the tube and distally advancing the collection vial within the tube, such that the tube prevents the collection vial from reaching a distal end of the tube, such that the collection vial slides up a portion of the filter shaft as the filter is exposed from the collection vial and is positioned near the distal end of the tube.

Inventive Concept 155. The method according to Inventive Concept 151,
wherein testing the portion of the liquid comprises squeezing the bunched-up portion of the filter to squeeze the portion of the liquid from the filter, by inserting the bunched-up portion of the filter into a channel in fluid communication with a sample pad of a lateral flow test strip disposed partially within a housing that defines the channel, and
wherein the channel and the collection vial are configured such that upon insertion of the collection vial at least partially into the channel and upon distal advancement of the collection vial within the channel, the channel prevents the collection vial from reaching a distal end of the channel, such that the collection vial slides up a portion of the filter shaft as the filter is exposed from the collection vial and is positioned near the distal end of the channel.

Inventive Concept 156. The method according to Inventive Concept 144,
wherein passing the at least a portion of the liquid specimen sample through the filter comprises using a filtration assembly while the filter is removably disposed in the filtration assembly and the filter shaft is removably disposed partially within the filtration assembly, and
wherein bunching up the at least a portion of the filter comprises withdrawing the filter shaft out of the filtration assembly such that the withdrawing removes the filter shaft and the filter from the filtration assembly and bunches up at least a portion of the filter.

Inventive Concept 157. The method according to Inventive Concept 156, wherein bunching up the at least a portion of the filter comprises withdrawing the filter shaft out of the filtration assembly such that the withdrawing of the filter shaft out of the filtration assembly removes the filter shaft and the filter from the filtration assembly and bunches up the at least a portion of the filter into a flower-like arrangement.

Inventive Concept 158. The method according to Inventive Concept 156, wherein bunching up the at least a portion of the filter comprises withdrawing the filter shaft out of the filtration assembly such that the withdrawing of the filter shaft out of the filtration assembly removes the filter shaft and the filter from the filtration assembly and bunches up the at least a portion of the filter such that an entirety of a perimeter of the filter extends distally away from the distal portion of the filter shaft.

Inventive Concept 159. The method according to Inventive Concept 156, wherein bunching up the at least a portion of the filter comprises withdrawing the filter shaft out of the filtration assembly such that the withdrawing of the filter shaft out of the filtration assembly removes the filter shaft and the filter from the filtration assembly and bunches up the at least a portion of the filter such that an entirety of a perimeter of the filter points distally.

Inventive Concept 160. The method according to Inventive Concept 156, wherein withdrawing the filter shaft out of the filtration assembly pulls the central portion of the filter into a filter receptacle via a distal receptacle opening, thereby causing the remainder of the filter to become bunched up and be disposed at least partially outside the filter receptacle, wherein the filter receptacle is coupled to a distal portion of the filter shaft.

Inventive Concept 161. The method according to Inventive Concept 160, wherein the filter receptacle is slidably coupled to the distal portion of the filter shaft.

Inventive Concept 162. The method according to Inventive Concept 156, wherein passing the at least a portion of the liquid specimen sample through the filter comprises:
placing the liquid sample in a tubular container of the filtration assembly, the tubular container shaped so as to define an inner wall and a proximal container opening for receiving the liquid specimen sample;
while the filter is removably disposed on a support surface of a filter support of the filtration assembly and the filter shaft is removably disposed partially within the filtration assembly, inserting a plunger into the tubular container via the proximal container opening, such that a lateral surface of a plunger head of the plunger forms a fluid-tight movable seal with the inner wall, wherein the filter support is shaped so as to define a plurality of filtrate-passage openings through the filter support; and
pushing at least a portion of the liquid specimen sample through the filter by moving the plunger head within the tubular container.

Inventive Concept 163. The method according to Inventive Concept 162, wherein inserting the plunger into the tubular container comprises inserting the plunger into the tubular container while the filter is removably disposed in a flat shape on the support surface of the filter support.

Inventive Concept 164. The method according to Inventive Concept 162, wherein pushing the at least a portion of the liquid specimen sample through the filter comprises pushing the at least a portion of the liquid specimen sample through the filter in a distal-to-proximal direction, thereby trapping, on a distal surface of the filter, a portion of biological particulate present in the liquid specimen.

Inventive Concept 165. The method according to Inventive Concept 162, wherein withdrawing the filter shaft out of the filtration assembly comprises removing the filter from the tubular container while the plunger head, including the filter support thereof, remains within the tubular container.

Inventive Concept 166. The method according to Inventive Concept 162, further comprising advancing the filter into a collection vial while the collection vial is disengageably coupled to the filtration assembly.

Inventive Concept 167. The method according to Inventive Concept 166, further comprising decoupling the collection vial from the filtration assembly while the plunger head remains within the tubular container.

Inventive Concept 168. The method according to Inventive Concept 167,
wherein advancing the filter into the collection vial comprises advancing the filter into the collection vial via a vial opening of the collection vial while the collection vial is disengageably coupled to the filtration assembly,
wherein the filter shaft is disposed partially within the collection vial within the filtration assembly, and includes a proximal portion that is slidably disposed passing through a shaft-passage hole at an end of the collection vial opposite the vial opening, such that the distal portion of the filter shaft passes through the vial opening, and
wherein withdrawing the filter shaft, while the plunger head remains within the tubular container, pulls the filter into the collection vial via the vial opening.

Inventive Concept 169. The method according to Inventive Concept 168, wherein further proximal withdrawing of the filter shaft out of the filtration assembly, while the plunger head remains within the tubular container, pulls the collection vial out of the filtration assembly.

Inventive Concept 170. The method according to Inventive Concept 169, further comprising, after pulling the collection vial out of the filtration assembly, inserting the collection vial at least partially into the tube and distally advancing the collection vial within the tube, such that the tube prevents the collection vial from reaching a distal end of the tube, such that the collection vial slides up a portion of the filter shaft as the filter is exposed from the collection vial and positioned near the distal end of the tube.

Inventive Concept 171. The method according to any one of Inventive Concepts 59-78, wherein the biological particulate is selected from the group consisting of: a virus, a bacterium, a microorganism, a fungus, a spore, a mite, a biological cell, a biological antigen, a protein, a protein antigen, and a carbohydrate antigen.

Inventive Concept 172. The method according to Inventive Concept 171, wherein the biological particulate is the biological antigen.

Inventive Concept 173. A sampling device for concentrating a liquid specimen sample, the sampling device comprising:
(a) a filter;
(b) a filtration assembly, which comprises:
   (i) a tubular container, which is shaped so as to define an inner wall and a proximal container opening for receiving the liquid specimen sample;
   (ii) a plunger, which (A) comprises a plunger head and (B) is insertable into the tubular container via the proximal container opening, such that a lateral surface of the plunger head forms a fluid-tight movable seal with the inner wall; and
   (iii) a filter support, which is shaped so as to define (A) a support surface on which the filter is removably disposed, and (B) a plurality of filtrate-passage openings through the filter support; and
(c) a withdrawer comprising a filter-withdrawal shaft, which is partially inserted in the filtration assembly,
wherein the filtration assembly is configured such that movement of the plunger head within the tubular container, when the liquid specimen sample is contained in the tubular container and the filter is disposed in the tubular container, pushes at least a portion of the liquid specimen sample through the filter, and
wherein the sampling device is configured such that removal of the filter-withdrawal shaft from the filtration assembly, while a distal portion of the filter-withdrawal shaft is coupled to the filter, while the plunger head remains within the tubular container, and while the filter support remains within the filtration assembly, removes the filter from the filter support and from the filtration assembly.

Inventive Concept 174. The sampling device according to Inventive Concept 173,
wherein the filter support is disposed within the tubular container, and
wherein the sampling device is configured such that the removal of the filter-withdrawal shaft from the filtration assembly, while the distal portion of the filter-withdrawal shaft is coupled to the filter, while the plunger head remains within the tubular container, and while the filter support remains within the tubular container, removes the filter from the filter support and from the filtration assembly.

Inventive Concept 175. The sampling device according to Inventive Concept 173,
wherein the filter support is shaped so as to define a central opening, in addition to the plurality of filtrate-passage openings, and
wherein the sampling device is configured such that removal of the filter-withdrawal shaft from the filtration assembly, while the plunger head remains within the tubular container and the filter support remains within the filtration assembly, pulls the filter through the central opening and removes the filter from the filter support and from the filtration assembly.

Inventive Concept 176. The sampling device according to Inventive Concept 173, wherein the filtration assembly further comprises a waste liquid receptacle, and wherein the filter support is shaped so as to define the plurality of filtrate-passage openings through the filter support into the waste liquid receptacle.

Inventive Concept 177. The sampling device according to Inventive Concept 173,
wherein the sampling device comprises a filter receptacle that is slidably coupled to a distal end portion of the filter-withdrawal shaft and shaped so as to define a distal receptacle opening, and
wherein the sampling device is configured such that withdrawal of the filter-withdrawal shaft from the filtration assembly pulls a portion of the filter into the filter receptacle via the distal receptacle opening.

Inventive Concept 178. The sampling device according to any one of Inventive Concepts 173-177,
wherein the plunger comprises a plunger rod, which (a) has a distal end portion to which the plunger head is coupled, and (b) is shaped so as to define an internal plunger space having a plunger-space proximal opening through a proximal end of the plunger rod,
wherein the plunger head is shaped so as to define a plunger-head opening through the plunger head and into the internal plunger space,
wherein the filter-withdrawal shaft is disposed passing through the internal plunger space, and
wherein the sampling device is configured such that proximal withdrawal of the filter-withdrawal shaft out of the internal plunger space, while the plunger head remains within the tubular container, pulls the filter into the internal plunger space via the plunger-head opening and out of the internal plunger space via the plunger-space proximal opening, and removes the filter-withdrawal shaft from the filtration assembly and the filter from the filter support and from the filtration assembly.

Inventive Concept 179. The sampling device according to Inventive Concept 178,
wherein the filtration assembly and the withdrawer are shaped so as to define corresponding screw threads, respectively, which (a) removably couple the filter-withdrawal shaft to the plunger rod while the filter-withdrawal shaft is disposed passing through the internal plunger space, and (b) prevent premature proximal withdrawal of the filter-withdrawal shaft out of the internal plunger space, and
wherein the sampling device is configured such that rotation of the filter-withdrawal shaft and the plunger-space proximal opening with respect to each other (a) causes at least an initial portion of the proximal withdrawal of the filter-withdrawal shaft out of the internal plunger space, and (b) decouples the screw threads from each other.

Inventive Concept 180. The sampling device according to Inventive Concept 179, wherein the sampling device is configured such that the filter-withdrawal shaft does not rotate during at least an initial portion of the proximal withdrawal of the filter-withdrawal shaft out of the internal plunger space.

Inventive Concept 181. The sampling device according to Inventive Concept 180,
wherein the withdrawer further comprises a shaft handle, which is coupled to a proximal portion of the filter-withdrawal shaft such that the shaft handle is free to rotate with respect to the filter-withdrawal shaft,
wherein the shaft handle of the withdrawer is shaped so as to define the screw thread of the withdrawer,
wherein the plunger comprises a plunger rod that comprises a hollow shaft that is shaped so as to define (a) the internal plunger space within the hollow shaft and (b) a non-circular inner surface along at least a longitudinal portion of the hollow shaft,
wherein the filter-withdrawal shaft is shaped so as to define a non-circular outer surface along at least a longitudinal portion of the filter-withdrawal shaft, and
wherein the sampling device is configured such that the non-circular outer surface engages the non-circular inner surface so as to prevent rotation of the filter-withdrawal shaft with respect to the hollow shaft during at least an initial portion of the proximal withdrawal of the filter-withdrawal shaft out of the internal plunger space.

Inventive Concept 182. The sampling device according to Inventive Concept 178, wherein the plunger rod is shaped so as to define therewithin a waste liquid receptacle, and wherein the filter support is shaped so as to define the plurality of filtrate-passage openings through the filter support into the waste liquid receptacle.

Inventive Concept 183. The sampling device according to any one of Inventive Concepts 173-177,
wherein the sampling device further comprises a collection vial, which is disengageably coupled to the filtration assembly, and wherein the sampling device is configured such that the filter is advanceable into the collection vial while the collection vial is disengageably coupled to the filtration assembly.

Inventive Concept 184. The sampling device according to Inventive Concept 183, wherein the sampling device is configured such that the collection vial is decouplable from the filtration assembly while the plunger head remains within the tubular container.

Inventive Concept 185. The sampling device according to Inventive Concept 184,
wherein the collection vial is shaped so as to define a vial opening and a shaft-passage hole at an end of the collection vial opposite the vial opening,
wherein the sampling device is configured such that the filter is advanceable into the collection vial via the vial opening while the collection vial is disengageably coupled to the filtration assembly,
wherein the filter-withdrawal shaft is disposed partially within the collection vial within the filtration assembly, and includes a proximal portion that is slidably disposed passing through the shaft-passage hole such that the distal portion of the filter-withdrawal shaft passes through the vial opening, and
wherein the sampling device is configured such that proximal withdrawal of the filter-withdrawal shaft, while the distal portion of the filter-withdrawal shaft is coupled to the filter and while the plunger head remains within the tubular container, pulls the filter into the collection vial via the vial opening.

Inventive Concept 186. The sampling device according to Inventive Concept 185, further comprising a seal that inhibits fluid leakage between the proximal portion of the filter-withdrawal shaft and the shaft-passage hole.

Inventive Concept 187. The sampling device according to Inventive Concept 185, wherein the sampling device is configured such that further proximal withdrawal of the filter-withdrawal shaft out of the filtration assembly, while the plunger head remains within the tubular container, pulls the collection vial out of the filtration assembly.

Inventive Concept 188. The sampling device according to Inventive Concept 187, further comprising a tube selected from the group of tubes consisting of: an extraction tube and a transport tube, wherein the tube and the collection vial are configured such that upon insertion of the collection vial at least partially into the tube and upon distal advancement of the collection vial within the tube, the tube prevents the collection vial from reaching a distal end of the tube, such that the collection vial slides up a portion of the filter-withdrawal shaft as the filter is exposed from the collection vial and positioned near the distal end of the tube.

Inventive Concept 189. The sampling device according to Inventive Concept 183,
wherein the plunger comprises a plunger rod, which (a) has a distal end portion to which the plunger head is coupled, and (b) is shaped so as to define an internal plunger space having a plunger-space proximal opening through a proximal end of the plunger rod, and
wherein the collection vial is removably disposed at least partially within the internal plunger space.

Inventive Concept 190. The sampling device according to Inventive Concept 189, wherein the sampling device is configured such that the collection vial is decouplable from the filtration assembly while the plunger head remains within the tubular container.

Inventive Concept 191. The sampling device according to Inventive Concept 189,
wherein the plunger head is shaped so as to define a plunger-head opening through the plunger head and into the internal plunger space,
wherein the collection vial is shaped so as to define a vial opening and a shaft-passage hole at an end of the collection vial opposite the vial opening,
wherein the sampling device is configured such that the filter is advanceable into the collection vial via the plunger-head opening and the vial opening while the collection vial is disengageably coupled to the filtration assembly,
wherein the filter-withdrawal shaft is disposed partially within the collection vial within the internal plunger space, and includes a proximal portion that is slidably disposed passing through the shaft-passage hole such that the distal portion of the filter-withdrawal shaft passes through the vial opening, and
wherein the sampling device is configured such that proximal withdrawal of the filter-withdrawal shaft, while the distal portion of the filter-withdrawal shaft is coupled to the filter and while the plunger head remains within the tubular container, pulls the filter into the collection vial via the plunger-head opening and the vial opening.

Inventive Concept 192. The sampling device according to Inventive Concept 191, further comprising a seal that inhibits fluid leakage between the proximal portion of the filter-withdrawal shaft and the shaft-passage hole.

Inventive Concept 193. The sampling device according to Inventive Concept 191, wherein the sampling device is configured such that further proximal withdrawal of the filter-withdrawal shaft out of the internal plunger space, while the plunger head remains within the tubular container, pulls the collection vial out of the internal plunger space via the plunger-space proximal opening.

Inventive Concept 194. The sampling device according to any one of Inventive Concepts 173-177,
wherein the filter-withdrawal shaft is disposed passing through a distal opening defined by a distal bottom surface of the tubular container, and wherein the sampling device is configured such that removal of the filter-withdrawal shaft from the filtration assembly, while a distal portion of the filter-withdrawal shaft is coupled to the filter, while the plunger head remains within the tubular container, and while the filter support remains within the filtration assembly, removes the filter from the filter support and from the filtration assembly via the distal opening defined by the bottom surface of the tubular container.

Inventive Concept 195. The sampling device according to Inventive Concept 194,
wherein the tubular container comprises a hollow shaft that is shaped so as to define an internal shaft space within the hollow shaft,
wherein the filter-withdrawal shaft is disposed passing through (a) the distal opening defined by the distal bottom surface of the tubular container and (b) the internal shaft space, and
wherein the sampling device is configured such that the removal of the filter-withdrawal shaft from the filtration assembly, while the distal portion of the filter-withdrawal shaft is coupled to the filter, while the plunger head remains within the tubular container, and while the filter support remains within the filtration assembly, removes the filter from the filter support and from the filtration assembly via (a) the distal opening defined by the bottom surface of the tubular container and (b) the internal shaft space.

Inventive Concept 196. A testing kit comprising the sampling device according to any one of Inventive Concepts 173-195, the testing kit further comprising a diagnostic test configured to detect the presence of a biological particulate trapped by the filter.

Inventive Concept 197. The testing kit according to Inventive Concept 196, wherein the diagnostic test comprises a lateral flow test strip.

Inventive Concept 198. The testing kit according to Inventive Concept 197, further comprising reagents for use with the lateral flow test strip.

Inventive Concept 199. The testing kit according to Inventive Concept 197, further comprising one or more reagents for use with the lateral flow test strip.

Inventive Concept 200. The testing kit according to Inventive Concept 196, wherein the diagnostic test comprises a rapid molecular test.

Inventive Concept 201. The testing kit according to Inventive Concept 196, wherein the biological particulate is selected from the group consisting of: a virus, a bacterium, a microorganism, a fungus, a spore, a mite, a biological cell, a biological antigen, a protein, a protein antigen, and a carbohydrate antigen.

Inventive Concept 202. The sampling device according to any one of Inventive Concepts 173-195, wherein the filter comprises a polyethersulfone (PES) membrane filter.

Inventive Concept 203. A method for concentrating a liquid specimen sample, the method comprising:
placing the liquid specimen sample in a tubular container of a filtration assembly of a sampling device;
inserting a plunger head of a plunger of the filtration assembly into the tubular container via a proximal container opening of the tubular container, such that a lateral surface of the plunger head forms a fluid-tight movable seal with an inner wall of the tubular container;
distally advancing the plunger head within the tubular container to drive at least a portion of the liquid specimen sample through a filter removably disposed in the tubular container on a support surface of a filter support, wherein the filter support is shaped so as to define a plurality of filtrate-passage openings through the filter support; and
removing the filter from the filter support and from the filtration assembly while the plunger head remains within the tubular container and the filter support remains within the filtration assembly.

Inventive Concept 204. The method according to Inventive Concept 203,
wherein the filter support is disposed within the tubular container, and
wherein removing the filter from the filter support and from the filtration assembly comprises removing the filter from the filter support and from the filtration assembly while the plunger head remains within the tubular container and the filter support remains within the tubular container.

Inventive Concept 205. The method according to Inventive Concept 203, wherein removing the filter from the filter support and from the filtration assembly comprises removing a filter-withdrawal shaft from being partially inserted in the filtration assembly, while a distal portion of the filter-withdrawal shaft is coupled to the filter, while the plunger head remains within the tubular container, and while the filter support remains within the filtration assembly.

Inventive Concept 206. The method according to Inventive Concept 205,
wherein the filter support is shaped so as to define a central opening, in addition to the plurality of filtrate-passage openings, and
wherein removing the filter from the filter support and from the filtration assembly comprises pulling the filter through the central opening by removing the filter-withdrawal shaft from being partially inserted in the filtration assembly, while the distal portion of the filter-withdrawal shaft is coupled to the filter, while the plunger head remains within the tubular container, and while the filter support remains within the filtration assembly.

Inventive Concept 207. The method according to any one of Inventive Concepts 203-206, further comprising, after the filter has been removed from the tubular container, detecting the presence of a biological particulate trapped by the filter.

Inventive Concept 208. The method according to Inventive Concept 207, wherein detecting the presence of the biological particulate trapped by the filter comprising using a lateral flow test strip to detect the presence of the biological particulate trapped by the filter.

Inventive Concept 209. The method according to Inventive Concept 207, wherein the biological particulate is selected from the group consisting of: a virus, a bacterium, a microorganism, a fungus, a spore, a mite, a biological cell, a biological antigen, a protein, a protein antigen, and a carbohydrate antigen.

Inventive Concept 210. The method according to any one of Inventive Concepts 203-206, wherein the liquid specimen sample includes gargled fluid.

Inventive Concept 211. The method according to any one of Inventive Concepts 203-206, wherein the filter comprises a polyethersulfone (PES) membrane filter.

Inventive Concept 212. A method comprising:
passing at least a portion of a liquid specimen sample through a filter;
inserting at least a portion of the filter into an extraction tube and bathing the at least a portion of the filter in the extraction tube in a liquid comprising one or more reagents;
thereafter, discarding some of the liquid; and
thereafter, squeezing the filter to squeeze some of the liquid from the filter.

Inventive Concept 213. The method according to Inventive Concept 212, wherein discarding some of the liquid comprises removing the filter from the extraction tube while some of the liquid remain in the extraction tube.

Inventive Concept 214. The method according to Inventive Concept 212, wherein discarding some of the liquid comprises draining some of the liquid from the extraction tube while the at least a portion of the filter remains in the tube.

Inventive Concept 215. The method according to Inventive Concept 212, further comprising, after bathing the at least a portion of the filter in the liquid and before discarding some of the liquid:

squeezing the filter to squeeze some of the liquid from the filter while the at least a portion of the filter is within the extraction tube.

Inventive Concept 216. The method according to any one of Inventive Concepts 212-215, further comprising, after squeezing the filter, testing for the presence of a biological particulate trapped by the filter.

Inventive Concept 217. The method according to Inventive Concept 216, wherein the biological particulate is selected from the group consisting of: a virus, a bacterium, a microorganism, a fungus, a spore, a mite, a biological cell, a biological antigen, a protein, a protein antigen, and a carbohydrate antigen.

Inventive Concept 218. The method according to Inventive Concept 216, wherein testing for the presence of the biological particular trapped by the filter comprises testing a portion of the liquid for the presence of a target analyte released into the liquid from the biological particulate trapped by the filter.

Inventive Concept 219. The method according to Inventive Concept 216, wherein testing for the presence of a biological particulate trapped by the filter comprises bringing the filter into direct contact with a sample pad of a lateral flow test strip.

Inventive Concept 220. The method according to Inventive Concept 219, wherein the lateral flow test strip is contained at least partially within a housing.

Inventive Concept 221. The method according to Inventive Concept 220, wherein the housing is selected from the group consisting of: a cartridge and a card.

Inventive Concept 222. The method according to Inventive Concept 220, wherein the lateral flow test strip is disposed at least partially within the housing such that a test area of the lateral flow test strip is visible through one or more result windows defined by the housing.

Inventive Concept 223. The method according to Inventive Concept 220,
wherein the housing is shaped so as to define a channel in fluid communication with the sample pad, and
wherein bringing the filter into the direct contact with the sample pad comprises inserting the filter into the channel and into the direct contact with the sample pad of the lateral flow test strip.

Inventive Concept 224. The method according to any one of Inventive Concepts 212-215, further comprising bunching up at least a portion of the filter after passing the at least a portion of the liquid specimen sample through the filter and before inserting the at least a portion of the filter into the extraction tube.

Inventive Concept 225. The method according to Inventive Concept 224, wherein bunching up the at least a portion of the filter comprises bunching up the at least a portion of the filter into a flower-like arrangement.

Inventive Concept 226. The method according to Inventive Concept 224, wherein passing the at least a portion of the liquid specimen sample through the filter comprises passing the at least a portion of the liquid specimen sample through the filter while the filter is in a flat shape.

Inventive Concept 227. The method according to Inventive Concept 224, wherein squeezing the filter comprises squeezing the bunched-up portion of the filter by longitudinally compacting the bunched-up portion of the filter against a distal blocking surface intercepted by a straight central longitudinal axis of the extraction tube.

Inventive Concept 228. The method according to Inventive Concept 224, wherein squeezing the filter comprises:
inserting the bunched-up portion of the filter into a second tube separate and distinct from the extraction tube; and
squeezing the bunched-up portion of the filter to squeeze some of the liquid from the filter while the at least a portion of the filter is within the second tube.

Inventive Concept 229. The method according to Inventive Concept 228, wherein squeezing the bunched-up portion of the filter by longitudinally compacting the bunched-up portion of the filter against a distal blocking surface intercepted by a straight central longitudinal axis of the second tube.

Inventive Concept 230. A method comprising:
collecting a liquid specimen sample potentially containing biological particulate; and
testing for the presence of the biological particulate in the liquid specimen sample, by:
   passing at least a portion of the liquid specimen sample through a filter;
   applying one or more reagents to the filter; and
   thereafter, bringing the filter into direct contact with a sample pad of a lateral flow test strip.

Inventive Concept 231. The method according to Inventive Concept 230, wherein the biological particulate is selected from the group consisting of: a virus, a bacterium, a microorganism, a fungus, a spore, a mite, a biological cell, a biological antigen, a protein, a protein antigen, and a carbohydrate antigen.

Inventive Concept 232. The method according to Inventive Concept 230, further comprising, after applying the one or more reagents to the filter and before bringing the filter into the direct contact with the sample pad, discarding a portion of the one or more reagents.

Inventive Concept 233. The method according to Inventive Concept 230, wherein bringing the filter into the direct contact with the sample pad comprises pressing at least a portion of the filter against the sample pad.

Inventive Concept 234. The method according to Inventive Concept 230, wherein bringing the filter into the direct contact with the sample pad comprises squeezing the filter against the sample pad of the lateral flow test strip.

Inventive Concept 235. The method according to Inventive Concept 234, wherein squeezing the filter against the sample pad comprises squeezing the filter by longitudinally compacting the bunched-up portion of the filter against the sample pad.

Inventive Concept 236. The method according to Inventive Concept 230, wherein bringing the filter into the direct contact with the sample pad comprises bringing the filter into the direct contact with the sample pad using a filter shaft that includes a distal portion that is coupled to the filter.

Inventive Concept 237. The method according to Inventive Concept 236, wherein the distal portion of the filter shaft is coupled to a central portion of the filter.

Inventive Concept 238. The method according to Inventive Concept 236, wherein bringing the filter into the direct contact with the sample pad comprises orienting the filter shaft so that a straight central longitudinal axis of the filter shaft forms an angle of 30-90 degrees with the sample pad.

Inventive Concept 239. The method according to Inventive Concept 238, wherein orienting the filter shaft comprises orienting the filter shaft such that the angle is 45-90 degrees.

Inventive Concept 240. The method according to any one of Inventive Concepts 230-239, further comprising bunching up at least a portion of the filter after passing the at least a portion of the liquid specimen sample through the filter and before bringing the filter into the direct contact with the sample pad.

Inventive Concept 241. The method according to Inventive Concept 240, wherein bunching up the at least a portion of the filter comprises bunching up the at least a portion of the filter into a flower-like arrangement.

Inventive Concept 242. The method according to Inventive Concept 240, wherein bringing the filter into the direct contact with the sample pad comprises squeezing the bunched-up portion of the filter against the sample pad of the lateral flow test strip. Inventive Concept 243. The method according to Inventive Concept 242, wherein squeezing the bunched-up portion of the filter against the sample pad comprises squeezing the bunched-up portion of the filter by longitudinally compacting the bunched-up portion of the filter against the sample pad.

Inventive Concept 244. The method according to Inventive Concept 242, wherein squeezing the bunched-up portion of the filter against the sample pad delivers to the sample pad both (a) some of the one or more reagents contained in or on the filter at locations of the filter that directly contact the sample pad, and (b) some of the one or more reagents contained in or on the filter at locations other than the locations of the filter that directly contact the sample pad.

Inventive Concept 245. The method according to Inventive Concept 240, wherein passing the at least a portion of the liquid specimen sample through the filter comprises passing the at least a portion of the liquid specimen sample through the filter while the filter is in a flat shape.

Inventive Concept 246. The method according to any one of Inventive Concepts 230-239, wherein the lateral flow test strip is contained at least partially within a housing.

Inventive Concept 247. The method according to Inventive Concept 246, wherein the housing is selected from the group consisting of: a cartridge and a card.

Inventive Concept 248. The method according to Inventive Concept 246, wherein the lateral flow test strip is disposed at least partially within the housing such that a test area of the lateral flow test strip is visible through one or more result windows defined by the housing.

Inventive Concept 249. The method according to Inventive Concept 246,
wherein the housing is shaped so as to define a channel in fluid communication with the sample pad, and
wherein bringing the filter into the direct contact with the sample pad comprises inserting the filter into the channel and into the direct contact with the sample pad of the lateral flow test strip.

Inventive Concept 250. The method according to Inventive Concept 249,
wherein the method further comprises bunching up at least a portion of the filter after passing the at least a portion of the liquid specimen sample through the filter and before bringing the filter into the direct contact with the sample pad, and
wherein bringing the filter into the direct contact with the sample pad comprises inserting the bunched-up portion of the filter into the channel and advancing a distal portion of the bunched-up portion of the filter through the channel and into the direct contact with the sample pad of the lateral flow test strip.

Inventive Concept 251. The method according to Inventive Concept 250, wherein bringing the filter into the direct contact with the sample pad comprises squeezing the bunched-up portion of the filter by longitudinally compacting the bunched-up portion of the filter against the sample pad of the lateral flow test strip.

Inventive Concept 252. The method according to Inventive Concept 250,
wherein bunching up the at least a portion of the filter comprises bunching up the at least a portion of the filter such that an entirety of a perimeter of the filter points distally,
wherein inserting the bunched-up portion of the filter into the channel comprises inserting, in a distal direction, the bunched-up portion of the filter into the channel while the entirety of the perimeter of the filter points distally, and
wherein advancing the distal portion of the bunched-up portion of the filter comprises advancing the distal portion of the bunched-up portion of the filter through the channel so that at least a portion of the perimeter makes direct contact with the sample pad of the lateral flow test strip.

Inventive Concept 253. The method according to Inventive Concept 246,
wherein the sample pad is disposed at an upstream end portion of the lateral flow test strip, and the lateral flow test strip further includes (a) an absorbent pad disposed at a downstream end portion of the lateral flow test strip, and (b) a membrane disposed longitudinally between the sample pad and the absorbent pad, the membrane comprising a test area, which comprises a test line and a control line, and
wherein the housing is configured such that when the housing is placed on a flat surface, the test area of the membrane is farther from flat surface than the sample pad is from the flat surface.

Inventive Concept 254. The method according to Inventive Concept 253, wherein the housing is shaped so as to define a raised portion at least under the test area of the membrane.

Inventive Concept 255. The method according to Inventive Concept 253, wherein the lateral flow test strip is oriented obliquely with respect to the flat surface when the housing is placed on the flat surface, such that the downstream end portion of the lateral flow test strip is more elevated from the flat surface than is the upstream end portion of the lateral flow test strip.

Inventive Concept 256. The method according to Inventive Concept 255, wherein the lateral flow test strip is oriented at an angle of 1-20 degrees with respect to the flat surface when the housing is placed on the flat surface.

Inventive Concept 257. A sampling device for concentrating a liquid specimen sample, the sampling device comprising:
a container;
a filter;
a filter support, which is shaped so as to define (a) a support surface on which the filter is removably disposed, and (b) a plurality of filtrate-passage openings through the filter support; and
a filter reinforcement, which is coupled to a surface of the filter so as to cover 1%-50% of a surface area of the surface.

Inventive Concept 258. The sampling device according to Inventive Concept 257, wherein the filter reinforcement has a greater tensile strength than the filter.

Inventive Concept 259. The sampling device according to Inventive Concept 257, wherein the filter reinforcement is not porous.

Inventive Concept 260. The sampling device according to Inventive Concept 257, wherein the filter reinforcement comprises metal.

Inventive Concept 261. The sampling device according to Inventive Concept 257, wherein the filter reinforcement comprises a polymer.

Inventive Concept 262. The sampling device according to Inventive Concept 257, wherein the filter comprises a polyethersulfone (PES) membrane filter.

Inventive Concept 263. The sampling device according to Inventive Concept 257, wherein the filter reinforcement is coupled to the surface of the filter so as to cover 1%-30% of the surface area of the surface.

Inventive Concept 264. The sampling device according to Inventive Concept 263, wherein the filter reinforcement is coupled to the surface of the filter so as to cover 5%-25% of the surface area of the surface.

Inventive Concept 265. The sampling device according to Inventive Concept 257, wherein the filter reinforcement is shaped so as to define a plurality of thin strips.

Inventive Concept 266. The sampling device according to Inventive Concept 257, wherein the filter reinforcement is shaped so as to define a central hub.

Inventive Concept 267. The sampling device according to Inventive Concept 266, wherein the central hub is shaped so as to define a central opening therethrough.

Inventive Concept 268. The sampling device according to Inventive Concept 257, wherein the filter reinforcement is shaped so as to define a peripheral rim.

Inventive Concept 269. The sampling device according to Inventive Concept 257, wherein the filter reinforcement is shaped so as to define a plurality of spokes.

Inventive Concept 270. The sampling device according to Inventive Concept 269, wherein the filter reinforcement is shaped so as to define a peripheral rim connected to the spokes.

Inventive Concept 271. The sampling device according to Inventive Concept 269, wherein the filter reinforcement is shaped so as to define a central hub connected to the spokes.

Inventive Concept 272. The sampling device according to Inventive Concept 271, wherein the filter reinforcement is shaped so as to define a peripheral rim connected to the spokes.

Inventive Concept 273. The sampling device according to Inventive Concept 257, wherein the filter is circular when flat.

Inventive Concept 274. The sampling device according to any one of Inventive Concepts 257-273,
wherein the container is tubular, and is shaped so as to define an inner wall and a proximal container opening for receiving the liquid specimen sample,
wherein the sampling device further comprises a plunger, which (a) comprises a plunger head and (b) is insertable into the tubular container via the proximal container opening, such that a lateral surface of the plunger head forms a fluid-tight movable seal with the inner wall, and
wherein the sampling device is configured such that movement of the plunger head within the tubular container, when the liquid specimen sample is contained in the tubular container and the filter is disposed in the tubular container, pushes at least a portion of the liquid specimen sample through the filter in an upstream-to-downstream direction.

Inventive Concept 275. The sampling device according to Inventive Concept 274, wherein the surface to which the filter reinforcement is coupled is an upstream surface of the filter.

Inventive Concept 276. The sampling device according to any one of Inventive Concepts 257-273, wherein the filter reinforcement is an upstream filter reinforcement, and wherein the sampling device further comprises a downstream filter reinforcement, which is coupled to a downstream surface of the filter.

Inventive Concept 277. The sampling device according to Inventive Concept 276, wherein the downstream filter reinforcement is shaped so as to define a central hub.

Inventive Concept 278. The sampling device according to any one of Inventive Concepts 257-273,
wherein the sampling device further comprises a withdrawer comprising a filter-withdrawal shaft, which is partially inserted in the container, wherein the sampling device is configured such that removal of the filter-withdrawal shaft from the container, while a distal portion of the filter-withdrawal shaft is coupled to the filter and the filter support remains within the sampling device, removes the filter from the sampling device.

Inventive Concept 279. The sampling device according to Inventive Concept 278,
wherein the filter support is shaped so as to define a central opening, in addition to the plurality of filtrate-passage openings, and
wherein the sampling device is configured such that removal of the filter-withdrawal shaft from the sampling device, while the filter support remains within the sampling device, pulls the filter through the central opening and removes the filter from the sampling device.

Inventive Concept 280. The sampling device according to any one of Inventive Concepts 257-273, wherein the sampling device is configured such that removal of the filter-withdrawal shaft from the container, while the filter support remains within the sampling device, removes the filter from the sampling device and bunches up at least a portion of the filter.

Inventive Concept 281. The sampling device according to Inventive Concept 280, wherein the sampling device is configured such that the removal of the filter-withdrawal shaft from the container, while the filter support remains within the sampling device, removes the filter from the sampling device and bunches up the at least a portion of the filter into a flower-like arrangement.

Inventive Concept 282. The sampling device according to any one of Inventive Concepts 257-273, further comprising a filter shaft having a distal portion coupled to the filter.

Inventive Concept 283. The sampling device according to Inventive Concept 282, wherein the distal portion of the filter shaft is indirectly coupled to the filter.

Inventive Concept 284. The sampling device according to Inventive Concept 282, wherein the distal portion of the filter shaft is directly coupled to the filter.

Inventive Concept 285. The sampling device according to Inventive Concept 284, further comprising a distal plate that is fixed to a distal end of the distal portion of the filter shaft such that a central portion of the filter is between the distal end and the distal plate, so as to directly couple the filter to the filter shaft.

Inventive Concept 286. A testing kit comprising the sampling device according to any one of Inventive Concepts 257-273, the testing kit further comprising a diagnostic test configured to detect the presence of a biological particulate trapped by the filter.

Inventive Concept 287. The testing kit according to Inventive Concept 286, wherein the diagnostic test comprises a lateral flow test strip.

Inventive Concept 288. The testing kit according to Inventive Concept 287, further comprising reagents for use with the lateral flow test strip.

Inventive Concept 289. The testing kit according to Inventive Concept 287, further comprising one or more reagents for use with the lateral flow test strip.

Inventive Concept 290. The testing kit according to Inventive Concept 286, wherein the diagnostic test comprises a rapid molecular test.

Inventive Concept 291. The testing kit according to Inventive Concept 286, wherein the biological particulate is selected from the group consisting of: a virus, a bacterium, a microorganism, a fungus, a spore, a mite, a biological cell, a biological antigen, a protein, a protein antigen, and a carbohydrate antigen.

Inventive Concept 292. A diagnostic test for use with a flat surface, the diagnostic test comprising:
a housing selected from the group consisting of: a cartridge and a card; and
a lateral flow test strip, which:
   comprises (a) a sample pad disposed at an upstream end portion of the lateral flow test strip, (b) an absorbent pad disposed at a downstream end portion of the lateral flow test strip, and (c) a membrane disposed longitudinally between the sample pad and the absorbent pad, the membrane comprising a test area, which comprises a test line and a control line, and
   is contained at least partially within the housing,
wherein the housing is configured such that when the housing is placed on the flat surface, the test area of the membrane is farther from flat surface than the sample pad is from the flat surface.

Inventive Concept 293. The diagnostic test according to Inventive Concept 292, wherein the housing is shaped so as to define a raised portion at least under the test area of the membrane.

Inventive Concept 294. The diagnostic test according to Inventive Concept 292, wherein the lateral flow test strip is oriented obliquely with respect to the flat surface when the housing is placed on the flat surface, such that the downstream end portion of the lateral flow test strip is more elevated from the flat surface than is the upstream end portion of the lateral flow test strip.

Inventive Concept 295. The diagnostic test according to Inventive Concept 294, wherein the lateral flow test strip is oriented at an angle of 1-20 degrees with respect to the flat surface when the housing is placed on the flat surface.

Inventive Concept 296. The diagnostic test according to Inventive Concept 292, wherein the housing is shaped so as to define a channel in fluid communication with the sample pad.

Inventive Concept 297. The diagnostic test according to Inventive Concept 296, wherein the channel has an internal length of 1-5 cm.

Inventive Concept 298. The diagnostic test according to Inventive Concept 292, wherein the housing is shaped so as to define one or more result windows, and wherein the lateral flow test strip is disposed at least partially within the housing such that a test area of the lateral flow test strip is visible through the one or more result windows defined by the housing.

Inventive Concept 299. A sampling device for concentrating a liquid specimen sample, the sampling device comprising a filtration assembly, which comprises:
a tubular container, which is shaped so as to define an inner wall and a proximal container opening for receiving the liquid specimen sample;
a plunger, which (i) comprises a plunger head and (ii) is insertable into the tubular container via the proximal container opening, such that a lateral surface of the plunger head forms a fluid-tight movable seal with the inner wall;
a filter;
a filter support, which is shaped so as to define (A) a support surface on which the filter is removably disposed, and (B) a plurality of filtrate-passage openings through the filter support; and
a clamping surface,
wherein sampling device is transitionable from:
   a filter-clamping state, in which the filter is removably disposed on the support surface, clamped axially between the clamping surface and a peripheral portion of the support surface, to
   a filter-release state, in which the filter is not clamped axially between the clamping surface and the peripheral portion of the support surface,
wherein the filtration assembly is configured such that movement of the plunger head within the tubular container, when the liquid specimen sample is contained in the tubular container and the sampling device is in the filter-clamping state, pushes at least a portion of the liquid specimen sample through the filter and the filtrate-passage openings, and
wherein the sampling device is configured such that the filter is removable from the tubular container while the plunger head and the filter support remain within the tubular container and the sampling device is in the filter-release state.

Inventive Concept 300. The sampling device according to Inventive Concept 299, wherein the filtration assembly is configured such that:
a first portion of a stroke of the movement of the plunger head within the tubular container, when the liquid specimen sample is contained in the tubular container and the sampling device is in the filter-clamping state, pushes the at least a portion of the liquid specimen sample through the filter and the filtrate-passage openings, and
a second portion of the stroke of the movement of the plunger head within the tubular container transitions the sampling device from the filter-clamping state to the filter-release state.

Inventive Concept 301. The sampling device according to Inventive Concept 300,
wherein the plunger head comprises one or more tabs, which restrain the clamping surface such that the filter is clamped axially between the clamping surface and the peripheral portion of the support surface, and
wherein the filtration assembly is configured such that the second portion of the stroke of the movement of the plunger head within the tubular container displaces the one or more tabs such that the filter is not clamped axially between the clamping surface and the peripheral portion of the support surface, thereby transitioning the sampling device from the filter-clamping state to the filter-release state.

Inventive Concept 302. The sampling device according to Inventive Concept 299,
wherein the filtration assembly further comprises a waste liquid receptacle,
wherein the filter support is shaped so as to define the plurality of filtrate-passage openings through the filter support into the waste liquid receptacle, and
wherein the filtration assembly is configured such that the movement of the plunger head within the tubular container, when the liquid specimen sample is contained in the tubular container and the sampling device is in the filter-clamping state, pushes the at least a portion of the liquid specimen sample through the filter and the filtrate-passage openings and into the waste liquid receptacle.

Inventive Concept 303. The sampling device according to Inventive Concept 302, wherein the sampling device is configured to move the filter support within the waste liquid receptacle, so as to transition the sampling device from the filter-clamping state to the filter-release state.

Inventive Concept 304. The sampling device according to Inventive Concept 303, wherein the sampling device is configured to proximally move the filter support within the waste liquid receptacle, so as to transition the sampling device from the filter-clamping state to the filter-release state.

Inventive Concept 305. The sampling device according to Inventive Concept 302,
wherein the plunger further comprises a plunger rod, which (a) has a distal end portion to which the plunger head is coupled, and (b) is shaped so as to define the waste liquid receptacle within the plunger rod, and
wherein the filter support and the clamping surface are coupled to the plunger head.

Inventive Concept 306. The sampling device according to Inventive Concept 305, wherein the plunger head comprises an internal shelf, which projects radially inwardly and defines the clamping surface.

Inventive Concept 307. The sampling device according to Inventive Concept 306, wherein the shelf is annular.

Inventive Concept 308. The sampling device according to Inventive Concept 306, wherein the clamping surface runs intermittently around the peripheral portion of the support surface.

Inventive Concept 309. The sampling device according to Inventive Concept 305,
wherein the plunger rod is shaped so as to define an internal plunger space having a plunger-space proximal opening through a proximal end of the plunger rod, and
wherein the sampling device is configured such that the filter is removable from the tubular container via the plunger-space proximal opening while the plunger head and the filter support remain within the tubular container.

Inventive Concept 310. The sampling device according to Inventive Concept 309,
wherein the filter support is shaped so as to define a plunger-head opening through the plunger head and into the internal plunger space,
wherein the sampling device comprises a withdrawer comprising a filter-withdrawal shaft, which is disposed passing through the internal plunger space, and
wherein the sampling device is configured such that proximal withdrawal of the filter-withdrawal shaft out of the internal plunger space, while a distal portion of the filter-withdrawal shaft is coupled to the filter and the plunger head remains within the tubular container, pulls the filter into the internal plunger space via the plunger-head opening and out of the internal plunger space via the plunger-space proximal opening, and removes the filter-withdrawal shaft from the filtration assembly and the filter from the filter support and from the filtration assembly.

Inventive Concept 311. The sampling device according to Inventive Concept 310, wherein the sampling device is configured to proximally move the filter support within the waste liquid receptacle, so as to transition the sampling device from the filter-clamping state to the filter-release state.

Inventive Concept 312. The sampling device according to Inventive Concept 311,
wherein the plunger head comprises a hollow shaft that is coupled to the filter support and shaped so as to define the internal plunger space within the hollow shaft, and
wherein the sampling device is configured such that proximal movement of the hollow shaft proximally moves the filter support within the waste liquid receptacle.

Inventive Concept 313. The sampling device according to Inventive Concept 311, wherein the sampling device is configured such that proximal movement of the filter support within the waste liquid receptacle transitions the sampling device from the filter-clamping state to the filter-release state.

Inventive Concept 314. The sampling device according to Inventive Concept 313,
wherein the filtration assembly and the withdrawer are shaped so as to define corresponding screw threads, and
wherein the sampling device is configured such that rotation of the withdrawer and the plunger-space proximal opening with respect to each other:
   causes at least an initial portion of the proximal withdrawal of the filter-withdrawal shaft out of the internal plunger space, and
   proximally moves the filter support within the waste liquid receptacle, so as to transition the sampling device from the filter-clamping state to the filter-release state.

Inventive Concept 315. The sampling device according to Inventive Concept 299,
wherein a distal bottom surface of the tubular container is shaped so as to define a distal opening, and
wherein the sampling device is configured such that the filter is removable from the tubular container via the distal opening while the plunger head and the filter support remain within the tubular container.

Inventive Concept 316. The sampling device according to Inventive Concept 315,
wherein the sampling device comprises a withdrawer comprising a filter-withdrawal shaft, which is disposed passing through the distal opening defined by the distal bottom surface of the tubular container, and
wherein the sampling device is configured such that removal of the filter-withdrawal shaft from the filtration assembly, while a distal portion of the filter-withdrawal shaft is coupled to the filter, while the plunger head remains within the tubular container, and while the filter support remains within the filtration assembly, removes the filter from the filtration assembly via the distal opening defined by the bottom surface of the tubular container.

Inventive Concept 317. The sampling device according to Inventive Concept 316,
wherein the tubular container comprises a hollow shaft that is shaped so as to define an internal shaft space within the hollow shaft,
wherein the filter-withdrawal shaft is disposed passing through (a) the distal opening defined by the distal bottom surface of the tubular container and (b) the internal shaft space, and
wherein the sampling device is configured such that the removal of the filter-withdrawal shaft from the filtration assembly, while the distal portion of the filter-withdrawal shaft is coupled to the filter, while the plunger head remains within the tubular container, and while the filter support remains within the filtration assembly, removes the filter from the filtration assembly via (a) the distal opening defined by the bottom surface of the tubular container and (b) the internal shaft space.

Inventive Concept 318. A testing kit comprising the sampling device according to any one of Inventive Concepts 299-317, the testing kit further comprising a diagnostic test configured to detect the presence of a biological particulate trapped by the filter.

Inventive Concept 319. The testing kit according to Inventive Concept 318, wherein the diagnostic test comprises a lateral flow test strip.

Inventive Concept 320. The testing kit according to Inventive Concept 319, further comprising reagents for use with the lateral flow test strip.

Inventive Concept 321. The testing kit according to Inventive Concept 319, further comprising one or more reagents for use with the lateral flow test strip.

Inventive Concept 322. The testing kit according to Inventive Concept 318, wherein the diagnostic test comprises a rapid molecular test.

Inventive Concept 323. The testing kit according to Inventive Concept 318, wherein the biological particulate is selected from the group consisting of: a virus, a bacterium, a microorganism, a fungus, a spore, a mite, a biological cell, a biological antigen, a protein, a protein antigen, and a carbohydrate antigen.

Inventive Concept 324. A method for concentrating a liquid specimen sample, the method comprising:
placing the liquid specimen sample in a tubular container of a filtration assembly of a sampling device;
inserting a plunger head of a plunger of the filtration assembly into the tubular container via a proximal container opening of the tubular container, such that a lateral surface of the plunger head forms a fluid-tight movable seal with an inner wall of the tubular container;
distally advancing the plunger head within the tubular container to drive at least a portion of the liquid specimen sample through a filter, while the sampling device is in a filter-clamping state, in which the filter is removably disposed on a support surface of a filter support, clamped axially between a clamping surface of the filtration assembly and a peripheral portion of the support surface, wherein the filter support is shaped so as to define a plurality of filtrate-passage openings through the filter support; and
removing the filter from the tubular container via the plunger-space proximal opening while the plunger head and the filter support remain within the tubular container and the sampling device is in a filter-release state, in which the filter is not clamped axially between the clamping surface and the peripheral portion of the support surface.

Inventive Concept 325. The method according to Inventive Concept 324, further comprising, after the filter has been removed from the tubular container, detecting the presence of a biological particulate trapped by the filter.

Inventive Concept 326. The method according to Inventive Concept 325, wherein detecting the presence of the biological particulate trapped by the filter comprising using a lateral flow test strip to detect the presence of the biological particulate trapped by the filter.

Inventive Concept 327. The method according to Inventive Concept 325, wherein the biological particulate is selected from the group consisting of: a virus, a bacterium, a microorganism, a fungus, a spore, a mite, a biological cell, a biological antigen, a protein, a protein antigen, and a carbohydrate antigen.

Inventive Concept 328. The method according to any one of Inventive Concepts 324-327, wherein the liquid specimen sample includes gargled fluid.

Inventive Concept 329. A sampling device for concentrating a liquid specimen sample, the sampling device comprising a filtration assembly, which comprises:
a container housing, which is shaped so as to define (a) a cylindrical space within the container housing, and (b) one or more first threads;
a tubular container, which (a) is shaped so as to define an inner wall and a proximal container opening for receiving the liquid specimen sample, and (b) is disposed at least partially within the cylindrical space of the container housing, such that the tubular container is rotatable with respect to the cylindrical space;
a plunger support, which is shaped so as to define one or more second threads, shaped so as to engage the one or more first threads;
a plunger, which (a) comprises a plunger head, (b) is insertable into the tubular container via the proximal container opening, such that a lateral surface of the plunger head forms a fluid-tight movable seal with the inner wall, and (c) is coupled to the plunger support, such that rotation of the plunger support with respect to the container housing, when the one or more second threads are engaged with the one or more first threads, distally advances the plunger support with respect to the container housing and thus the plunger within the tubular container as the tubular container rotates with respect to the container housing; and
a filter,
wherein the filtration assembly is configured such that movement of the plunger head within the tubular container, when the liquid specimen sample is contained in the tubular container and the filter is disposed in the tubular container, pushes at least a portion of the liquid specimen sample through the filter.

Inventive Concept 330. The sampling device according to Inventive Concept 329, wherein the one or more first threads face radially outward, and the one or more second threads face radially inward.

Inventive Concept 331. The sampling device according to Inventive Concept 329, wherein the one or more first threads face radially inward, and the one or more second threads face radially outward.

Inventive Concept 332. The sampling device according to Inventive Concept 329, wherein the proximal container opening is shaped as a funnel.

Inventive Concept 333. The sampling device according to any one of Inventive Concepts 329-332, wherein the container housing includes a proximal portion that is proximal to the cylindrical space, has a greatest internal diameter that is greater than an internal diameter of the cylindrical space, and is shaped so as to define a funnel-shaped portion.

Inventive Concept 334. The sampling device according to Inventive Concept 333, wherein a distal end of the funnel-shaped portion is within 2 cm of the cylindrical space, measured along a central longitudinal axis of the cylindrical space.

Inventive Concept 335. The sampling device according to Inventive Concept 333, wherein the proximal container opening is shaped as a funnel.

Inventive Concept 336. The sampling device according to any one of Inventive Concepts 329-332, further comprising a rotary bearing to facilitate rotation of the tubular container within the container housing.

Inventive Concept 337. The sampling device according to Inventive Concept 336, wherein the rotary bearing comprises a radial ball bearing.

Inventive Concept 338. A testing kit comprising the sampling device according to any one of Inventive Concepts 329-332, the testing kit further comprising a diagnostic test configured to detect the presence of a biological particulate trapped by the filter.

Inventive Concept 339. The testing kit according to Inventive Concept 338, wherein the diagnostic test comprises a lateral flow test strip.

Inventive Concept 340. The testing kit according to Inventive Concept 339, further comprising reagents for use with the lateral flow test strip.

Inventive Concept 341. The testing kit according to Inventive Concept 339, further comprising one or more reagents for use with the lateral flow test strip.

Inventive Concept 342. The testing kit according to Inventive Concept 338, wherein the diagnostic test comprises a rapid molecular test.

Inventive Concept 343. The testing kit according to Inventive Concept 338, wherein the biological particulate is selected from the group consisting of: a virus, a bacterium, a microorganism, a fungus, a spore, a mite, a biological cell, a biological antigen, a protein, a protein antigen, and a carbohydrate antigen.

Inventive Concept 344. A method for concentrating a liquid specimen sample, the method comprising:
placing the liquid specimen sample in a tubular container of a filtration assembly of a sampling device, wherein the tubular container is shaped so as to define an inner wall and is disposed at least partially within a cylindrical space within a container housing of the filtration assembly, such that the tubular container is rotatable with respect to the cylindrical space;
inserting a plunger head of a plunger of the filtration assembly into the tubular container via a proximal container opening of the tubular container, such that a lateral surface of the plunger head forms a fluid-tight movable seal with an inner wall of the tubular container, wherein the plunger is coupled to a plunger support of the filtration assembly; and
rotating the plunger support with respect to the container housing while one or more second threads defined by the plunger support are engaged with one or more first threads defined by the container housing, so as to distally advance the plunger support with respect to the container housing and thus the plunger within the tubular container as the tubular container rotates with respect to the container housing, thereby pushing at least a portion of the liquid specimen sample through a filter removably disposed in the tubular container.

Inventive Concept 345. The method according to Inventive Concept 344, further comprising, after the filter has been removed from the tubular container, detecting the presence of a biological particulate trapped by the filter.

Inventive Concept 346. The method according to Inventive Concept 345, wherein detecting the presence of the biological particulate trapped by the filter comprising using a lateral flow test strip to detect the presence of the biological particulate trapped by the filter.

Inventive Concept 347. The method according to Inventive Concept 345, wherein the biological particulate is selected from the group consisting of: a virus, a bacterium, a microorganism, a fungus, a spore, a mite, a biological cell, a biological antigen, a protein, a protein antigen, and a carbohydrate antigen.

Inventive Concept 348. The method according to any one of Inventive Concepts 344-347, wherein the liquid specimen sample includes gargled fluid.

Inventive Concept 349. A sampling device for concentrating a liquid specimen sample, the sampling device comprising a filtration assembly, which comprises:
a tubular container, which is shaped so as to define an inner wall and a proximal container opening for receiving the liquid specimen sample;
a plunger, which (i) comprises a plunger head and a plunger rod, which (a) has a distal end portion to which the plunger head is coupled, and (b) is shaped so as to define a waste liquid receptacle within the plunger rod, and (ii) is insertable into the tubular container via the proximal container opening, such that a lateral surface of the plunger head forms a fluid-tight movable seal with the inner wall;
a filter; and
a mechanical energy storage element,
wherein the plunger head comprises filter support, which is shaped so as to define (a) a support surface on which the filter is removably disposed, and (b) a plurality of filtrate-passage openings through the filter support into the waste liquid receptacle, and
wherein the filtration assembly is configured such that movement of the plunger head within the tubular container, when the liquid specimen sample is contained in the tubular container and the filter is disposed on the support surface:
   pushes at least a portion of the liquid specimen sample through the filter and the filtrate-passage openings and into the waste liquid receptacle, and
   stores energy in the energy storage element.

Inventive Concept 350. The sampling device according to Inventive Concept 349,
wherein the sampling device further comprises a container housing,
wherein the tubular container is disposed at least partially within the container housing, such that the tubular container is moveable with respect to the container housing, and
wherein the energy storage element is disposed within the container housing, outside the tubular container and in direct or indirect contact with an external surface of the tubular container, such that movement of the plunger head within the tubular container moves the tubular container with respect to the container housing, thereby storing energy in the energy storage element.

Inventive Concept 351. The sampling device according to Inventive Concept 350,
wherein the energy storage element comprises a mechanical energy storage element, and
wherein the filtration assembly is configured such that the movement of the plunger head within the tubular container, when the liquid specimen sample is contained in the tubular container and the filter is disposed on the support surface:
   pushes the at least a portion of the liquid specimen sample through the filter and the filtrate-passage openings and into the waste liquid receptacle, and
   stores mechanical energy in the mechanical energy storage element.

Inventive Concept 352. The sampling device according to Inventive Concept 351, wherein the mechanical energy storage element comprises an elastic element configured to store the mechanical energy.

Inventive Concept 353. The sampling device according to Inventive Concept 352, wherein the elastic element comprises a spring.

Inventive Concept 354. The sampling device according to any one of Inventive Concepts 349-353, wherein the energy storage element is disposed within the tubular container.

Inventive Concept 355. The sampling device according to Inventive Concept 354, wherein the energy storage element comprises a flexible container containing a gas.

Inventive Concept 356. A method for concentrating a liquid specimen sample, the method comprising:
placing the liquid specimen sample in a tubular container of a filtration assembly of a sampling device;
inserting a plunger head of a plunger of the filtration assembly into the tubular container via a proximal container opening of the tubular container, such that a lateral surface of the plunger head forms a fluid-tight movable seal with an inner wall of the tubular container, wherein the plunger further comprises a plunger rod, which (a) has a distal end portion to which the plunger head is coupled, and (b) is shaped so as to define a waste liquid receptacle within the plunger rod; and
distally advancing the plunger head within the tubular container so as to:
   drive at least a portion of the liquid specimen sample through a filter removably disposed in the tubular container on a support surface of a filter support, wherein the filter support is shaped so as to define a plurality of filtrate-passage openings through the filter support into the waste liquid receptacle, and
   stores energy in an energy storage element of the filtration assembly.

Inventive Concept 357. The method according to Inventive Concept 356, further comprising, after the filter has been removed from the tubular container, detecting the presence of a biological particulate trapped by the filter.

Inventive Concept 358. The method according to Inventive Concept 357, wherein detecting the presence of the biological particulate trapped by the filter comprising using a lateral flow test strip to detect the presence of the biological particulate trapped by the filter.

Inventive Concept 359. The method according to Inventive Concept 357, wherein the biological particulate is selected from the group consisting of: a virus, a bacterium, a microorganism, a fungus, a spore, a mite, a biological cell, a biological antigen, a protein, a protein antigen, and a carbohydrate antigen.

Inventive Concept 360. The method according to any one of Inventive Concepts 356-359, wherein the liquid specimen sample includes gargled fluid.

The present invention will be more fully understood from the following detailed description of embodiments thereof, taken together with the drawings, in which:

### BRIEF DESCRIPTION OF THE DRAWINGS

FIGS. 1A-B are schematic illustrations of a sampling device for concentrating a liquid specimen sample, and a portion of the sampling device, respectively, in accordance with an application of the present invention;

FIGS. 2A-D are schematic illustrations of the sampling device of FIGS. 1A-B and a method of using the sampling device, in accordance with respective applications of the present invention;

FIGS. 3A-I are schematic cross-sectional illustrations of the sampling device of FIGS. 1A-B and the method of using the sampling device, in accordance with respective applications of the present invention;

FIGS. 3J-K are schematic illustrations of another method of using the sampling device of FIGS. 1A-B, in accordance with an application of the present invention;

FIG. 4 is an enlarged schematic illustration of a portion of the sampling device of FIGS. 1A-B, in accordance with an application of the present invention;

FIGS. 5A-B are schematic illustrations of another sampling device for concentrating a liquid specimen sample, and a portion of the sampling device, respectively, in accordance with an application of the present invention;

FIGS. 6A-E are schematic illustrations of the sampling device of FIGS. 5A-B and a method of using the sampling device, in accordance with respective applications of the present invention;

FIGS. 7A-E are schematic cross-sectional illustrations of the sampling device of FIGS. 5A-B and the method of using the sampling device, in accordance with respective applications of the present invention;

FIGS. 8A-E are schematic illustrations of yet another sampling device and a method of using the sampling device, in accordance with respective applications of the present invention;

FIGS. 9A-D are schematic illustrations of a portion of another sampling device and a method of using the sampling device, in accordance with respective applications of the present invention;

FIG. 9E is a schematic illustration of an alternative configuration of an extraction tube of the sampling device of FIGS. 9A-D, in accordance with an application of the present invention;

FIGS. 10A-B are schematic illustrations of a distal portion of a plunger head, in accordance with an application of the present invention;

FIGS. 11A-B are schematic illustrations of still another sampling device for concentrating a liquid specimen sample, and a portion of the sampling device, respectively, in accordance with an application of the present invention;

FIGS. 12A-F are schematic illustrations of the sampling device of FIGS. 11A-B and a method of using the sampling device, in accordance with respective applications of the present invention;

FIGS. 13A-F are schematic cross-sectional illustrations of the sampling device of FIGS. 11A-B and the method of using the sampling device, in accordance with respective applications of the present invention;

FIGS. 14A-B are schematic illustrations of another sampling device for concentrating a liquid specimen sample, and a portion of the sampling device, respectively, in accordance with an application of the present invention;

FIGS. 15A-E are schematic illustrations of the sampling device of FIGS. 14A-B and a method of using the sampling device, in accordance with respective applications of the present invention;

FIGS. 16A-E are schematic cross-sectional illustrations of the sampling device FIGS. 14A-B and the method of using the sampling device, in accordance with respective applications of the present invention;

FIGS. 17A-B are schematic illustrations of yet another sampling device for concentrating a liquid specimen sample, and a portion of the sampling device, respectively, in accordance with an application of the present invention;

FIGS. 18A-E are schematic illustrations of the sampling device of FIGS. 17A-B and a method of using the sampling device, in accordance with respective applications of the present invention;

FIGS. 19A-E are schematic cross-sectional illustrations of the sampling device of FIGS. 17A-B and the method of using the sampling device, in accordance with respective applications of the present invention;

FIGS. 20A-B are schematic illustrations of still another sampling device for concentrating a liquid specimen sample, and a portion of the sampling device, respectively, in accordance with an application of the present invention;

FIGS. 21A-E are schematic illustrations of the sampling device of FIGS. 20A-B and a method of using the sampling device, in accordance with respective applications of the present invention;

FIGS. 22A-E are schematic cross-sectional illustrations of the sampling device of FIGS. 20A-B and the method of using the sampling device, in accordance with respective applications of the present invention;

FIGS. 23A-E are schematic cross-sectional illustrations of another sampling device and the method of using the sampling device, in accordance with respective applications of the present invention;

FIGS. 24A-B are schematic illustrations of yet another sampling device for concentrating a liquid specimen sample, and a portion of the sampling device, respectively, in accordance with an application of the present invention;

FIGS. 25A-E are schematic illustrations of the sampling device of FIGS. 24A-B and a method of using the sampling device, in accordance with respective applications of the present invention;

FIGS. 26A-E are schematic cross-sectional illustrations of the sampling device of FIGS. 24A-B and the method of using the sampling device, in accordance with respective applications of the present invention;

FIGS. 27A-B are schematic illustrations of still another sampling device for concentrating a liquid specimen sample, and a portion of the sampling device, respectively, in accordance with an application of the present invention;

FIGS. 28A-E are schematic illustrations of the sampling device of FIGS. 27A-B and a method of using the sampling device, in accordance with respective applications of the present invention;

FIGS. 29A-E are schematic cross-sectional illustrations of the sampling device of FIGS. 27A-B and the method of using the sampling device, in accordance with respective applications of the present invention;

FIG. 30 is a schematic illustration of a kit, in accordance with an application of the present invention;

FIG. 31 is a schematic illustration of a testing kit, in accordance with an application of the present invention;

FIG. 32A is a schematic illustration of a kit for testing for presence of a particulate, in accordance with an application of the present invention;

FIGS. 32B-K are schematic illustrations of a method of using the kit of FIG. 32A for testing for presence of a particulate, in accordance with an application of the present invention;

FIGS. 33A and 33B are schematic illustrations of respective configurations of a housing, in accordance with respective applications of the present invention;

FIG. 33C is a schematic illustration of another housing, in accordance with an application of the present invention;

FIGS. 34A-F are schematic illustrations of a kit and a method for testing for presence of a particulate, in accordance with an application of the present invention;

FIGS. 35A-D are schematic illustrations of a portion of a method for testing for presence of a particulate, in accordance with an application of the present invention;

FIGS. 36A-B are schematic isometric illustrations of a kit and a method of using the kit for testing for presence of a particulate, respectively, in accordance with an application of the present invention;

FIGS. 37A-B are schematic cross-sectional illustrations of the kit and the method of using the kit of FIGS. 36A-B, in accordance with an application of the present invention;

FIG. 38 is a schematic illustration of a filtration assembly, in accordance with an application of the present invention;

FIG. 39 is a schematic illustration of a filter reinforcement coupled to a filter, in accordance with an application of the present invention;

FIG. 40 is a schematic illustration of another filter reinforcement coupled to a filter, in accordance with an application of the present invention;

FIG. 41 is a schematic illustration of yet another filter reinforcement coupled to a filter, in accordance with an application of the present invention;

FIGS. 42A-B are schematic illustrations of additional filter reinforcements coupled to a filter, in accordance with an application of the present invention; and

FIG. 43 is a photograph of one implementation of the kit of FIGS. 36A-B and 37A-B, in accordance with an application of the present invention.

### DETAILED DESCRIPTION OF APPLICATIONS

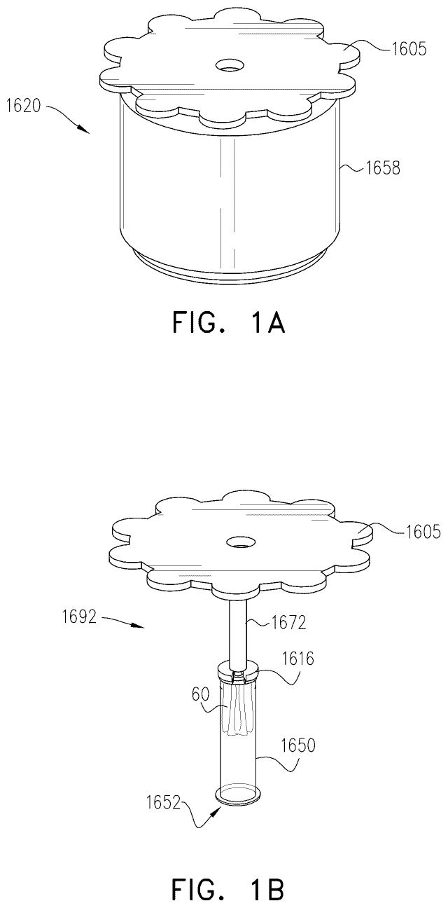

FIGS. 1A-B are schematic illustrations of a sampling device 1620 for concentrating a liquid specimen sample 22, and a portion of the sampling device, respectively, in accordance with an application of the present invention.

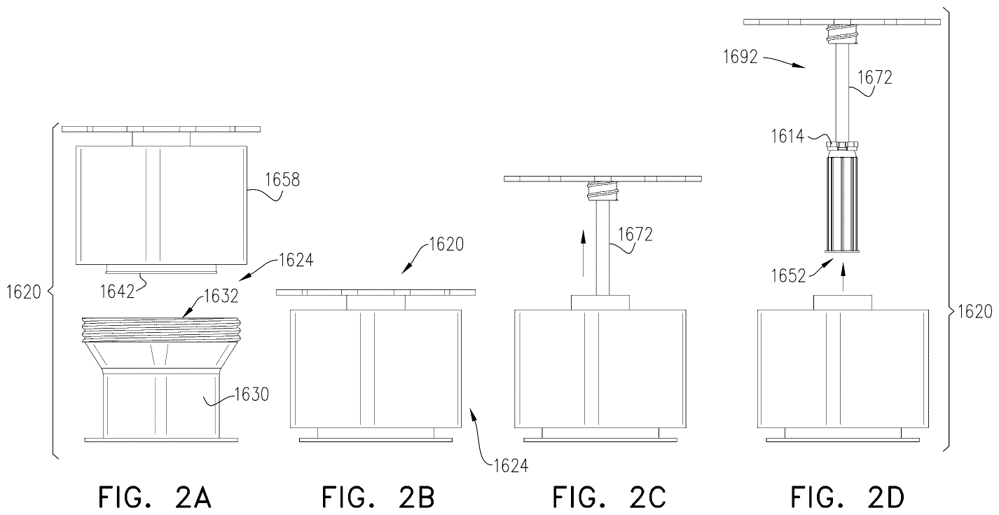

Reference is also made to FIGS. 2A-D, which are schematic illustrations of sampling device 1620 and a method of using sampling device 1620, in accordance with respective applications of the present invention.

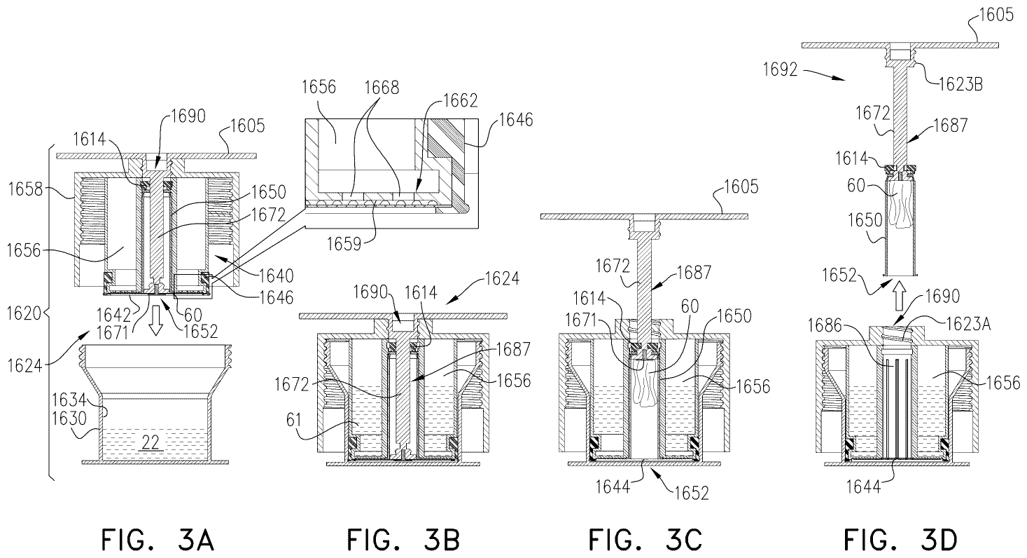

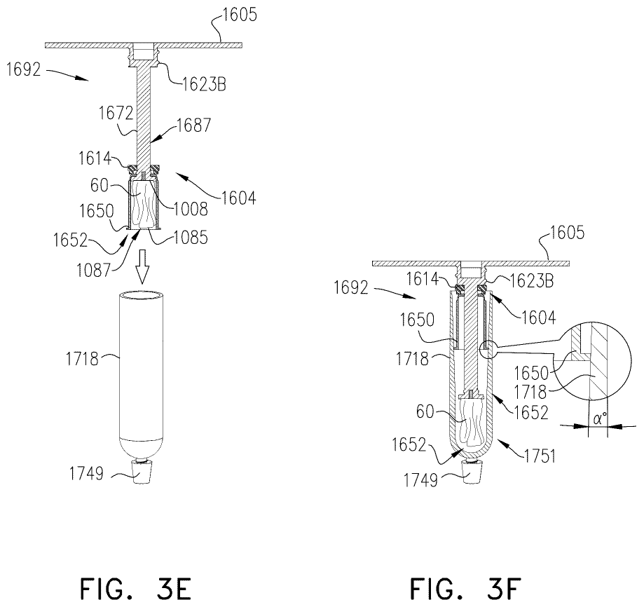

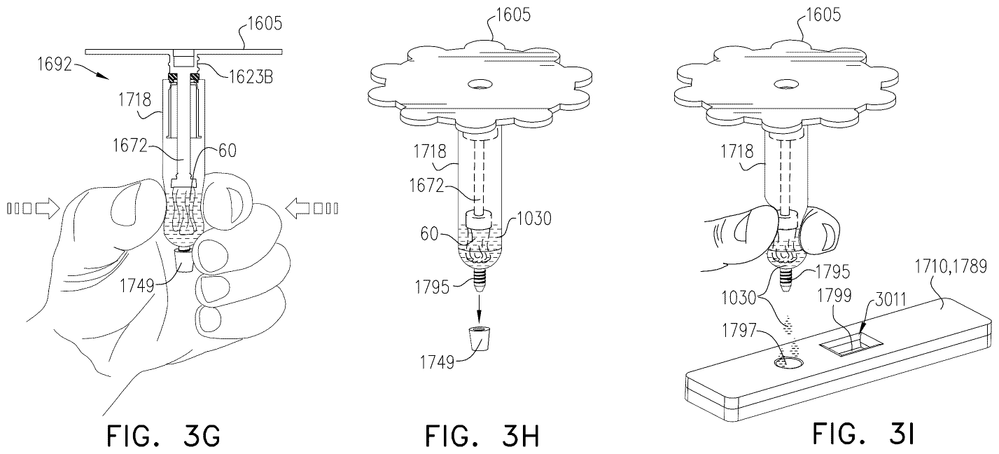

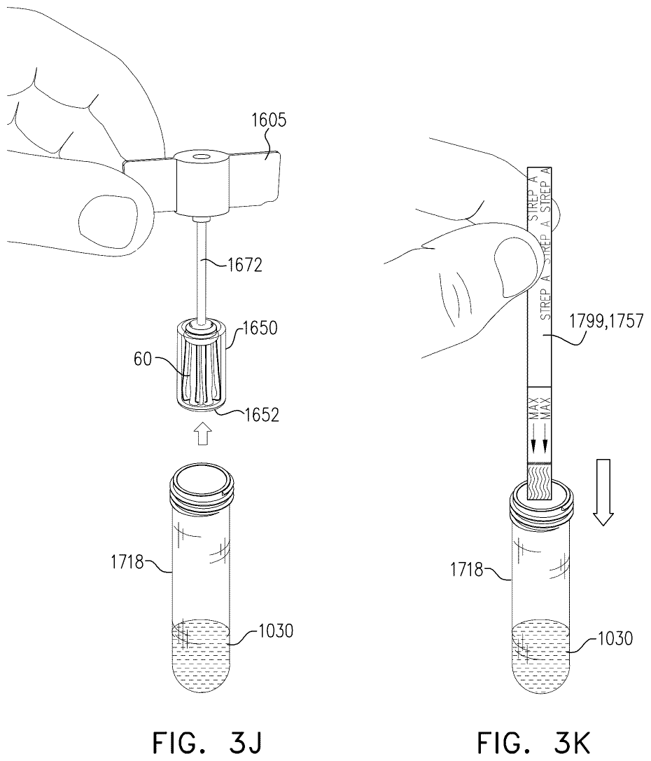

Reference is also made to FIGS. 3A-I, which are schematic cross-sectional illustrations of sampling device 1620 and a method of using sampling device 1620, in accordance with respective applications of the present invention. Reference is also made to FIGS. 3J-K, which are schematic illustrations of another method of using sampling device 1620, in accordance with an application of the present invention.

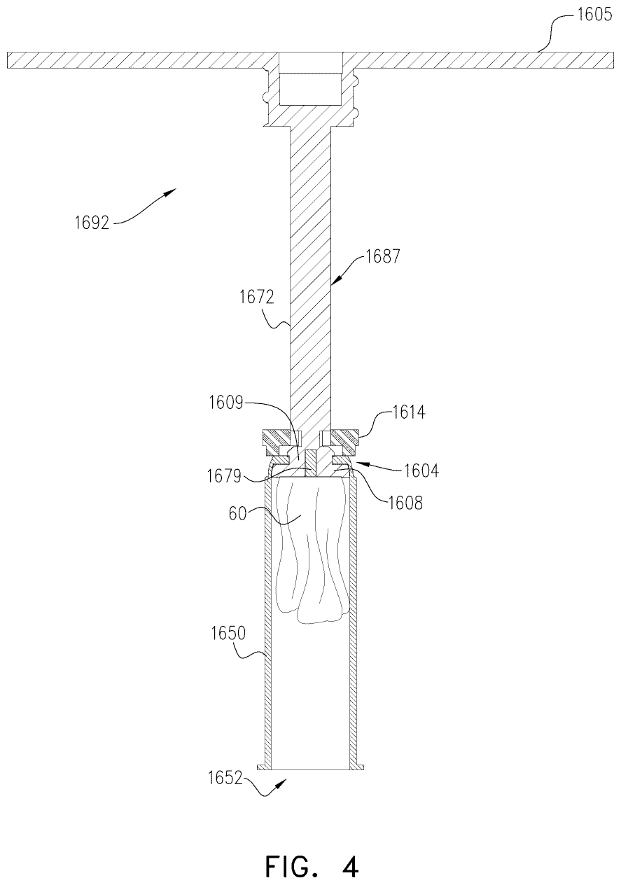

Reference is further made to FIG. 4, which is an enlarged schematic illustration of a portion of sampling device 1620 in the state shown in FIGS. 2D and 3D, in accordance with an application of the present invention.

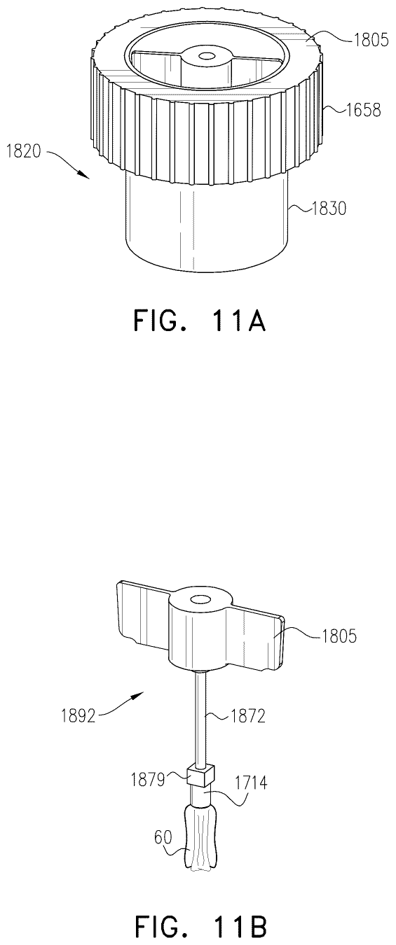

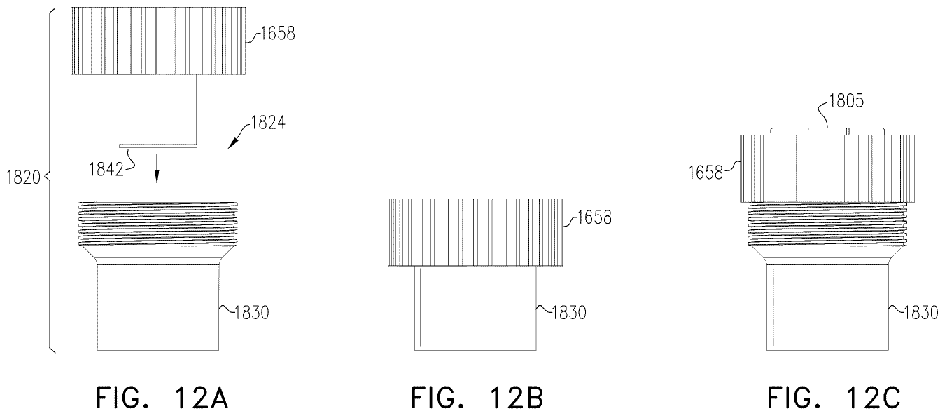

Sampling device 1620 comprises a filtration assembly 1624 and a collection vial 1650. The features of sampling device 1620, including but not limited to collection vial 1650, may be implemented in any of the other sampling devices described hereinbelow, mutatis mutandis. Similarly, sampling device 1620 may be implemented in combination with any of the features of the other sampling devices described hereinbelow, mutatis mutandis, including, by way of example and not limitation, the reversible filter-clamping techniques of sampling devices 1820 or 1920, described hereinbelow with reference to FIGS. 11A-13F and FIGS. 14A-16E, respectively.

Filtration assembly 1624 comprises tubular container 1630, a plunger 1640 (labeled in FIG. 3A), and a filter 60.

Tubular container 1630 is shaped so as to define a proximal container opening 1632 (labeled in FIG. 2A) for receiving liquid specimen sample 22, after or during collection of liquid specimen sample 22 from the subject. Tubular container 1630 is also shaped so as to define an inner wall 1634 (labeled in FIG. 3A). At least a portion of tubular container 1630, such as a distal portion, may define a syringe barrel.

As labeled in FIGS. 2A and 3A, plunger 1640 comprises a plunger head 1642 and a plunger rod 1682. Plunger 1640 is insertable into tubular container 1630 via proximal container opening 1632, such that a lateral surface 1646 of plunger head 1642 (labeled in FIG. 3A) forms a fluid-tight movable seal with inner wall 1634. To this end, lateral surface 1646 may comprise an elastomeric material, such as natural rubber, synthetic rubber, a thermoplastic elastomer, or a combination thereof. Optionally, filtration assembly 1624 further comprises a plunger support 1658, which is coupled to a proximal portion of plunger 1640. Plunger support 1658 may be configured to be coupled to tubular container 1630. A portion of plunger support 1658 may serve as a handle to enable easy manipulation of plunger 1640, including insertion of plunger 1640 into tubular container 1630. Optionally, a portion of plunger support 1658 surrounds plunger 1640.

For some of these applications, plunger rod 1682 is shaped so as to define an internal plunger space 1686 (labeled in FIG. 3D). For some of these applications, a proximal end of plunger rod 1682 is shaped so as to define a plunger-space proximal opening 1690 of internal plunger space 1686. Plunger head 1642 is shaped so as to define a plunger-head opening 1644 (labeled in FIG. 3C) through plunger head 1642 and into internal plunger space 1686.

Typically, collection vial 1650 is removably disposed at least partially within internal plunger space 1686.

Typically, collection vial 1650 is positioned proximal to plunger head 1642.

Reference is made to FIGS. 2A and 3A. Optionally, proximal container opening 1632 is shaped as a funnel to facilitate receipt of liquid specimen sample 22 during collection of the liquid specimen sample. For example, liquid specimen sample 22 may be expressed (e.g., spit) from subject's mouth into tubular container 1630, or transferred to tubular container 1630 from a collection container. Optionally, the funnel shape of proximal container opening 1632 is similar to funnel-shaped proximal opening 36 shown in FIG. 1 of US Patent Application Publication 2019/0381498 to Fruchter et al., which is incorporated herein by reference. Tubular container 1630 may be cylindrical, as shown, or may alternatively have another, non-circular cross-sectional shape. Alternatively or additionally, tubular container 1630 may have different cross-sectional shapes along respective different longitudinal portions of the tubular container; optionally, one or more of the cross-sectional shapes is circular.

Typically, tubular container 1630 has an internal volume of at least 0.5 ml (e.g., at least 1 ml, such as at least 5 ml), no more than 500 ml (e.g., no more than 70 ml), and/or 0.5 ml (e.g., 1 ml or 5 ml)-500 ml (e.g., 70 ml).

For some applications, tubular container 1630 does not comprise a Luer lock or any other type of needle-coupling mechanism.

As shown in FIGS. 2D, 3C, and 3D, collection vial 1650 is typically shaped so as to define a vial opening 1652.

For some applications, collection vial 1650 has a volume of at least 1 ml, no more than 50 ml, and/or 1-50 ml, such as at least 2 ml, no more than 20 ml, and/or 2-20 ml, e.g., at least 3 ml (e.g., at least 5 ml), no more than 15 ml, and/or 3 (e.g., 5)-15 ml. For some applications, collection vial 1650 has a greatest internal diameter of no more than 35 mm, e.g., no more than 20 mm, such as no more than 15 mm or no more than 10 mm.

Collection vial 1650 typically has a greatest outer diameter that is less than (e.g., less than 80%, such as less than 70%) an inner diameter of an axial portion of tubular container 1630 in which plunger head 1642 is distally advanceable.

Collection vial 1650 typically is not shaped so as to define any pressure-release openings and does not comprise any pressure-release valves.

Reference is made to FIGS. 3B-D. Typically, filtration assembly 1624 further comprises a waste liquid receptacle 1656 for receiving a filtrate 61. For some of these applications, plunger rod 1682 is shaped so as to define therewithin waste liquid receptacle 1656. Typically, waste liquid receptacle 1656 partially or entirely surrounds internal plunger space 1686, such as shown.

For some applications, plunger head 1642 is shaped so as to define a filter support 1662 (labeled in FIG. 3A), which is shaped so as to define:
a support surface 1659, which may be perpendicular to a central longitudinal axis of plunger head 1642 (as shown), or may be angled with respect to the central longitudinal axis (configuration not shown),
a plurality of filtrate-passage openings 1668 through filter support 1662 into waste liquid receptacle 1656, and
a central opening that defines plunger-head opening 1644 (labeled in FIGS. 3C and 3D).

Filter 60 is (removably) disposed on support surface 1659, typically on an upstream side of support surface 1659 (which, in the configuration of sampling device 1620, is a distal side of support surface 1659).

Reference is made to FIGS. 2A-B and 3A-B. Filtration assembly 1624 is configured such that movement (typically distal advancement) of plunger head 1642 within tubular container 1630, when liquid specimen sample 22 is contained in tubular container 1630 and filter 60 is disposed in tubular container 1630, pushes at least a portion of liquid specimen sample 22 through filter 60. Filter 60 is configured to concentrate at least a portion of liquid specimen sample 22 onto filter 60, while allowing filtrate 61 to pass through filter 60. Typically, distal advancement of plunger 1640 within tubular container 1630 applies pressure to drive (e.g., push) at least a portion of liquid specimen sample 22 contained in tubular container 1630 through filter 60, such as shown in the transitions between FIGS. 2A and 2B and between FIGS. 3A and 3B.

Filtration assembly 1624 is configured such that movement of plunger head 1642 within tubular container 1630, when liquid specimen sample 22 is contained in tubular container 1630 and filter 60 is disposed in tubular container 1630, pushes at least a portion of liquid specimen sample 22 through filter 60 and filtrate-passage openings 1668 and into waste liquid receptacle 1656.

Optionally, waste liquid receptacle 1656 is shaped so as to define an opening through an external wall of waste liquid receptacle 1656 to release displaced air. For example, the opening may be located on a proximal portion of the external wall, typically above the highest level that filtrate 61 is expected to reach during ordinary use of the device. For some applications, waste liquid receptacle 1656 comprises an air filter (e.g., an N98 filter) that is disposed to filter air that passes out of waste liquid receptacle 1656 through the opening. Alternatively or additionally, for some applications, waste liquid receptacle 1656 comprises a one-way pressure-sensitive valve disposed in the opening.

Filter 60 comprises synthetic or natural materials formed, for example, as a matrix, membrane, fabric, beads, or other configuration. For some applications, filter 60 comprises a mechanical filter, which is configured to mechanically filter particulate from liquid specimen sample 22 by size-based filtration. Optionally, filter 60 comprises a depth filter.

Alternatively or additionally, for some applications, filter 60 comprises fixed antibodies configured to capture the particulate (e.g., free viral particles) by affinity-based filtration.

For some applications, for example, when filter 60 is used for capturing free virus, virions, or viral particles by size-based filtration, filter 60 may have a pore size of 0.01-0.3 microns and/or a molecular weight cut off of 10 kDa-500 kDa. For some applications, filter 60 has a pore size of 0.2-5.0 microns, such as 0.2-2.0 microns (e.g., 0.8 to 1.5 microns, such as 1.2 microns), for example, when filter 60 is used for capturing bacteria by size-based filtration.

For some applications, filter 60 comprises a polyethersulfone (PES) membrane filter.

Alternatively or additionally, for some applications, filter 60 has a nominal pore size of 30 microns-1.5 mm, the nominal pore size representative of a minimum size of spherical particles necessary for the filter to retain 85% of the spherical particles when H2O containing the spherical particles is passed through the filter at 20 degrees C. under pressure supplied by a 10 cm water column. For these applications, filter 60 may implement techniques described in U.S. Provisional Application 63/117,294, filed Nov. 23, 2020, is assigned to the assignee of the present application and incorporated herein by reference, and/or in PCT Publication WO 2021/224925 to Levitz et al., which is incorporated herein by reference.

For example, the nominal pore size may be at least 40 microns, such as at least 60 microns, e.g., at least 100 microns, at least 120 microns, at least 150 microns, at least 200 microns, or at least 500 microns. Alternatively or additionally, for example, the nominal pore size may be less than 1 mm, such as less than 750 microns, less than 500 microns, or less than 250 microns.

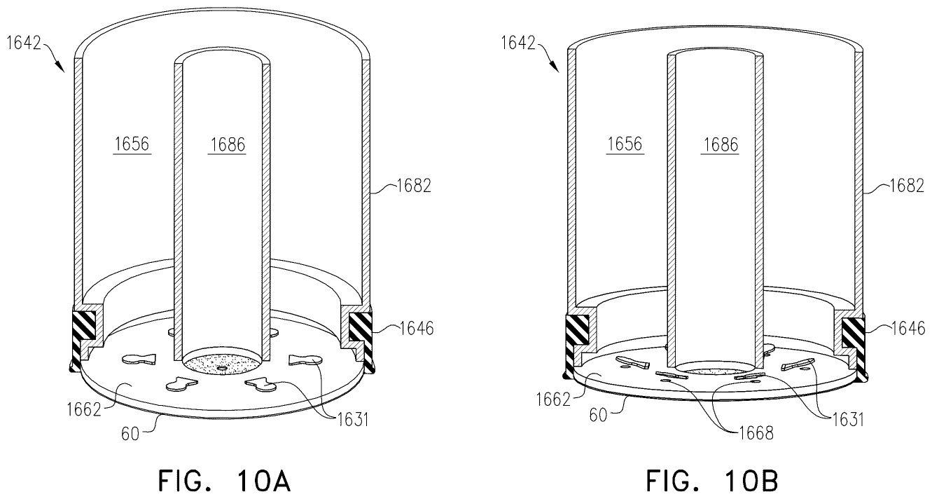

For some applications, filtration assembly 1624 comprises a plurality of filters, such as described with reference to FIGS. 10A-B in PCT Publication WO 2022/149135 to Feldman et al., which is assigned to the assignee of the present application and incorporated herein by reference. Optionally, two or more of the plurality of filters touch one another, such as shown in FIGS. 10A-B of the '024 publication, or are separated by one another by one or more thin spacers, e.g., having a thickness of at least 0.05 mm, no more than 1 mm, and/or 0.05-1 mm (configuration not shown). Alternatively or additionally, two or more of the plurality of filters are spaced apart from another, which case filtration assembly 1624 optionally comprises a corresponding number of filter supports 1662, some or all of which may have some or all of the characteristics of filter support 1662 (configuration not shown). Further alternatively or additionally, filtration assembly 1624 comprises one or more additional filters downstream of filter 60 (configuration not shown).

Reference is made to FIGS. 2D and 3D. Sampling device 1620 is typically configured such that filter 60 is removable from tubular container 1630 via a plunger-space proximal opening 1690 while plunger head 1642 of plunger 1640 (and typically filter support 1662) remains within tubular container 1630 (filter 60 is also removable from tubular container 1630 via plunger-space proximal opening 1690 if plunger head 1642 has been removed from tubular container 1630). As mentioned above, plunger head 1642 is shaped so as to define plunger-head opening 1644 through plunger head 1642 and into internal plunger space 1686 of plunger 1640.

Sampling device 1620 is configured such that filter 60 is advanceable into (e.g., entirely into) collection vial 1650 via vial opening 1652 while collection vial 1650 is disengageably coupled to filtration assembly 1624, such as shown in FIGS. 2C and 3C.

Reference is made to FIGS. 2C and 3C. For some applications, sampling device 1620 is configured such that filter 60 is advanceable into collection vial 1650 via vial opening 1652 while plunger head 1642 (and typically filter support 1662) remains within tubular container 1630, such as shown in FIGS. 2C and 3C. For some of these applications, sampling device 1620 is configured such that filter 60 is advanceable into collection vial 1650 via vial opening 1652 while plunger head 1642 is advanced as far as possible within tubular container 1630, such as shown in FIGS. 2C and 3C. Alternatively or additionally, for some applications, sampling device 1620 is configured such that filter 60 is advanceable into collection vial 1650 via vial opening 1652 without any proximal withdrawal of plunger head 1642 within tubular container 1630, such as shown in FIGS. 2C and 3C.

Reference is made to FIGS. 2D and 3D. For some applications, sampling device 1620 is configured such that collection vial 1650 is decouplable from filtration assembly 1624 while plunger head 1642 remains within tubular container 1630, such as shown in FIGS. 2D and 3D, typically, but not necessarily, via a proximal end of plunger 1640. For some of these applications, sampling device 1620 is configured such that collection vial 1650 is decouplable from filtration assembly 1624 while plunger head 1642 is advanced as far as possible within tubular container 1630, such as shown in FIGS. 2D and 3D. Alternatively or additionally, for some applications, sampling device 1620 is configured such that collection vial 1650 is decouplable from filtration assembly 1624 without any proximal withdrawal of plunger head 1642 within tubular container 1630, also such as shown in FIGS. 2D and 3D.

Collection vial 1650 is disengageably coupled to filtration assembly 1624. Once collection vial 1650 has been decoupled from filtration assembly 1624, a diagnostic test may be performed for the presence of particulate trapped by filter 60, which is now in collection vial 1650. For some applications, such as for transporting collection vial 1650 to a remote diagnostic laboratory, sampling device 1620 further comprises collection vial cap, which is configured to seal vial opening 1652.

For some applications, collection vial 1650 is disengageably coupled to plunger 1640. Such as described hereinbelow, once collection vial 1650 has been removed from plunger 1640, a diagnostic test may be performed for the presence of particulate trapped by filter 60, which is now in collection vial 1650. For some applications, such as for transporting collection vial 1650 to a remote diagnostic laboratory, sampling device 1620 further comprises a collection vial cap, which is configured to seal vial opening 1652.

Reference is still made to FIGS. 1A-4. For some applications, such as shown in FIGS. 3A-4, sampling device 1620 comprises a withdrawer 1692, which comprises a filter-withdrawal shaft 1672. Filter-withdrawal shaft 1672:
is disposed partially within collection vial 1650 within internal plunger space 1686,
includes a proximal portion 1687 that is slidably disposed passing through a shaft-passage hole 1609 through an end 1604 of collection vial 1650 opposite vial opening 1652 (labeled in FIG. 4), and
includes a distal portion 1608 (labeled in FIG. 4) that is coupled or couplable (directly or indirectly) to filter 60, typically via an end 1604 of collection vial 1650 opposite a vial opening 1652.

Sampling device 1620 is configured such that proximal movement (e.g., withdrawal) of filter-withdrawal shaft 1672, while plunger head 1642 (and typically filter support 1662) remains within tubular container 1630, collects filter 60 in collection vial 1650 by pulling filter 60 at least partially into (such as entirely into) collection vial 1650 via plunger-head opening 1644 (which, as mentioned above, is defined by the central opening of filter support 1662) via vial opening 1652 (as shown in the transitions between FIGS. 2B and 2C and between FIGS. 3B and 3C). At least a portion of filter 60 is typically bunched up within collection vial 1650, such as into a flower-like arrangement, from the filter's initial flat shape while disposed on filter support 1662.

Typically, sampling device 1620 is configured such that further proximal withdrawal of filter-withdrawal shaft 1672 out of internal plunger space 1686, while plunger head 1642 (and typically filter support 1662) remains within tubular container 1630, pulls collection vial 1650 out of internal plunger space 1686 via plunger-space proximal opening 1690 (as shown in the transitions between FIGS. 2C and 2D and between FIGS. 3C and 3D). It is noted that filter-withdrawal shaft 1672 of sampling device 1620 is not an element of filtration assembly 1624, but instead is removable therefrom, as shown in FIGS. 2D and 3D.

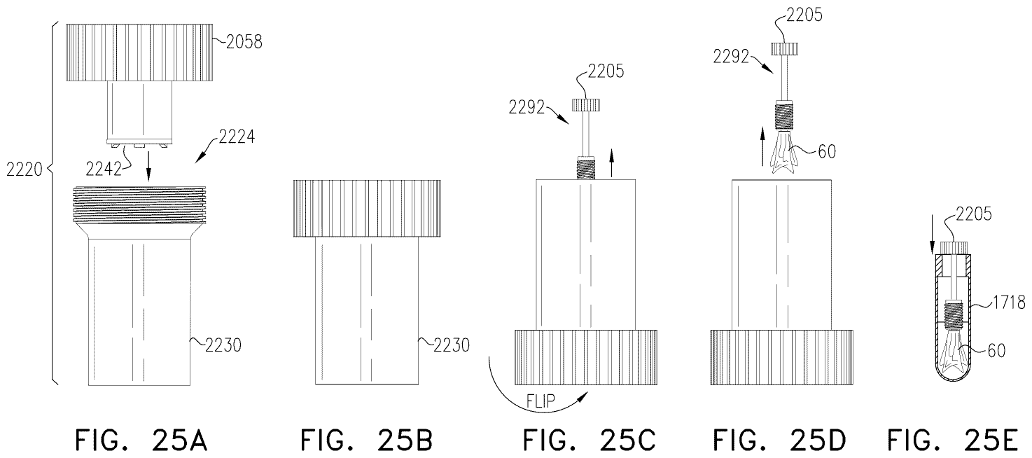

For some applications, distal portion 1608 of filter-withdrawal shaft 1672 is coupled to filter 60 before use of filter 60 (typically during manufacture), while for other applications, distal portion 1608 of filter-withdrawal shaft 1672 is couplable to filter 60 during use of filter 60, such as, by way of example and not limitation, described hereinbelow with reference to FIGS. 25A-E and 26A-E.

For some applications, sampling device 1620 comprises a distal plate 1671 (labeled in FIG. 3A), which is disposed in contact with a distal surface of filter 60, and is coupled (directly or indirectly) to filter-withdrawal shaft 1672 through end 1604 of collection vial 1650. For example, distal plate 1671 may be circular, i.e., shaped as a disc, or any other shape. Distal plate 1671 may be flexible, e.g., comprise silicone, or may be rigid, e.g., comprise metal or a polymer.

For some applications, withdrawer 1692 further comprises a shaft handle 1605, which is coupled to a proximal portion of filter-withdrawal shaft 1672. Optionally, shaft handle 1605 is shaped as a wing nut.

For some applications, filtration assembly 1624 (e.g., plunger-space proximal opening 1690 and/or plunger support 1658) and withdrawer 1692 (either shaft handle 1605 or filter-withdrawal shaft 1672) are shaped so as to define corresponding screw threads 1623A and 1623B (e.g., female and male screw threads 1623A and 1623B) (labeled in FIG. 3D), respectively, which (a) removably couple filter-withdrawal shaft 1672 to plunger rod 1682, such as shown in FIG. 3B, while filter-withdrawal shaft 1672 is disposed passing through internal plunger space 1686, and (b) prevent the premature proximal withdrawal of filter-withdrawal shaft 1672 out of internal plunger space 1686. Sampling device 1620 is configured such that rotation of filter-withdrawal shaft 1672 and plunger-space proximal opening 1690 with respect to each other (a) causes at least an initial portion of the proximal withdrawal of filter-withdrawal shaft 1672 out of internal plunger space 1686, such as shown in the transition between FIG. 3B and FIG. 3C, and (b) decouples female and male screw threads 1623A and 1623B from each other, thereby allowing the continuation of the proximal withdrawal of filter-withdrawal shaft 1672 out of internal plunger space 1686, such as shown in FIGS. 3C-D. Alternatively, shaft handle 1605 may be shaped so as to define screw thread 1623B, such as described hereinbelow regarding shaft handle 1805 and withdrawer 1892, mutatis mutandis.

Optionally, in configurations in which plunger support 1658 and/or plunger 1640 and tubular container 1630 are threadingly coupled to each other, (a) the threading between plunger-space proximal opening 1690 and filter-withdrawal shaft 1672 or shaft handle 1603 and (b) the threading between plunger 1640 and tubular container 1630 have opposite handedness.

For other applications, filtration assembly 1624 and filter-withdrawal shaft 1672 are not threadingly coupled together, and plunger-space proximal opening 1690 and shaft handle 1605 are not threadingly coupled together.

Reference is made to FIGS. 2D, 3A-F, and 4. For some applications, sampling device 1620 further comprises a seal 1614 that inhibits fluid leakage between proximal portion 1687 of filter-withdrawal shaft 1672 and shaft-passage hole 1609.

Optionally, an inner portion of seal 1614 may snap into an external circumferential groove of proximal portion 1687 of filter-withdrawal shaft 1672 upon the proximal withdrawal of most or all of filter-withdrawal shaft 1672 from collection vial 1650, such as shown in FIG. 4.

Reference is again made to FIGS. 1A-4. As described hereinabove, filter-withdrawal shaft 1672 include distal portion 1608 that is directly or indirectly coupled to filter 60. Exemplary ways in which the distal portion of the filter-withdrawal shaft may be directly or indirectly coupled to filter 60 include, but are not limited to:
the distal portion of filter-withdrawal shaft 1672 may be directly coupled to filter 60, such as shown in FIG. 4, e.g., by an adhesive and/or by distal plate 1671 (labeled in FIG. 3A, which may be fixed, e.g., pinned, to the distal end of the distal portion of filter-withdrawal shaft 1672; in these configurations, the distal portion of the filter-withdrawal shaft passes through the end of the collection vial opposite the vial opening; and
the distal portion of filter-withdrawal shaft 1672 may be indirectly coupled to the filter, such as shown in FIG. 4, e.g., by a rod 1679 (labeled in FIG. 4) that (a) is fixed to the distal end of the distal portion of filter-withdrawal shaft 1672 and (b) passes through the end of the collection vial opposite the vial opening, and optionally further by an adhesive and/or by distal plate 1671 which may be integral with rod 1679 or fixed to rod 1679.

It will be appreciated by persons skilled in the art who have read the present application that the distal portions of filter-withdrawal shaft 1672 may be directly or indirectly coupled to the filter in additional ways, all of which are within the scope of the present invention.

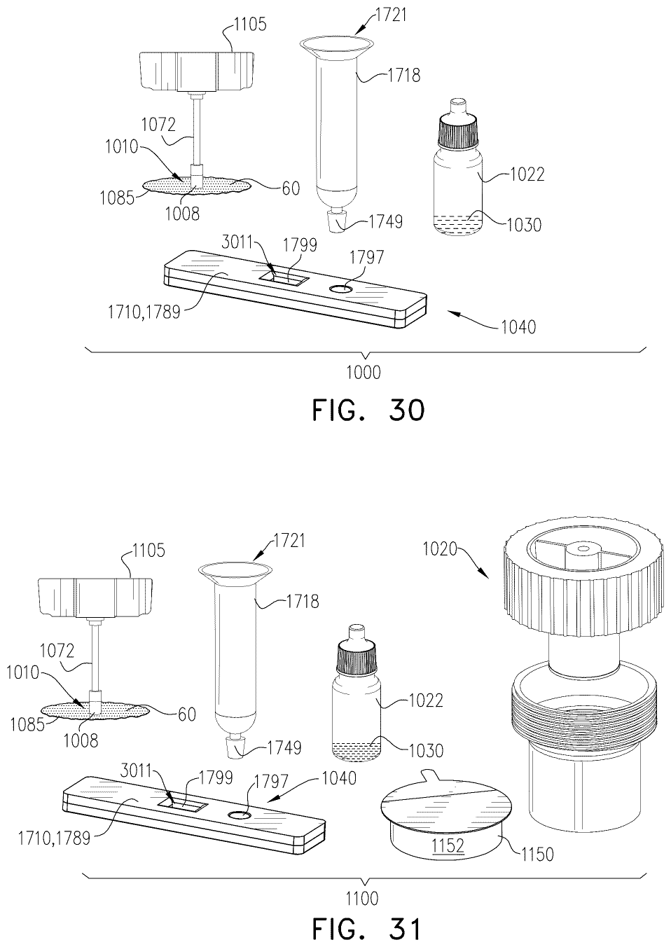

Optionally, filter-withdrawal shaft 1672 is coupled to filter 60 in ways described hereinbelow with reference to FIGS. 30 and 31.

Reference is now made to FIGS. 3E-I. For some applications, the method optionally continues as shown in FIGS. 3E-I. By way of example and not limitation, collection vial 1650 is shown as being shorter in FIGS. 3E-I than in FIGS. 1A-3D. In actual use, the collection vial has the same length throughout its use, i.e., the same length in FIGS. 1A-3D as in FIGS. 3E-I. If the collection vial is shorter than shown in FIGS. 1A-3D, the collection vial occupies only a proximal portion of internal plunger space 1686.

As shown in FIGS. 3E-F, collection vial 1650 is inserted at least partially into an extraction tube 1718, and distally advanced within extraction tube 1718 until bunched-up filter 60 is positioned near a distal end 1751 of extraction tube 1718 opposite a proximal end opening 1721. Extraction tube 1718 is shaped so as to prevent collection vial 1650 from reaching distal end 1751 of extraction tube 1718, such that collection vial 1650 slides up a portion of filter-withdrawal shaft 1672 as bunched-up filter 60 is positioned near distal end 1751, thereby ejecting bunched-up filter 60 from vial opening 1652 of collection vial 1650 and exposing bunched-up filter 60 to a liquid 1030, such as described hereinbelow, within the distal portion of extraction tube 1718.

This technique may aid with the insertion of bunched-up filter 60 into extraction tube 1718. Collection vial 1650 is readily inserted into extraction tube 1718, thereby inserting bunched-up filter 60 into extraction tube 1718 while the bunched-up filter is initially within collection vial 1650. Collection vial 1650 also may serve to cover and/or shield bunched-up filter 60 when filter 60 is exposed to the environment, such as before insertion into extraction tube 1718 and/or after optional removal from extraction tube 1718, such as described hereinbelow with reference to FIGS. 3J, 32G, and 34D; in this sense, collection vial 1650 may also function as a sleeve for covering and/or shielding bunched-up filter 60.

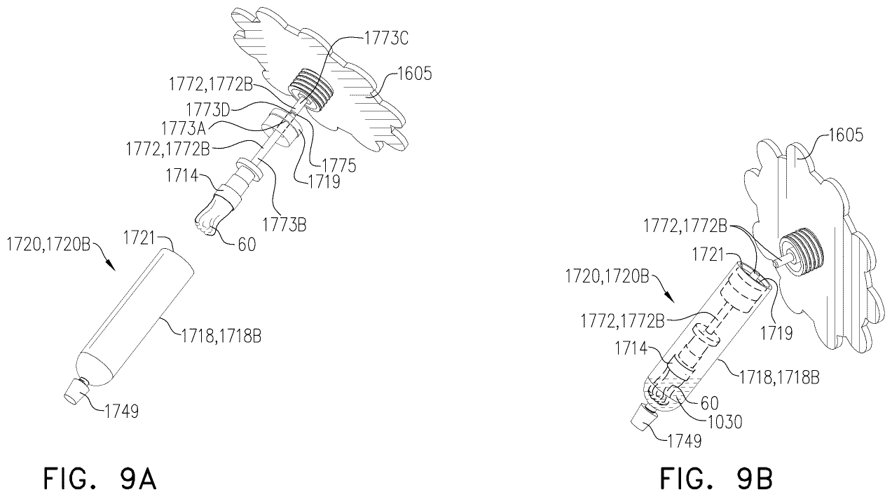

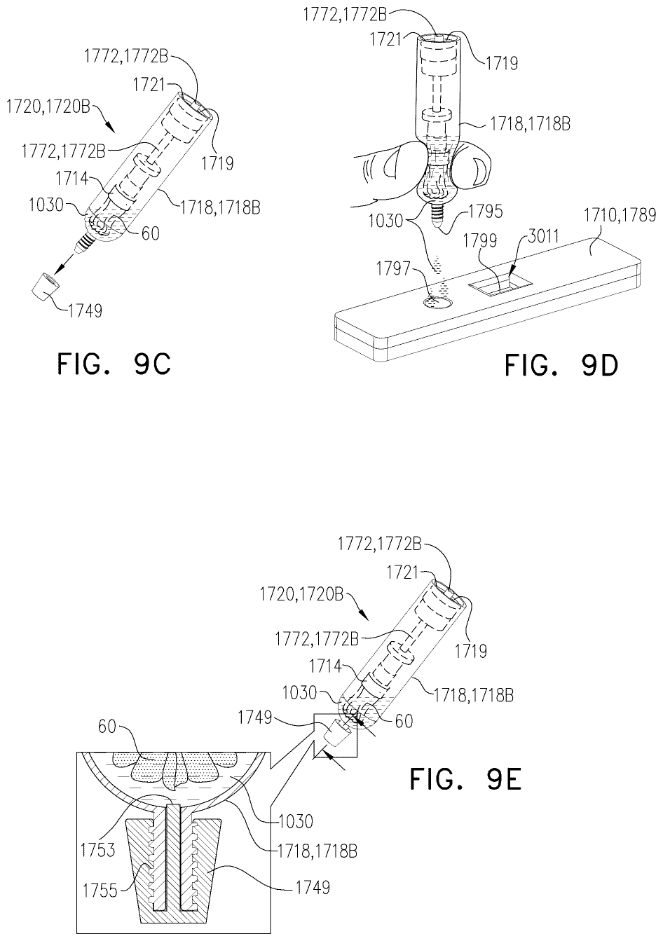

Optionally, as shown in FIGS. 3E-H, extraction tube 1718 may comprise a screw-off distal tip cap 1749 that removably seals distal end 1751 of extraction tube 1718 opposite proximal end opening 1721. The liquid within extraction tube 1718 may be expelled (e.g., squeezed or dripped out) of extraction tube 1718, such as described hereinbelow with reference to FIG. 31 or FIGS. 9C-D.

For some applications, such as shown in FIG. 3G, while bunched-up filter 60 is exposed to liquid 1030 in extraction tube 1718, filter 60 is squeezed (e.g., by squeezing flexible extraction tube 1718) at least one time without expelling any of liquid 1030 from extraction tube 1718. Screw-off distal tip cap 1749, if provided, is then removed from extraction tube 1718, such as shown in FIG. 3H.

For some applications, extraction tube 1718 is oriented horizontally (rather than vertically) during all or a portion of the exposure of bunched-up filter 60 to liquid 1030 in extraction tube 1718. For example, filter 60 may be rotated in extraction tube 1718, optionally while only partially immersed in liquid 1030.

As shown in FIG. 3I, liquid 1030 within extraction tube 1718 is expelled (e.g., squeezed or dripped out) from extraction tube 1718, such as onto a sample pad 1797 of a lateral flow test strip 1799, such as a lateral flow immunoassay test strip. Alternatively, lateral flow test strip 1799 may comprise another type of lateral flow test strip, such as a CRISPR/Cas9-based lateral flow assay. Optionally, liquid 1030 is expelled from extraction tube 1718 by squeezing extraction tube 1718 at least one time, so as to both squeeze a portion of liquid 1030 from filter 60 and expel the portion of the liquid from the extraction tube. In the technique illustrated in FIG. 3I, liquid 1030 within extraction tube 1718 is expelled from extraction tube 1718 while filter 60 remains within extraction tube 1718.

The techniques of FIG. 3E, FIG. 3F, FIG. 3G, FIG. 3H, and/or FIG. 3I may optionally be implemented in combination with the techniques of any of the sampling devices described herein.

For some applications, collection vial 1650 is flexible. Bunched-up filter 60 is exposed to liquid 1030 in extraction tube 1718, as described above. The bunched-up filter is then withdrawn from extraction tube 1718 and pulled at least partially into collection vial 1650 via vial opening 1652, by proximally moving filter shaft 1672 with respect to collection vial 1650. Thereafter, the bunched-up portion of filter 60 is squeezed by squeezing collection vial 1650, while at least a portion of the bunched-up filter 60 is covered and/or shielded by collection vial 1650, to squeeze a portion of liquid 1030 from filter 60 (configuration not shown).

Reference is made to FIGS. 3J-K. The step illustrated in FIG. 3J may optionally be performed after the step illustrated in FIG. 3F or after the step illustrated in FIG. 3G (i.e., with or without squeezing filter 60 while it is in extraction tube 1718). After bunched-up filter 60 has been exposed to liquid 1030 in extraction tube 1718 (typically bathed in liquid 1030 for a certain amount of time), filter-withdrawal shaft 1672 is withdrawn from extraction tube 1718. This proximal movement of filter-withdrawal shaft 1672 with respect collection vial 1650 pulls filter 60 at least partially into (such as entirely into) collection vial 1650 via vial opening 1652.

For some applications, bunched-up filter 60 is removed from extraction tube 1718 while extraction tube 1718 is squeezed. For example, extraction tube 1718 may be squeezed before removing bunched-up filter 60, such as shown in FIG. 3G, and extraction tube 1718 may continue to be squeezed during removal of bunched-up filter 60 from extraction tube 1718.

For some applications, after removal of filter 60 from extraction tube 1718, liquid 1030 is tested for the presence of a target analyte released into liquid 1030 from particulate trapped by filter 60. For example, a lateral flow test strip, such as a lateral flow immunoassay test strip, optionally implemented as a dipstick 1757, may be inserted into liquid 1030 in extraction tube 1718, such as shown in FIG. 3K.

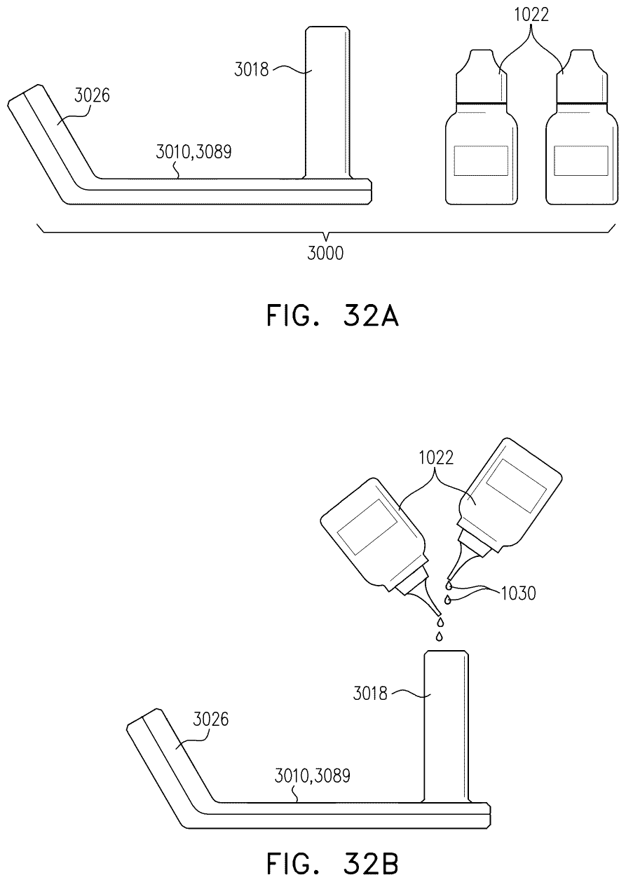

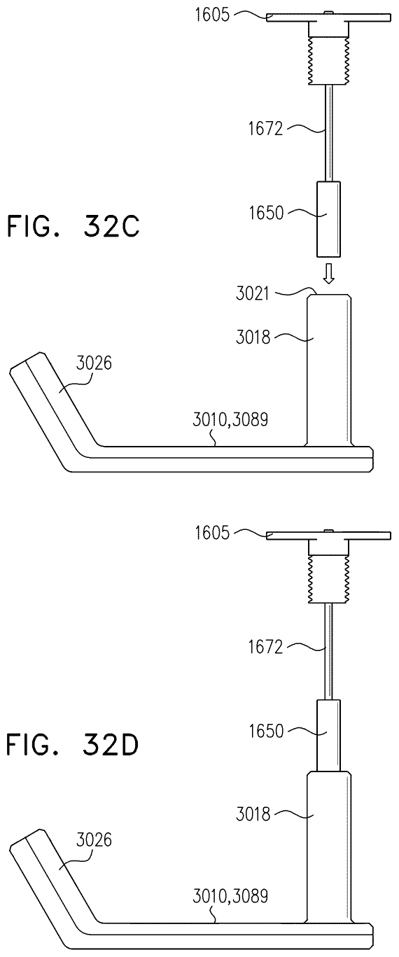

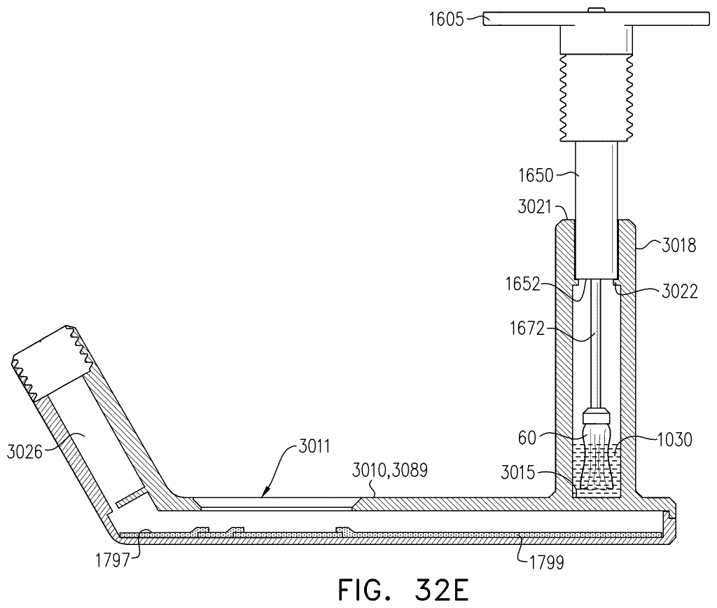

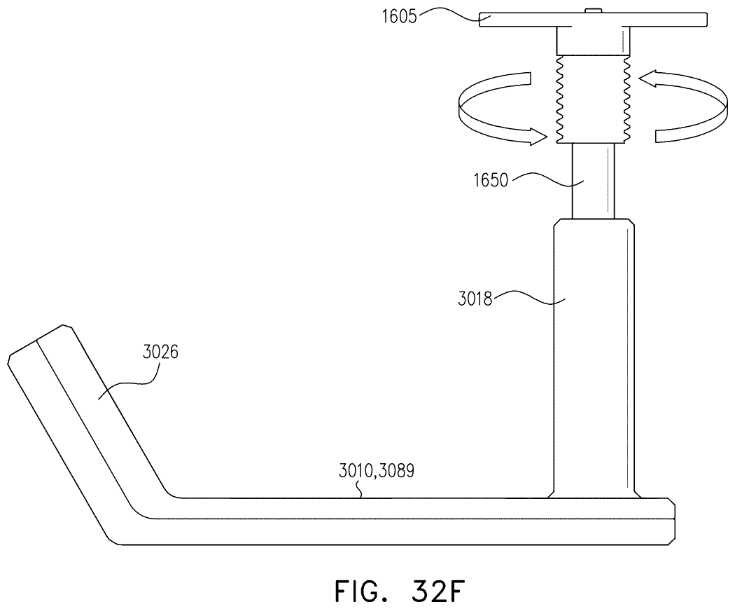

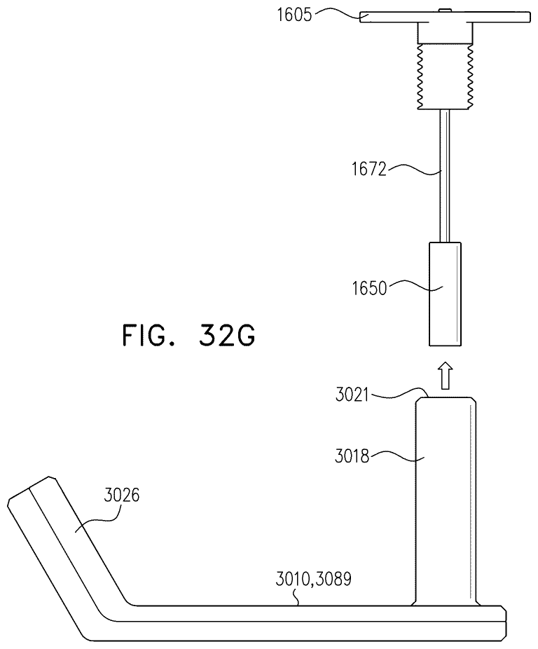

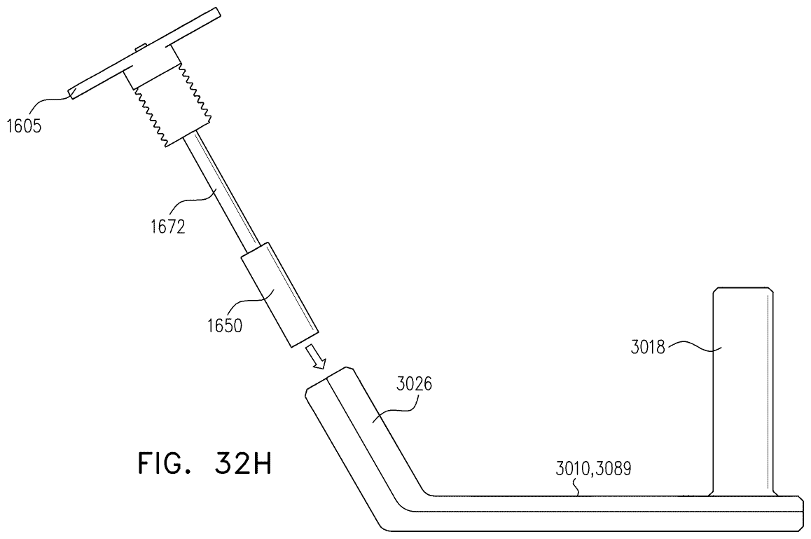

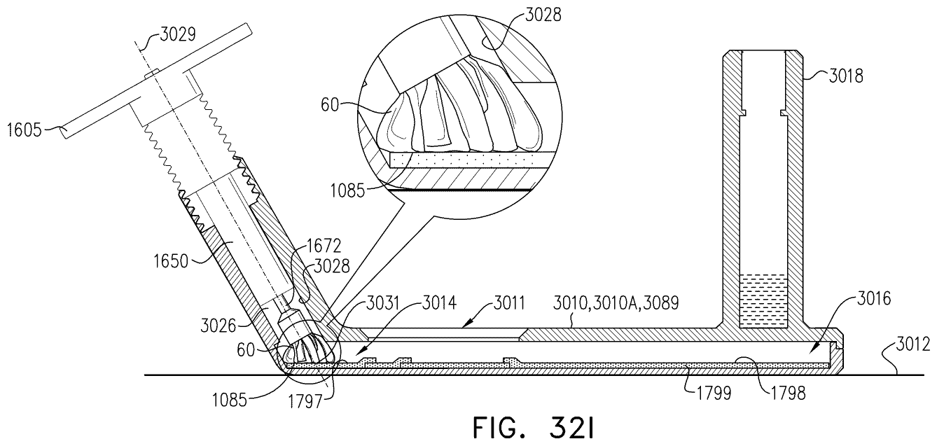

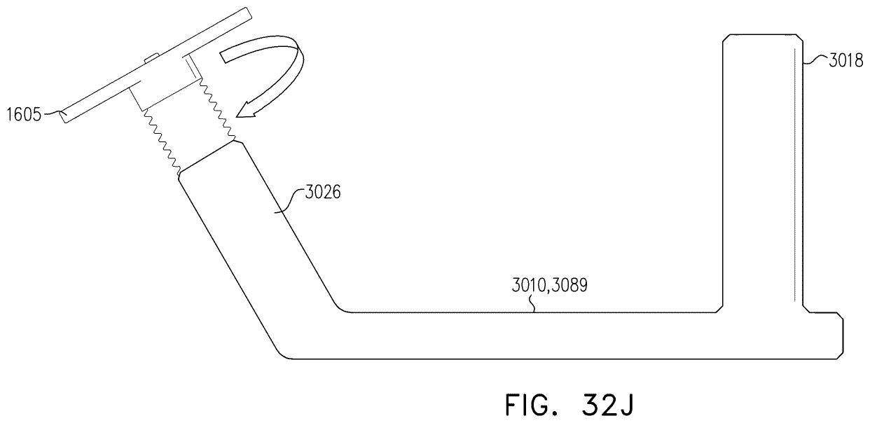

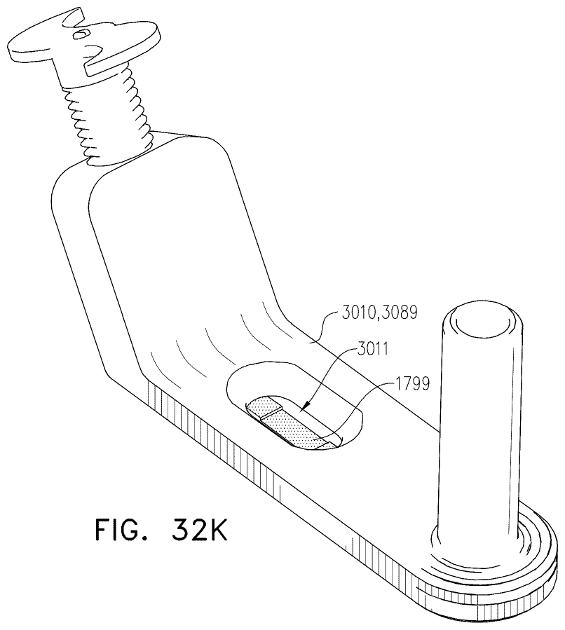

Alternatively, the step illustrated in FIG. 3J may be followed by the techniques of FIG. 32H-K, 34E-F, or 35A-D.

The techniques of FIG. 3J and/or FIG. 3K may optionally be implemented in combination with the techniques of any of the sampling devices described herein.

Alternatively, such as described hereinbelow with reference to FIG. 32H-K or 34E-F, after bunched-up filter 60 is exposed to liquid 1030 in extraction tube 1718 (typically bathed in liquid 1030 for a certain amount of time), collection vial 1650 is removed from extraction tube 1718, and filter 60 is squeezed to partially release the portion of liquid 1030 from filter 60, such as onto sample pad 1797 of lateral flow test strip 1799, such as a lateral flow immunoassay test strip. Alternatively, filter 60 is squeezed by squeezing flexible collection vial 1650 (configuration not shown).

Reference is again made to FIG. 3J. For some applications, after bunched-up filter 60 is bathed in liquid 1030 within extraction tube (and optionally squeezed within extraction tube 1718), a portion of liquid 1030 is discarded, such as by draining the portion of liquid 1030 from extraction tube 1718. For example, extraction tube 1718 may have the configuration described hereinabove with reference to FIGS. 3E-H, and screw-off distal tip cap 1749 may be removed from extraction tube 1718, such as shown in FIG. 3H, in order to discard the portion of liquid 1030.

For some applications, liquid specimen sample 22 is received from a subject's mouth. For some applications, liquid specimen sample 22 comprises gargled fluid, i.e., a gargle fluid that the subject has gargled in his or her mouth and spit out, perhaps along with some saliva. In the present application, including in the claims and Inventive Concepts, “gargled fluid” means “gargle fluid” that has been gargled by a subject. Typically, the gargle fluid includes water, carbonated water, saline (e.g., phosphate buffered saline), pelargonium sidoides extract, tannic acid, balloon flower platycodon grandiflorus, berberine sulfate, S-carboxymethylcysteine, curcumin, coloring, flavoring, a detergent (such as Polysorbate 20 (e.g., Tween® 20)), or any combination thereof. In some applications, the gargle fluid is carbonated. Alternatively or additionally, for some applications, a detergent, such as Polysorbate 20 (e.g., Tween® 20) is added to the gargled fluid after being gargled by the subject. Alternatively, liquid specimen sample 22 may comprise another type of biological fluid, such as blood (e.g., diluted blood), urine, stool (e.g., diluted stool), gastrointestinal (GI) fluid, or bronchoalveolar lavage fluid.

Alternatively, liquid specimen sample 22 comprises saliva not swabbed from the throat of a subject (i.e., the saliva was collected without swabbing the subject's throat). (The distinction between “swab” as a verb and as a noun is noted. A “swab” (as a noun) may be used to obtain saliva without “swabbing” (as a verb) the subject's throat. For example, the subject may suck on a swab, or a swab may be dipped in a container into which gargle fluid or saliva has been placed.) By contrast, in commonly-practiced techniques for testing for strep, the tonsils are swabbed. Further alternatively, liquid specimen sample 22 comprises liquid from a cultured medium containing a biological sample which had been incubated within tubular container 30 or incubated separately from the device and then added to tubular container 30.

Liquid specimen sample 22 (e.g., saliva) may be spit directly by the subject into tubular container 30 or transferred by a healthcare worker from another container into which the subject spit. Alternatively, in the case of saliva, the saliva may be collected from the subject's mouth by having the subject suck on a swab or other absorbent collecting element, such as flocked swabs or cotton rolls.

For some applications in which the method does not comprise swabbing the throat of the subject, liquid specimen sample 22 is collected by drawing liquid specimen sample 22 out of an oral cavity of the subject via an anterior opening of the oral cavity by contacting one or more portions of the oral cavity with an absorbent material, e.g., a flocked or cotton swab, or a sponge (e.g., at a tip of a collector shaft), without swabbing the oropharynx of the subject. (For example, an ORAcollect®. RNA Saliva Collection Device (DNA Genotek Inc., a subsidiary of OraSure Technologies, Inc. (Bethlehem, PA, USA)) may be used.) Optionally, the absorbent material is located on a tip of a collector shaft, and liquid specimen sample 22 is drawn out of the oral cavity via the anterior opening of the oral cavity using the absorbent material by inserting the tip of the collector shaft into the oral cavity. For some of these applications, liquid specimen sample 22 is drawn out of the oral cavity via the anterior opening of the oral cavity using the absorbent material by the subject sucking on the absorbent material. For example, the one or more portions of the oral cavity may include one or more of buccal mucosa, the tongue (e.g., under the tongue), the gums (e.g., the lower gums), and/or the palatal mucosa. For example, for swabbing the lower gums, absorbent material (e.g., at a tip of a collector shaft) may be rubbed back and forth along the lower gums several times. (The anterior opening of the oral cavity is the opening of the mouth between the lips, between outside the oral cavity and inside the oral cavity.)

Alternatively, liquid specimen sample 22 comprises an incubated culture medium containing a biological sample.

Reference is still made to FIGS. 1A-4. In some applications of the present invention, a method for concentrating liquid specimen sample 22 is provided. The method comprises:
placing liquid specimen sample 22 in tubular container 1630 of filtration assembly 1624, such as shown in FIGS. 2A and 3A;
inserting plunger head 1642 of plunger 1640 into tubular container 1630 via proximal container opening 1632 of tubular container 1630, such as shown in the transition between FIGS. 2A and 2B and between FIGS. 3A and 3B;
distally advancing plunger head 1642 within tubular container 1630 to drive at least a portion of liquid specimen sample 22 through filter 60 disposed in tubular container 1630, such as shown in FIGS. 2B and 3B (it is noted that in this configuration, filter 60 is not initially disposed in tubular container 1630 when liquid specimen sample 22 is placed in tubular container 1630, and is inserted into tubular container 1630 as plunger head 1642 is inserted into tubular container); and
removing filter 60 from tubular container 1630 via plunger-space proximal opening 1690 while plunger head 1642 and filter support 1662 remain within tubular container 1630, as shown in FIGS. 2C-D and 3C-D.

For some applications, liquid specimen sample 22 may be acquired and/or may have any of the characteristics described hereinabove.

For some applications, the method further comprises sealing vial opening 1652 with a collection vial cap after filter 60 has been advanced into collection vial 1650.

For some applications, the method further comprises, after filter 60 has been removed from tubular container 1630, detecting the presence of a biological particulate trapped by filter 60. For example, the biological particulate may be selected from the group consisting of: a virus, a bacterium, a microorganism, a fungus, a spore, a mite, a biological cell, a biological antigen, a protein, a protein antigen, and a carbohydrate antigen.

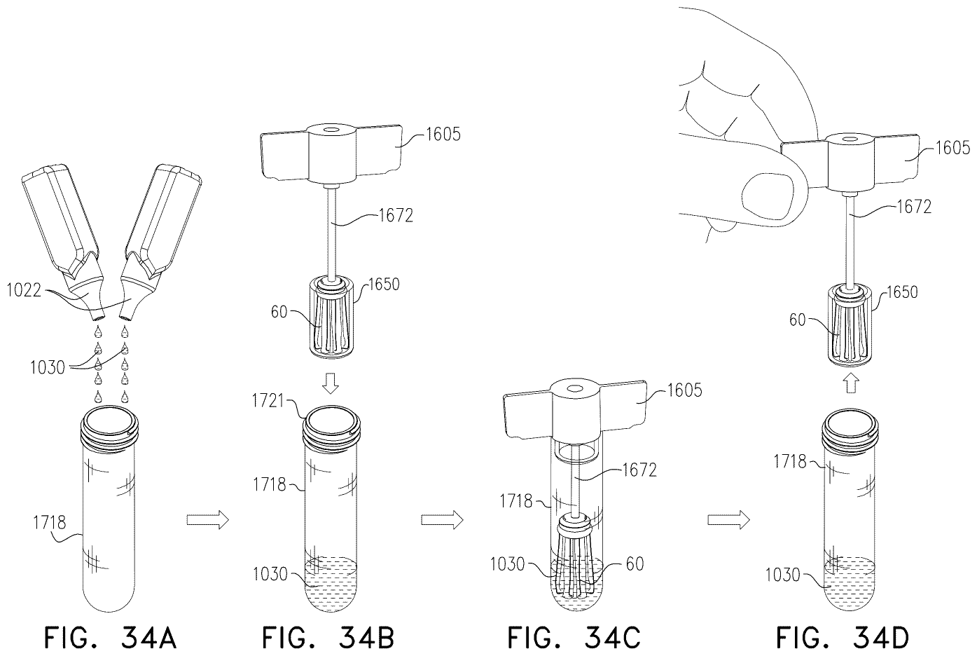

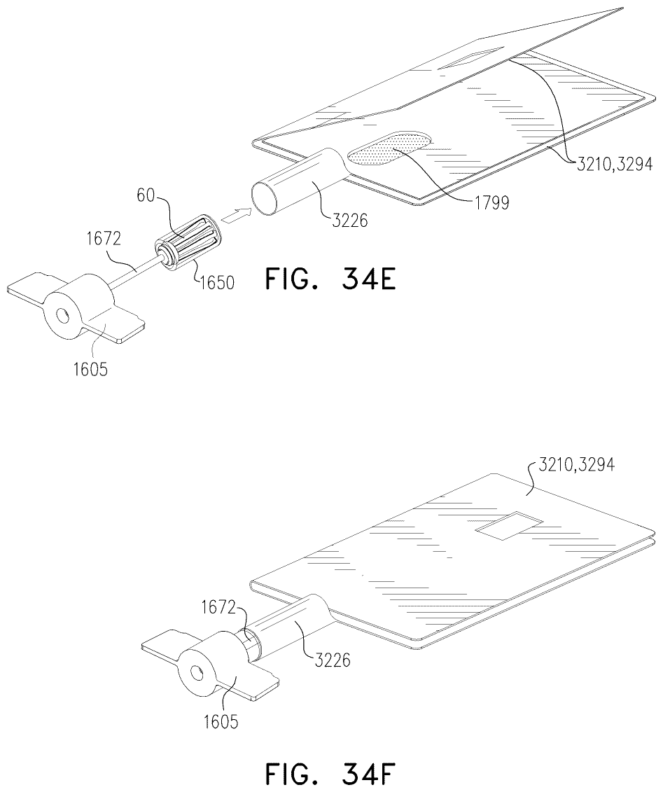

For some applications, such as shown in FIGS. 3I, 3K, 9D, 32K, 33A-C, and 34E-F, detecting the presence of the biological particulate trapped by filter 60 comprising using lateral flow test strip 1799 (e.g., a lateral flow immunoassay test strip) to detect the presence of the biological particulate trapped by filter 60. The lateral flow test strip is optionally contained in a housing 1710 (e.g., comprising a cartridge 1789 (also known as a cassette) or a card 3294, such as described hereinbelow with reference to FIG. 34E-F), or implemented as a dipstick 1757, as is known in the lateral flow art, such as shown in FIG. 3K.

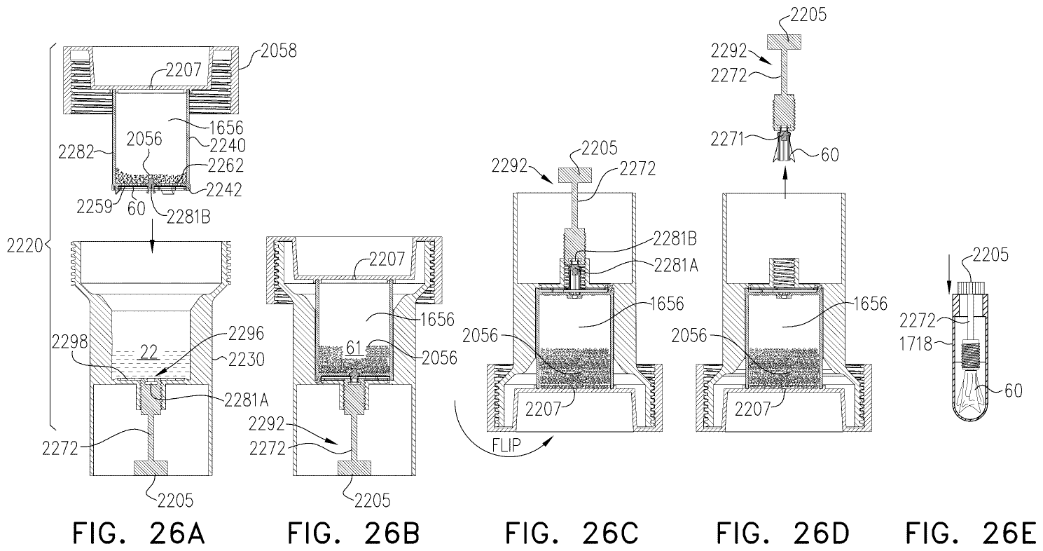

For some applications, filter-withdrawal shaft 1672 is not pre-coupled to filter 60 (configuration not shown, but optionally may be similar to the configuration described hereinbelow with reference to FIGS. 26A-E, mutatis mutandis). Instead, the filter-withdrawal shaft is advanced within internal plunger space 1686 (and optionally inserted into internal plunger space 1686) and coupled to filter 60 after plunger 1640 has been inserted into tubular container 1630 (and optionally been moved within tubular container 1630 to push the at least a portion of liquid specimen sample 22 through filter 60).

For some applications, the method further comprises bathing filter 60 with liquid 1030 within collection vial 1650 (or extraction tube 1718, if provided) after filter 60 has been advanced into collection vial 1650 (and into extraction tube 1718, if provided). For example, the liquid 1030 may be selected from the group consisting of: a lysis buffer, an extraction buffer, saline solution, a transport medium, and one or more reagents, such as one or more reagents for use in a lateral flow test.

In any of the applications of the present invention described herein, liquid 1030 may comprise two or more liquids that are combined (and optionally mixed together), typically during the testing procedure, for example as described hereinbelow with reference to FIGS. 32B and 34A. In any of the applications of the present invention described herein, liquid 1030 may comprise one or more liquids that are combined with a solid, such as a powder (and optionally mixed together), typically during the testing procedure; for example, the powder may be provided contained within one of the tubes described herein, such as one of the extraction tubes described herein.

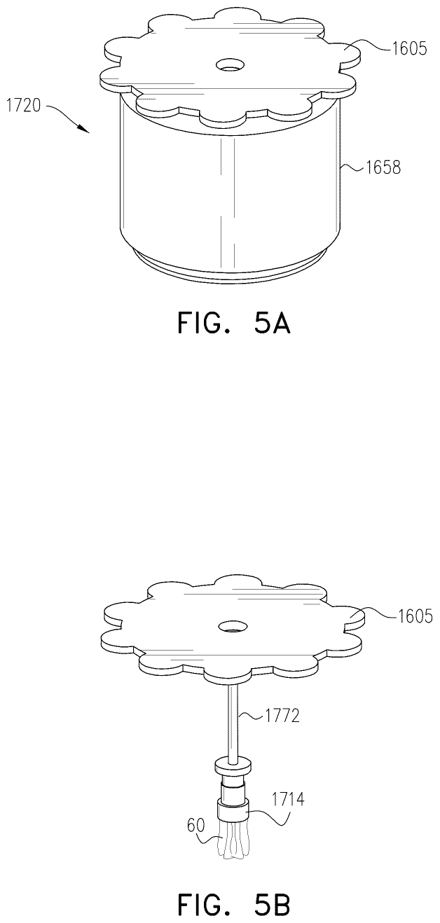

Reference is now made to FIGS. 5A-B, which are schematic illustrations of a sampling device 1720 for concentrating liquid specimen sample 22, and a portion of the sampling device, respectively, in accordance with an application of the present invention.

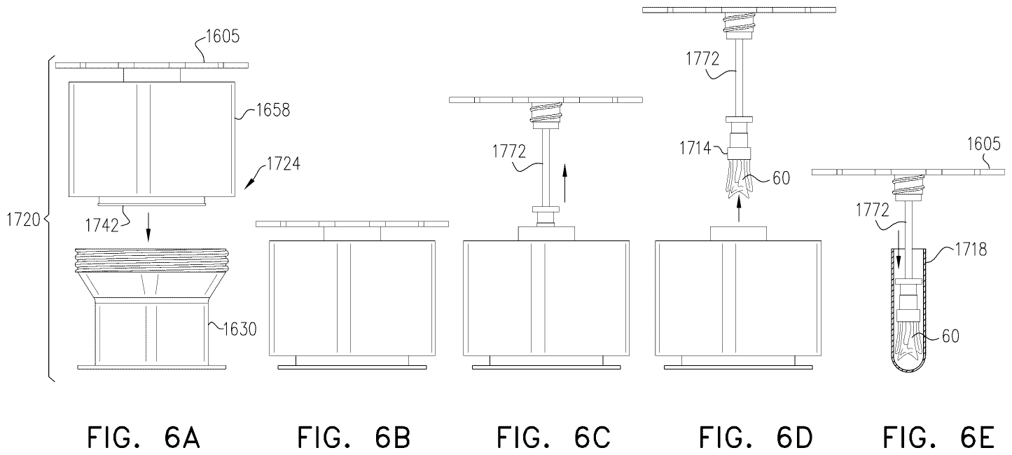

Reference is also made to FIGS. 6A-E, which are schematic illustrations of sampling device 1720 and a method of using sampling device 1720, in accordance with respective applications of the present invention.

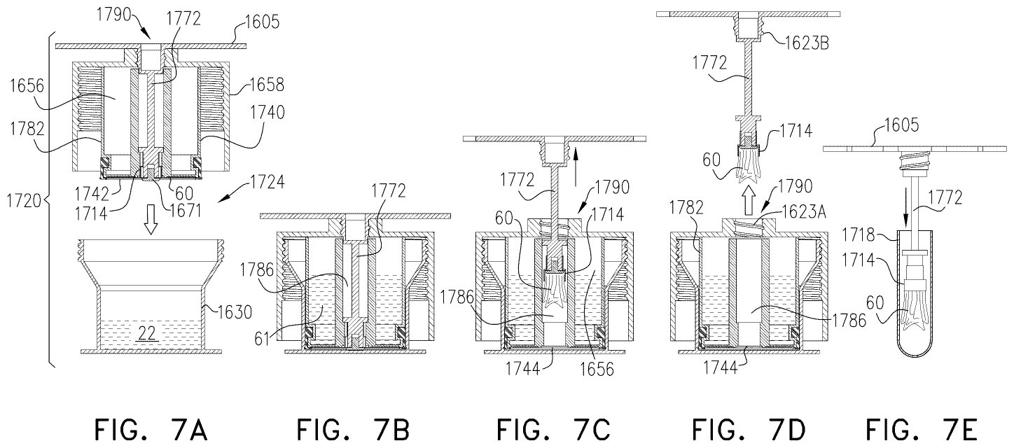

Reference is also made to FIGS. 7A-E, which are schematic cross-sectional illustrations of sampling device 1720 and the method of using sampling device 1720, in accordance with respective applications of the present invention.

Other than as described hereinbelow, sampling device 1720 is generally similar to sampling device 1620 described hereinabove with reference to FIGS. 1A-4, and may implement any of the features thereof, mutatis mutandis. Like reference numerals refer to like parts.

Similar to sampling device 1620, sampling device 1720 typically comprises a filtration assembly 1724, which may have any of the properties described hereinabove. Filtration assembly 1724 comprises tubular container 1630, a plunger 1740, and filter 60, which may have any of the properties described hereinabove with reference to FIGS. 1A-4. However, sampling device 1720 does not comprise a collection vial configured or disposed in a similar manner to collection vial 1650. Nevertheless, sampling device 1720 may optionally comprise extraction tube 1718, such as described hereinbelow with reference to FIGS. 6E and 7E.

Plunger head 1742 is shaped so as to define a plunger-head opening 1744 through plunger head 1742 and into an internal plunger space 1786 of a plunger rod 1782. A proximal end of plunger rod 1782 is shaped so as to define a plunger-space proximal opening 1790 of internal plunger space 1786.

Sampling device 1720 comprises a filter-withdrawal shaft 1772, which includes a distal portion 1708 (labeled in FIG. 7D) that is directly or indirectly coupled to filter 60, and which is disposed passing through internal plunger space 1786. Exemplary ways in which distal portion 1708 of filter-withdrawal shaft 1772 may be directly or indirectly coupled to filter 60 include, but are not limited to:
distal portion 1708 of filter-withdrawal shaft 1772 may be directly coupled to filter 60, such as shown in FIG. 4, e.g., by an adhesive and/or by distal plate 1671, which may be fixed, e.g., pinned, to the distal end of the distal portion of the filter-withdrawal shaft; and
distal portion 1708 of filter-withdrawal shaft 1772 may be indirectly coupled to filter 60, such as shown in FIG. 4 for filter-withdrawal shaft 1672, e.g., by a rod that is fixed to the distal end of the distal portion of the filter-withdrawal shaft and optionally further by an adhesive and/or by distal plate 1671 which may be integral with the rod or fixed to the rod.

It will be appreciated by persons skilled in the art who have read the present application that the distal portion of the filter-withdrawal shaft may be directly or indirectly coupled to the filter in additional ways, all of which are within the scope of the present invention.

Sampling device 1720 is configured such that proximal withdrawal of filter-withdrawal shaft 1772 out of internal plunger space 1786, while plunger head 1742 and filter support 1662 remain within tubular container 1630, pulls filter 60 into internal plunger space 1786 via plunger-head opening 1744 (which is defined by a central opening of filter support 1662) and out of internal plunger space 1786 via plunger-space proximal opening 1790, and removes filter-withdrawal shaft 1772 and filter 60 from filtration assembly 1724. At least a portion of filter 60 is typically bunched up, such as into a flower-like arrangement, from the filter's initial flat shape while disposed on filter support 1662.

It is noted that filter-withdrawal shaft 1772 of sampling device 1720 is not an element of filtration assembly 1724, but instead is removable therefrom, as shown in FIGS. 6D-E and 7D-E.

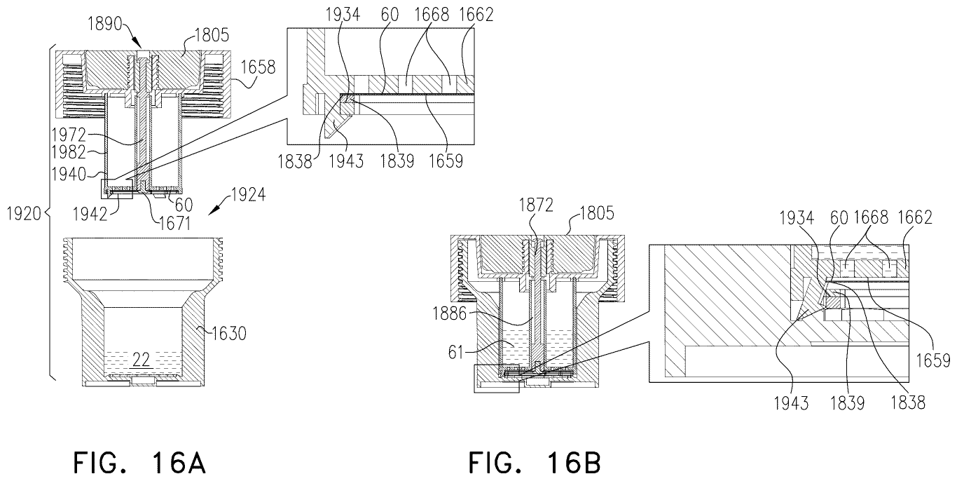

Typically, but not necessarily, after filter-withdrawal shaft 1772 and filter 60 have been removed from filtration assembly 1724, filter 60 and at a portion of filter-withdrawal shaft 1772 are inserted into extraction tube 1718, such as shown in FIGS. 6E and 7E. As mentioned below, the bunching up of at least a portion of filter 60 may help facilitate this insertion; in some respects, the bunched-up filter may function somewhat analogously to a conventional swab. One or more reagents may also be placed in the extraction tube 1718, before or after insertion of filter 60, as known in the diagnostic testing arts. Optionally, extraction tube 1718 implements all or a portion of the techniques described hereinabove with reference to FIGS. 16A-C in PCT Publication WO 2022/149135 to Feldman et al., mutatis mutandis.

For some applications, sampling device 1720 comprises a filter receptacle 1714 that is slidably coupled to a distal end portion of filter-withdrawal shaft 1772 and shaped so as to define a distal receptacle opening 1716. Sampling device 1720 is configured such that the proximal withdrawal of filter-withdrawal shaft 1772 out of internal plunger space 1786, while plunger head 1742 remains within tubular container 1630, pulls a portion of filter 60 into filter receptacle 1714 via distal receptacle opening 1716. For example, the portion of filter 60 may include a central portion (as viewed when filter 60 is flat). This pulling of the portion of filter 60 into filter receptacle 1714 typically causes the remainder of filter 60 to become bunched up, such as into a flower-like arrangement, and be disposed at least partially outside filter receptacle 1714, from the filter's initial flat shape while disposed on filter support 1662. The bunched-up filter may function somewhat analogously to a conventional swab, and, because of the reduced diameter because of the bunching up, may be readily inserted into extraction tube 1718, such as described above and shown in FIGS. 6E and 7E.

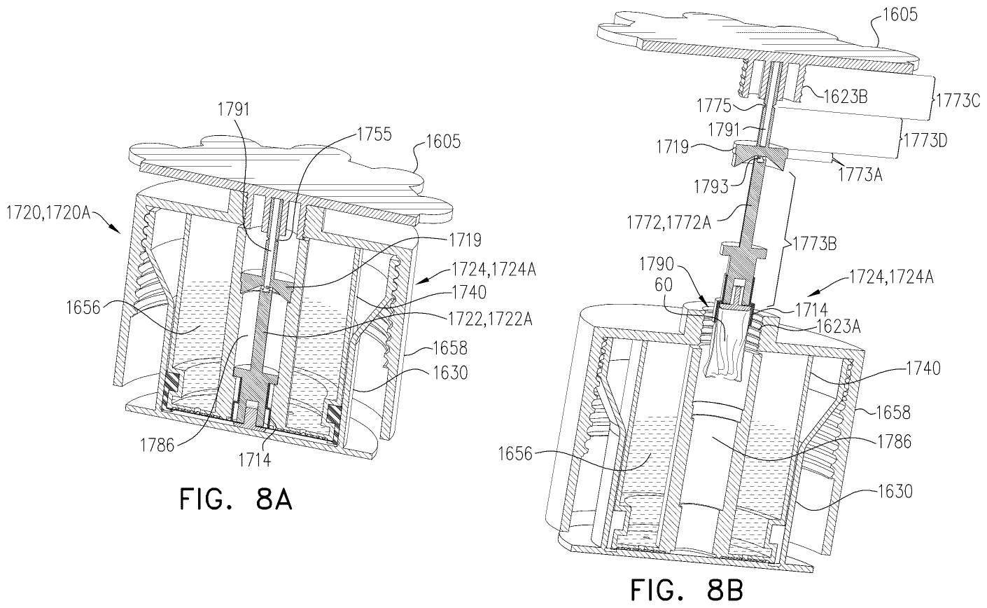

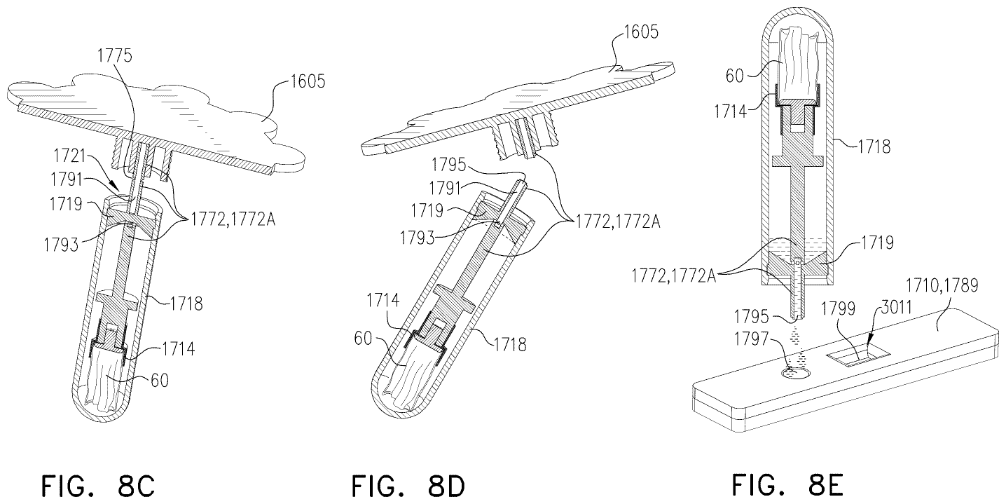

Reference is still made to FIGS. 5A-7E, and is further made to FIGS. 8A-E, which are schematic illustrations of a sampling device 1720, 1720A and a method of using sampling device 1720, 1720A, in accordance with respective applications of the present invention. Sampling device 1720A is one implementation of sampling device 1720, described hereinabove with reference to FIGS. 5A-7E.

Reference is additionally made to FIGS. 9A-D, which are schematic illustrations of a portion of a sampling device 1720, 1720B and a method of using sampling device 1720, 1720B, in accordance with respective applications of the present invention. Sampling device 1720B is one implementation of sampling device 1720, described hereinabove with reference to FIGS. 5A-7E.

Sampling device 1720, 1720A, 1720B further comprises an extraction-tube cap 1719, which is disposed radially surrounding filter-withdrawal shaft 1772 along a first longitudinal portion 1773A of filter-withdrawal shaft 1772, such that a distal second longitudinal portion 1773B of filter-withdrawal shaft 1772 is distal to extraction-tube cap 1719 (labeled in FIGS. 8B and 9A). Typically, extraction-tube cap 1719 has an outer diameter that is greater than an outer diameter of filter-withdrawal shaft 1772. Extraction-tube cap 1719 and filter-withdrawal shaft 1772 may be integral with each other, or may comprise separate pieces that are coupled together, typically during manufacture.

Reference is still made to FIGS. 8A-E and 9A-D. For some applications, filter-withdrawal shaft 1772, 1772A, 1772B is shaped so as to define, proximal to extraction-tube cap 1719, a proximal third longitudinal portion 1773C that is configured to provide a predetermined separation border 1775 between proximal third longitudinal portion 1773C and filter-withdrawal shaft 1772 distal to predetermined separation border 1775. For these applications, a method of using sampling device 1720, 1720A, 1720B further comprises separating, at predetermined separation border 1775, proximal third longitudinal portion 1773C from filter-withdrawal shaft 1772, 1772A, 1772B distal to predetermined separation border 1775, such as shown in the transition between FIG. 8C8C and FIG. 8D.

For some applications, such as shown, proximal third longitudinal portion 1773C is longitudinally connected to first longitudinal portion 1773A of filter-withdrawal shaft 1772 by a fourth longitudinal portion 1773D of filter-withdrawal shaft 1772 longitudinally between predetermined separation border 1775 and extraction-tube cap 1719. For other applications, proximal third longitudinal portion 1773C is directly longitudinally adjacent extraction-tube cap 1719 (configuration not shown).

For some applications, predetermined separation border 1775 is defined by one or more of the following features:
a lesser cross-sectional area of filter-withdrawal shaft 1772 at predetermined separation border 1775 than longitudinally adjacent to predetermined separation border 1775, such as shown in the figures,
scoring, such as shown in the figures,
perforation (configuration not shown),
corresponding male and female screw threads (configuration not shown), and/or
corresponding male and female tapered friction-fitting surfaces (configuration not shown).

For some applications, a method of using sampling device 1720 comprises separating, at predetermined separation border 1775, proximal third longitudinal portion 1773C from filter-withdrawal shaft 1772 distal to predetermined separation border 1775. For applications in which predetermined separation border 1775 is defined by the lesser cross-sectional area of filter-withdrawal shaft 1772 at predetermined separation border 1775 than longitudinally adjacent to predetermined separation border 1775, separating comprises breaking filter-withdrawal shaft 1772 at predetermined separation border 1775. For applications in which predetermined separation border 1775 is defined by scoring or perforation, separating comprises breaking filter-withdrawal shaft 1772 at predetermined separation border 1775. For applications in which predetermined separation border 1775 is defined by corresponding male and female screw threads, separating comprises unscrewing the male and the female screw threads from each other. For applications in which predetermined separation border 1775 is defined by corresponding male and female tapered friction-fitting surfaces, separating comprises separating the male and the female tapered friction-fitting surfaces from each other.

Reference is again made to FIGS. 8A-E. For some applications, filter-withdrawal shaft 1772, 1772A is shaped so as to define an internal channel 1791 that passes longitudinally through first longitudinal portion 1773A and has:
one or more distal openings 1793 (e.g., lateral openings, as shown) distal to extraction-tube cap 1719, and
a proximal opening 1795 that is disposed at predetermined separation border 1775 and that is open to outside filter-withdrawal shaft 1772 upon separation of proximal third longitudinal portion 1773C at predetermined separation border 1775 (and is typically sealed to outside filter-withdrawal shaft 1772 before separation of proximal third longitudinal portion 1773C at predetermined separation border 1775).

Reference is still made to FIGS. 8A-E. For some applications in which sampling device 1720, 1720A further comprises extraction tube 1718 having proximal end opening 1721, filter-withdrawal shaft 1772, 1772A, filter 60, extraction tube 1718, and extraction-tube cap 1719 are configured such that filter 60 and distal second longitudinal portion 1773B of filter-withdrawal shaft 1772 are disposable within extraction tube 1718 via proximal end opening 1721 of extraction tube 1718, such that extraction-tube cap 1719 seals at least a portion of extraction tube 1718 other than allowing fluid flow through internal channel 1791 via one or more distal openings 1793 and proximal opening 1795. For these applications, a method of using sampling device 1720, 1720A further comprises expelling (e.g., squeezing or dripping) a liquid through internal channel 1791 and out of proximal opening 1795. For example, the liquid may comprise one or more reagents in which filter 60 was inserted. For example, the liquid may be expelled (e.g., squeezed or dripped) onto a diagnostic test, such as onto sample pad 1797 of lateral flow test strip 1799, such as a lateral flow immunoassay test strip.

For some applications (configuration not shown), filter-withdrawal shaft 1772, 1772A is not shaped so as to define predetermined separation border 1775. For some of these applications (configuration not shown), filter-withdrawal shaft 1772, 1772A is shaped so as to define an internal channel that passes longitudinally through filter-withdrawal shaft 1772, 1772A and is open at a proximal end of the filter-withdrawal shaft. Typically, the proximal opening of the internal channel is sealed by a removable plug.

Reference is made to FIGS. 9A-D. For some applications in which sampling device 1720 further comprises extraction tube 1718 having proximal end opening 1721, filter-withdrawal shaft 1772B, filter 60, extraction tube 1718, and extraction-tube cap 1719 are configured such that filter 60 and distal second longitudinal portion 1773B of filter-withdrawal shaft 1772 are disposable within extraction tube 1718 via proximal end opening 1721 of extraction tube 1718, such that extraction-tube cap 1719 seals at least a portion of extraction tube 1718. Optionally, extraction tube 1718 comprises a screw-off distal tip cap 1749 that removably seals a distal end 1751 of extraction tube 1718 opposite proximal end opening 1721. Upon removal of distal tip cap 1749, such as shown in FIG. 9C, liquid can be expelled (e.g., squeezed or dripped out) of extraction tube 1718 via an opening through distal end 1751, such as shown in FIG. 9D.

Reference is now made to FIG. 9E, which is a schematic illustration of an alternative configuration of extraction tube 1718, in accordance with an application of the present invention. This configuration may optionally be used in combination with any of the extraction tubes described herein. In this configuration, screw-off distal tip cap 1749 comprises a pin 1753 that extends out of the threaded end of the cap and is sized to be inserted into a channel defined through a threaded nipple 1755 of extraction tube 1718. When screw-off distal tip cap 1749 is coupled to threaded nipple 1755, pin 1753 occupies most or all of the volume of the channel, thereby reducing the amount of liquid 1030 that enters the channel. This reduces the volume of liquid 1030 that must be placed in extraction tube 1718 in order to bathe filter 60. If pin 1753 were not provided, and instead a small additional volume were provided to fill the channel as well as bathe the filter results, the additional volume would further dilute the target analyte released from biological particulate trapped by the filter, possibly reducing the sensitivity of the diagnostic test performed on the portion of the liquid.

Reference is now made to FIGS. 10A-B, which are schematic illustrations of a distal portion of plunger head 1642, in accordance with an application of the present invention. The features of this configuration may also be implemented in plunger heads 1642 and/or 1742, mutatis mutandis, described herein with reference to FIGS. 1A-4 and 5A-9B, respectively. In this configuration, plunger head 1642 further comprises one or more one-way valves 1631 that are configured to provide one-way flow through filtrate-passage openings 1668 into waste liquid receptacle 1656. For example, one-way valves 1631 may comprise flaps. Pressure generated distal to one-way valves 1631 by distal advancement of plunger head 1642 opens the valve and allows filtrate 61 to pass into waste liquid receptacle 1656.

Reference is now made to FIGS. 11A-B, which are schematic illustrations of a sampling device 1820 for concentrating liquid specimen sample 22, and a portion of the sampling device, respectively, in accordance with an application of the present invention.

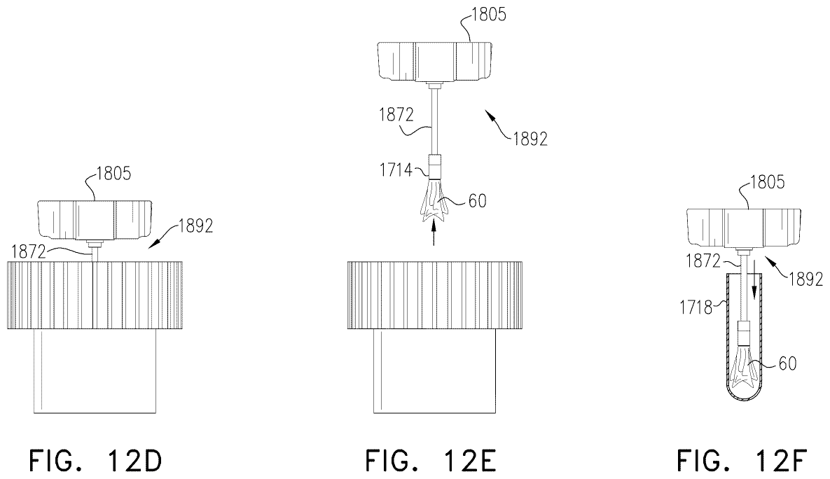

Reference is also made to FIGS. 12A-F, which are schematic illustrations of sampling device 1820 and a method of using sampling device 1820, in accordance with respective applications of the present invention.

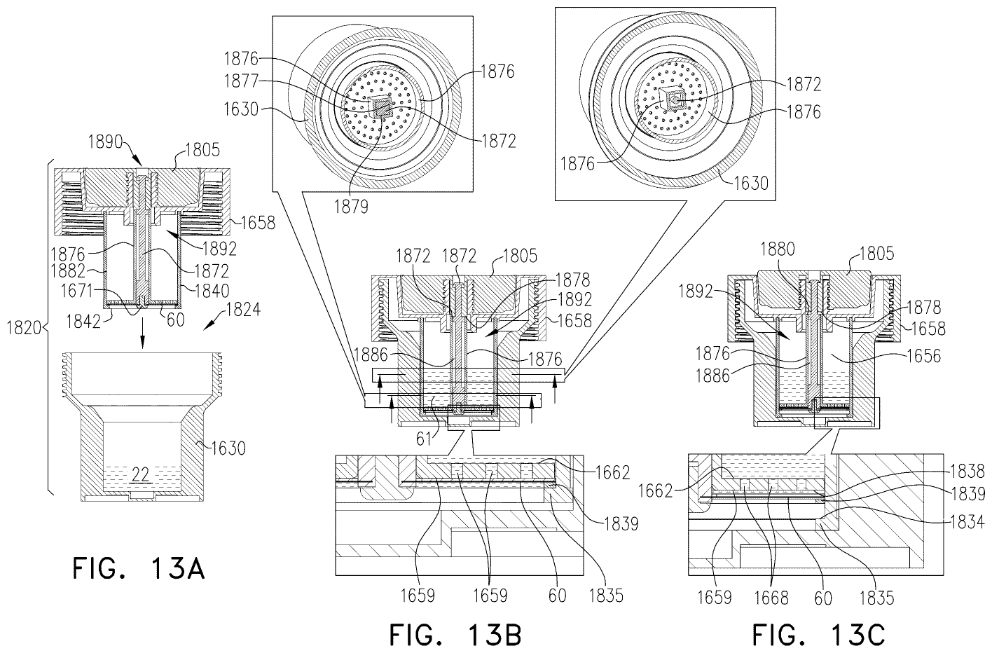

Reference is also made to FIGS. 13A-F, which are schematic cross-sectional illustrations of sampling device 1820 and the method of using sampling device 1820, in accordance with respective applications of the present invention.

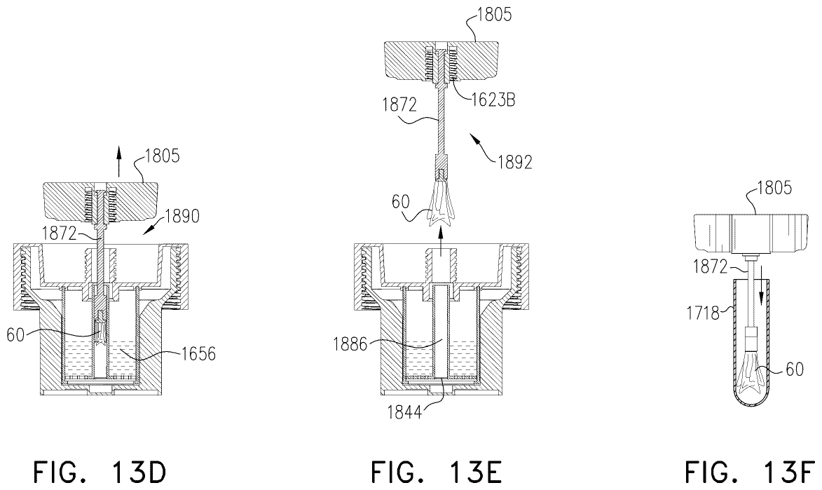

Reference is further made to FIGS. 14A-B, which are schematic illustrations of a sampling device 1920 for concentrating liquid specimen sample 22, and a portion of the sampling device, respectively, in accordance with an application of the present invention.

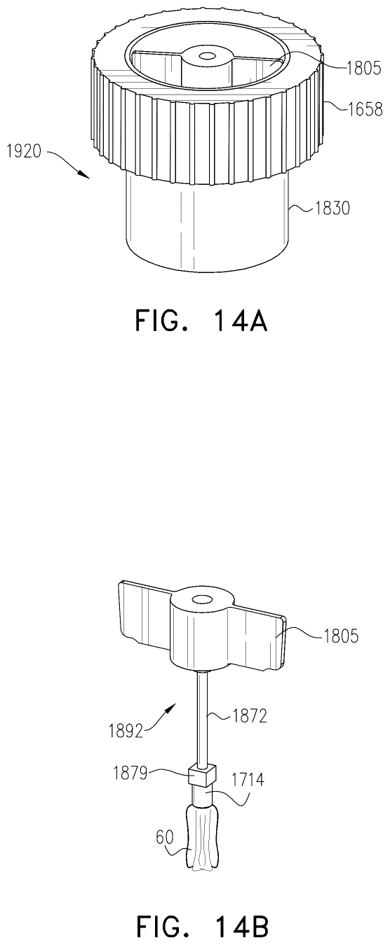

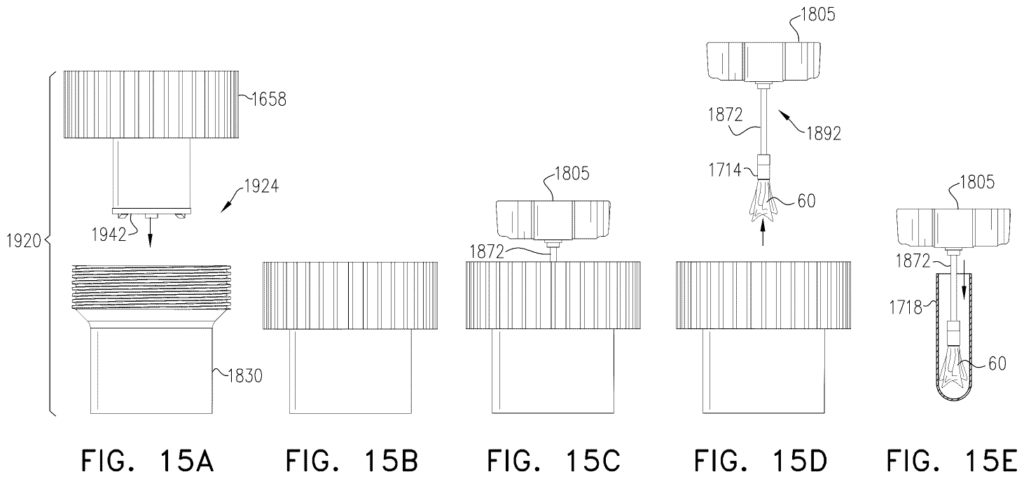

Reference is also made to FIGS. 15A-E, which are schematic illustrations of sampling device 1920 and a method of using sampling device 1920, in accordance with respective applications of the present invention.

Reference is also made to FIGS. 16A-E, which are schematic cross-sectional illustrations of sampling device 1920 and the method of using sampling device 1920, in accordance with respective applications of the present invention.

Other than as described hereinbelow, sampling devices 1820 and 1920 are generally similar to sampling device 1720 described hereinabove with reference to FIGS. 5A-7E, and may implement any of the features thereof, mutatis mutandis. Like reference numerals refer to like parts. The techniques of sampling devices 1820 and 1920 may be implemented in combination with the techniques of any of the other sampling devices described herein, mutatis mutandis. By way of example and not limitation, sampling device 1620, described hereinabove with reference to FIGS. 1A-4, may implement any of the techniques of sampling devices 1820 and 1920, mutatis mutandis.

Similar to sampling device 1720, sampling device 1820 and sampling device 1920 comprise a filtration assembly 1824 and a filtration assembly 1924, respectively, which may have any of the properties described hereinabove. Filtration assembly 1824 and filtration assembly 1924 comprise a tubular container 1830 and filter 60, which may have any of the properties described hereinabove with reference to FIGS. 1A-7E. Filtration assembly 1824 and filtration assembly 1924 further comprise a plunger 1840 and a plunger 1940, respectively. Plungers 1840 and 1940 comprise plunger heads 1842 and 1942, respectively, and plunger rods 1882 and 1982, respectively.

Sampling device 1820 and sampling device 1920 comprise a filter-withdrawal shaft 1872, which includes a distal portion that is coupled (directly or indirectly) to filter 60, and which is disposed passing through an internal plunger space 1886.

For some applications, sampling devices 1820 and 1920 further comprise a shaft handle 1805, which is coupled to a proximal portion of filter-withdrawal shaft 1872. Shaft handle 1805 may have any appropriate shape, for example the shape of wingnut (as shown) or a circular shape (configuration not shown).

Sampling devices 1820 and 1920 are configured such that proximal withdrawal of filter-withdrawal shaft 1872 out of internal plunger space 1886, while plunger head 1842 or 1942 remains within tubular container 1830, pulls filter 60 into internal plunger space 1886 via a plunger-head opening 1844 (labeled in FIG. 13E and defined by a central opening of filter support 1662) and out of internal plunger space 1886 via a plunger-space proximal opening 1890, and removes filter-withdrawal shaft 1872 and filter 60 from filtration assembly 1824. At least a portion of filter 60 is typically bunched up, such as into a flower-like arrangement, from the filter's initial flat shape while disposed on filter support 1662.

It is noted that filter-withdrawal shaft 1872 of sampling devices 1820 and 1920 is not an element of filtration assembly 1824, but instead is removable therefrom, as shown in FIGS. 12E-F, 13E-F, 15D-E, and 16D-E.

Reference is made to FIGS. 11A-13F and 14A-16E. For some applications, filter support 1662 is shaped so as to define plunger-head opening 1844 through plunger head 1842 or 1942 and into internal plunger space 1886. Sampling devices 1820 and 1920 comprise a withdrawer 1892 comprising filter-withdrawal shaft 1872, which (a) includes a distal portion that is coupled (directly or indirectly) to filter 60, and (b) which is disposed passing through internal plunger space 1886. Sampling devices 1820 and 1920 are configured such that proximal withdrawal of filter-withdrawal shaft 1872 out of internal plunger space 1886, while the plunger head remains within tubular container 1630, pulls filter 60 into internal plunger space 1886 via the plunger-head opening (which is defined by a central opening of filter support 1662) and out of internal plunger space 1886 via the plunger-space proximal opening, and removes filter-withdrawal shaft 1872 and filter 60 from the filtration assembly. Optionally, withdrawer 1892 further comprises shaft handle 1805.

For some applications, filtration assembly 1824 (e.g., plunger-space proximal opening 1890 and/or plunger support 1658) and withdrawer 1892 (either shaft handle 1805 or filter-withdrawal shaft 1872 thereof) are shaped so as to define corresponding screw threads. Sampling devices 1820 and 1920 are configured such that rotation of withdrawer 1892 and plunger-space proximal opening 1890 with respect to each other causes at least an initial portion of the proximal withdrawal of filter-withdrawal shaft 1872 out of internal plunger space 1886. For some applications, the remainder of the proximal withdrawal is performed by simply axially withdrawing withdrawer 1892 once the screw threads have entirely separately (as shown), while for other applications, the screw threads are longer and the remainder of the proximal withdrawal is performed by continuing to rotate withdrawer 1892 (configuration not shown).

For some of these applications, plunger rod 1882 comprises a hollow shaft 1876 that is shaped so as to define internal plunger space 1886 within hollow shaft 1876. As mentioned above, filter-withdrawal shaft 1872 is disposed passing through internal plunger space 1886.

For some of these applications in which sampling devices 1820 and 1920 are configured such that rotation of withdrawer 1892 and plunger-space proximal opening 1890 with respect to each other causes at least an initial portion of the proximal withdrawal of filter-withdrawal shaft 1872 out of internal plunger space 1886, sampling devices 1820 and 1920 are configured such that filter-withdrawal shaft 1872 does not rotate during at least an initial portion of the proximal withdrawal of filter-withdrawal shaft 1872 out of internal plunger space 1886. For example (labeled in FIG. 13B):
shaft handle 1805 of withdrawer 1892 is shaped so as to define the screw thread of withdrawer 1892,
shaft handle 1805 and filter-withdrawal shaft 1872 may be coupled together such that shaft handle 1805 is free to rotate with respect to filter-withdrawal shaft 1872,
hollow shaft 1876 may be shaped so as to define a non-circular inner surface 1877 along at least a longitudinal portion of hollow shaft 1876,
filter-withdrawal shaft 1872 may be shaped so as to define a non-circular outer surface 1879 along at least a longitudinal portion of filter-withdrawal shaft 1872, and
non-circular outer surface 1879 engages non-circular inner surface 1877 so as to prevent rotation of filter-withdrawal shaft 1872 with respect to hollow shaft 1876 during at least an initial portion of the proximal withdrawal of filter-withdrawal shaft 1872 out of internal plunger space 1886.

The non-circular shapes of non-circular inner surface 1877 and non-circular outer surface 1879 are defined in cross-section perpendicular to a longitudinal axis of filter-withdrawal shaft 1872.

For example, non-circular inner surface 1877 may be polygonal (e.g., square, as shown) or elliptical (configuration not shown). Similarly, for example, non-circular outer surface 1879 may be polygonal (e.g., square, as shown) or elliptical (configuration not shown). Non-circular inner surface 1877 and non-circular outer surface 1879 may have the same or different non-circular shapes.

For other applications, sampling devices 1820 and 1920 are configured such that shaft handle 1805 of withdrawer 1892 is axially fixed to plunger-space proximal opening 1890 (and thus to the housing of the sampling device), such that shaft handle 1805 does not rise with respect to plunger-space proximal opening 1890 (and the housing) upon rotation of shaft handle 1805 with respect to plunger-space proximal opening 1890. Shaft handle 1805 and filter-withdrawal shaft 1872 are threadingly coupled to each other, such that rotation of shaft handle 1805 moves filter-withdrawal shaft 1872 proximally with respect shaft handle 1805 and internal plunger space 1886 (configuration not shown).

Typically, but not necessarily, after filter-withdrawal shaft 1872 and filter 60 have been removed from filtration assembly 1824 or 1924, filter 60 and at a portion of filter-withdrawal shaft 1872 are inserted into extraction tube 1718, such as shown in FIGS. 12F, 13F, 15E, and 16E. As mentioned below, the bunching up of at least a portion of filter 60 may help facilitate this insertion; in some respects, the bunched-up filter may function somewhat analogously to a conventional swab. One or more reagents may also be placed in the extraction tube 1718, before or after insertion of filter 60, as known in the diagnostic testing arts. Optionally, extraction tube 1718 implements all or a portion of the techniques described hereinabove with reference to FIGS. 16A-C in PCT Publication WO 2022/149135 to Feldman et al., mutatis mutandis.

Plunger head 1842 and plunger head 1942 are shaped so as to define a clamping surface 1834 and a clamping surface 1934, respectively. Typically, clamping surfaces 1834 and 1934 face proximally, toward support surface 1659 of filter support 1662. Sampling devices 1820 and 1920 are transitionable from:
a filter-clamping state, such as shown in FIGS. 12A-B, 13A-B, 15A, and 16A, in which filter 60 is removably disposed on support surface 1659 of filter support 1662, clamped axially between clamping surface 1834 or 1934 and a peripheral portion 1838 of support surface 1659, to
a filter-release state, such as shown in FIGS. 12C-E, 13C-E, 15B-D, and 16B-D, in which filter 60 is not clamped axially between clamping surface 1834 and peripheral portion 1838 of support surface 1659.

As shown in FIGS. 12B, 13B, 15A, and 16A, filtration assemblies 1824 and 1942 are configured such that movement of plunger head 1842 or 1942 within tubular container 1830, when liquid specimen sample 22 is contained in tubular container 1830 and the sampling device 1820 or 1920 is in the filter-clamping state, pushes at least a portion of liquid specimen sample 22 through filter 60 and filtrate-passage openings 1668 and into waste liquid receptacle 1656.

As shown in FIGS. 12C-E, 13C-E, 15D, and 16D, sampling devices 1820 and 1920 are configured such that filter 60 is removable from tubular container 1630, such as via plunger-space proximal opening 1890, while plunger head 1842 or 1942, including filter support 1662 thereof, remains within tubular container 1630 and sampling device 1820 or 1920 is in the filter-release state.

Clamping surface 1834 may run partially (e.g., 180-355 degrees), intermittently, or completely (i.e., 360 degrees) around the circumference of support surface 1659 of filter support 1662. For some applications, clamping surface 1834 runs intermittently around peripheral portion 1838 of support surface 1659. In configurations in which clamping surface 1834 runs less than 360 degrees around the circumference of support surface 1659 of filter support 1662, the lack of the clamping surface at one or more circumferential locations may allow pressure release between filter 60 and distal filter support surface 1659, such that any excessive build-up of pressure in liquid specimen sample 22 in tubular container 1830 (for example, resulting from clogging of the filter) escapes around the filter rather than possibly tearing the filter, which may comprise a fine material. This configuration effectively functions as a bypass valve around the filter.

For some applications, peripheral portion 1838 of support surface 1659 includes an outer perimeter of support surface 1659 (as shown), while for other applications, peripheral portion 1838 of support surface 1659 is spaced away from the outer perimeter of support surface 1659 (configuration not shown).

Reference is made to FIGS. 11A-13F. For some applications, plunger head 1842 comprises an internal shelf 1835, which projects radially inwardly and defines clamping surface 1834. Internal shelf 1835 may run partially (e.g., 180-355 degrees), intermittently, or completely (i.e., 360 degrees) around the circumference of plunger head 1842. In configurations in which internal shelf 1835 runs less than 360 degrees around the circumference of plunger head 1842, the lack of the internal shelf at one or more circumferential locations may allow pressure release between filter 60 and distal filter support surface 1659, such that any excessive build-up of pressure in liquid specimen sample 22 in tubular container 1830 (for example, resulting from clogging of the filter) escapes around the filter rather than possibly tearing the filter, which may comprise a fine material. This configuration effectively functions as a bypass valve around the filter.

Reference is still made to FIGS. 11A-13F. For some applications, sampling device 1820 is configured to proximally move filter support 1662 within waste liquid receptacle 1656, so as to transition the sampling device from the filter-clamping state to the filter-release state.

Reference is made to FIGS. 11A-13F. For some of these applications, plunger head 1842 comprises hollow shaft 1876, and hollow shaft 1876 is coupled to filter support 1662. As mentioned above, filter-withdrawal shaft 1872 is disposed passing through internal plunger space 1886. When sampling device 1820 is in the filter-clamping state, hollow shaft 1876 holds filter support 1662 stationary, pressed distally against clamping surface 1834 (e.g., against internal shelf 1835). For example, an internal distal surface 1878 of a shaft handle 1805 may press distally against a proximal end portion 1880 of hollow shaft 1876, such as shown in FIG. 13B. Upon proximal withdrawal of shaft handle 1805 by a certain distance, internal distal surface 1878 moves proximally sufficiently to no longer be pressing against proximal end portion 1880, such as shown in FIG. 13C. As a result, hollow shaft 1876 no longer holds filter support 1662 pressed distally against clamping surface 1834 and the filter support is free to separate from clamping surface 1834, thereby transitioning sampling device 1820 to the filter-release state, such as shown in FIG. 13C. For some application, shaft handle 1805 is configured to be proximally withdrawn by the certain distance by rotation of shaft handle 1805 by a first number of turns, such as in configurations in which shaft handle 1805 is threaded, as described immediately below.

As mentioned above, for some applications, filtration assembly 1824 (e.g., plunger-space proximal opening 1890 and/or plunger support 1658) and withdrawer 1892 (either shaft handle 1805 or filter-withdrawal shaft 1872 thereof) are shaped so as to define corresponding screw threads. Sampling device 1820 is configured such that rotation of withdrawer 1892 and plunger-space proximal opening 1890 with respect to each other:
causes at least an initial portion of the proximal withdrawal of filter-withdrawal shaft 1872 out of internal plunger space 1886, and
proximally moves filter support 1662 within waste liquid receptacle 1656, so as to transition sampling device 1820 from the filter-clamping state to the filter-release state.

Reference is made to FIGS. 11A-13F and 14A-16E. For some applications, plunger head 1842 or 1942 comprises a gasket seal ring 1839, disposed axially between clamping surface 1834 or 1934 (e.g., internal shelf 1835 in FIGS. 11A-13F) and a distal surface of filter 60, in order to better seal filter 60 to peripheral portion 1838 of support surface 1659. Alternatively, gasket seal ring 1839 is not provided, such as to allow pressure release around filter 60, such as described hereinbelow.

Reference is made to FIGS. 14A-16E. For some applications, shelf 1835 (and/or clamping surface 1934) is annular, e.g., defined by a thin washer.

Reference is still made to FIGS. 14A-16E. For some applications, filtration assembly 1924 is configured such that:
as shown in FIGS. 15A and 16A, a first portion of a stroke of the movement of plunger head 1942 within tubular container 1630, when liquid specimen sample 22 is contained in tubular container 1630 and sampling device 1920 is in the filter-clamping state, pushes the at least a portion of liquid specimen sample 22 through filter 60 and filtrate-passage openings 1668 and into waste liquid receptacle 1656, and
as shown in FIGS. 15B and 16B, a second portion of the stroke of the movement of plunger head 1942 within tubular container 1630 transitions sampling device 1920 from the filter-clamping state to the filter-release state.

For some of these applications:
plunger head 1942 comprises one or more tabs 1943, which restrain clamping surface 1934 such that filter 60 is clamped axially between clamping surface 1934 and peripheral portion 1838 of support surface 1659, such as shown, and
filtration assembly 1924 is configured such that the second portion of the stroke of the movement of plunger head 1942 within tubular container 1630 displaces the one or more tabs 1943 such that filter 60 is not clamped axially between clamping surface 1834 and peripheral portion 1838 of support surface 1659, thereby transitioning the sampling device from the filter-clamping state to the filter-release state, as shown in FIGS. 15B and 16B.

For example, filtration assembly 1924 may comprise a protrusion 1945 positioned at or near the distal end of tubular container 1630, which pushes against the one or more tabs 1943 during the second portion of the stroke.

For some applications, filter support 1662 comprises a flexible material that is sufficiently flexible that if pressure should build up too quickly (for example, if energy storage element 2102 cannot absorb the pressure increase quickly enough), a perimeter of filter support 1662 bends upwards away from an internal wall of plunger rod 1882 or 1982 at one or more locations around the wall, and allows a small amount of liquid specimen sample 22 to pass around filter 60. This allows the excessive build-up of pressure in liquid specimen sample 22 (for example, resulting from clogging of the filter) to escape around the filter rather than possibly tearing the filter, which may comprise a fine material. This configuration effectively functions as a bypass valve around the filter.

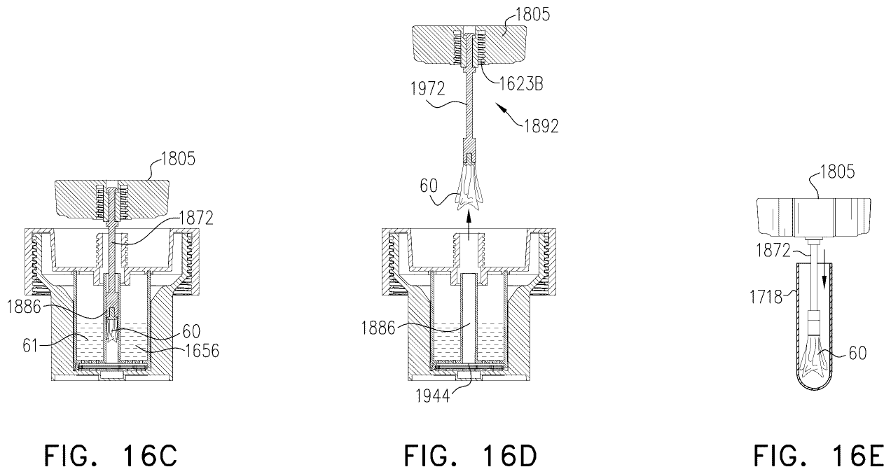

Reference is now made to FIGS. 17A-B, which are schematic illustrations of a sampling device 2020 for concentrating liquid specimen sample 22, and a portion of the sampling device, respectively, in accordance with an application of the present invention.

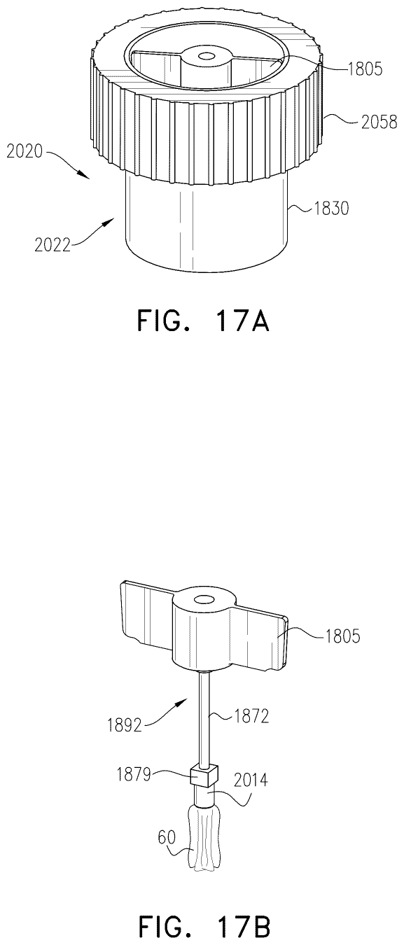

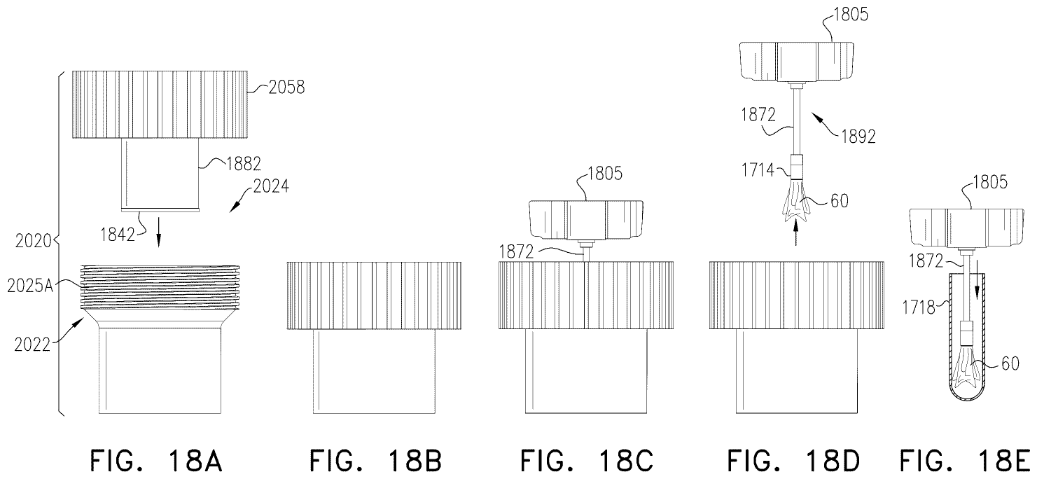

Reference is also made to FIGS. 18A-E, which are schematic illustrations of sampling device 2020 and a method of using sampling device 2020, in accordance with respective applications of the present invention.

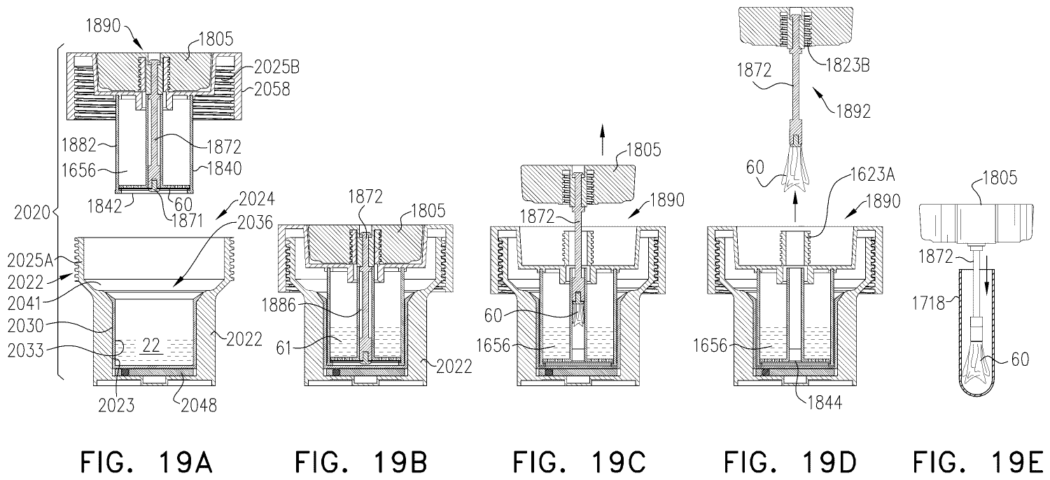

Reference is also made to FIGS. 19A-E, which are schematic cross-sectional illustrations of sampling device 2020 and the method of using sampling device 2020, in accordance with respective applications of the present invention.

Other than as described hereinbelow, sampling device 2020 is generally similar to sampling device 1820 described hereinabove with reference to FIGS. 11A-13F, and may implement any of the features thereof, mutatis mutandis. Like reference numerals refer to like parts. Alternatively or additionally, the features of sampling device 2020 may be implemented in combination with any of the other sampling devices described herein, mutatis mutandis, including, but not limited to sampling device 1620, described hereinabove with reference to FIGS. 1A-4, and/or sampling device 1920, described hereinabove with reference to FIGS. 14A-16E.

Sampling device 2020 comprises a filtration assembly 2024 that comprises a container housing 2022, which is shaped so as to define (a) a cylindrical space 2023 within container housing 2022, and (b) one or more first threads 2025A.

Filtration assembly 2024 comprises, instead of tubular container 1830, a tubular container 2030, which is shaped so as to define an inner wall 2033 and a proximal container opening 2036 for receiving liquid specimen sample 22. Inner wall 2033 is typically not threaded, so as to make a good seal with plunger head 1842, as described hereinbelow; alternatively, the inner wall is threaded. Tubular container 2030 is disposed at least partially within cylindrical space 2023 of container housing 2022, such that tubular container 2030 is rotatable with respect to cylindrical space 2023.

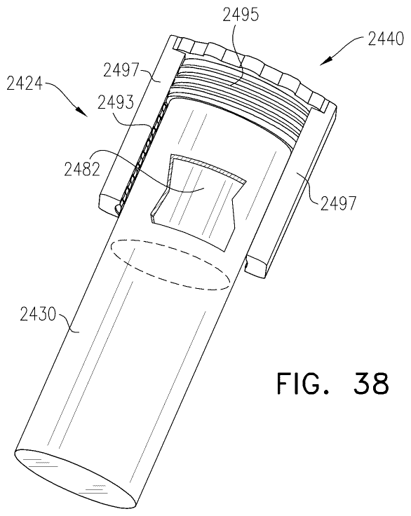

Filtration assembly 2024 further comprises a plunger support 2058, which is coupled to a proximal portion of plunger 1840, and which is shaped so as to define one or more second threads 2025B, shaped so as to engage the one or more first threads 2025A. The one or more first threads 2025A and/or the one or more second threads 2025B may each be a single entire thread, or a plurality of thread segments that do or do not include complete turns, such as described, for example, with reference to FIG. 38 hereinbelow. A portion of plunger support 2058 may serve as a handle to enable easy manipulation of plunger 1840, including insertion of plunger 1840 into tubular container 2030.

Filtration assembly 2024 still further comprises plunger 1840, which (a) comprises plunger head 1842, (b) is insertable into tubular container 2030 via proximal container opening 2036, such that a lateral surface of plunger head 1842 forms a fluid-tight movable seal with the inner wall, and (c) is coupled to plunger support 2058, such that rotation of plunger support 2058 with respect to container housing 2022, when the one or more second threads 2025B are engaged with the one or more first threads 2025A, distally advances plunger support 2058 with respect to container housing 2022 and thus plunger 1840 within tubular container 2030 as tubular container 2030 rotates with respect to container housing 2022.

Filtration assembly 2024 is configured such that movement of plunger head 1842 within tubular container 2030, when liquid specimen sample 22 is contained in tubular container 2030 and filter 60 is disposed in tubular container 2030, pushes at least a portion of liquid specimen sample 22 through filter 60.

For some applications, the one or more first threads 2025A face radially outward, and the one or more second threads 2025B face radially inward, such as show in the figures. For other applications, the one or more first threads 2025A face radially inward, and the one or more second threads 2025B face radially outward (configuration not shown).

Optionally, proximal container opening 2036 is shaped as a funnel, such as shown.

Optionally, container housing 2022 includes a proximal portion 2041 that is proximal to cylindrical space 2023, has a greatest internal diameter that is greater than an internal diameter of cylindrical space 2023, and is shaped so as to define a funnel-shaped portion, such as shown. For example, a distal end of the funnel-shaped portion may be is within 2 cm, such as within 1 cm, of cylindrical space 2023, measured along a central longitudinal axis of cylindrical space 2023.

For some applications, sampling device 2020 further comprises a rotary bearing 2048 to facilitate rotation of tubular container 2030 within container housing 2022. For example, rotary bearing 2048 may comprise a radial ball bearing.

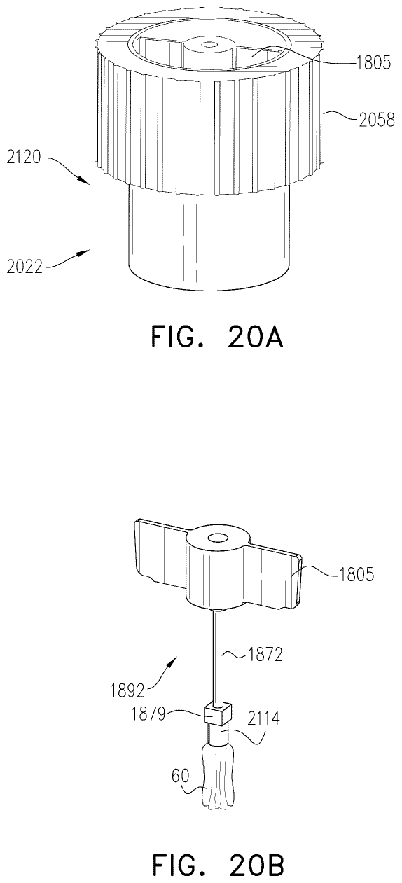

Reference is now made to FIGS. 20A-B, which are schematic illustrations of a sampling device 2120 for concentrating liquid specimen sample 22, and a portion of the sampling device, respectively, in accordance with an application of the present invention.

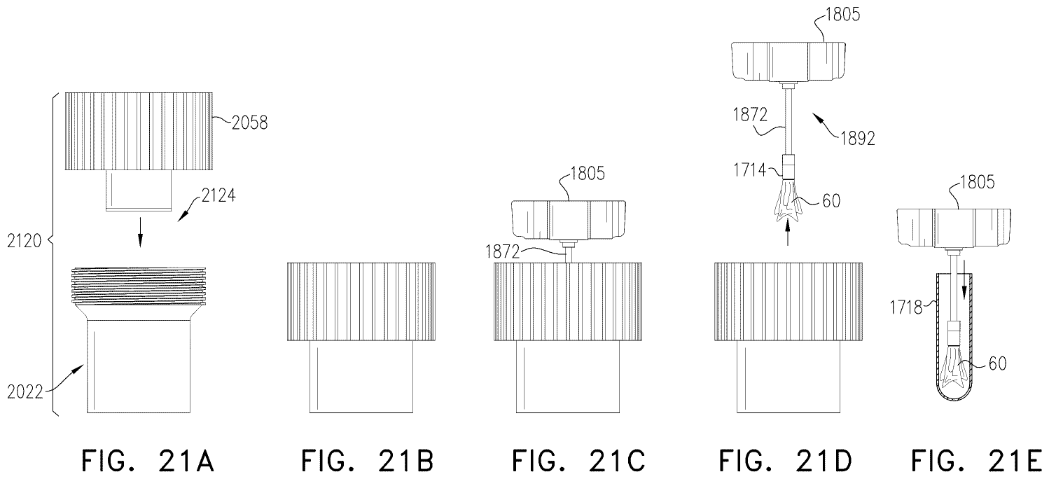

Reference is also made to FIGS. 21A-E, which are schematic illustrations of sampling device 2120 and a method of using sampling device 2120, in accordance with respective applications of the present invention.

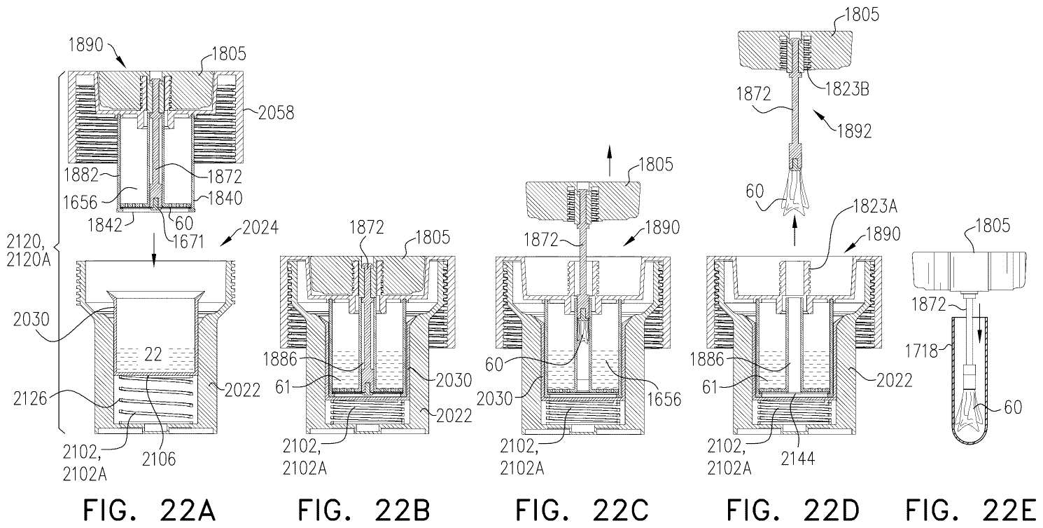

Reference is also made to FIGS. 22A-E, which are schematic cross-sectional illustrations of sampling device 2120, 2120A and the method of using sampling device 2120, 2120A, in accordance with respective applications of the present invention.

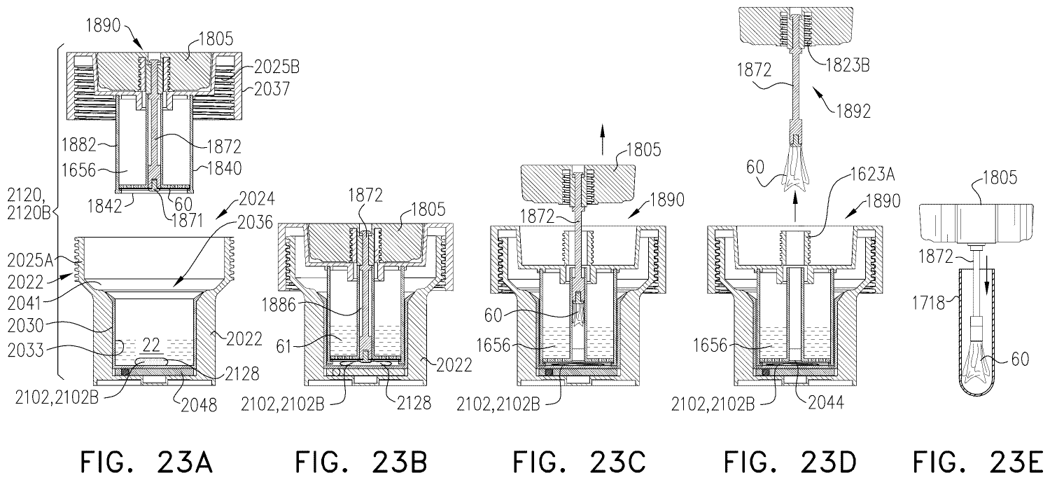

Reference is further made to FIGS. 23A-E, which are schematic cross-sectional illustrations of sampling device 2120, 2120B and the method of using sampling device 2120, 2120B, in accordance with respective applications of the present invention.

Other than as described hereinbelow, sampling device 2120 is generally similar to sampling device 2020 described hereinabove with reference to FIGS. 17A-19E, and may implement any of the features thereof, mutatis mutandis. Like reference numerals refer to like parts. Alternatively or additionally, the features of sampling device 2120 may be implemented in combination with any of the other sampling devices described herein, mutatis mutandis, including, but not limited to sampling device 1620, described hereinabove with reference to FIGS. 1A-4, and/or sampling device 1920, described hereinabove with reference to FIGS. 14A-16E.

Sampling device 2120 further comprises an energy storage element 2102. A filtration assembly 2124 of sampling device 2120 is configured such that movement of plunger head 1842 within tubular container 2030, when liquid specimen sample 22 is contained in tubular container 2030 and filter 60 is disposed on support surface 1659:
pushes at least a portion of liquid specimen sample 22 through filter 60 and filtrate-passage openings 1668 and into waste liquid receptacle 1656, and
stores energy in energy storage element 2102.

Energy storage element 2102 may function as a sort of shock absorber in the event that plunger 1840 is advanced within tubular container 2030 more quickly than liquid specimen sample 22 can pass through filter 60. Energy storage element 2102 transiently reduces the pressure that liquid specimen sample 22 is exerting on the filter. Without this technique, the excessive build-up of pressure in liquid specimen sample 22 in tubular container might possibly clog or tear the filter (which may comprise a fine material) and/or liquid specimen sample 22 may escape tubular container 2030 without passing through filter 60 using techniques for fluid escape described herein.

Reference is made to FIGS. 22A-E. In this configuration, sampling device 2120, 2021A further comprises container housing 2022, and tubular container 2030 is disposed at least partially within container housing 2022, such that tubular container 2030 is moveable with respect to container housing 2022, e.g., axially and/or rotationally moveable with respect to container housing 2022. Energy storage element 2102 is disposed within container housing 2022, outside tubular container 2030 and in direct or indirect contact with an external surface 2106 of tubular container 2030, such that movement of plunger head 1842 within tubular container 2030 moves tubular container 2030 with respect to container housing 2022, thereby storing energy in energy storage element 2012.

For some applications, energy storage element 2102 comprises a mechanical storage element, which comprises an elastic element 2126, such as a spring, a balloon, or soft beads (e.g., comprising silicone), configured to store mechanical energy.

Reference is made to FIGS. 23A-E. In this configuration of sampling device 2120, 2120B, energy storage element 2102 is disposed within tubular container 2030. For some applications, energy storage element 2102 comprises a flexible container 2128 containing a gas, such as air. Flexible container 2128 may or may not be elastic. Optionally, flexible container 2128 is coupled to an internal surface of tubular container 2030, such as a bottom thereof, to prevent the flexible container from floating up and contacting filter 60. Optionally, flexible container 2128 bursts at the end of the plunger stroke when the flexible container is squeezed between plunger head 1842 and the bottom of tubular container 2030.

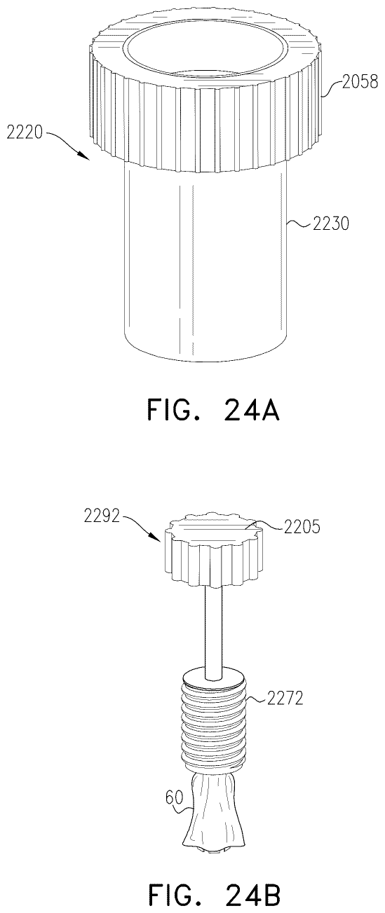

Reference is now made to FIGS. 24A-B, which are schematic illustrations of a sampling device 2220 for concentrating liquid specimen sample 22, and a portion of the sampling device, respectively, in accordance with an application of the present invention.

Reference is also made to FIGS. 25A-E, which are schematic illustrations of sampling device 2220 and a method of using sampling device 2220, in accordance with respective applications of the present invention.

Reference is also made to FIGS. 26A-E, which are schematic cross-sectional illustrations of sampling device 2220 and the method of using sampling device 2220, in accordance with respective applications of the present invention.

Other than as described hereinbelow, sampling device 2220 is similar in many respects to sampling device 1720 described hereinabove with reference to FIGS. 5A-7E, and may implement any of the features thereof, mutatis mutandis. Like reference numerals refer to like parts. The techniques of sampling device 2220 may be implemented in combination with the techniques of any of the other sampling devices described herein, mutatis mutandis, including, but not limited to, comprising collection vial 1650, described hereinabove with reference to FIGS. 1A-4, mutatis mutandis. Although sampling device 2220 is shown, by way of example, as implementing the reversible filter-clamping techniques of sampling device 1920, described hereinabove with reference to FIGS. 14A-16E, sampling device 2220 may alternatively implement other reversible filter-clamping techniques, such as those of sampling device 1820, described hereinabove with reference to FIGS. 11A-13F, mutatis mutandis, or may not implement any reversible filter-clamping techniques.

Similar to sampling device 1720, sampling device 2220 comprises a filtration assembly 2224, which may have any of the properties described hereinabove. Filtration assembly 2224 comprises a tubular container 2230 and filter 60, which may have any of the properties described hereinabove with reference to FIGS. 1A-7E. Filtration assembly 2224 further comprises a plunger 2240, which comprises a plunger head 2242 and a plunger rod 2282.

Sampling device 2220 comprises a withdrawer 2292 comprising a filter-withdrawal shaft 2272, which is disposed passing through a distal opening 2296 defined by a distal bottom surface 2298 of tubular container 2230. Filter-withdrawal shaft 2272 includes a distal portion that is couplable (directly or indirectly) to filter 60. Unlike in some configurations of other sampling devices described, filter-withdrawal shaft 2272 is not initially coupled to filter 60 before use of filtration assembly 2224. Instead, filter-withdrawal shaft 2272 and filter 60 comprise respective couplers 2281A and 2281B, which are configured to be coupled to each other, such as by snapping together, when plunger head 2242 is fully distally advanced within tubular container 2230, pushing filter 60 against a distal end of filter-withdrawal shaft 2272, such as shown in the transition between FIG. 26A and FIG. 26B.

For some applications, withdrawer 2292 further comprises a shaft handle 2205, which is coupled to a proximal portion of filter-withdrawal shaft 2272. Shaft handle 2205 may have any appropriate shape, for example the shape of wingnut (as shown) or a circular shape (configuration not shown).

Plunger head 2242 is shaped so as to define a filter support 2262, which is shaped so as to define:
a support surface 2259, which may be perpendicular to a central longitudinal axis of plunger head 2242 (as shown), or may be angled with respect to the central longitudinal axis (configuration not shown), and
a plurality of filtrate-passage openings through filter support 2262 into waste liquid receptacle 1656.

Unlike filter support 1662, described hereinabove, filter support 2262 is not necessarily shaped so as to define a central opening into waste liquid receptacle 1656.

Filter 60 is (removably) disposed on support surface 2259, typically on an upstream side of support surface 2259 (which, in the configuration of sampling device 2220, is a distal side of support surface 2259).

Filtration assembly 2224 is configured such that movement (typically distal advancement) of plunger head 2242 within tubular container 2230, when liquid specimen sample 22 is contained in tubular container 2230 and filter 60 is disposed in tubular container 2230, pushes at least a portion of liquid specimen sample 22 through filter 60. Filter 60 is configured to concentrate at least a portion of liquid specimen sample 22 onto filter 60, while allowing filtrate 61 to pass through filter 60. Typically, distal advancement of plunger 2240 within tubular container 2230 applies pressure to drive (e.g., push) at least a portion of liquid specimen sample 22 contained in tubular container 2230 through filter 60 and the filtrate-passage openings and into waste liquid receptacle 1656, such as shown in the transitions between FIGS. 25A and 25B and between FIGS. 26A and 26B.

Sampling device 2220 is configured such that distal withdrawal of filter-withdrawal shaft 2272 out of filtration assembly 2224, typically while plunger head 2242 remains within tubular container 2230, pulls filter 60 out of filtration assembly 2224 via distal opening 2296 defined by distal bottom surface 2298 of tubular container 2230, thereby removing filter-withdrawal shaft 2272 and filter 60 from filtration assembly 2224. At least a portion of filter 60 is typically bunched up, such as into a flower-like arrangement, from the filter's initial flat shape while disposed on filter support 2262.

It is noted that filter-withdrawal shaft 2272 of sampling device 2220 is not an element of filtration assembly 2224, but instead is removable therefrom.

For some applications, filtration assembly 2224 (e.g., distal opening 2296) and withdrawer 2292 (either shaft handle 2205 or filter-withdrawal shaft 2272 thereof) are shaped so as to define corresponding screw threads. Sampling device 2220 is configured such that rotation of withdrawer 2292 and distal opening 2296 with respect to each other causes at least an initial portion of the distal withdrawal of filter-withdrawal shaft 2272 out of filtration assembly 2224. For some applications, the remainder of the proximal withdrawal is performed by simply axially withdrawing withdrawer 2292 once the screw threads have entirely separately (as shown), while for other applications, the screw threads are longer and the remainder of the proximal withdrawal is performed by continuing to rotate withdrawer 2292 (configuration not shown).

As mentioned above, waste liquid receptacle 1656 is optionally shaped so as to define an opening 2207 through an external wall of waste liquid receptacle 1656 to release displaced air. For example, the opening may be located on a proximal portion of the external wall, typically above the highest level that filtrate 61 is expected to reach during ordinary use of the device for filtration of liquid specimen sample 22.

For some applications, waste liquid receptacle 1656 contains (a) a disinfectant and/or (b) a liquid-absorbing material 2056, such as sodium polyacrylate or a gel. Liquid-absorbing material 2056, when mixed with filtrate 61 within the waste liquid receptable, absorbs filtrate 61 (e.g., resulting in a gel or solid), in order to reduce risk of leakage of the filtrate from the filtration assembly. Although illustrated only for waste liquid receptacle 1656 of filtration assembly 2224, this feature may be implemented in the waste liquid receptacles of any of the filtration assemblies described herein.

For some applications, as shown in the transition between FIG. 25B and FIG. 25C and between FIG. 26B and FIG. 26C, filtration assembly 2224 is turned over before distal withdrawal of filter-withdrawal shaft 2272 out of filtration assembly 2224. In configurations in which waste liquid receptacle 1656 contains liquid-absorbing material 2056, filtrate 61 is typically absorbed by liquid-absorbing material 2056 before a material amount of liquid can leak through opening 2207.

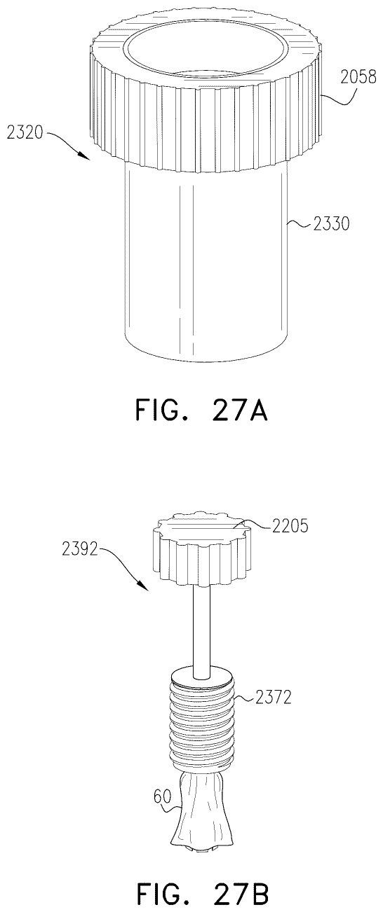

Reference is now made to FIGS. 27A-B, which are schematic illustrations of a sampling device 2320 for concentrating liquid specimen sample 22, and a portion of the sampling device, respectively, in accordance with an application of the present invention.

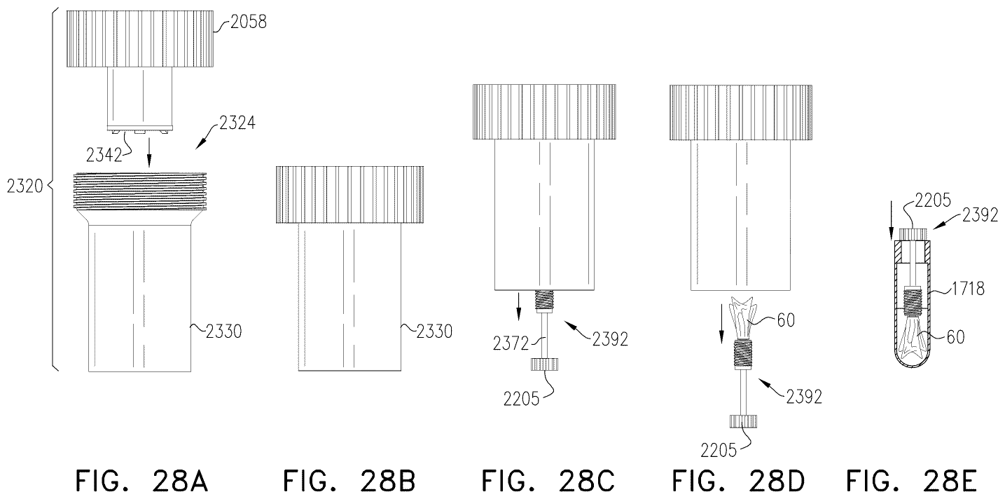

Reference is also made to FIGS. 28A-E, which are schematic illustrations of sampling device 2320 and a method of using sampling device 2320, in accordance with respective applications of the present invention.

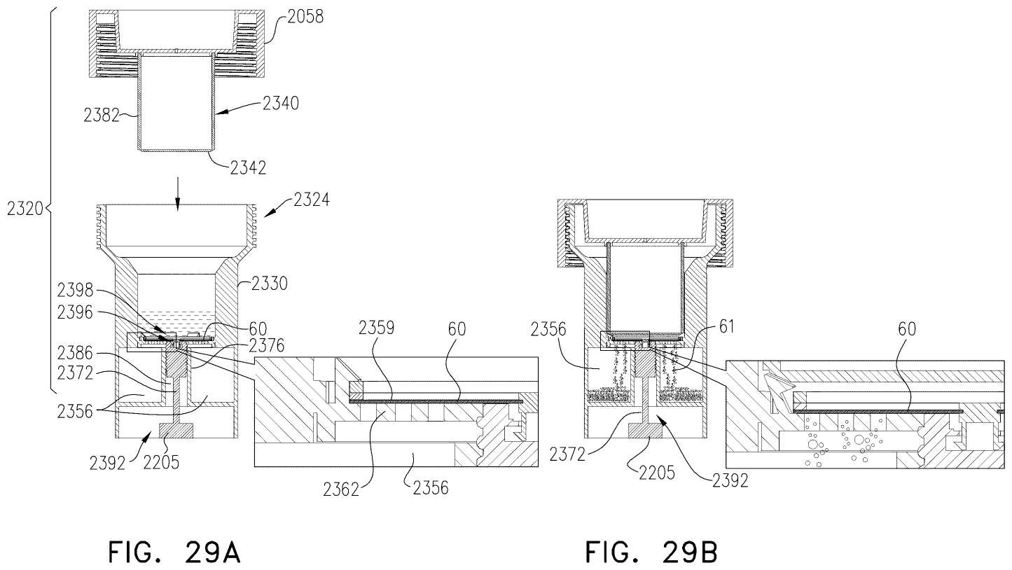

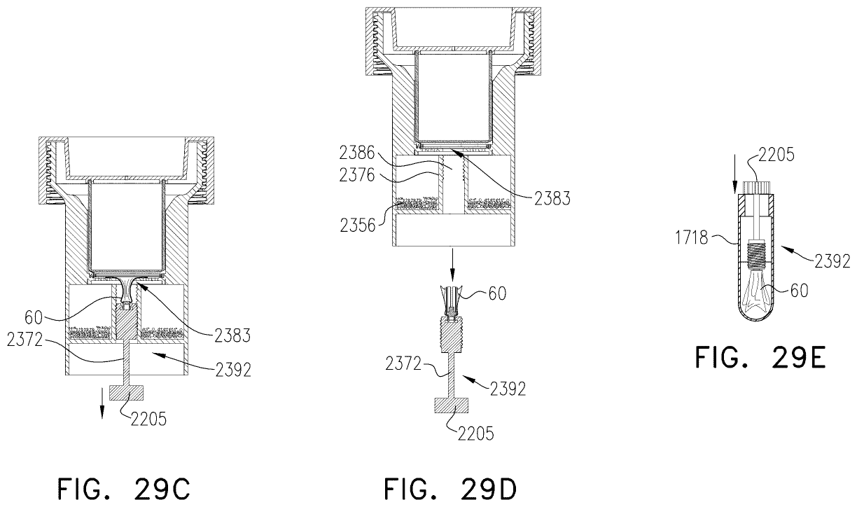

Reference is also made to FIGS. 29A-E, which are schematic cross-sectional illustrations of sampling device 2320 and the method of using sampling device 2320, in accordance with respective applications of the present invention.

Other than as described hereinbelow, sampling device 2320 is similar in many respects to sampling device 2220 described hereinabove with reference to FIGS. 24A-26E, and may implement any of the features thereof, mutatis mutandis. Like reference numerals refer to like parts. The techniques of sampling device 2320 may be implemented in combination with the techniques of any of the other sampling devices described herein, mutatis mutandis, including, but not limited to, comprising collection vial 1650, described hereinabove with reference to FIGS. 1A-4, mutatis mutandis. Although sampling device 2220 is shown, by way of example, as implementing reversible filter-clamping techniques similar to those of sampling device 1920, described hereinabove with reference to FIGS. 14A-16E, sampling device 2220 may alternatively implement other reversible filter-clamping techniques, such as those of sampling device 1820, described hereinabove with reference to FIGS. 11A-13F, mutatis mutandis, or may not implement any reversible filter-clamping techniques.

Sampling device 2320 comprises a filtration assembly 2324, which may have any of the properties described hereinabove. Filtration assembly 2324 comprises a tubular container 2330 and filter 60, which may have any of the properties described hereinabove with reference to FIGS. 1A-7E. Filtration assembly 2324 further comprises a plunger 2340, which comprises a plunger head 2342 and a plunger rod 2382.

Unlike in sampling device 2220, in sampling device 2320 plunger head 2342 is not shaped so as to define a filter support. Instead, tubular container 2330 comprises a filter support 2362, which is disposed near or on a distal bottom surface 2398 of tubular container 2330, or is defined by distal bottom surface 2398. Filter support 2362 is shaped so as to define:
a support surface 2359, which may be perpendicular to a central longitudinal axis of tubular container 2330 (as shown), or may be angled with respect to the central longitudinal axis (configuration not shown),
a plurality of filtrate-passage openings through filter support 2362 into a waste liquid receptacle 2356, which is disposed distal to filter support 2362 and distal bottom surface 2398 of tubular container 2330, and
a central opening 2383 (labeled in FIGS. 29C and 29D).

For some applications, filtration assembly 2324 comprises a hollow shaft 2376, which extends distally from tubular container 2330, and is shaped so as to define an internal shaft space 2386 within hollow shaft 2376 (labeled in FIGS. 29A and 29D). Central opening 2383 is open to internal shaft space 2386.

Sampling device 2320 comprises a withdrawer 2392 comprising a filter-withdrawal shaft 2372, which includes a distal portion that is couplable (directly or indirectly) to filter 60 (for example, as described hereinabove for the other filter-withdrawal shafts described herein, mutatis mutandis). Filter-withdrawal shaft 2372 is disposed passing (a) through a distal opening 2396 defined by distal bottom surface 2398 of tubular container 2330 and (b) optionally, in configurations in which filtration assembly 2324 comprises hollow shaft 2376, through internal shaft space 2386.

For some applications, withdrawer 2392 further comprises shaft handle 2205, which is coupled to a proximal portion of filter-withdrawal shaft 2372. Shaft handle 2305 may have any appropriate shape, for example the shape of wingnut (as shown) or a circular shape (configuration not shown).

Filter 60 is (removably) disposed on support surface 2359, typically on an upstream side of support surface 2359 (which, in the configuration of sampling device 2320, is a proximal side of support surface 2359).

Filtration assembly 2324 is configured such that movement (typically distal advancement) of plunger head 2342 within tubular container 2330, when liquid specimen sample 22 is contained in tubular container 2330 and filter 60 is disposed in tubular container 2330, pushes at least a portion of liquid specimen sample 22 through filter 60. Filter 60 is configured to concentrate at least a portion of liquid specimen sample 22 onto filter 60, while allowing filtrate 61 to pass through filter 60. Typically, distal advancement of plunger 2340 within tubular container 2230 applies pressure to drive (e.g., push) at least a portion of liquid specimen sample 22 contained in tubular container 2330 through filter 60 and the filtrate-passage openings and into waste liquid receptacle 2356, such as shown in the transitions between FIGS. 29A and 29B.

Sampling device 2320 is configured such that distal withdrawal of filter-withdrawal shaft 2372 out of filtration assembly 2324, typically while plunger head 2342 remains within tubular container 2330, pulls filter 60 out of filtration assembly 2324 via distal opening 2396 defined by distal bottom surface 2398 of tubular container 2330 (and optionally via central opening 2383 and/or via internal shaft space 2386, in configurations in which filtration assembly 2324 comprises hollow shaft 2376), thereby removing filter-withdrawal shaft 2372 and filter 60 from filtration assembly 2324. At least a portion of filter 60 is typically bunched up, such as into a flower-like arrangement, from the filter's initial flat shape while disposed on filter support 2362.

It is noted that filter-withdrawal shaft 2372 of sampling device 2320 is not an element of filtration assembly 2324, but instead is removable therefrom.

For some applications, filtration assembly 2324 (e.g., distal opening 2396) and withdrawer 2392 (either shaft handle 2205 or filter-withdrawal shaft 2372 thereof) are shaped so as to define corresponding screw threads. Sampling device 2320 is configured such that rotation of withdrawer 2392 and distal opening 2396 with respect to each other causes at least an initial portion of the distal withdrawal of filter-withdrawal shaft 2372 out of filtration assembly 2324. For some applications, the remainder of the proximal withdrawal is performed by simply axially withdrawing withdrawer 2392 once the screw threads have entirely separately (as shown), while for other applications, the screw threads are longer and the remainder of the proximal withdrawal is performed by continuing to rotate withdrawer 2392 (configuration not shown).

Reference is now made to FIG. 30, which is a schematic illustration of a kit 1000, in accordance with an application of the present invention. Kit 1000 comprises extraction tube 1718, filter 60, liquid 1030 for bathing filter 60 within extraction tube 1718. Liquid 1030 may, for example, comprise one or more reagents, or may, for example, be selected from the group consisting of: a lysis buffer, an extraction buffer, saline solution (e.g., phosphate buffered saline (PBS)), and a transport medium (e.g., a universal transport medium or a viral transport medium). For example, the one or more reagents, which are also known as extraction reagents, may comprise saline, sodium nitrite, acid (e.g., acetic acid or citric acid), detergents, and/or preservatives, which may, for example, release a target analyte (e.g., an antigen) from the particulate, e.g., the cell, bacterium, or virus.

For some applications, kit 1000 comprises a container 1022 that contains liquid 1030; alternatively, extraction tube 1718 contains liquid 1030. For some applications, liquid 1030 has a volume of at least 150 microliters (e.g., at least 200 microliters), no more than 500 microliters (e.g., no more than 300 microliters), and/or 150-500 microliters (e.g., 150-300 microliters, such as 200-300 microliters). The bunched-up shape of filter 60 described herein may facilitate bathing filter 60 in a small amount of liquid 1030. Smaller volumes of liquid 1030 generally result in less dilution of the target analyte released into liquid 1030 from the biological particulate trapped by filter 60.

Kit 1000 further comprises a filter shaft 1072, which includes a distal portion 1008 that is coupled to or couplable to a central portion 1010 of filter 60, and which is configured to insert filter 60 into extraction tube 1718 for bathing filter 60 in the liquid 1030. (It will be appreciated that distal portion 1008 of filter shaft 1072 would not be considered coupled to central portion 1010 of filter 60 if distal portion 1008 of filter shaft 1072 were coupled to a non-central portion of filter 60, such as a peripheral portion of the filter.) For some applications, distal portion 1008 of filter shaft 1072 is coupled to central portion 1010 of filter 60 before use of filter 60 (typically during manufacture), while for other applications, distal portion 1008 of filter shaft 1072 is couplable to central portion 1010 of filter 60 during use of filter 60, such as, by way of example and not limitation, described hereinabove with reference to FIGS. 25A-E and 26A-E.

For some applications, central portion 1010 includes a centroid of filter 60 (which is a center of filter 60 in configurations in which filter 60 is circular). Optionally, central portion 1010 is centered on filter 60.

For some applications, distal portion 1008 of filter shaft 1072 is coupled to central portion 1010 of filter 60 such a closest distance of distal portion 1008 of filter shaft 1072 to any point on a perimeter 1085 of filter 60 is at least 50% (e.g., at least 60%, such as at least 75%) of the square root of the quotient of the surface area of filter 60 divided by π (pi). For configurations in which filter 60 is circular in these applications, the closest distance is at least 50% of the radius of filter 60, e.g., at least 60%, such as at least 75%, of the radius of filter 60.

For some applications, distal portion 1008 of filter shaft 1072 is coupled to central portion 1010 of filter 60 such distal portion 1008 of filter shaft 1072 either covers or is within a distance of a centroid of filter 60 defined by perimeter 1085 of filter 60, the distance less than 50% (e.g., less than 60%, such as less than 75%) of the square root of the quotient of the surface area of filter 60 divided by π (pi). For configurations in which filter 60 is circular in these applications, the distance is less than 50% of the radius of filter 60, e.g., less than 60%, such as less than 75%, of the radius of filter 60.

Optionally, filter shaft 1072 comprises any of the filter-withdrawal shafts described herein and/or in the patent applications or patent application publications incorporated herein by reference; likewise, any of the filter-withdrawal shafts described herein may implement any of the features of filter shaft 1072. For example, filter shaft 1072 may comprise: filter-withdrawal shaft 1672 (and optionally collection vial 1650 may additionally be provided), described hereinabove with reference to FIGS. 1A-4; filter-withdrawal shaft 1772, described hereinabove with reference to FIGS. 5A-7E; filter-withdrawal shaft 1872, described hereinabove with reference to FIGS. 13A-16E; filter-withdrawal shaft 2272, described hereinabove with reference to FIGS. 23A-26E; or filter-withdrawal shaft 2372, described hereinabove with reference to FIGS. 27A-29E.

The configurations described hereinbelow with reference to FIGS. 32A-K, 33A-C, 34A-D, 34E-F, 35A-D, 36A-B, and 37A-B are shown as implementing filter-withdrawal shaft 1672 by way of example and not limitation. These configurations may alternatively implement (a) any of the other filter-withdrawal shafts described herein, (b) filter shaft 1072, or (c) any other appropriately-shaped shaft; the filter shafts in these configurations are not necessarily used for withdrawing the filter from anything, including a sampling device.

Optionally, kit 1000 further comprises a shaft handle 1105, which is coupled to a proximal portion of filter shaft 1072. For example, shaft handle 1105 may have the shape of shaft handle 1605 or shaft handle 1805, described hereinabove.

For some applications, filter shaft 1072 has a length of at least 2 cm, such as at least 3 cm.

For some applications, an average cross-sectional area of filter shaft 1072, measured perpendicular to a longitudinal axis of the filter shaft, equals 1%-15%, such as 1%-10% or 1%-5%, of a surface area of filter 60, measured when filter 60 is flat.

Filter shaft 1072 may be cylindrical, as shown, or may alternatively have another, non-circular cross-sectional shape, such as a polygonal shape (e.g., a rectangular, square, or triangular shape), an “x” shape, or an asterisk shape. Alternatively or additionally, filter shaft 1072 may have different cross-sectional shapes along respective different longitudinal portions therealong; optionally, one or more of the cross-sectional shapes is circular.

For some applications, filter 60 is circular when flat. Alternatively, filter 60 has another shape.

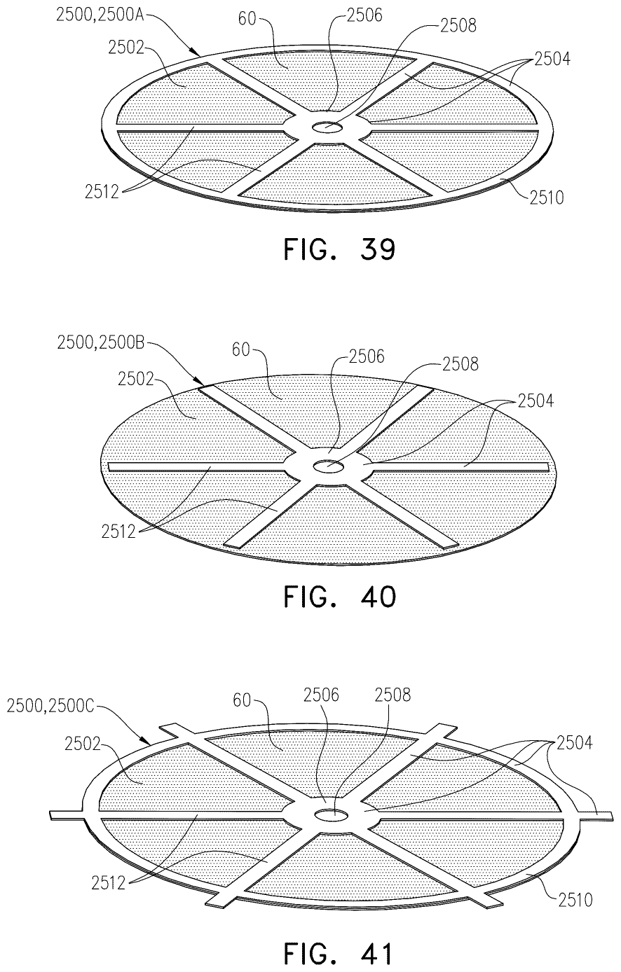

For some applications, filter 60 comprises filter reinforcement 2500, as described hereinbelow with reference to FIGS. 39, 40, 41, and/or 42A-B.

For some applications, extraction tube 1718 comprises a flexible material.

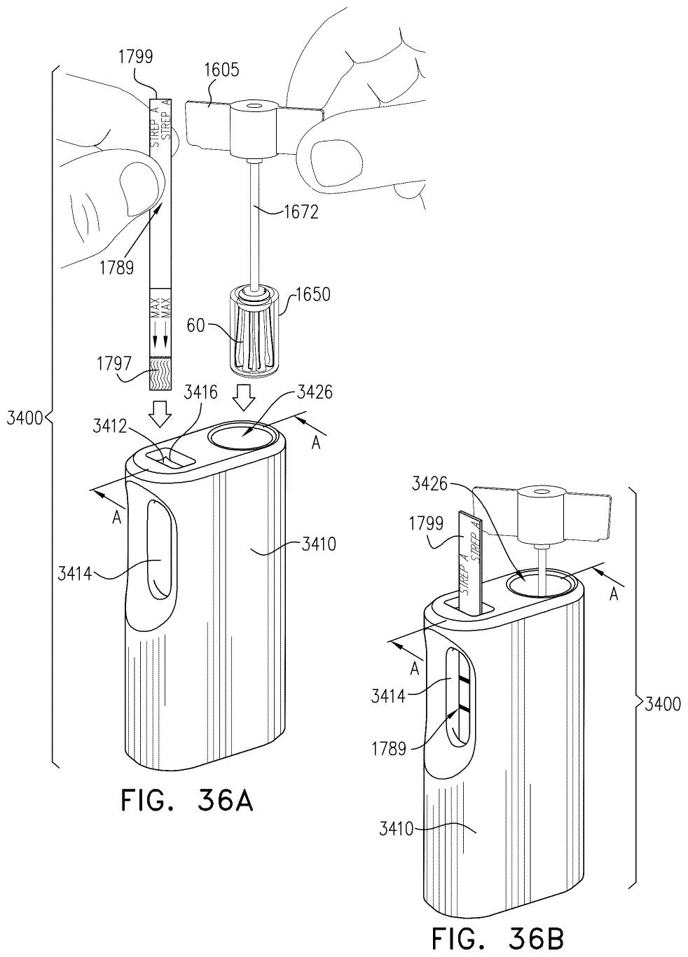

For some applications, sterile packaging is provided, in which one or more elements of kit 1000 (and/or of testing kit 1100, described hereinbelow with reference to FIG. 31; kit 3000, described hereinbelow with reference to FIGS. 32A-K; and/or kit 3400, described hereinbelow with reference to FIGS. 36A-B and 37A-B) are removably disposed.

Optionally, extraction tube 1718 comprises screw-off distal tip cap 1749 that removably seals a distal end of extraction tube 1718 opposite proximal end opening 1721. Upon removal of distal tip cap 1749, liquid can be expelled (e.g., squeezed or dripped out) of extraction tube 1718 via an opening through distal end 1751, for example as described hereinabove with reference to FIGS. 9C-D. Alternatively, extraction tube 1718 does not comprise a screw-off distal tip cap, such as shown, for example, in FIGS. 3J-K, described hereinabove.

For some applications, proximal end opening 1721 of extraction tube 1718 has a funnel shape, such as shown in FIG. 30. Alternatively, extraction tube 1718 is cylindrical near proximal end opening 1721, as is conventional in the extraction tube art, and as shown in FIGS. 9A-D and many of the other figures.

For some applications, kit 1000 further comprises a diagnostic test 1040 for testing a portion of liquid 1030 for the presence of a target analyte released into liquid 1030 from biological particulate trapped by filter 60.

For some applications, diagnostic test 1040 comprises a lateral flow test strip 1799 (in which case liquid 1030 typically comprises one or more reagents), such as a lateral flow immunoassay test strip. Lateral flow test strip 1799 is configured to detect the presence of the particulate (such as by detecting a target analyte, e.g., protein antigen, e.g., from a bacterium or from a virus). For some applications in which the lateral flow test strip comprises a lateral flow immunoassay test strip, lateral flow immunoassay test strip 1799 is an element of a chromatographic digital immunoassay, such as the BD Veritor™ system, mentioned immediately below), which is configured to detect the presence of the particulate (such as by detecting a target analyte, e.g., a protein antigen, e.g., from a bacterium or from a virus), and, optionally, one or more reagents. (Lateral flow immunoassay test strips (optionally as an element of a chromatographic digital immunoassay) are well known in the art. For example, they typically contain an antibody specific to an antigen, and the specimen sample fluid migrates up the test strip and reacts with the antibody, thus generating a line on the test strip; the presence of this line indicates a positive test result. For example, the BD Veritor™ system for Rapid Detection of SARS-CoV-2 (Becton, Dickinson and Company, Maryland, USA, Ref: 256082) is a chromatographic digital immunoassay intended for the direct and qualitative detection of SARS-CoV-2 nucleocapsid antigens in nasal swabs.) Alternatively, for some applications, diagnostic test 1040 comprises a rapid molecular test, for example, an isothermal nucleic acid amplification (iNAAT) test, such as a rapid molecular test kit that uses a real-time loop mediated amplification reaction, such as the Lucira COVID-19 All-In-One Test Kit (Lucira Health, Inc., Emeryville, CA, USA), or a nicking enzyme amplification reaction (NEAR) technology, such as the ID NOW™ (Abbott Laboratories, Abbott Park, Illinois, USA), or a molecular test kit manufactured by Visby. Further alternatively, test 302 may comprise a CRISPR-based diagnostic test, an ELISA diagnostic test, or a spectroscopy-based diagnostic test.

Kit 1000 may be used, for example, for collecting liquid specimen sample 22 from the subject and testing at least a portion of the specimen sample using diagnostic test 1040, or sending at least a portion of the specimen sample to a remote laboratory in liquid 1030 in extraction tube 1718, such as for performing Polymerase Chain Reaction (PCR) testing for particulate in liquid specimen sample 22.

The particulate may, for example, be a virus (e.g., an Influenza virus, or a coronavirus, such as SARS-CoV-2), a bacterium (e.g., Streptococcus bacteria, such as Streptococcus pyogenes (Strep A)), a microorganism, an antigen, a human cell, a cellular biomarker, a hormone, a chemical mediator from cells (e.g., a mediator of inflammation), a pollen, mucous, saliva, sputum, a respiratory particle, droplets derived from the upper and lower airways, a nucleic acid including DNA and RNA, and a chemical originating from external vapors. When the particulate is a microorganism, the microorganism may be either a pathogenic microorganism or a non-pathogenic microorganism or both, for example, viruses, bacteria, protozoa, and fungi. When the particulate is a human cell, the human cell may be an epithelial cell, for example, a columnar epithelial cell primarily derived from the nasal cavity and a squamous epithelial cell primarily derived from the oral cavity. The human cell may also be a cellular responder of the immune system, for example, neutrophils, eosinophils, lymphocytes, monocytes, macrophages, mast cells, and histocytes.

For some applications, kit 1000 further comprises distal plate 1671 that is fixed to a distal end of distal portion 1008 of filter shaft 1072 such that central portion 1010 of filter 60 is between the distal end and the distal plate, so as to directly couple filter 60 to filter shaft 1072, such as described hereinabove with reference to FIG. 3A.

Reference is now made to FIG. 31, which is a schematic illustration of a testing kit 1100, in accordance with an application of the present invention. Testing kit 1100 typically comprises all of the elements of kit 1000, described hereinabove with reference to FIG. 30. Testing kit 1100 further comprises a sampling device 1020, such as any of the sampling devices described herein and/or in any of the patent applications or patent application publications incorporated herein by reference. Filter 60 is typically disposed within a portion of the sampling device.

Testing kit 1100 may be used, for example, for collecting liquid specimen sample 22 from the subject and testing at least a portion of the specimen sample using diagnostic test 1040, or sending at least a portion of the specimen sample to a remote laboratory in liquid 1030 in extraction tube 1718, such as for performing Polymerase Chain Reaction (PCR) testing for particulate in liquid specimen sample 22.

For some applications, testing kit 1100 further comprises a container 1150 containing oral wash fluid 1152. The subject may optionally gargle the oral wash fluid, and use sampling device 1020 to filter the gargled oral wash fluid. Typically, the oral wash fluid comprises a non-irritant solution; for example, the non-irritant solution may comprise or consist of water. In some applications, the non-irritant solution comprises saline solution that may be hypertonic, isotonic, or hypotonic, for example, a phosphate-buffered saline solution. For example, container 1150 may contain 5-15 ml, such as 5-10 ml, of oral wash fluid 1152.

Sampling device 1020 may comprise any of the sampling device described herein, and/or may implement any of the features of any of the sampling devices described herein. Any of the filter-withdrawal shafts described herein may be replaced with filter shaft 1072.

For some applications, sampling device 1020 is configured such that withdrawal of filter shaft 1072 out of a filtration assembly of sampling device 1020 removes filter shaft 1072 and filter 60 from the filtration assembly and bunches up at least a portion of filter 60, thereby facilitating insertion of the bunched-up filter into extraction tube 1718. Bunched-up filter 60 may function somewhat analogously to a conventional swab, and, because of the reduced diameter caused by the bunching up, may be readily inserted into extraction tube 1718, such as described above and shown in FIGS. 6E and 7E, FIGS. 12F and 13F, FIGS. 15E and 16E, FIGS. 18E and 19E, FIGS. 21E and 22E, FIG. 23E, FIGS. 25E and 26E, and FIGS. 28E and 29E.

For some of these applications, sampling device 1020 is configured such that the withdrawal of filter shaft 1072 out of the filtration assembly removes filter shaft 1072 and filter 60 from the filtration assembly and bunches up the at least a portion of filter 60 into a flower-like arrangement, such as shown in many of the figures. Alternatively or additionally, the at least a portion of filter 60 is bunched up so as to have a plurality of folds, which optionally are soft folds (i.e., not creased). In this sense, the bunched-up filter may be considered to be shaped generally as a soft-pleated skirt without an opening at the waist. In general, the flower-like bunched-up shape of filter 60 may create open soft folds, rather than crumpled and/or closed folds that would create closed spaces that liquid 1030 cannot reach.

For some applications, sampling device 1020 is configured such that the withdrawal of filter shaft 1072 out of the filtration assembly removes filter shaft 1072 and filter 60 from the filtration assembly and bunches up the at least a portion of filter 60 such that:
an entirety of perimeter 1085 of filter 60 extends distally away from distal portion 1008 of filter shaft 1072 (such as shown and labeled in FIG. 3E by way of example and not limitation),
an entirety of perimeter 1085 of filter 60 points distally (such as shown and labeled in FIG. 3E by way of example and not limitation), and/or
the bunched-up portion of filter 60 defines an internal space 1087 open distally (such as shown and labeled in FIG. 3E by way of example and not limitation).

Typically, filter 60 is removably disposed in a flat shape on the support surface of the filter support of the filtration assembly of sampling device 1020, as shown in many of the figures illustrating sampling devices.

For some applications, the filtration assembly of sampling device 1020 is configured such that movement of a plunger head within a tubular container of the filtration assembly, when liquid specimen sample 22 is contained in the tubular container and filter 60 is disposed on the support surface of the filter support, pushes the at least a portion of liquid specimen sample 22 through filter 60 in an upstream-to-downstream direction, thereby trapping, on an upstream surface of filter 60, a portion of biological particulate present in liquid specimen sample 22, such as shown in many of the figures illustrating sampling devices.

For some of these applications, sampling device 1020 is configured such that the withdrawal of filter shaft 1072 out of the filtration assembly removes filter shaft 1072 and filter 60 from the filtration assembly and bunches up the at least a portion of filter 60 such that the upstream surface of the filter is inside the bunched-up filter, such as shown in FIGS. 3C-D, 7C-D, 13D-E, 16C-D, 19C-D, 22C-D, and 29C-D. As a result, any biological particulate present in liquid specimen sample 22 and trapped by filter 60 is positioned within bunched-up filter 60. When bunched-up filter 60 is inserted into extraction tube 1718, this position of the biological particulate within bunched-up filter 60 may help bring the biological particulate into good contact with liquid 1030 and maintain the good contact, by creating a contained area for liquid 1030 to interact with the biological particulate.

For others of these applications, sampling device 1020 is configured such that the withdrawal of filter shaft 1072 out of the filtration assembly removes filter shaft 1072 and filter 60 from the filtration assembly and bunches up the at least a portion of filter 60 such that a downstream surface of the filter is inside the bunched-up filter, such as shown in FIGS. 26C-D. As a result, any biological particulate present in liquid specimen sample 22 and trapped by filter 60 is positioned on an outer folded surface of bunched-up filter 60. This position of the biological particulate may help increase the contact of the biological particulate with liquid 1030 outside bunched-up filter 60 when bunched-up filter 60 is inserted into extraction tube 1718.

Reference is made to FIGS. 30 and 5A-8E. For some applications, kit 1000 further comprises filter receptacle 1714 coupled to a distal portion of filter shaft 1072 and shaped so as to define distal receptacle opening 1716, such as described hereinabove with reference to FIGS. 5A-8E. In these applications, sampling device 1020 is configured such that the withdrawal of filter shaft 1072 out of the filtration assembly pulls central portion 1010 of filter 60 into filter receptacle 1714 via distal receptacle opening 1716, thereby causing the remainder of filter 60 to become bunched up and be disposed at least partially outside filter receptacle 1714. Optionally, filter receptacle 1714 is slidably coupled to a distal portion of filter shaft 1072.

Reference is made to FIGS. 30 and 31. In an application of the present invention, a method is provided comprising:
passing at least a portion of liquid specimen sample 22 through filter 60, using (a) techniques described with reference to many of the figures herein, (b) techniques described in the patent applications or patent application publications incorporated hereinbelow by reference, such as, by way of example and not limitation, with reference to FIGS. 14A-B of PCT Publication WO 2021/224925 to Levitz et al., or (c) conventional filtering techniques and devices known in the art; for some applications, the at least a portion of liquid specimen sample 22 is passed through filter 60 while filter 60 is in a flat shape, such as shown in many of the figures;
bunching up at least a portion of filter 60 while central portion 1010 of filter 60 is coupled to distal portion 1008 of filter shaft 1072, such as described with reference to many of the figures herein; this bunching up of filter 60 may help facilitate insertion of filter 60 into extraction tube 1718; filter 60 may be coupled to distal portion 1008 of filter shaft 1072 either before or after liquid specimen sample 22 is passed through filter 60, such as described herein;
placing liquid 1030 in extraction tube 1718 (for example, as shown in FIG. 9B); alternatively, extraction tube 1718 is provided already containing liquid 1030;
before or after placing liquid 1030 in extraction tube 1718, inserting the bunched-up portion of filter 60 into extraction tube 1718 (via proximal end opening 1721 of extraction tube 1718), such as described with reference to many of the figures herein;
bathing filter 60 in liquid 1030 in extraction tube 1718 (for example, as shown in FIG. 9B); and
thereafter, testing a portion of liquid 1030 for the presence of a target analyte released into liquid 1030 from biological particulate trapped by filter 60 (for example, liquid 1030 may be expelled (e.g., dripped or squeezed) onto a diagnostic test, such as onto a sample pad 1797 of a lateral flow test strip 1799, as shown in FIG. 9D).

This method may or may not include separating portion of filter-withdrawal shaft 1772 from each other, as shown in FIG. 9B.

Reference is made to FIG. 31. For some applications, the at least a portion of liquid specimen sample 22 is passed through filter 60 using filtration assembly of sampling device 1020 while filter 60 is removably disposed the filtration assembly and filter shaft 1072 is removably disposed partially within the filtration assembly. The filtration assembly of sampling device 1020 may implement any of (a) the techniques of the filtrations assemblies described herein, (b) techniques described in the patent applications or patent application publications incorporated hereinbelow by reference, such as, by way of example and not limitation, with reference to FIGS. 14A-B of PCT Publication WO 2021/224925, or (c) conventional filtering techniques and devices known in the art.

For some applications, the bunched-up portion of filter 60 is inserted into extraction tube 1718 using the techniques described hereinabove with reference to FIGS. 3E-F.

Reference is again made to FIGS. 30 and 31. For some applications, the at least a portion of filter 60 is bunched up by passing filter 60 through a tube, such as a cylindrical tube. The tube is typically separate from the filtration assembly. Alternatively, for some applications, the at least a portion of filter 60 is bunched up by passing filter 60 into and/or through extraction tube 1718. These two techniques may be particularly useful when a filtration assembly is used that does not bunch up the filter, such as a conventional filtration assembly or, by way of example and not limitation, the filtration assembly described with reference to FIGS. 14A-B of PCT Publication WO 2021/224925.

Alternatively, filter shaft 1072 comprises filter-withdrawal shaft 1672 and collection vial 1650 is additionally provided, such as described hereinabove with reference to FIGS. 1A-4. For some of these applications, the at least a portion of filter 60 is bunched up by passing filter 60 at least partially into collection vial 1650, by sliding proximal portion 1687 of filter-withdrawal shaft 1672 through shaft-passage hole 1609 of collection vial 1650. This technique may be used even in configurations in which a filtration assembly is used that does not bunch up the filter, in which case collection vial 1650 is optionally not initially disposed within a filtration assembly as described hereinabove with reference to FIGS. 1A, 2A-C, and 3A-C. Alternatively or additionally, this technique may be used more than once during a testing procedure, such as a first time in a filtration assembly, as described hereinabove with reference to FIGS. 2B-D and 3B-D, and a second time after removing filter 60 from extraction tube 1718, such as described hereinbelow with reference to FIGS. 32G and 34D.

Reference is still made to FIGS. 30 and 31. For some applications, liquid 1030 is selected from the group consisting of: a lysis buffer, an extraction buffer, saline solution, and a transport medium. For some applications, liquid 1030 comprises one or more reagents, and the portion of liquid 1030 is tested by testing the portion of the one or more reagents for the presence of the target analyte released into the one or more reagents from the biological particulate trapped by filter 60. For some applications, the portion of the one or more reagents is tested by applying the portion of the one or more reagents to lateral flow test strip 1799, such as shown in FIG. 9D.

Reference is made to FIG. 9D. For some applications, testing the portion of liquid 1030 comprises squeezing the bunched-up portion of filter 60 to squeeze the portion of liquid 1030 from filter 60, such as shown in FIG. 9D (and FIGS. 3I, 32I-J, and 34D-E). For some applications, the bunched-up portion of filter 60 is squeezed while the bunched-up portion of filter 60 is within extraction tube 1718, such as shown in FIG. 9D (and FIG. 3I). In a number of configurations described herein, a portion of liquid 1030 is described as being expelled from filter 60 (e.g., a bunched-up portion thereof), such as by squeezing and/or rotating the filter; it is noted that optionally a portion of any of liquid specimen sample 22 trapped by filter 60 may also be expelled with liquid 1030.

For some applications, the bunched-up portion of filter 60 is not rotated before, during, and/or after squeezing.

For some applications, the bunched-up portion of filter 60 is rotated before, during, and/or after squeezing. For some of these applications, an inner surface of a side wall of extraction tube 1718 is shaped so as to define one or more protrusions that may help change the direction in which the folds of bunched-up filter 60 point (from clockwise to counterclockwise and back) as the filter is rotated clockwise and counterclockwise). For example, extraction tube 1718 may implement configurations described with reference to FIGS. 13A-E of PCT Publication WO 2022/044002 to Levitz et al. for receptacle 1124 thereof, mutatis mutandis.

Typically, filter 60 is not agitated in extraction tube 1718, although it may optionally be agitated.

For some applications, extraction tube 1718 comprises a flexible material, and the bunched-up portion of filter 60 is squeezed by squeezing a longitudinal portion of extraction tube 1718 along which longitudinal portion the bunched-up portion of filter 60 is disposed, such as shown in FIG. 9D (and FIG. 3I).

For some applications, after filter 60 is bathed in liquid 1030 in extraction tube 1718 and before the bunched-up portion of filter 60 is squeezed, excess liquid 1030 is removed (e.g., drained) from extraction tube 1718. Removal of the excess liquid may help reduce the dilution of the liquid absorbed in and expelled from filter 60.

For some applications, the bunched-up portion of filter 60 is removed from extraction tube 1718 before the bunched-up portion of filter 60 is squeezed (while outside extraction tube 1718). During this removal, excess liquid 1030 remains in extraction tube 1718, which may help reduce the dilution of the liquid absorbed in and expelled from filter 60.

Reference is again made to FIGS. 30 and 31. Typically, filter-withdrawal shaft 1772 extends away from a first side of filter 60 (the top side shown in FIGS. 30 and 31) and does not extend away from a second side of filter 60 opposite the first side (the bottom side in FIGS. 30 and 31) (as shown), or extends away from the second side of filter 60 by less than 10 mm, such as less than 2 mm. As a result, when the portion of filter 60 is bunched up, filter-withdrawal shaft 1772 does not extend, or extends by less than 10 mm (such as less than 2 mm), into the internal space defined by the bunched-up portion of filter 60. The absence of filter-withdrawal shaft 1772 within the bunched-up portion of filter 60 leaves the internal space free for liquid 1030 to fill and make good contact with a large portion of the surface area of the inner surface of the bunched-up portion of filter 60. In addition, the absence of filter-withdrawal shaft 1772 within the bunched-up portion of filter 60 may make it easier to thoroughly squeeze filter 60 than if the relative rigid shaft were within the bunched-up portion of filter 60.

Alternatively, for some applications, the bunched-up portion of filter 60 is squeezed by inserting an object into extraction tube 1718 and using the object to squeeze the bunched-up portion of filter 60 (configuration not shown).

Alternatively or additionally, for some applications, the portion of liquid 1030 is tested using a diagnostic test to test the portion of liquid 1030 for the presence of a target analyte released into liquid 1030 from the biological particulate, and the bunched-up portion of filter 60 is squeezed by inserting a portion of the diagnostic test into extraction tube 1718 and using the portion of the diagnostic test to squeeze the bunched-up portion of filter 60 (configuration not shown).

Further alternatively, the bunched-up portion of filter 60 is removed from extraction tube 1718 after bathing in liquid 1030, and thereafter the bunched-up portion of filter 60 is squeezed to squeeze the portion of liquid 1030 from filter 60 (configuration not shown). Optionally, the bunched-up portion of filter 60 is inserted into another tube before squeezing and squeezed while in this second tube (configuration not shown).

Optionally, excess liquid 1030 is drained from extraction tube 1718 before the bunched-up portion of filter 60 is squeezed (configuration not shown), such as while the bunched-up portion of the filter 60 is in extraction tube 1718.

For some applications, the at least a portion of filter 60 is bunched up into a flower-like arrangement, such as shown in many of the figures.

Reference is made to FIGS. 12F, 30, and 31. For some applications, the at least a portion of filter 60 is bunched up such that:
an entirety of a perimeter 1085 of filter 60 extends distally away from distal portion 1008 of filter shaft 1072 (such as shown and labeled in FIG. 12F by way of example and not limitation), and the bunched-up portion of filter 60 is inserted into extraction tube 1718 by inserting, in a distal direction, the bunched-up portion of filter 60 into extraction tube 1718 (via proximal end opening 1721 of extraction tube 1718) while the entirety of the perimeter of filter 60 extends distally away from distal portion 1008 of filter shaft 1072,
an entirety of perimeter 1085 of filter 60 points distally (such as shown and labeled in FIG. 12F by way of example and not limitation), and the bunched-up portion of filter 60 is inserted into extraction tube 1718 by inserting, in a distal direction, the bunched-up portion of filter 60 into extraction tube 1718 (via proximal end opening 1721 of extraction tube 1718) while the entirety of the perimeter of filter 60 points distally, and/or
the bunched-up portion of filter 60 defines an internal space open distally (such as shown and labeled in FIG. 12F by way of example and not limitation), and the bunched-up portion of filter 60 is inserted into extraction tube 1718 by inserting, in a distal direction, the bunched-up portion of filter 60 into extraction tube 1718 (via proximal end opening 1721 of extraction tube 1718) while the bunched-up portion of filter 60 defines the internal space open distally.

The at least a portion of liquid specimen sample 22 is passed through filter 60 in an upstream-to-downstream direction, thereby trapping, on an upstream surface of filter 60, a portion of biological particulate present in the liquid specimen. For some applications, the at least a portion of filter 60 is bunched up such that the upstream surface of filter 60 is inside the bunched-up filter 60, such as described hereinabove and such as shown in FIGS. 3C-D, 7C-D, 13D-E, 16C-D, 19C-D, 22C-D, and 29C-D. For other applications, the at least a portion of filter 60 is bunched up such that a downstream surface of the filter is inside the bunched-up filter 60, such as described hereinabove and such as shown in FIGS. 26C-D.

Reference is now made to FIG. 32A and FIGS. 32B-K, which are schematic illustrations of a kit 3000 and a method of using kit 3000 for testing for presence of a particulate, respectively, in accordance with an application of the present invention. The method of using kit 3000 may optionally be implemented after filter 60 has been covered and/or shielded by collection vial 1650, optionally using any of the sampling devices described herein or another sampling device.

Kit 3000 comprises a housing 3010 and, as can be seen in FIGS. 32E, 32J, and 32K, lateral flow test strip 1799, which optionally comprises a lateral flow immunoassay test strip, and which is contained at least partially within housing 3010. For some applications, such as shown, housing 3010 is shaped as a cartridge 3089 (also known as a cassette). Kit 3000 further comprises liquid 1030, which comprises one or more reagents, and one or more containers 1022 that contain liquid 1030. Optionally, liquid 1030 comprises two or more liquids, which are contained in two or more respective containers 1022, and are combined during the testing procedure, such as shown in FIG. 32B.

For some applications, housing 3010 is shaped so as to define an extraction tube 3018. The location of extraction tube 3018 with respect to other elements of housing 3010 is shown in the figures by way of example and not limitation. Extraction tube 3018 may implement any of the features of extraction tube 1718 or any of the other extraction tubes described herein, and extraction tube 3018 may be used for performing any of the methods described herein as being performed using extraction tube 1718 or any of the other extraction tubes described herein, mutatis mutandis. Similarly, extraction tube 1718 or any of the other extraction tubes described herein may implement any of the features of extraction tube 3018, and extraction tube 1718 or any of the other extraction tubes described herein may be used for performing any of the methods described herein as being performed using extraction tube 3018, mutatis mutandis.

Alternatively or additionally, for some applications, housing 3010 is shaped so as to define a channel 3026 in fluid communication with sample pad 1797 of lateral flow test strip 1799 (labeled in FIGS. 32E and 32J). Channel 3026 has a straight central longitudinal axis 3029.

For some applications, channel 3026 has one or more of the following dimensions:
an internal length of 1-5 cm, e.g., 2-5 cm,
an average internal diameter of 4-7 cm, and/or
an average internal cross-sectional area, measured perpendicular to a central longitudinal axis of channel 3026, of 12-40 cm2.

As shown in FIG. 32B, liquid 1030 is dispensed from the one or more containers 1022 into extraction tube 3018. Alternatively, extraction tube 3018 may comprise one or more internal containers that contain liquid 1030 and are ruptured upon insertion of collection vial 1650 and/or insertion or distal advancement of bunched-up filter 60 into extraction tube 3018, such as described hereinbelow with reference to FIGS. 32C-E (configuration not shown).

As shown in FIGS. 32C-E, collection vial 1650 (including the bunched-up portion of filter 60) is inserted at least partially into extraction tube 3018 (via a proximal end opening 3021 of extraction tube 3018), and distally advanced within extraction tube 3018 until the bunched-up portion of filter 60 is positioned near a distal end 3015 of extraction tube 3018 opposite proximal end opening 3021 (labeled in FIG. 32E). (Filter 60 was earlier covered and/or shielded by collection vial 1650, optionally using any of the sampling devices described herein or another sampling device.) Extraction tube 3018 is shaped so as to prevent collection vial 1650 from reaching distal end 3015 of extraction tube 3018. For example:
an internal wall of extraction tube 3018 may define one or more internal protrusions 3022, e.g., ridges,
an internal wall of extraction tube 3018 may narrow in a proximal-to-distal direction, e.g., may include a conical portion,
extraction tube 3018 may comprise an internal o-ring, or
proximal end opening 3021 of extraction tube 3018 may have an internal diameter that is less than an outer diameter of collection vial 1650.

For some applications in which the internal wall of extraction tube 3018 narrows in the proximal-to-distal direction, an internal diameter of channel 3026 at the distal end is 4-5 mm, and an internal diameter of channel 3026 at the proximal opening is 5-7 cm. (The other extractions tubes and channels described herein may also implement any of these features). As a result, collection vial 1650 slides up a portion of filter-withdrawal shaft 1672 as bunched-up filter 60 is positioned near distal end 3015, thereby ejecting bunched-up filter 60 from vial opening 1652 of collection vial 1650 and exposing bunched-up filter 60 to liquid 1030 within the distal portion of extraction tube 3018.

Optionally, distal end 3015 of extraction tube 3018 may be curved or conical to increase contact between liquid 1030 and bunched-up filter 60.

Optionally, as shown in FIG. 32F, the bunched-up portion of filter 60 is rotated one or more times, optionally both clockwise and counterclockwise, such as by rotating shaft handle 1605.

After bunched-up filter 60 has been exposed to liquid 1030 in extraction tube 3018 (typically bathed in liquid 1030 for a certain amount of time), filter-withdrawal shaft 1672 is withdrawn from extraction tube 3018. This proximal movement of filter-withdrawal shaft 1672 with respect collection vial 1650 pulls filter 60 at least partially into (such as entirely into) collection vial 1650 via vial opening 1652, such as shown in FIG. 32G, thereby at least partially (e.g., entirely) covering and/or shielding filter 60.

As shown in FIGS. 32H and 32I, the bunched-up portion of filter 60 is squeezed to squeeze a portion of liquid 1030 from filter 60 by inserting the bunched-up portion of filter 60 into channel 3026 (via a proximal end opening of channel 3026). This squeezing is typically performed by longitudinally compacting the bunched-up portion of filter 60 against a distal blocking surface 3031 intercepted by straight central longitudinal axis 3029 of channel 3026, such as by distally pushing the bunched-up portion of filter 60 by filter-withdrawal shaft 1672 (in which case straight central longitudinal axis 3029 of channel 3026 is typically also a straight longitudinal axis of filter-withdrawal shaft 1672). (“Longitudinally compacting” the bunched-up portion of filter 60 means shortening a length of the bunched-up portion of filter 60, the length measured along a longitudinal axis of the bunched-up portion of filter 60.) The longitudinal compacting of the bunched-up portion of filter 60 against distal blocking surface 3031 generally crushes the bunched-up portion of filter 60 against distal blocking surface 3031. (Distal blocking surface 3031 faces at least partially in a proximal direction.) Generally, an internal wall 3028 of channel 3026 constrains horizontal expansion of the bunched-up portion as the bunched-up portion is longitudinally compacted, thereby preventing the bunched-up portion of filter 60 from horizontally spreading out rather than becoming compacted. Straight central longitudinal axis 3029 may form an oblique angle (a non-right angle) with distal blocking surface 3031, such as shown, or may form a right angle (configuration not shown); for example, the angle may be 30-90 degrees, such as 45-90 degrees, e.g., 60-90 degrees. Typically, distal blocking surface 3031 is upstream of a test area 1788 of lateral flow test strip 1799.

Typically, the bunched-up portion of filter 60 is inserted into channel 3026 by inserting collection vial 1650 at least partially into channel 3026 (via the proximal end opening of the channel), and distally advancing the collection vial within channel 3026 until bunched-up filter 60 is advanced out of the collection vial, through channel 3026, and into direct contact with sample pad 1797 of lateral flow test strip 1799. Optionally, at least a portion of filter 60 is pressed against sample pad 1797. Sample pad 1797 thus serves as the above-mentioned distal blocking surface 3031 and blocks further distal advancement of the bunched-up portion of filter 60, in order to longitudinally compact the bunched-up portion. Straight central longitudinal axis 3029 may form an oblique angle (a non-right angle) with sample pad 1797, such as shown, or may form a right angle (configuration not shown); for example, the angle may be 30-90 degrees, such as 45-90 degrees, e.g., 60-90 degrees. The distal advancing of bunched-up filter 60 is typically performed using filter-withdrawal shaft 1672; in this case, straight central longitudinal axis 3029 of channel 3026 is typically also a straight longitudinal axis of filter-withdrawal shaft 1672, which may form an oblique angle (a non-right angle) with sample pad 1797, such as shown, or may form a right angle (configuration not shown); for example, the angle may be 30-90 degrees, such as 45-90 degrees, e.g., 60-90 degrees.

Collection vial 1650 may aid with the insertion of bunched-up filter 60 into channel 3026. Collection vial 1650 is readily inserted into channel 3026, thereby inserting bunched-up filter 60 into channel 3026 while the bunched-up filter is initially within collection vial 1650.

Optionally, such as shown in FIG. 32J, filter 60 is rotated one or more times, optionally both clockwise and counterclockwise, such as by rotating shaft handle 1605, in order to facilitate squeezing liquid from filter 60.

As described hereinabove with reference to FIGS. 12F, 30, and 31, for some applications, the at least a portion of filter 60 is bunched up such that an entirety of perimeter 1085 of filter 60 points distally. For some of these applications, such as shown in FIG. 32I, the bunched-up portion of filter 60 is inserted into channel 3026 (via the proximal end opening of the channel) in a distal direction while the entirety of perimeter 1085 of filter 60 points distally.

Optionally, the distal portion of the bunched-up portion of filter 60 is advanced through channel 3026 so that at least a portion of perimeter 1085 of filter 60 makes direct contact with sample pad 1797 of lateral flow test strip 1799. Typically, internal wall 3028 is shaped so as to hold perimeter 1085 pointing distally and direct the perimeter to sample pad 1797.

Generally, bringing filter 60 into direct contact with sample pad 1797 provides good test results, typically in part because particulate trapped by filter 60 remains on filter 60 after liquid 1030 (e.g., one or more reagents) is applied to filter 60. Typically, more liquid 1030 (and particulate in the liquid) remains in or on filter 60 than in or on a conventional swab. For example, this may be the case because the bunched-up portion of filter 60 defines an internal space open distally, and liquid 1030 is trapped within this internal space by the bunching up of the filter. Squeezing filter 60 against sample pad 1797 releases some of liquid 1030 from this internal space onto sample pad 1797. Thus, squeezing filter 60 against sample pad 1797 delivers to sample pad 1797 both (a) some of liquid 1030 contained in or on filter 60 at locations of filter 60 that directly contact sample pad 1797, and (b) some of liquid 1030 contained in or on filter 60 at locations other than the locations of filter 60 that directly contact sample pad 1797 (including liquid 1030 in the internal space). (In general, liquid 1030 may be contained in or on filter 60 by being trapped by, absorbed in, or adhering to the filter.)

Although filter 60 is shown as being bunched-up when brought into direct contact with sample pad 1797, filter 60 alternatively is not bunched up when brought into contact with sample pad 1797 (configuration not shown).

As shown in FIG. 32K, lateral flow test strip 1799 is used to test for the presence of a target analyte released into liquid 1030 from particulate trapped by filter 60.

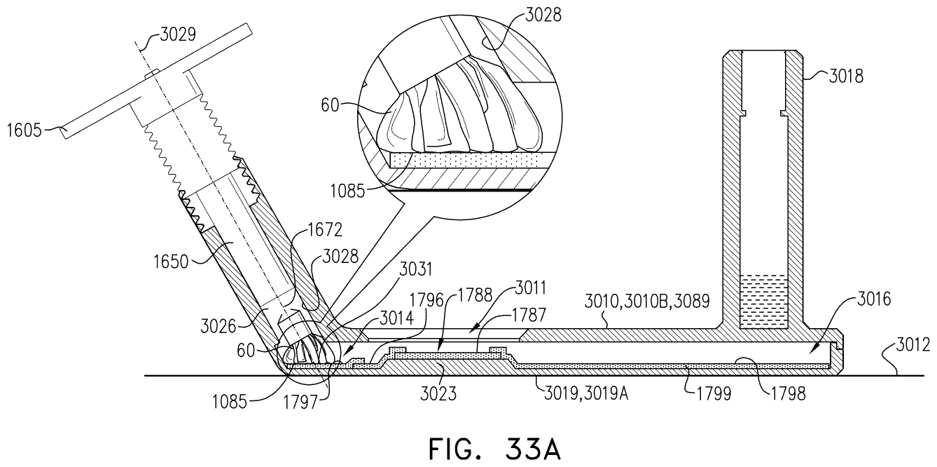

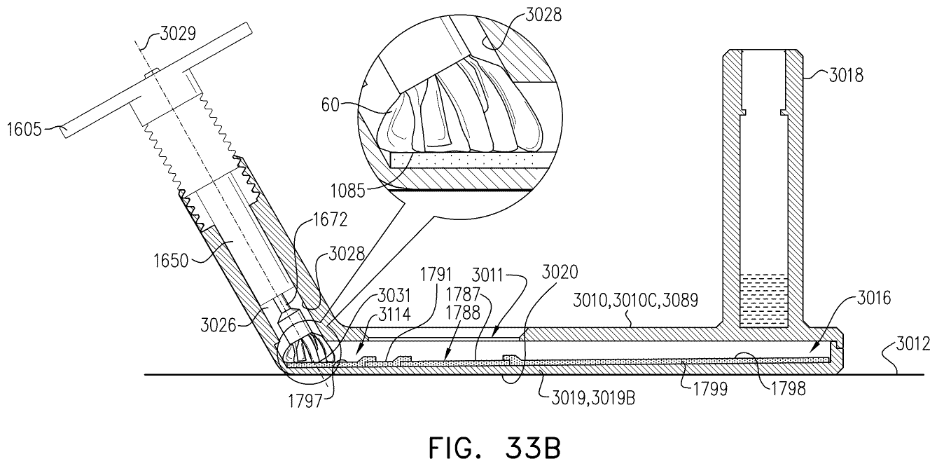

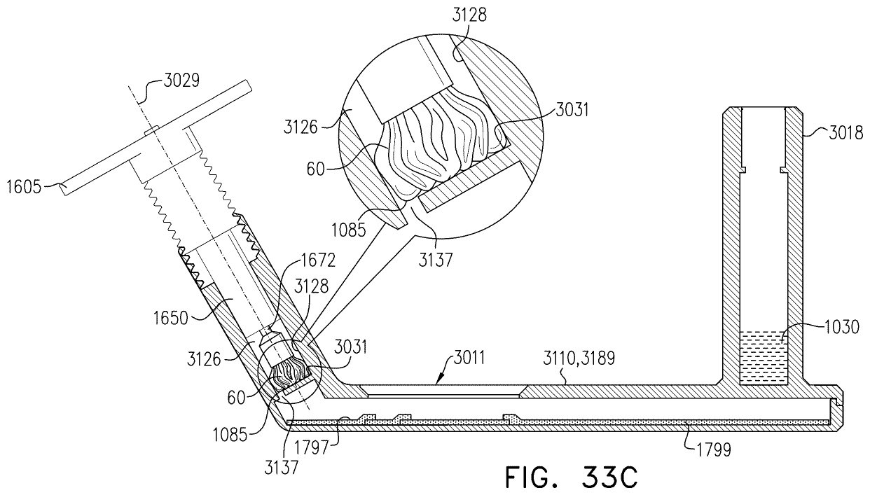

Reference is made to FIG. 281 and is also made to FIGS. 33A-B, which are schematic illustrations of respective alternative configurations of a housing 3010, in accordance with respective applications of the present invention. Except as described below, these configurations of housing 3010 is identical to the configuration of housing 3010 described hereinabove with reference to FIGS. 32A-K, and may implement any features thereof, mutatis mutandis. Like reference numerals refer to like parts. Housing 3010 is shaped as cartridge 3089. Housing 3010 is shaped so as to define channel 3026 in fluid communication with sample pad 1797 of lateral flow test strip 1799.

Sample pad 1797 is disposed at an upstream end portion 3014 of lateral flow test strip 1799, and an absorbent pad 1798 of lateral flow test strip 1799 is disposed at a downstream end portion 3016 of lateral flow test strip 1799, as is known in the lateral flow test strip art. As is also known in the lateral flow test strip art, a membrane 1787 is disposed longitudinally between sample pad 1797 and absorbent pad 1798. Membrane 1787 comprises test area 1788, which comprises a test line and a control line. Membrane 1787 typically comprises nitrocellulose. Typically, a conjugate pad 1796 is disposed between sample pad 1797 and membrane 1787, as is known in the lateral flow test strip art. Typically housing 3010 is shaped so as to define one or more result windows 3011, and lateral flow test strip 1799 is disposed at least partially within the housing such that test area 1788 is visible through the one or more result windows 3011.

Reference is made to FIG. 32I, which schematically illustrates a housing 3010A, which is one configuration of housing 3010. In this configuration, lateral flow test strip 1799 is oriented parallel to a flat surface 3012 when housing 3010A is placed on flat surface 3012 (and thus is oriented horizontally when housing 3010A is placed on flat surface 3012 that is horizontal with respect to the Earth).

Reference is made to FIGS. 33A and 33B, which schematically illustrate a housing 3010B and a housing 3010C, which are additional configurations of housing 3010. In these configurations, housing 3010B, 3010C is configured such that test area 1788 of membrane 1787 is farther from flat surface 3012 than sample pad 1797 is from flat surface 3012, when housing 3010B, 3010C is placed on flat surface 3012. Thus, test area 1788 of membrane 1787 is more elevated than sample pad 1797 with respect to the Earth when housing 3010B, 3010C is placed on flat surface 3012 that is horizontal with respect to the Earth.

This raised arrangement of test area 1788 of lateral flow test strip 1799 may slow down lateral flow of the portion of liquid 1030 expelled from the bunched-up portion of filter 60, thereby preventing flooding of the test strip and enabling proper capillary action for controlled flow downstream along the test strip. Optionally, this raised arrangement of test area 1788 is implemented in housing 3110, described hereinbelow with reference to FIG. 33C.

Reference is made to FIG. 33A. In this configuration, in order to provide this raised arrangement of test area 1788, housing 3010B is shaped so as to define a raised portion 3023 at least under test area 1788 of membrane 1787. For example, a bottom wall 3019, 3019A of housing 3010B may be shaped so as to define raised portion 3023, as shown in FIG. 33A; alternatively, housing 3010B may comprise a separate piece that defines raised portion 3023 (configuration not shown).

Reference is made to FIG. 33B. In this configuration, lateral flow test strip 1799 is oriented obliquely (slanted) with respect to flat surface 3012 when housing 3010B is placed on flat surface 3012 (and thus is oriented obliquely with respect to the Earth when housing 3010A is placed on flat surface 3012 that is horizontal with respect to the Earth), such that downstream end portion 3116 of lateral flow test strip 1799 is more elevated from flat surface 3012 than is upstream end portion 3114 of lateral flow test strip 1799. For example, lateral flow test strip 1799 may be oriented at an angle of 1-20 degrees, e.g., 1-15 degrees, such as 1-10 degrees, with respect to flat surface 3012 when housing 3010B is placed on flat surface 3012.

Optionally, bottom wall 3019, 3019B of housing 3010B has a variable thickness therealong in order to provide this orientation of lateral flow test strip 1799, in which case lateral flow test strip 1799 is oriented at the above-mentioned angle with respect to a bottom surface 3120 of bottom wall 3019, 3019B of housing 3010B.

Reference is now made to FIG. 33C, which is a schematic illustration of a housing 3110, in accordance with an application of the present invention. Except as described below, housing 3110 is identical to housing 3010, described hereinabove with reference to FIGS. 32I and 33A-B, and may implement any features thereof, mutatis mutandis. Like reference numerals refer to like parts. Housing 3110 is shaped as a cartridge 3189. Housing 3110 is shaped so as to define a channel 3126 in fluid communication with sample pad 1797 of lateral flow test strip 1799. Channel 3126 may optionally implement any of the features of channel 3026, including, but not limited to, the dimensions of channel 3026 described hereinabove with reference to FIG. 32A and FIGS. 32B-K.

In this configuration, at the step of the testing method described hereinabove with reference to 32I, a distal portion of the bunched-up portion of filter 60 is advanced within channel 3126 until bunched-up filter 60 is pressed against distal blocking surface 3031 defined by channel 3126 (upstream of sample pad 1797), so as to longitudinally compact the bunched-up portion of filter 60.

The portion of liquid 1030 expelled from the bunched-up portion of filter 60 comes into contact with sample pad 1797 of lateral flow test strip 1799, optionally via an opening 3137 through a distal portion of channel 3126, such as shown in FIG. 33C.

Typically, an internal wall 3128 of channel 3126 is shaped so as to squeeze the bunched-up portion of filter 60 upon insertion and distal advancement of the bunched-up portion of filter 60 in channel 3126; typically, internal wall 3128 is sufficiently rigid so as not to deform while squeezing the bunched-up portion of filter 60.

Reference is made to FIGS. 32A-K and 33A-C. In some applications of the present invention, housing 3010, 3110 is not shaped so as to define extraction tube 3018. Kit 3000 comprises a separate extraction tube 1718, for example as described hereinbelow with reference to FIGS. 34A-D. For example, as shown in FIGS. 34B-C, collection vial 1650 is inserted at least partially into extraction tube 1718.

Reference is now made to FIGS. 34A-F, which are schematic illustrations of a kit and a method for testing for presence of a particulate, in accordance with an application of the present invention. This method may optionally be implemented after filter 60 has been covered and/or shielded by collection vial 1650, optionally using any of the sampling devices described herein or another sampling device. This method may optionally be implemented in combination with any of the techniques described hereinabove with reference to FIGS. 32A, 32B-K, 33A-B, and/or 33C, mutatis mutandis.

As shown in FIG. 34A, liquid 1030 is dispensed from the one or more containers 1022 into extraction tube 3018.

As shown in FIGS. 34B-C, collection vial 1650 is inserted at least partially into extraction tube 1718 (via proximal end opening 1721), and distally advanced within extraction tube 1718 until bunched-up filter 60 is positioned near a distal end of extraction tube 1718 opposite proximal end opening 1721. (Filter 60 was earlier covered and/or shielded by collection vial 1650, optionally using any of the sampling devices described herein or another sampling device.) Extraction tube 1718 is shaped so as to prevent collection vial 1650 from reaching the distal end of extraction tube 1718, such that collection vial 1650 slides up a portion of filter-withdrawal shaft 1672 as bunched-up filter 60 is positioned near the distal end of extraction tube 1718, thereby ejecting bunched-up filter 60 from vial opening 1652 of collection vial 1650 and exposing bunched-up filter 60 to liquid 1030 within the distal portion of extraction tube 1718.

Optionally, such as shown in FIG. 32F, the bunched-up portion of filter 60 is rotated one or more times, optionally both clockwise and counterclockwise, such as by rotating shaft handle 1605.

After bunched-up filter 60 has been exposed to liquid 1030 in extraction tube 1718 (typically bathed in liquid 1030 for a certain amount of time), filter-withdrawal shaft 1672 is withdrawn from extraction tube 1718. This proximal movement of filter-withdrawal shaft 1672 with respect collection vial 1650 pulls filter 60 at least partially into (such as entirely into) collection vial 1650 via vial opening 1652, such as shown in FIG. 34D, thereby at least partially (e.g., entirely) covering and/or shielding filter 60.

As shown in FIGS. 34A-F, a housing 3210 and lateral flow test strip 1799, which optionally comprises a lateral flow immunoassay test strip, are provided. Lateral flow test strip 1799 is contained at least partially within housing 3210. For some applications, such as shown, housing 3210 is shaped as a card 3294, as known in the rapid immunoassay testing arts.

Housing 3210 is shaped so as to define a channel 3226 in fluid communication with sample pad 1797 of lateral flow test strip 1799 (sample pad 1797 is not visible in FIGS. 34E-F, but can be seen in FIGS. 32E and 32J).

As shown in FIGS. 34E and 34F, the bunched-up portion of filter 60 is squeezed to squeeze and expel a portion of liquid 1030 from the bunched-up portion of filter 60 by inserting the bunched-up portion of filter 60 into channel 3226 (via a proximal end opening of the channel) so that the bunched-up portion of filter 60 is squeezed by an internal wall of channel 3226; typically, the internal wall is sufficiently rigid so as not to deform while squeezing the bunched-up portion of filter 60. Thus, the internal wall of channel 3226 is shaped so as to squeeze the bunched-up portion of filter 60 upon insertion and distal advancement of the bunched-up portion of filter 60 in channel 3226, typically without channel 3226 being externally squeezed.

Typically, the bunched-up portion of filter 60 is inserted into channel 3226 by inserting collection vial 1650 at least partially into channel 3226, and distally advancing the collection vial within channel 3226 until bunched-up filter 60 is positioned near a distal end of channel 3226 opposite a proximal end opening of the channel. Collection vial 1650 may aid with the insertion of bunched-up filter 60 into channel 3226. Collection vial 1650 is readily inserted into channel 3226, thereby inserting bunched-up filter 60 into channel 3226 while the bunched-up filter is initially within collection vial 1650.

Optionally, such as shown in FIG. 32J, filter 60 is rotated one or more times, optionally both clockwise and counterclockwise, such as by rotating shaft handle 1605, in order to facilitate squeezing liquid from filter 60.

As described hereinabove with reference to FIGS. 12F, 30, and 31, for some applications, the at least a portion of filter 60 is bunched up such that an entirety of perimeter 1085 of filter 60 points distally. For some of these applications, such as shown in FIG. 34E, the bunched-up portion of filter 60 is inserted into channel 3226 in a distal direction while the entirety of perimeter 1085 of filter 60 points distally.

As shown in FIG. 34F, the portion of liquid 1030 expelled from the bunched-up portion of filter 60 comes into contact with the sample pad of lateral flow test strip 1799, and lateral flow test strip 1799 is used to test for the presence of a target analyte released into liquid 1030 from particulate trapped by filter 60.

Reference is made to FIGS. 34E-F. For some applications, housing 3210 is shaped so as to define an extraction tube, similar to the configuration described hereinabove for housing 3010 and extraction tube 3018.

Reference is made to FIGS. 34A-F. In some applications of the present invention, extraction tube 3018 is not provided for insertion of bunched-up filter 60 into liquid 1030. Instead, channel 3226 is additionally used for bathing bunched-up filter 60 in liquid 1030. Liquid may be dispensed from the one or more containers 1022 into channel 3226, such as described hereinabove with reference to FIG. 34A, mutatis mutandis. Alternatively, channel 3226 may comprise one or more internal containers that are ruptured upon insertion and/or distal advancement of bunched-up filter 60 into channel 3226.

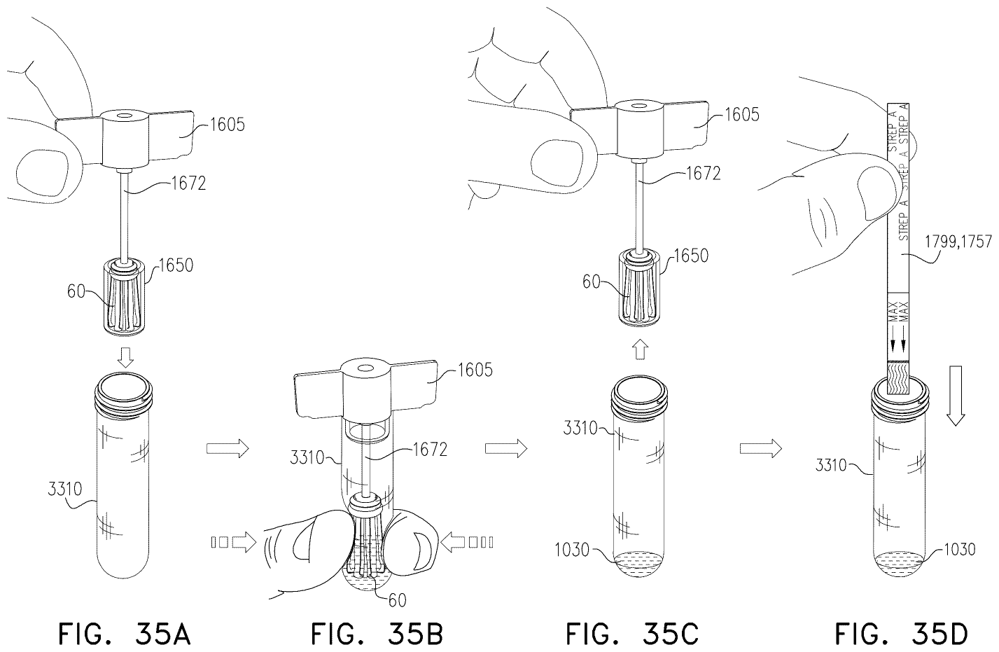

Reference is now made to FIGS. 35A-D, which are schematic illustrations of a portion of a method for testing for presence of a particulate, in accordance with an application of the present invention. This portion of the method may optionally be implemented after filter 60 has been covered and/or shielded by collection vial 1650, optionally using any of the sampling devices described herein or another sampling device. For example, this portion of the method may be performed after the step of the method described hereinabove with reference to FIG. 3J or after the step of the method described hereinabove with reference to FIG. 34D. This method may optionally be implemented in combination with any of the techniques described hereinabove with reference to FIGS. 32A, 32B-K, 33A-B, and/or 33C, mutatis mutandis.

As described hereinabove with reference to FIGS. 3J and 34D, after bunched-up filter 60 has been exposed to liquid 1030 in extraction tube 1718 (typically bathed in liquid 1030 for a certain amount of time), filter-withdrawal shaft 1672 is withdrawn from extraction tube 1718. This proximal movement of filter-withdrawal shaft 1672 with respect collection vial 1650 pulls filter 60 at least partially into (such as entirely into) collection vial 1650 via vial opening 1652. Typically, bunched-up filter 60 is not squeezed while in extraction tube 1718 in order to avoid removing particulate from filter 60; however, bunched-up filter 60 may be alternatively squeezed in extraction tube 1718, such as described hereinabove with reference to FIG. 3G.

This removal of bunched-up filter 60 from extraction tube 1718 discards a portion of liquid 1030 by leaving the liquid 1030 in extraction tube 1718. Typically, the concentration of the target analyte in the portion of liquid 1030 that remains trapped in bunched-up filter 60 is higher than the concentration of target analyte in the discarded portion of liquid 1030.

As shown in FIG. 35A, bunched-up filter 60 is inserted into a testing tube 3310 (via a proximal end opening of the testing tube), which is separate and distinct from extraction tube 1718. Before insertion of bunched-up filter 60, testing tube 3310 is typically empty of liquid 1030, and typically entirely empty of any liquid.

For some applications, in order to insert bunched-up filter 60 into testing tube 3310, collection vial 1650 is inserted at least partially into testing tube 3310, and distally advanced within testing tube 3310 until bunched-up filter 60 is positioned near a distal end of testing tube 3310 opposite a proximal end opening of testing tube 3310. (As mentioned above, filter 60 was earlier covered and/or shielded by collection vial 1650, optionally using any of the sampling devices described herein or another sampling device.) Testing tube 3310 is shaped so as to prevent collection vial 1650 from reaching the distal end of testing tube 3310, such that collection vial 1650 slides up a portion of filter-withdrawal shaft 1672 as bunched-up filter 60 is positioned near the distal end of testing tube 3310, thereby ejecting bunched-up filter 60 from vial opening 1652 of collection vial 1650 and exposing bunched-up filter 60 to an interior of testing tube 3310.

For some applications, as shown in FIG. 35B, filter 60 is squeezed, typically by squeezing testing tube 3310, which is flexible in these applications. Squeezing of filter 60 squeezes a portion of liquid 1030 (and optionally a portion of any of liquid specimen sample 22 trapped by filter 60) from filter 60 into testing tube 3310.

Optionally, the bunched-up portion of filter 60 is rotated one or more times, optionally both clockwise and counterclockwise, such as by rotating shaft handle 1605.

As shown in FIG. 35C, bunched-up filter 60 is removed from testing tube 3310, optionally by withdrawing filter-withdrawal shaft 1672 from testing tube 3310. Typically, this proximal movement of filter-withdrawal shaft 1672 with respect collection vial 1650 pulls filter 60 at least partially into (such as entirely into) collection vial 1650 via vial opening 1652.

For some applications, bunched-up filter 60 is removed from testing tube 3310 while testing tube 3310 is squeezed. For example, testing tube 3310 may be squeezed before removing bunched-up filter 60, such as shown in FIG. 35B, and testing tube 3310 may continue to be squeezed during removal of bunched-up filter 60 from testing tube 3310.

For some applications, such as shown in FIG. 35D, lateral flow test strip 1799 is inserted into the liquid in testing tube 3310, in order to test for the presence of a target analyte released from the particulate. In these applications, lateral flow test strip 1799 is typically implemented as dipstick 1757.

Optionally, any of the kits described herein may alternatively or additionally comprise testing tube 3310.

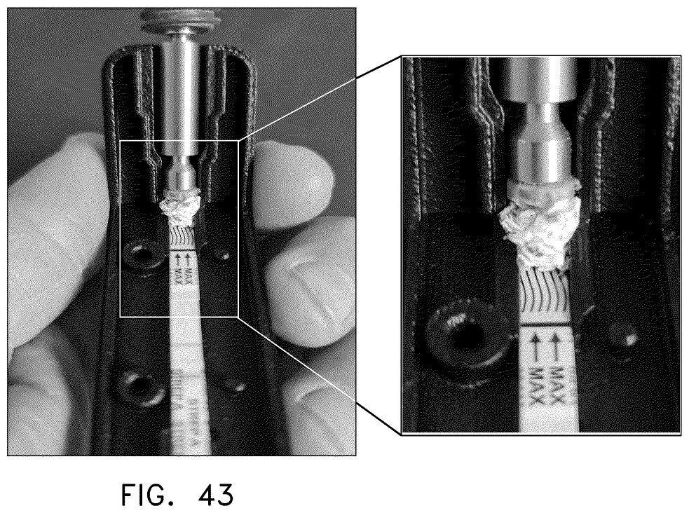

Reference is now made to FIGS. 36A-B and 37A-B, which are schematic isometric and cross-sectional illustrations of a kit 3400 and a method of using kit 3400 for testing for presence of a particulate, respectively, in accordance with an application of the present invention. Reference is also made to FIG. 43, which is a photograph of one implementation of kit 3400, in accordance with an application of the present invention. The method of using kit 3400 may optionally be implemented after filter 60 has been covered and/or shielded by collection vial 1650, optionally using any of the sampling devices described herein or another sampling device. Optionally, kit 3400 may implement any of the features of kit 3000, described hereinabove, mutatis mutandis.

Kit 3400 comprises a housing 3410 and lateral flow test strip 1799, which optionally comprises a lateral flow immunoassay test strip. Housing 3410 is typically shaped so as to define a test-strip receptacle 3412 for placement of lateral flow test strip 1799 therein, such as via a proximal end opening 3416 of test-strip receptacle 3412. Test-strip receptacle 3412 is typically elongate. Housing 3410 typically is shaped so as to define one or more result windows 3414 through which a portion of the lateral flow test strip is visible, the portion typically including test area 1788.

Kit 3400 may be configured to allow insertion of lateral flow test strip 1799 into test-strip receptacle 3412 during the test procedure, without disassembling housing 3410, such that lateral flow test strip 1799 is contained at least partially within housing 3410, such as shown. Alternatively, kit 3400 may be provided with lateral flow test strip 1799 pre-inserted at least partially into housing 3410. In either case, kit 3400 may be configured to allow removal of lateral flow test strip 1799 from housing 3410 after testing, which may enable easier reading of the test line of test area 1788. For applications in which one or more elements of kit 3400 are provided in sterile packaging, optionally separate sterile packaging is provided for lateral flow test strip 1799. Typically, lateral flow test strip 1799 may be inserted into test-strip receptacle 3412 with the strip's top surface pointing in either direction, without affecting the quality of the test results (the top surface includes sample pad 1797).

Kit 3400 further comprises liquid 1030, which comprises one or more reagents, and one or more containers 1022 that contain liquid 1030 (not shown in FIGS. 36A-B and 37A-B, but shown in FIG. 32A for kit 3000). Optionally, liquid 1030 comprises two or more liquids, which are contained in two or more respective containers 1022, and are combined during the testing procedure, such as shown in FIG. 32B.

Housing 3410 is shaped so as to define a channel 3426 in fluid communication with sample pad 1797 of lateral flow test strip 1799, when lateral flow test strip 1799 is disposed within test-strip receptacle 3412. Optionally, channel 3426 has one or more of the dimensions provided hereinabove with reference to FIGS. 32A-K for channel 3026.

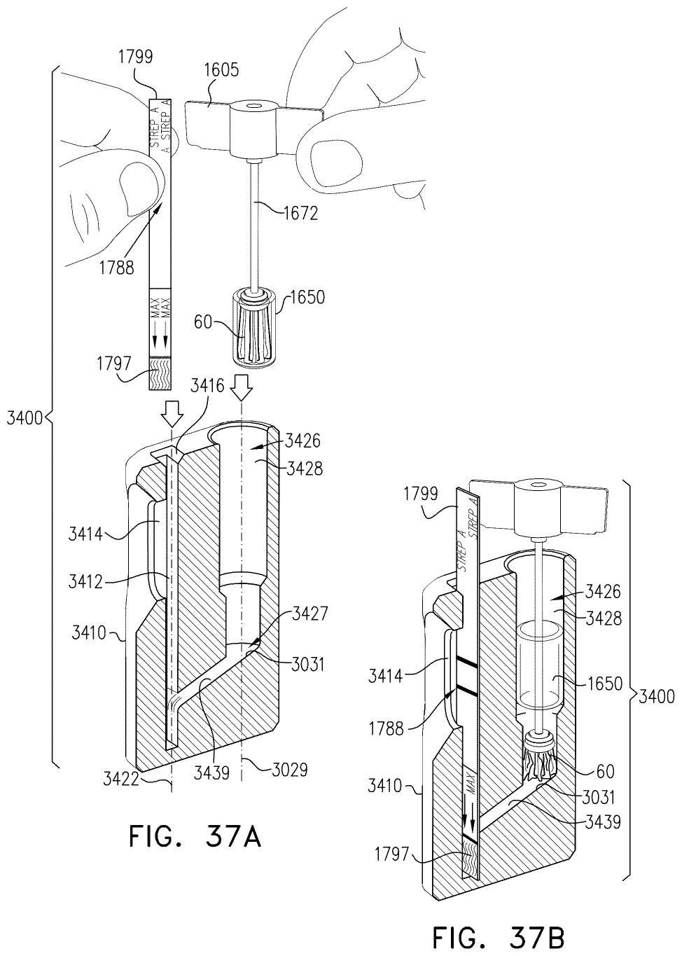

For some applications, a central longitudinal axis 3422 of test-strip receptacle 3412 (a) defines an angle of less than 60 degrees, such as less than 45 degrees, e.g., less than 30 degrees, with straight central longitudinal axis 3029 of channel 3426 (labeled in FIG. 37A), or (b) is parallel with straight central longitudinal axis 3029, such as shown. Typically, housing 3410 is configured to be used while straight central longitudinal axis 3029 of channel 3426 defines an angle of 45-90 degrees, such as 60-90 degrees, with a surface that is horizontal with respect to the Earth, e.g., is perpendicular with a surface that is horizontal with respect to the Earth.

As shown in FIGS. 36B and 37B, the bunched-up portion of filter 60 is squeezed to squeeze a portion of liquid 1030 from filter 60 by inserting the bunched-up portion of filter 60 into channel 3426 (via a proximal end opening of the channel) so that the bunched-up portion of filter 60 is squeezed by an internal wall 3428 of channel 3426; typically, internal wall 3428 is sufficiently rigid so as not to deform while squeezing the bunched-up portion of filter 60. Thus, internal wall 3428 of channel 3426 is shaped so as to squeeze the bunched-up portion of filter 60 upon insertion and distal advancement of the bunched-up portion of filter 60 in channel 3426, typically without channel 3426 being externally squeezed.

A distal portion of the bunched-up portion of filter 60 is advanced within channel 3426 until bunched-up filter 60 is pressed against distal blocking surface 3031 defined by channel 3426 (upstream of sample pad 1797), so as to longitudinally compact the bunched-up portion of filter 60. The portion of liquid 1030 expelled from the bunched-up portion of filter 60 comes into contact with sample pad 1797 of lateral flow test strip 1799, optionally via a passageway 3439 to test-strip receptacle 3412, the passageway downstream of distal blocking surface 3031, such as shown in FIG. 37B. (As mentioned above regarding channel 3026, straight central longitudinal axis 3029 may form an oblique angle (a non-right angle) with distal blocking surface 3031, such as shown, or may form a right angle (configuration not shown).)

Optionally, filter 60 is rotated one or more times, optionally both clockwise and counterclockwise, such as by rotating shaft handle 1605, in order to facilitate squeezing liquid from filter 60.

As shown in FIGS. 36B and 37B, lateral flow test strip 1799 is used to test for the presence of a target analyte released into liquid 1030 from particulate trapped by filter 60.

As described hereinabove with reference to FIGS. 12F, 30, and 31, for some applications, the at least a portion of filter 60 is bunched up such that an entirety of perimeter 1085 of filter 60 points distally (labeled in FIGS. 30 and 31). For some of these applications, such as shown in FIGS. 36A and 37A, the bunched-up portion of filter 60 is inserted into channel 3426 in a distal direction while the entirety of perimeter 1085 of filter 60 points distally.

In an alternative configuration (not shown, but similar in some respect to the configuration shown above in FIG. 32I), the bunched-up portion of filter 60 is inserted into channel 3426 by inserting collection vial 1650 at least partially into channel 3426, and distally advancing the collection vial within channel 3426 until bunched-up filter 60 is advanced out of the collection vial, through channel 3426, and into direct contact with sample pad 1797 of lateral flow test strip 1799. Collection vial 1650 may aid with the insertion of bunched-up filter 60 into channel 3426. Collection vial 1650 is readily inserted into channel 3426, thereby inserting bunched-up filter 60 into channel 3426 while the bunched-up filter is initially within collection vial 1650.

For some of these applications, the distal portion of the bunched-up portion of filter 60 is advanced through channel 3426 so that at least a portion of perimeter 1085 of filter 60 (labeled in FIGS. 30 and 31) makes direct contact with sample pad 1797 of lateral flow test strip 1799. Typically, internal wall 3428 is shaped so as to hold perimeter 1085 pointing distally and direct the perimeter to sample pad 1797.

For some applications, housing 3410 is not shaped so as to define an extraction tube (similar to extraction tube 3018, described hereinabove with reference to FIGS. 32A-K). Instead, kit 3400 comprises a separate extraction tube 1718, for example as described hereinabove with reference to FIGS. 34A-D. For example, as shown in FIGS. 34B-C, collection vial 1650 is inserted at least partially into extraction tube 1718 (via proximal end opening 1721 of extraction tube 1718).

For some applications, housing 3410 is not shaped so as to define an extraction tube (similar to extraction tube 3018, described hereinabove with reference to FIGS. 32A-K). Instead, channel 3426 is additionally used for bathing bunched-up filter 60 in liquid 1030. Liquid 1030 may be dispensed from the one or more containers 1022 into channel 3426, such as described hereinabove with reference to FIG. 32B, mutatis mutandis. Alternatively, channel 3426 may comprise one or more internal containers that contain liquid 1030 and are ruptured upon insertion and/or distal advancement of bunched-up filter 60 into channel 3426. Optionally, channel 3426 is configured to allow excess liquid 1030 to drain from the channel after bunched-up filter 60 has been bathed in the liquid. Alternatively or additionally, optionally, excess liquid 1030 is discarded by further advancing bunched-up filter 60 out of channel 3426 and leaving a portion of liquid 1030 in channel 3426.

For some applications, housing 3410 is shaped so as to define an extraction tube, which may, for example, be similar to extraction tube 3018, described hereinabove with reference to FIGS. 32A-K. As shown for kit 3000 in FIG. 32B, liquid 1030 is dispensed from the one or more containers 1022 into extraction tube 3018. As shown for kit 3000 in FIGS. 32C-E, collection vial 1650 is inserted at least partially into extraction tube 3018, and distally advanced within extraction tube 3018 until bunched-up filter 60 is positioned near a distal end 3015 of extraction tube 3018 opposite a proximal end opening 3021 (labeled in FIG. 32E). (Filter 60 was earlier covered and/or shielded by collection vial 1650, optionally using any of the sampling devices described herein or another sampling device.) Extraction tube 3018 may have any of the characteristics described hereinabove with reference to FIGS. 32A-K. Optionally, as shown for kit 3000 in FIG. 32F, the bunched-up portion of filter 60 is rotated one or more times, optionally both clockwise and counterclockwise, such as by rotating shaft handle 1605. After bunched-up filter 60 has been exposed to liquid 1030 in extraction tube 3018 (typically bathed in liquid 1030 for a certain amount of time), filter-withdrawal shaft 1672 is withdrawn from extraction tube 3018. This proximal movement of filter-withdrawal shaft 1672 with respect collection vial 1650 pulls filter 60 at least partially into (such as entirely into) collection vial 1650 via vial opening 1652, such as shown for kit 3000 in FIG. 32G, thereby at least partially (e.g., entirely) covering and/or shielding filter 60.

Reference is made to FIGS. 32I, 33A-C, 36A-B, and 37A-B. Although the channels of these configurations are described as being defined by respective housings containing at least a portion of lateral flow test strip 1799, these channels may alternatively be implemented not in combination with these particular housings. For example, separate tubes may be provided that are shaped so as to define any of these channels.

A number of experiments were performed by or on behalf of the inventors using some of the techniques described herein. These experiments demonstrate inter alia the efficacy of some of the filter-elution techniques described herein.

### Experiment 1

### Section 1: Materials and Methods

### Section 1.1: Clinical Pilot Study

A pilot study was conducted with subjects belonging to the health care service provider Leumit (Tel-Aviv Israel) (IRB approval through Assaf Harofeh [Shamir] Medical Center, Be'er Yaakov, Israel; ClinicalTrials.gov identifier: NCT05223634) comparing the Group A Streptococcus (GAS) detection efficacy of conventional throat cultures with a filter concentration device utilizing a filter elution method including pounding the filter, as described hereinbelow in Section 1.1.3.

### Section 1.1.1: Clinical Pilot Study: Subject Inclusion and Exclusion

The study population included 61 subjects, 28 of which were determined to be positive for the presence of GAS by throat culture, with the remaining 33 subjects negative for pharyngeal GAS. In order to enrich the number of positive patients, in some instances subjects that produced a positive result from a throat swab that underwent rapid GAS antigen testing were preferentially enrolled. Exclusion criteria consisted of 1) the absence of a gargling sound during sample procurement, 2) antibiotics taken within 48 hours prior to sampling, 3) eating or drinking during sample procurement, 4) procedural deviations during sample collection or processing, and 5) exceeding defined transport conditions. In one instance, a subject was excluded due to discrepant result analysis generating conflicting results after patient re-sampling (a subject that was originally culture negative was re-visited on a subsequent day, and throat culture and PCR results from the secondary samples were positive for GAS [concordant with results from our device]). In a single instance, a sample was discarded due to health and safety concerns after determining the subject was infected with COVID-19.

### Section 1.1.2: Clinical Pilot Study: Specimen Collection

Throat swab specimens were collected by medical professionals, using two throat swabs simultaneously to bilaterally swab the tonsillar regions and the posterior oral pharynx. One of the two throat swab specimens were used for a rapid GAS antigen test for the purpose of immediate patient management and in some cases to determine subject enrollment (as stated in Section 1.1.1), and the second throat swab was sent to the Leumit Health Services Central Laboratory (Or Yehuda, Israel), the New Rambam Laboratories (Jerusalem, Israel), or Hero Scientific (Jerusalem, Israel), in Amies transport medium (Copan, Brescia, Italy, Reference #108C) for culturing. Subjects were then asked to gargle 10 mL of sterile 0.9% saline for approximately 10 seconds, and then spit the gargle sample out into a sterile sample collection cup. Gargle samples were then transferred to and passed through concentration devices containing 1.2 μm Polyethersulfone (PES) membrane filters (Sterlitech, Kent, WA, USA, Catalog number: PES122005) cut to a diameter of 25 mm. Following initial sample processing, concentration devices were transported on ice and stored at 4° C. for up to 72 hours prior to further processing. Alternatively, gargle samples were transported to Hero Scientific (Jerusalem, Israel) on ice, and then passed through concentration devices, as above.

### Section 1.1.3: Clinical Pilot Study: Throat Culturing

Throat cultures sent to the Leumit Health Services Central Laboratory (Or Yehuda, Israel) were plated onto selective Group A Streptococci Agar plates (Hylabs, Israel, Reference #PD214) with Bacitracin disks, and incubated for a maximum of 16 hours at 37° C. with 5% CO₂. Requirements for a positive culture included colony growth on the GAS selective plates, sensitivity to Bacitracin, and the presence of β-hemolysis.

Throat cultures sent to the New Rambam Laboratories (Jerusalem, Israel) were plated onto Tryptic Soy Agar plates containing 5% sheep blood with Bacitracin disks and incubated overnight at 37° C. with 5% CO₂. In the event of β-hemolytic, Bacitracin sensitive growth, colonies were sampled, gram stained, and examined by light microscopy for streptococcal morphology to confirm a presumptive GAS diagnosis.

Throat cultures sent to Hero Scientific (Jerusalem, Israel) were plated onto Tryptic Soy Agar plates containing 5% sheep blood (Hylabs, Israel, Reference #PD049) with Bacitracin disks for 16-48 hours, and examined for the presence of β-hemolytic, Bacitracin sensitive growth. Presumptive GAS positive colonies were further sampled and subjected to a rapid GAS antigen test for diagnostic confirmation.

### Section 1.1.3: Clinical Pilot Study: Gargle Sample Processing Using the Pounding Method

Filters were removed from the concentration devices with forceps, folded into quadrants, and placed into sterile 5 mL culture tubes (Labcon, California, USA, Reference #LC335-335). Four drops of 2M Sodium Nitrite (Consult Diagnostics, Richmond, VA, USA., Catalog Number: L060209-02) and 4 drops of 0.2M Acetic Acid (Consult Diagnostics, Richmond, VA, USA., Catalog Number: L060210-02) were added to the culture tubes, and filters were pounded for 30 seconds using the blunt end of a microbial loop (Heathrow Scientific, Illinois, USA, Reference #HS81121D). In some instances, the microbial loop was used to turn the filter intermittently to allow for homogenous pounding depending on filter orientation. Following pounding, filter samples were left in the reagents for a subsequent 2 minutes and 30 seconds, and then a Mckesson Strep A Dipstick (Consult Diagnostics, Richmond, VA, USA., Reference #5003) was inserted into the tube. The dipstick was removed from the tube 5 minutes later, and control and test bands were examined after a second 5-minute period. This facilitation of filter elution by pounding is referred to hereinbelow as the “Pounding Method.”

### Section 1.2: Comparative Testing of Elution Methodologies

Healthy individuals were asked to gargle 11 mL of Dulbecco's Phosphate Buffered Saline DPBS without calcium and magnesium (DPBS) (Biological Industries, Israel, Reference #02-023-1A) for approximately 10 seconds, and gargle samples were pooled together, and vortexed for approximately 1 minute. Bacterial inoculum was made by sampling multiple colonies of GAS (ATCC 19615) following at least 16 hours of growth on Tryptic Soy Agar plates containing 5% sheep blood (Hylabs, Israel, Reference #PD049) at 35.5° C. with a cotton swab, and eluting the contents into 1-2 mL of DPBS (Biological Industries, Israel, Reference #02-023-1A). Fourteen aliquots of pooled gargle between 22 mL and 40 mL were made, spiked with bacterial inoculum, and vortexed for approximately 45 seconds. Paired samples each of 10 mL of spiked gargle were sampled twice from the same aliquot and passed simultaneously through two concentration devices (described in Section 1.1.2) containing respective identical filters (1.2 μm Polyethersulfone (PES) membrane filters. Paired filters were then removed from the concentration devices for elution and testing.

### Section 1.2.1: Comparative Testing of Elution Methodologies: Filter Elution

14 individual filter comparisons were made, 8 of which compared the Pounding Method as described hereinabove in Section 1.1.3 to either a Turning Method or Pooling Method respectively, as described hereunder; and 6 of which compared the Turning Method to a Stationary Method, as described hereunder.

In 7 of the pairs of filters compared to the Pounding Method, the second paired filter was perforated in the center by spinning the blunt end of a microbial loop (Heathrow Scientific, Illinois, USA, Reference #HS81121D) while pressing down against the upstream side of the filter. A ring-shaped sticker with an outer diameter of 7 mm was placed over the hole to reinforce the contact point, a screw was inserted into the hole, and screwed into the shaft such that the upstream side of the filter was oriented away from the shaft. An extraction tube included in the OSOM Strep A Test kit (Sekisui Diagnostics, LLC, San Diego, CA, USA, Reference #141) was cut lengthwise with a scalpel. A second cut was then made along the width of the tube, severing the tube at around half its length. The tube half that was open from both sides was placed over the shaft, positioned proximally to the filter, and pushed down, folding the filter inwards. Pressure was then applied to the cut extraction tube while it was still in contact with the folded filter, pushing the two ends formed by the lengthwise cut past one another, tightening the folding of the filter. A fresh OSOM strep A extraction tube was filled with 4 drops of each reagent as described in Section 1.1.3, the filter was positioned over the extraction tube, and pushed into the extraction tube, bathing the filter in the extraction reagents. The shaft was spun 10 times to the right and 10 times to the left, and an additional 5 times to the right and 5 times to the left in the extraction reagents while gently squeezing the extraction tube. After a total of 3 minutes of contact between the filter and extraction reagents, the filter was removed from the tube by pulling up on the shaft while simultaneously squeezing the filter through the walls of the soft extraction tube prior to dipstick insertion in order to remove any fluid contained on or within the filter. This filter elution method is referred to hereinbelow as the “Turning Method.”

For one of the pairs of filters compared to the Pounding Method, the second paired filter was removed from the concentration device with forceps and placed on the underside of a cap from a sterile urine cup (FL MEDICAL S.R.L., Italy, Reference #25032) oriented such that the upstream side of the filter was facing upwards. Four drops of each reagent (described in Section 1.1.3) were added directly to the middle of the filter and incubated together for 3 minutes without any physical manipulation. The cap was then placed at an approximate 45° incline to facilitate the pooling of extraction reagent and antigen, and the dipstick was inserted into the pooled reagent/antigen mixture. This filter elution method is referred to hereinbelow as the “Pooling Method.”

In 6 of the filter comparisons, one of the pairs was processed utilizing the Turning Method as detailed above, and the other pair was prepared exactly as described above for the Turning Method, except, however, unlike in the Turning Method, the shaft, once inserted within the lysing tube, was not turned from side to side but rather left stationary for 3 minutes (this filter elution method is referred to hereinbelow as the “Stationary Method”). The sides of each tube were pinched to squeeze out the filter upon removal. Test strips were inserted and removed from each tube after 5 minutes and compared for test line intensity.

### Section 1.2.2: Comparative Testing of Elution Methodologies: Lateral Flow Antigen Testing

Mckesson Strep A Dipsticks (Consult Diagnostics, Richmond, VA, USA, Reference #5003) were simultaneously inserted into both extraction tubes of each pair noted above (or the extraction tube and the pooled reagent/antigen mixture at the bottom of the sterile urine cup cap), and incubated for 5-minutes prior to removal from the reaction mixtures. Dipsticks were examined after a second 5-minute wait period, and band intensity was scored on a scale of 0-5 to determine semi-quantitative differences in GAS extraction.

### Section 2: Results

### Section 2.1: Clinical Pilot Study Results

24 of the 28 throat-culture-positive subjects and 31 of the 33 throat-culture-negative subjects were concordant when testing the concentration device and using the Pounding Method described hereinabove in Section 1.1.3, corresponding to a sensitivity and specificity of 85.7% and 93.9%, respectively (Table 1).

TABLE 1
Clinical Pilot Study Results
Clinical Sensitivity and Specificity of Concentrated Gargle Samples

| | | Throat Culture | | |
|---|---|---|---|---|
| | | Positive | Negative | Total |
| Concentrated | Positive | 24 | 2 | 26 |
| Gargle | Negative | 4 | 31 | 35 |
| | Total | 28 | 33 | |
| | | Sensitivity (%), | 85.71 (72.75- | |
| | | (95% CI) | 98.68) | |
| | | Specificity (%), | 93.94 (85.80- | |
| | | (95% CI) | 100) | |

### Section 2.2: Results from Comparative Testing of Elution Methodologies

In all 7 paired filters comparing the Pounding Method and the Turning Method of filter elution, the Turning Method produced stronger band intensities when compared to the Pounding Method, with an average band intensity increase of 1.57 on a scale of 0-5. This difference was statistically significant, with the p value from a two-tailed paired t-test of 0.0059.

In the single paired filter comparing the Pounding Method and the Pooling Method of filter elution, the Pounding Method produced a stronger band intensity than the Pooling Method, with an increase of 0.75.

In the 6 paired filter comparisons between the Turning Method and the Stationary Method, the average line intensity of the Turning Method resulted in a 0.675 higher average band intensity as compared to the Stationary Method.

### Section 3: Discussion

Considering the comparison between the Pounding Method and the Pooling Method of filter elution, it appears that elution methodologies involving more rigorous physical manipulation correspondingly increase band intensity and lateral flow antigen testing sensitivity. However, the Pounding Method generally involved substantially more physical manipulation then that of the Turning Method, and so the significant increase in band intensity observed with the utilization of the novel Turning Method of elution over that of the Pounding Method (as described in Section 2.2) is surprising. In consideration of the superior results obtained by the Turning Method compared to the Pounding Method, as shown during the comparative testing, one would expect a significant increase in diagnostic sensitivity over that of the 85.71% sensitivity observed in the clinical pilot study using the Pounding Method (Section 2.1) with the implementation of the Turning Method of filter elution in subsequent clinical trials.

Additionally, while the Stationary Method of elution resulted in a slightly lower average band intensity when compared to the Turning Method, even so, it is still far superior to the Pounding Method which yielded an average band intensity of 1.57 lower than the Turning Method. Accordingly, both the Turning Method and Stationary Method would be expected to yield higher performance results in subsequent clinical trials.

### Experiment 2

### A Direct Comparison Between a Single Tube and Two Tubed Elution Method for Rapid Strep Antigen Tests

### Section 1: Methods and Materials

Group A Streptococcus (GAS) (ATCC 19615) colonies were picked with a sterile cotton swab from a Tryptic Soy Blood Agar plate (TSBA) containing 5% defibrillated sheep blood (Hylabs, Rehovot, Israel, Ref: PD-049) following incubation at 35.5° C. The cotton swab was then immersed into a 5 mL culture tube (Labcon, CA, USA., Ref: 3335-335-000-9) containing 1-2 mL of Dulbeco's Phosphate Buffered Saline (DPBS) (Sartorius, Beit HaEmek, Israel, Ref: 02-023-1A). The head of the swab was pressed against the side of the culture tube and rotated 5-10 times to promote elution of GAS from the swab. The GAS inoculated DPBS was then vortexed for approximately 45 seconds. Laboratory personnel were asked to pre-rinse their mouths by repeatedly swishing a mouthful of water 3-5 times, and repeating until approximately 250 ml of water had been used. Swished water was spat out, and 11 mL of sterile DPBS was subsequently gargled for approximately 10 seconds by laboratory personnel. Gargle samples were pooled together and spiked with GAS inoculated DPBS. The spiked pooled gargle sample was vortexed for approximately 30 seconds, and 10 mL of spiked gargle was transferred into two identical Hero Scientific Ltd. Swabless™ concentration devices, similar to sampling device 1620, described hereinabove with reference to FIGS. 1A-B, 2A-D, and 3A-D. Each concentration device contained a 1.2 μm Polyethersulfone (PES) filter (Sterlitech, WA, USA., Ref: PES122005) cut to a diameter of 29.3 mm. The aliquoted spiked gargle was passed through the filters, and filters were removed from the concentration devices by turning a wing nut at the top of the device, and pulling up on the wing nut, such as described hereinabove with reference to FIGS. 2B-D and 3B-D.

Two extraction tubes included in the Mckesson Strep A Dipstick test kit (Consult Diagnostics, Richmond, VA, USA., Reference #5003) were cut to accommodate the length of the filter and filter-withdrawal shaft, and 4 drops of both 2M Sodium Nitrite and 0.2M Acetic acid (included in the Mckesson Strep A Dipstick test kit as Reagents 1 and 2, respectively) were added to each tube. The filters were inserted into respective extraction tubes, rotated 5 times clockwise and 5 times counterclockwise, and then bathed in the extraction reagents for 3 minutes. The filters were then eluted, by one of the following methods:
by squeezing in the extraction tube, and pinching during removal from the extraction tube (hereinbelow, the Single Tube Method); or
by being removed from the extraction tube, inserted into an empty extraction tube, squeezed out, and then pinched during removal (hereinbelow, the Two Tube Method).

Mckesson Strep A Dipsticks (Consult Diagnostics, Richmond, VA, USA., Reference #5003) were simultaneously inserted into both the Single Tube Method extraction tube and the Two Tube Method secondary extraction tube, and bathed in the extraction reagents for 5-minutes prior to removal from the reaction mixtures. Dipsticks were examined after a second 5-minute wait period, wherein, line intensity was scored on a scale of 0-5 to determine semi-quantitative differences in GAS extraction.

### Section 2: Results and Discussion

Line intensity for the sample eluted using the Single Tube Method was 1 compared to a line intensity of 2 for the paired sample eluted using the Two Tube Method. It is hypothesized by the inventors that the chemical elution of antigen off of the filter may be incomplete without the addition of physical manipulation such as squeezing. When squeezing into the original volume of extraction reagents, there may be an unnecessary dilution of the relatively high concentration of antigen still associated with the filter, which can be mitigated by squeezing into a separate tube prior to testing (i.e., by using the Two Tube Method).

### Experiment 3

### The Utilization of a Two Tube Elution Method in Testing for Group a Streptococcal Pharyngitis

### Section 1: Methods and Materials

An 8-year-old male presenting with a sore throat and fever tested positive for Group A Streptococcus (GAS) by a rapid antigen test performed on a throat swab sample. The subject was asked to pre-rinsed his mouth by swishing a mouthful of water 3-5 times and then spit the water out. The subject then gargled 10 mL of 0.9% Saline (Braun, Melsungen, Germany, Ref: 3642828) for approximately 10 seconds 4 times in succession, and spat each gargle into a separate sterile sample collection cup (FL Medical s.r.l., Torreglia, Italy, Ref: 25036). Samples were transported on ice for approximately 1 hour and then stored at 4° C. Samples were transferred into Hero Scientific Ltd. Swabless™ concentration devices, similar to sampling device 1620, described hereinabove with reference to FIGS. 1A-B, 2A-D, and 3A-D. Each concentration device contained a 1.2 μm Polyethersulfone (PES) filter (Sterlitech, WA, USA., Ref: PES122005) cut to a diameter of 29.3 mm. The samples were then passed through the filters. The filters were then removed from the concentration devices by turning a wing nut at the top of the device, and pulling up on the wing nut, such as described hereinabove with reference to FIGS. 2B-D and 3B-D. The first and second gargle samples were filtered in quick succession approximately 5 hours after sample procurement, while the third and fourth gargle samples were filtered approximately 8 and 10 hours following sample procurement, respectively.

Testing housings containing an indentation for the insertion of a rapid antigen test strip, a Reagent Tube, and a Testing Channel (FIG. 1.1) were 3D printed out of Nylon (Laser Modeling) and fitted with Mckesson Strep A dipsticks (Consult Diagnostics, Richmond, VA, USA., Reference #5003). The testing housings were similar to housing 3010 described hereinabove with reference to FIGS. 32A-K.

For antigen testing, 4 drops of 2M Sodium Nitrite and 0.2M Acetic acid (included in the Mckesson Strep A Dipstick test kit as Reagents 1 and 2, respectively) were added to the extraction tube of the housing, which was similar to extraction tube 3018, described hereinabove with reference to FIGS. 32A-K, and filters were inserted into the extraction tube and bathed in the extraction reagents for 3 minutes. The filters were removed from the extraction tube and inserted into a testing channel, similar to channel 3026 described hereinabove with reference to FIGS. 32A-K, and fully pushed down into direct contact with the test strip. Rapid antigen test strips were then examined after 10 minutes, and line intensity was scored on a scale of 0-5 to determine semi-quantitative differences in the presence of GAS antigen.

### Section 2: Results and Discussion

In all cases, there was sufficient liquid adhered to the filter to facilitate strip migration and generate visible control and test lines. It was encouraging that test line intensity was visible for all samples (Table 2), indicating the successful detection of GAS antigen despite the extended transport and storage time (as detailed in Section 1), as previous experiments have demonstrated significant degradation of GAS viability and antigenicity in contrived gargle samples (data not shown). This data supports the feasibility of an antigen testing method for the detection of pharyngeal GAS wherein the sample and test strip are brought in contact in a chamber isolated from the principal volume of extraction reagents used during testing.

TABLE 2
Test Line Intensity

| Gargle Number (According | Test Line Intensity |
|---|---|
| to Procurement Order) | at 10 minutes |
| 1 | 4 |
| 2 | 3 |
| 3 | 3 |
| 4 | 1.5 |

### Experiment 4

In an experiment conducted on behalf of the inventors, gargle fluid was spiked with Group A Streptococcus (GAS), and filtered onto two filters using two respective sampling devices similar to sampling device 1620, described hereinabove with reference to FIGS. 1A-3D. The two filters were removed from their respective sampling devices and separately bathed in extraction reagents in respective extraction tubes. Subsequently, one of the filters was tested in a housing similar to housing 3410, described hereinabove with reference to FIGS. 36A-B and 37A-B, and the other filter was tested in a housing similar to the configuration of housing 3010 described hereinabove with reference to FIG. 33A. Very similar migration of the reagents along the lateral flow test strip was observed for the two different housings, and very similar test line and control line intensity was observed for the two different housings.

Reference is now made to FIGS. 3E-I, 4, 8A-E, 9A-E, 25E, 26E, 28E, and 29E. In some applications of the present invention, extraction tube 1718 and the withdrawer are configured to limit maximum distal advancement of bunched-up filter 60 within extraction tube 1718, such that bunched-up filter 60 is not crushed against the distal end of extraction tube 1718, e.g., such that bunched-up filter 60 does not touch the distal end of extraction tube 1718. Such crushing of the filter against the distal end of the tube would inhibit entry of liquid 1030 to the distally-open internal space defined by bunched-up filter 60, thereby reducing contact between liquid 1030 and biological particulate trapped on the inner surface of bunched-up filter 60. For example, the withdrawer may comprise an element that functions as a stopper to prevent over-insertion of the filter-withdrawal shaft and thus filter into extraction tube 1718. For example, extraction-tube cap 1719, seal 1614, or shaft handle 2205 may function as the stopper.

For some of these applications, extraction tube 1718 and the withdrawer are configured to set an extent of the distal advancement of bunched-up filter 60 within extraction tube 1718 to a predetermined distance of the distal advancement, so as to set an axial location of bunched-up filter 60 within extraction tube 1718, such as near the distal end of the extraction tube, and hold and immobilize the filter at this location.

These configurations may be implemented in combination with any of the techniques described herein, including, but not limited to, those described hereinabove with reference to FIGS. 30 and 31.

Reference is again made to FIGS. 32A-K and 33A-C. In some applications of the present invention, housing 3010, 3110 is not shaped so as to define extraction tube 3018. Instead, channel 3026, 3126 is additionally used for bathing bunched-up filter 60 in liquid 1030. Liquid 1030 may be dispensed from the one or more containers 1022 into channel 3026, 3126, such as described hereinabove with reference to FIG. 32B, mutatis mutandis. Alternatively, channel 3026, 3126 may comprise one or more internal containers that contain liquid 1030 and are ruptured upon insertion and/or distal advancement of bunched-up filter 60 into channel 3026, 3126. Optionally, channel 3026, 3126 is configured to allow excess liquid 1030 to drain from the channel after bunched-up filter 60 has been bathed in the liquid. Alternatively or additionally, optionally, excess liquid 1030 is discarded by further advancing bunched-up filter 60 out of channel 3026 and leaving a portion of liquid 1030 in channel 3026.

Reference is now made to FIG. 38, which is a schematic illustration of a filtration assembly 2424, in accordance with an application of the present invention. The features of filtration assembly 2424 may implemented in combination with any of the filtration assemblies described herein, mutatis mutandis.

Filtration assembly 2424 comprises a tubular container 2430 and a plunger 2440, which comprises a plunger rod 2482. Plunger 2440 is shaped so as to define a first thread (configuration not shown) or one or more first thread segments 2493 (as shown) that face radially inwardly and are configured to engage a corresponding second thread 2495 that is defined by an external surface of tubular container 2430 and faces radially outward. For some applications, plunger 2440 comprises one or more thread supports 2497, which extend proximally alongside at least a portion of plunger rod 2482, separated from plunger rod 2482 to provide a space for insertion of tubular container 2430. The one or more thread supports 2497 are shaped so as to define the first thread (configuration not shown) or the one or more first thread segments 2493, respectively (as shown). Providing the threads may help the user apply sufficient and/or correct pressure to liquid specimen sample 22 for pushing liquid specimen sample 22 through a filter within tubular container 2430.

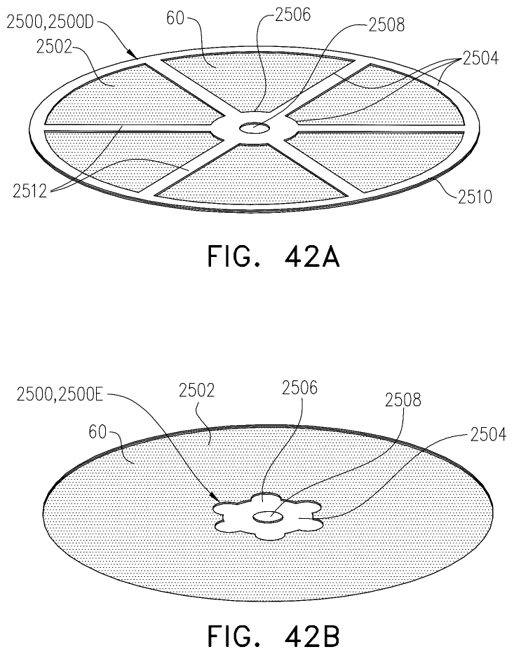

Reference is now made to FIGS. 39, 40, 41, and 42A-B, which are schematic illustrations of a filter reinforcement 2500 coupled (e.g., adhered by an adhesive) to filter 60, in accordance with respective applications of the present invention. Filter reinforcement 2500 may implemented in combination with filter 60 in any of the configurations and applications described herein. FIGS. 39, 40, 41, 42A, and 42B illustrate filter reinforcements 2500A, 2500B, 2500C, 2500D, and 2500E, respectively, all of which are implementations of filter reinforcement 2500. FIGS. 42A and 42B show opposites sides of filter 60, as described below.

Filter reinforcement 2500 may help prevent tearing of filter 60 as filter 60 is removed from the filtration assembly, such as when filter 60 is pulled through the central opening of the filter support. This prevention may be particular helpful in configurations in which filter 60 comprises a very fine material (such as a polyethersulfone (PES) membrane). In addition, filter 60 may help main the bunched-up shape of filter 60 after removal from the filtration assembly, which may aid with insertion of filter 60 into extraction tube 1718, such as described hereinabove.

Typically, filter reinforcement 2500 is coupled to a surface 2502 of filter 60 so as to cover at least 1%, such as at least 2% or at least 5%; no more than 50%, such as no more than 30%, e.g., no more than 20%; and/or 1%-50%, e.g., 1%-30%, e.g., 5%-25%, 5%-15%, or 5%-12%, of a surface area of surface 2502.

For some applications, filter reinforcement 2500 has a greater tensile strength than filter 60.

For some applications, filter reinforcement 2500 comprises metal. For other applications, filter reinforcement 2500 comprises a polymer. For still other applications, filter reinforcement 2500 comprises a curable material, which is applied to filter 60 before curing and then cured on the filter, such as by light or UV curing; for example, filter reinforcement 2500 may be printed onto filter 60.

For some applications, filter reinforcement 2500 is printed onto filter 60.

For some applications, filter reinforcement 2500 is not porous, while for other applications, filter reinforcement 2500 is porous, e.g., comprises a mesh.

For some applications, filter reinforcement 2500 has an average thickness of at least 0.01 millimeters (e.g., at least 0.02 millimeters), no more than 0.1 millimeters, and/or 0.01-0.1 millimeters.

For some applications, such as shown in FIGS. 39, 40, 41, and 42A-B, filter reinforcement 2500 is shaped so as to define a plurality of thin strips 2504. Thin strips 2504 may have constant or varying widths, and may have the same or differing width from one another. For example, thin strips 2504 may have a width of 0.1-1 mm, such as 0.1-0.7 mm.

For some applications, filter reinforcement 2500 (e.g., thin strips 2504 thereof) is shaped so as to define a central hub 2506. Optionally, central hub 2506 is shaped so as to define a central opening 2508 therethrough, such as to facilitate coupling to filter shaft 1072 (described with reference to FIGS. 30 and 31) or any of the filter-withdrawal shafts described herein.

For some applications, filter reinforcement 2500, 2500A, 2500C, 2500D is shaped so as to define a peripheral rim 2510.

For some applications, filter reinforcement 2500, 2500A, 2500B, 2500C, 2500D is shaped so as to define a plurality of spokes 2512. For some of these applications in which peripheral rim 2510 is provided, peripheral rim 2510 is connected to spokes 2512 (spokes 2512 may terminate at peripheral rim 2510, such as shown in FIGS. 39 and 42A, or extend radially outward beyond peripheral rim 2510, such as shown in FIG. 41). Alternatively or additionally, for some of these applications in which central hub 2506 is provided, central hub 2506 is connected to spokes 2512.

For some applications, the filtration assembly is configured such that movement of the plunger head within the tubular container, when the liquid specimen sample is contained in the tubular container and filter 60 is disposed in the tubular container, pushes at least a portion of the liquid specimen sample through filter 60 in an upstream-to-downstream direction. For some of these applications, surface 2502 to which filter reinforcement 2500 is coupled is an upstream surface of filter 60. For others of these applications, surface 2502 to which filter reinforcement 2500 is coupled is a downstream surface of filter 60.

For some of these applications, surface 2502 to which filter reinforcement 2500 is coupled is an upstream surface of filter 60, and the sampling device further comprises a downstream filter reinforcement, which is coupled to a downstream surface of filter 60, such as shown in FIGS. 42A-B, which, as mentioned above, show the two sides of filter 60.

Reference is now made to FIGS. 1A-8B and 11A-29E. In some applications of the present invention, a sampling device is provided for concentrating liquid specimen sample 22, the sampling device comprising filter 60, a filtration assembly, and a withdrawer.

The filtration assembly comprises:
a tubular container, which is shaped so as to define an inner wall and a proximal container opening for receiving liquid specimen sample 22;
a plunger, which (a) comprises a plunger head and (b) is insertable into the tubular container via the proximal container opening, such that a lateral surface of the plunger head forms a fluid-tight movable seal with the inner wall; and
a filter support, which is shaped so as to define (a) a support surface on which filter 60 is removably disposed, and (b) a plurality of filtrate-passage openings through the filter support.

The withdrawer comprises a filter-withdrawal shaft, which is partially inserted in the filtration assembly.

The filtration assembly is configured such that movement of the plunger head within the tubular container, when liquid specimen sample 22 is contained in the tubular container and filter 60 is disposed in the tubular container, pushes at least a portion of liquid specimen sample 22 through filter 60.

The sampling device is configured such that removal of the filter-withdrawal shaft from the filtration assembly, while a distal portion of the filter-withdrawal shaft is coupled to filter 60, while the plunger head remains within the tubular container, and while the filter support remains within the filtration assembly (optionally within the tubular container), removes filter 60 from the filter support and from the filtration assembly.

For some applications, the filter support is shaped so as to define a central opening, in addition to the plurality of filtrate-passage openings. The sampling device is configured such that removal of the filter-withdrawal shaft from the filtration assembly, while the plunger head remains within the tubular container and the filter support remains within the filtration assembly, pulls filter 60 through the central opening and removes filter 60 from the filter support and from the filtration assembly.

For some applications, a cross-sectional area of the central opening of the any of the filter supports described herein is 10-30 mm2, e.g., 15-25 mm2. For some applications in which the central opening is circular, a circumference of the central opening of the any of the filter supports described herein is 3-8, e.g., 4-6, e.g., 5 mm.

In any of the configurations described herein, another type of tube may be substituted for extraction tube 1718, such as a transport tube (in which case liquid 1030 may comprise a transport medium). (Although a transport tube is not labeled as such in the figures, any of the extractions tubes 1718 with caps shown in the figures may also serve as transport tubes.) For example, the transport tube may be transported to a remote laboratory, such as for performing Polymerase Chain Reaction (PCR) testing for particulate in the specimen sample. The particulate may be a virus (e.g., an Influenza virus or SARS-CoV-2), a bacterium (e.g., Streptococcus bacterium), any of the other particulates described hereinabove, or any of the other biological materials described hereinabove. For example, the particulate may be tested for using nucleic acid amplification, such as PCR, e.g., qPCR, and/or by performing an immunoassay, such as a lateral flow immunoassay, e.g., a chromatographic digital immunoassay, or by performing a rapid molecular test, for example one that uses a real-time loop mediated amplification reaction, such as the Lucira COVID-19 All-In-One Test Kit, or a NEAR technology, such as the ID NOW™, or a molecular test kit manufactured by Visby.

In an embodiment, the techniques and apparatus described herein are combined with techniques and apparatus described in one or more of the following patent applications, which are assigned to the assignee of the present application and are incorporated herein by reference:
PCT Publication WO 2018/158768 to Fruchter et al., and US Patent Application Publication 2019/0381498 in the national stage thereof;
U.S. Provisional Application 62/727,208, filed Sep. 5, 2018;
U.S. Provisional Application 62/727,268, filed Sep. 5, 2018;
PCT Publication WO 2020/049566 to Fruchter et al.;
PCT Publication WO 2020/049569 to Fruchter et al., and US Patent Application Publication 2021/0215585 in the national stage thereof;
U.S. Provisional Application 62/896,295, filed Sep. 5, 2019;
U.S. Provisional Application 62/988,145, filed Mar. 11, 2020;
U.S. Provisional Application 62/988,259, filed Mar. 11, 2020;
U.S. Provisional Applications 63/020,723, filed May 6, 2020; 63/037,707, filed Jun. 11, 2020; 63/067,535, filed Aug. 19, 2020; 63/117,294, filed Nov. 23, 2020; 63/156,843, filed Mar. 4, 2021; 63/158,005, filed Mar. 8, 2021; 63/166,378, filed Mar. 26, 2021; and 63/176,565, filed Apr. 19, 2021;
U.S. Provisional Application 63/071,529, filed Aug. 28, 2020;
PCT Publication WO 2021/044417 to Holtz et al.;
US Patent Application Publication 2021/0102876 to Fruchter et al.;
PCT Publication WO 2021/181338 to Fruchter et al.;
PCT Publication WO 2021/181339 to Feldman et al.;
PCT Publication WO 2021/224925 to Levitz et al.;
PCT Publication WO 2022/044002 to Levitz et al.;
U.S. Provisional Application 63/134,282, filed Jan. 6, 2021;
PCT Publication WO 2022/149135 to Feldman et al.;
U.S. Provisional Application 63/275,130, filed Nov. 3, 2021;
U.S. Provisional Application 63/277,238, filed Nov. 9, 2021;
U.S. Provisional Application 63/283,577, filed Nov. 29, 2021;
U.S. Provisional Application 63/388,851, filed Jul. 13, 2022; and/or
U.S. application Ser. No. 17/980,200, filed Nov. 3, 2022.

In an embodiment, the techniques and apparatus described herein are combined with techniques and apparatus described in PCT Publication WO 2022/149135 to Feldman et al., with reference to FIGS. 1A-35B thereof.

It will be appreciated by persons skilled in the art that the present invention is not limited to what has been particularly shown and described hereinabove. Rather, the scope of the present invention includes both combinations and subcombinations of the various features described hereinabove, as well as variations and modifications thereof that are not in the prior art, which would occur to persons skilled in the art upon reading the foregoing description.

1. A kit comprising:
a tube selected from the group of tubes consisting of: an extraction tube and a transport tube;
a filter;
a liquid for bathing the filter within the tube; and
a filter shaft, (a) which includes a distal portion that is coupled to a central portion of the filter, and (b) which is configured to insert the filter into the tube for bathing the filter in the liquid.

2. The kit according to claim 1, wherein the filter is circular when flat.

3. The kit according to claim 1, further comprising a filter reinforcement, which is coupled to a surface of the filter so as to cover 1%-50% of a surface area of the surface.

4. The kit according to claim 1, wherein the filter shaft extends away from a first side of the filter and does not extend away from a second side of the filter opposite the first side, or extends away from the second side of the filter by less than 2 mm.

5. The kit according to claim 1, wherein the tube comprises a flexible material.

6. The kit according to claim 1, wherein a proximal end opening of the tube is shaped as a funnel.

7. The kit according to claim 1, wherein the liquid is selected from the group consisting of: a lysis buffer, an extraction buffer, saline solution, and a transport medium.

8. The kit according to claim 1, wherein the distal portion of the filter shaft is indirectly coupled to the central portion of the filter.

9. The kit according to claim 1, wherein the distal portion of the filter shaft is directly coupled to the central portion of the filter.

10. The kit according to claim 9, further comprising a distal plate that is fixed to a distal end of the distal portion of the filter shaft such that the central portion of the filter is between the distal end and the distal plate, so as to directly couple the filter to the filter shaft.

11. The kit according to any one of claims 1-10, wherein the kit is configured to limit maximum distal advancement of the filter within the tube.

12. The kit according to claim 11, wherein the kit is configured to set an extent of the distal advancement of the filter within the tube to a predetermined distance of the distal advancement, so as to set an axial location of the filter within the tube.

13. The kit according to any one of claims 1-10, wherein the liquid comprises reagents and the tube is the extraction tube.

14. The kit according to claim 13, further comprising a diagnostic test for testing for the presence of a biological particulate trapped by the filter.

15. The kit according to claim 14, wherein the diagnostic test is configured to test a portion of the reagents for the presence of a target analyte released into the reagents from the biological particulate trapped by the filter.

16. The kit according to claim 14, wherein the diagnostic test comprises a lateral flow test strip.

17. The kit according to claim 16, wherein the diagnostic test comprises a housing.

18. The kit according to claim 17, wherein the housing comprises a housing selected from the group consisting of: a cartridge and a card, and wherein the lateral flow test strip is contained at least partially within the housing.

19. The kit according to claim 17,
wherein the lateral flow test strip comprises a sample pad,
wherein the housing is shaped so as to define a channel in fluid communication with the sample pad,
wherein the kit comprises a distal blocking surface that is intercepted by a straight longitudinal axis of the channel, and
wherein the kit is configured such that upon insertion and distal advancement of a bunched-up portion of the filter in the channel, the bunched-up portion of the filter is compacted against the distal blocking surface.

20. The kit according to claim 19, wherein the straight longitudinal axis of the channel forms an angle of 30-90 degrees with the distal blocking surface.

21. The kit according to claim 19, wherein the channel is shaped such that the insertion and the distal advancement of the bunched-up portion of the filter through the channel, while the at least a portion of the filter is bunched up, brings the bunched-up portion of the filter into direct contact with the sample pad of the lateral flow test strip, the sample pad defining the distal blocking surface.

22. The kit according to claim 21, wherein the channel is shaped such that the insertion and the distal advancement of the bunched-up portion of the filter through the channel, while the at least a portion of the filter is bunched up such that an entirety of a perimeter of the filter points distally, brings at least a portion of the perimeter into direct contact with the sample pad of the lateral flow test strip.

23. The kit according to claim 19,
wherein the sample pad is disposed at an upstream end portion of the lateral flow test strip, and the lateral flow test strip further comprises (a) an absorbent pad disposed at a downstream end portion of the lateral flow test strip, and (b) a membrane disposed longitudinally between the sample pad and the absorbent pad, the membrane comprising a test area, which comprises a test line and a control line, and
wherein the housing is configured such that when the housing is placed on a flat surface, the test area of the membrane is farther from flat surface than the sample pad is from the flat surface.

24. The kit according to claim 19, wherein the channel has an internal length of 1-5 cm.

25. The kit according to claim 17, wherein the housing is shaped so as to define an elongate test-strip receptacle for placement of the lateral flow test strip therein.

26. The kit according to claim 25, wherein the kit is configured to allow insertion of the lateral flow test strip into the elongate test-strip receptacle during a test procedure, without disassembling the housing.

27. The kit according to claim 25,
wherein the lateral flow test strip comprises a sample pad,
wherein the housing is shaped so as to define a channel in fluid communication with the sample pad when the lateral flow test strip is placed within the elongate test-strip receptacle,
wherein the kit comprises a distal blocking surface that is intercepted by a straight longitudinal axis of the channel,
wherein the kit is configured such that upon insertion and distal advancement of a bunched-up portion of the filter in the channel, the bunched-up portion of the filter is compacted against the distal blocking surface, and
wherein a central longitudinal axis of the elongate test-strip receptacle (a) defines an angle of less than less than 45 degrees with a straight central longitudinal axis of the channel, or (b) is parallel with the straight central longitudinal axis of the channel.

28. The kit according to claim 27, wherein the housing is configured to be used while the straight central longitudinal axis of the channel defines an angle of 60-90 degrees with a surface that is horizontal with respect to the Earth.

29. The kit according to claim 16, wherein the lateral flow test strip is implemented as a dipstick.

30. The kit according to claim 14, wherein the diagnostic test comprises a rapid molecular test.

31. The kit according to claim 14, further comprising a testing tube separate and distinct from the extraction tube.

32. The kit according to any one of claims 1-10,
wherein the kit further comprises a collection vial, which is shaped so as to define a vial opening and a shaft-passage hole at an end of the collection vial opposite the vial opening,
wherein the filter shaft is disposed partially within the collection vial, and includes a proximal portion that is slidably disposed passing through the shaft-passage hole such that the distal portion of the filter shaft passes through the vial opening, and
wherein the kit is configured such that proximal movement of the filter shaft with respect to the collection vial pulls the filter at least partially into the collection vial via the vial opening.

33. The kit according to claim 32, wherein the kit is configured such that the proximal movement of the filter shaft with respect to the collection vial pulls the filter at least partially into the collection vial via the vial opening and bunches up at least a portion of the filter.

34. The kit according to claim 32, further comprising a seal that inhibits fluid leakage between the proximal portion of the filter shaft and the shaft-passage hole.

35. The kit according to claim 32, wherein the extraction tube and the collection vial are configured such that upon insertion of the collection vial at least partially into the extraction tube and upon distal advancement of the collection vial within the extraction tube, the tube prevents the collection vial from reaching a distal end of the extraction tube, such that the collection vial slides up a portion of the filter shaft as the filter is exposed from the collection vial and is positioned near the distal end of the extraction tube.

36. The kit according to claim 32,
further comprising a diagnostic test comprising (a) a lateral flow test strip, which comprises a sample pad; and (b) a housing, wherein the lateral flow test strip is contained at least partially within the housing, and wherein the housing is shaped so as to define a channel in fluid communication with the sample pad, and
wherein the channel and the collection vial are configured such that upon insertion of the collection vial at least partially into the channel and upon distal advancement of the collection vial within the channel, the channel prevents the collection vial from reaching a distal end of the channel, such that the collection vial slides up a portion of the filter shaft as the filter is exposed from the collection vial and is positioned near the distal end of the channel.

37. The kit according to claim 36, wherein the housing comprises a housing selected from the group consisting of: a cartridge and a card.

38. The kit according to claim 32, further comprising a testing tube separate and distinct from the extraction tube, wherein the testing tube and the collection vial are configured such that upon insertion of the collection vial at least partially into the testing tube and upon distal advancement of the collection vial within the testing tube, the testing tube prevents the collection vial from reaching a distal end of the testing tube, such that the collection vial slides up a portion of the filter shaft as the filter is exposed from the collection vial and is positioned near the distal end of the testing tube.

39. The kit according to any one of claims 1-10, further comprising a sampling device for concentrating a liquid specimen sample, the sampling device comprising a filtration assembly, which comprises:
a container, which is shaped so as to define an inner wall and a proximal container opening for receiving the liquid specimen sample; and
a filter support, which is shaped so as to define (a) a support surface on which the filter is removably disposed, and (b) a plurality of filtrate-passage openings through the filter support,
wherein the filter shaft is removably disposed partially within the filtration assembly,
wherein the filtration assembly is configured to, when the liquid specimen sample is contained in the container and the filter is disposed on the support surface, push at least a portion of the liquid specimen sample through the filter, and
wherein the sampling device is configured such that withdrawal of the filter shaft out of the filtration assembly removes the filter shaft and the filter from the filtration assembly and bunches up at least a portion of the filter, thereby facilitating insertion of the bunched-up filter into the tube.

40. The kit according to claim 39,
wherein the container is tubular,
wherein the filtration assembly further comprises a plunger, which (i) comprises a plunger head and (ii) is insertable into the tubular container via the proximal container opening, such that a lateral surface of the plunger head forms a fluid-tight movable seal with the inner wall, and
wherein the filtration assembly is configured such that movement of the plunger head within the tubular container, when the liquid specimen sample is contained in the tubular container and the filter is disposed on the support surface, pushes at least a portion of the liquid specimen sample through the filter.

41. The kit according to claim 40, wherein the sampling device is configured such that the filter is removable from the tubular container while the plunger head, including the filter support thereof, remains within the tubular container.

42. The kit according to claim 39, wherein the sampling device is configured such that the withdrawal of the filter shaft out of the filtration assembly removes the filter shaft and the filter from the filtration assembly and bunches up the at least a portion of the filter into a flower-like arrangement.

43. The kit according to claim 39, wherein the sampling device is configured such that the withdrawal of the filter shaft out of the filtration assembly removes the filter shaft and the filter from the filtration assembly and bunches up the at least a portion of the filter such that an entirety of a perimeter of the filter extends distally away from the distal portion of the filter shaft.

44. The kit according to claim 39, wherein the sampling device is configured such that the withdrawal of the filter shaft out of the filtration assembly removes the filter shaft and the filter from the filtration assembly and bunches up the at least a portion of the filter such that an entirety of a perimeter of the filter points distally.

45. The kit according to claim 39, wherein the sampling device is configured such that the withdrawal of the filter shaft out of the filtration assembly removes the filter shaft and the filter from the filtration assembly and bunches up the at least a portion of the filter such that the bunched-up portion of the filter defines an internal space open distally.

46. The kit according to claim 39, wherein the filter is removably disposed in a flat shape on the support surface of the filter support.

47. The kit according to claim 39,
wherein the filtration assembly is configured to, when the liquid specimen sample is contained in the container and the filter is disposed on the support surface, push the at least a portion of the liquid specimen sample through the filter in an upstream-to-downstream direction, thereby trapping, on an upstream surface of the filter, a portion of biological particulate present in the liquid specimen, and
wherein the sampling device is configured such that the withdrawal of the filter shaft out of the filtration assembly removes the filter shaft and the filter from the filtration assembly and bunches up the at least a portion of the filter such that the upstream surface of the filter is inside the bunched-up filter.

48. The kit according to claim 39,
wherein the filtration assembly to, when the liquid specimen sample is contained in the container and the filter is disposed on the support surface, push the at least a portion of the liquid specimen sample through the filter in an upstream-to-downstream direction, thereby trapping, on an upstream surface of the filter, a portion of biological particulate present in the liquid specimen, and
wherein the sampling device is configured such that the withdrawal of the filter shaft out of the filtration assembly removes the filter shaft and the filter from the filtration assembly and bunches up the at least a portion of the filter such that a downstream surface of the filter is inside the bunched-up filter.

49. The kit according to claim 39,
wherein the kit further comprises a filter receptacle that is coupled to a distal portion of the filter shaft and shaped so as to define a distal receptacle opening, and
wherein the sampling device is configured such that the withdrawal of the filter shaft out of the filtration assembly pulls the central portion of the filter into the filter receptacle via the distal receptacle opening, thereby causing the remainder of the filter to become bunched up and be disposed at least partially outside the filter receptacle.

50. The kit according to claim 49, wherein the filter receptacle is slidably coupled to the distal portion of the filter shaft.

51. The kit according to claim 39,
wherein the sampling device further comprises a collection vial, which is disengageably coupled to the filtration assembly, and
wherein the sampling device is configured such that the filter is advanceable into the collection vial while the collection vial is disengageably coupled to the filtration assembly.

52. The kit according to claim 51,
wherein the container is tubular,
wherein the filtration assembly further comprises a plunger, which (i) comprises a plunger head and (ii) is insertable into the tubular container via the proximal container opening, such that a lateral surface of the plunger head forms a fluid-tight movable seal with the inner wall,
wherein the filtration assembly is configured such that movement of the plunger head within the tubular container, when the liquid specimen sample is contained in the tubular container and the filter is disposed on the support surface, pushes at least a portion of the liquid specimen sample through the filter, and
wherein the sampling device is configured such that the collection vial is decouplable from the filtration assembly while the plunger head remains within the tubular container.

53. The kit according to claim 52,
wherein the collection vial is shaped so as to define a vial opening and a shaft-passage hole at an end of the collection vial opposite the vial opening,
wherein the sampling device is configured such that the filter is advanceable into the collection vial via the vial opening while the collection vial is disengageably coupled to the filtration assembly,
wherein the filter shaft is disposed partially within the collection vial within the filtration assembly, and includes a proximal portion that is slidably disposed passing through the shaft-passage hole such that the distal portion of the filter shaft passes through the vial opening, and
wherein the sampling device is configured such that proximal withdrawal of the filter shaft, while the distal portion of the filter shaft is coupled to the filter and while the plunger head remains within the tubular container, pulls the filter into the collection vial via the vial opening.

54. The kit according to claim 53, further comprising a seal that inhibits fluid leakage between the proximal portion of the filter shaft and the shaft-passage hole.

55. The kit according to claim 53, wherein the sampling device is configured such that further proximal withdrawal of the filter shaft out of the filtration assembly, while the plunger head remains within the tubular container, pulls the collection vial out of the filtration assembly.

56. The kit according to claim 55, wherein the tube and the collection vial are configured such that upon insertion of the collection vial at least partially into the tube and upon distal advancement of the collection vial within the tube, the tube prevents the collection vial from reaching a distal end of the tube, such that the collection vial slides up a portion of the filter shaft as the filter is exposed from the collection vial and positioned near the distal end of the tube.

57. The kit according to any one of claims 1-56, wherein the filter comprises a polyethersulfone (PES) membrane filter.

58. A method comprising:
passing at least a portion of a liquid specimen sample through a filter;
bunching up at least a portion of the filter;
placing a liquid in a tube selected from the group of tubes consisting of: an extraction tube and a transport tube;
before or after placing the liquid in the tube, inserting the bunched-up portion of the filter into the tube;
bathing the filter in the liquid in the tube; and
thereafter, testing a portion of the liquid for the presence of a biological particulate released from the filter into the liquid.

59. The method according to claim 58, wherein the liquid is selected from the group consisting of: a lysis buffer, an extraction buffer, saline solution, and a transport medium.

60. The method according to claim 58, wherein the filter is circular when flat.

61. The method according to claim 58, wherein a filter reinforcement is coupled to a surface of the filter so as to cover 1%-50% of a surface area of the surface.

62. The method according to claim 58, wherein bunching up the at least a portion of the filter comprises bunching up the at least a portion of the filter into a flower-like arrangement.

63. The method according to claim 58,
wherein bunching up the at least a portion of the filter comprises bunching up the at least a portion of the filter such that an entirety of a perimeter of the filter points distally, and
wherein inserting the bunched-up portion of the filter into the tube comprises inserting, in a distal direction, the bunched-up portion of the filter into the tube while the entirety of the perimeter of the filter points distally.

64. The method according to claim 58,
wherein bunching up the at least a portion of the filter comprises bunching up the at least a portion of the filter such that the bunched-up portion of the filter defines an internal space open distally, and
wherein inserting the bunched-up portion of the filter into the tube comprises inserting, in a distal direction, the bunched-up portion of the filter into the tube while the bunched-up portion of the filter defines the internal space open distally.

65. The method according to claim 58, wherein a proximal end opening of the tube is shaped as a funnel, and wherein inserting the bunched-up portion of the filter into the tube comprises inserting the bunched-up portion of the filter into the tube via the proximal end opening of the tube.

66. The method according to claim 58, wherein passing the at least a portion of the liquid specimen sample through the filter comprises passing the at least a portion of the liquid specimen sample through the filter while the filter is in a flat shape.

67. The method according to claim 58,
wherein passing the at least a portion of the liquid specimen sample through the filter comprises passing the at least a portion of the liquid specimen sample through the filter in an upstream-to-downstream direction, thereby trapping, on an upstream surface of the filter, a portion of biological particulate present in the liquid specimen, and wherein bunching up the at least a portion of the filter comprises bunching up the at least a portion of the filter such that the upstream surface of the filter is inside the bunched-up portion of the filter.

68. The method according to claim 58,
wherein passing the at least a portion of the liquid specimen sample through the filter comprises passing the at least a portion of the liquid specimen sample through the filter in an upstream-to-downstream direction, thereby trapping, on an upstream surface of the filter, a portion of biological particulate present in the liquid specimen, and wherein bunching up the at least a portion of the filter comprises bunching up the at least a portion of the filter such that a downstream surface of the filter is inside the bunched-up portion of the filter.

69. The method according to claim 58, wherein testing the portion of the liquid comprises performing a rapid molecular test.

70. The method according to claim 58, wherein the liquid specimen sample includes gargled fluid.

71. The method according to claim 58, wherein the filter comprises a polyethersulfone (PES) membrane filter.

72. The method according to any one of claims 58-71,
wherein the liquid includes reagents and the tube is the extraction tube,
wherein bathing the filter in the liquid in the tube comprises bathing the filter in the reagents in the extraction tube, and
wherein testing the portion of the liquid comprises testing the portion of the reagents for the presence of the biological particulate released by the filter into the reagents.

73. The method according to claim 72, wherein testing comprises discarding a portion of the reagents after bathing the filter in the reagents in the extraction tube.

74. The method according to claim 72, wherein testing comprises draining excess reagents from the extraction tube and thereafter squeezing the bunched-up portion of the filter to squeeze the portion of the reagents from the filter.

75. The method according to claim 72, wherein testing comprises removing the bunched-up portion of the filter from the extraction tube and thereafter expelling the portion of the reagents from the bunched-up portion of the filter.

76. The method according to claim 75, wherein expelling the portion of the reagents from the bunched-up portion of the filter comprises squeezing the bunched-up portion of the filter to squeeze the portion of the reagents from the bunched-up portion of the filter after the bunched-up portion of the filter is removed from the extraction tube.

77. The method according to claim 76, wherein expelling the portion of the reagents from the bunched-up portion of the filter comprises inserting the filter into a testing tube separate and distinct from the extraction tube, and squeezing the bunched-up portion of the filter while the filter is within the testing tube.

78. The method according to claim 77, wherein testing the portion of the reagents comprises:
removing the filter from the testing tube; and
thereafter, inserting a lateral flow test strip into the reagents within the testing tube.

79. The method according to claim 76, wherein testing for the presence of the biological particulate comprises bringing the bunched-up portion of the filter into direct contact with a sample pad of a lateral flow test strip.

80. The method according to claim 79, wherein bringing the bunched-up portion of the filter into the direct contact with the sample pad comprises orienting a filter shaft so that a straight central longitudinal axis of the filter shaft forms an angle of 30-90 degrees with the sample pad, wherein the filter shaft includes a distal portion coupled to the filter.

81. The method according to claim 79, wherein bunching up the at least a portion of the filter comprises bunching up the at least a portion of the filter such that the bunched-up portion of the filter defines an internal space open distally.

82. The method according to claim 79, wherein bunching up the at least a portion of the filter comprises bunching up the at least a portion of the filter into a flower-like arrangement.

83. The method according to claim 79, wherein bringing the filter into the direct contact with the sample pad comprises squeezing the bunched-up portion of the filter against the sample pad of the lateral flow test strip.

84. The method according to claim 83, wherein squeezing the bunched-up portion of the filter against the sample pad comprises squeezing the bunched-up portion of the filter by longitudinally compacting the bunched-up portion of the filter against the sample pad.

85. The method according to claim 83, wherein squeezing the bunched-up portion of the filter against the sample pad delivers to the sample pad both (a) some of the reagents contained in or on the filter at locations of the filter that directly contact the sample pad, and (b) some of the reagents contained in or on the filter at locations other than the locations of the filter that directly contact the sample pad.

86. The method according to claim 76,
wherein the filter is coupled to a distal portion of a filter shaft that is disposed partially within a flexible collection vial that is shaped so as to define a vial opening and a shaft-passage hole at an end of the flexible collection vial opposite the vial opening,
wherein the filter shaft includes a proximal portion that is slidably disposed passing through the shaft-passage hole such that the distal portion of the filter shaft passes through the vial opening, and
wherein expelling the portion of the reagents from the filter comprises:
pulling the filter at least partially into the flexible collection vial via the vial opening, by proximally moving the filter shaft with respect to the flexible collection vial; and
squeezing the bunched-up portion of the filter by squeezing the flexible collection vial.

87. The method according to claim 75, wherein removing the filter from the extraction tube comprises removing the filter from the extraction tube without first squeezing the bunched-up portion of the filter within the extraction tube.

88. The method according to claim 72, wherein testing comprises testing the portion of the reagents by applying the portion of the reagents to a lateral flow test strip.

89. The method according to claim 88, wherein the lateral flow test strip is contained at least partially within a housing.

90. The method according to claim 89, wherein the housing is selected from the group consisting of: a cartridge and a card.

91. The method according to claim 88, wherein the lateral flow test strip is implemented as a dipstick.

92. The method according to claim 91, wherein applying the portion of the reagents to the lateral flow test strip comprises inserting at least a portion of the dipstick into the extraction tube.

93. The method according to claim 92, wherein applying the portion of the reagents to the lateral flow test strip comprises removing the filter from the extraction tube before inserting the at least a portion of the dipstick into the extraction tube.

94. The method according to claim 93, wherein testing the portion of the reagents comprises, before removing the filter from the extraction tube, squeezing the bunched-up portion of the filter to squeeze the portion of the reagents from the filter while the bunched-up portion of the filter is within the extraction tube.

95. The method according to any one of claims 58-71, wherein testing the portion of the liquid comprises squeezing the bunched-up portion of the filter to squeeze the portion of the liquid from the filter.

96. The method according to claim 95, wherein squeezing the bunched-up portion of the filter comprises squeezing the bunched-up portion of the filter while the bunched-up portion of the filter is within the tube.

97. The method according to claim 96, wherein squeezing the bunched-up portion of the filter while the bunched-up portion of the filter is within the tube comprises squeezing the bunched-up portion of the filter at least one time without expelling any of the liquid from the tube.

98. The method according to claim 96, wherein squeezing the bunched-up portion of the filter comprises squeezing the bunched-up portion of the filter at least one time to both squeeze the portion of the liquid from the filter and to expel some of the liquid from the tube.

99. The method according to claim 96, wherein squeezing the bunched-up portion of the filter while the bunched-up portion of the filter is within the tube comprises:
squeezing the bunched-up portion of the filter at least a first time without expelling any of the liquid from the tube; and
thereafter, squeezing the bunched-up portion of the filter at least a second time to both squeeze an additional portion of the liquid from the filter and to expel some of the liquid from the tube.

100. The method according to claim 96, wherein the tube includes a flexible material, and wherein squeezing the bunched-up portion of the filter comprises squeezing a longitudinal portion of the tube along which longitudinal portion the bunched-up portion of the filter is disposed.

101. The method according to claim 96, further comprising, after bathing the filter in the liquid in the tube and before squeezing the bunched-up portion of the filter, removing excess liquid from the tube.

102. The method according to claim 95, wherein squeezing the bunched-up portion of the filter comprises removing the filter from the tube and squeezing the bunched-up portion of the filter while the bunched-up portion of the filter is outside the tube.

103. The method according to claim 102, wherein testing for the presence of the biological particulate comprises bringing the bunched-up portion of the filter into direct contact with a sample pad of a lateral flow test strip.

104. The method according to claim 102, wherein squeezing the bunched-up portion of the filter to squeeze the portion of the liquid from the filter comprises, after removing the filter from the tube, inserting the bunched-up portion of the filter into a channel and longitudinally compacting the bunched-up portion of the filter against a distal blocking surface intercepted by a straight central longitudinal axis of the channel.

105. The method according to claim 104, wherein the channel is in fluid communication with a sample pad of a lateral flow test strip.

106. The method according to claim 105,
wherein the lateral flow test strip is disposed at least partially within a housing such that a test area of the lateral flow test strip is visible through one or more result windows defined by the housing, and
wherein the housing further defines the channel.

107. The method according to claim 106,
wherein bunching up the at least a portion of the filter comprises bunching up the at least a portion of the filter such that an entirety of a perimeter of the filter points distally, and
wherein inserting the bunched-up portion of the filter into the channel comprises inserting, in a distal direction, the bunched-up portion of the filter into the channel while the entirety of the perimeter of the filter points distally.

108. The method according to claim 106, wherein inserting the bunched-up portion of the filter into the channel comprises advancing a distal portion of the bunched-up portion of the filter through the channel and into direct contact with the sample pad of the lateral flow test strip, the sample pad defining the distal blocking surface.

109. The method according to claim 108,
wherein bunching up the at least a portion of the filter comprises bunching up the at least a portion of the filter such that an entirety of a perimeter of the filter points distally,
wherein inserting the bunched-up portion of the filter into the channel comprises inserting, in a distal direction, the bunched-up portion of the filter into the channel while the entirety of the perimeter of the filter points distally, and
wherein advancing the distal portion of the bunched-up portion of the filter comprises advancing the distal portion of the bunched-up portion of the filter through the channel so that at least a portion of the perimeter makes direct contact with the sample pad of the lateral flow test strip.

110. The method according to claim 106,
wherein the sample pad is disposed at an upstream end portion of the lateral flow test strip, and the lateral flow test strip further includes (a) an absorbent pad disposed at a downstream end portion of the lateral flow test strip, and (b) a membrane disposed longitudinally between the sample pad and the absorbent pad, the membrane comprising a test area, which comprises a test line and a control line, and
wherein the housing is configured such that when the housing is placed on a flat surface, the test area of the membrane is farther from flat surface than the sample pad is from the flat surface.

111. The method according to claim 102, wherein removing the filter from the tube comprises removing the filter from the tube without first squeezing the bunched-up portion of the filter within the tube.

112. The method according to claim 95, wherein squeezing the bunched-up portion of the filter comprises inserting an object into the tube and using the object to squeeze the bunched-up portion of the filter.

113. The method according to claim 95, wherein testing the portion of the liquid for the presence of the biological particulate comprises using a diagnostic test to test the portion of the liquid for the presence of the biological particulate, and wherein squeezing the bunched-up portion of the filter comprises inserting a portion of the diagnostic test into the tube and using the portion of the diagnostic test to squeeze the bunched-up portion of the filter.

114. The method according to any one of claims 58-71, wherein bunching up the at least a portion of the filter comprises bunching up the at least a portion of the filter while a central portion of the filter is coupled to a distal portion of a filter shaft.

115. The method according to claim 114, wherein the filter shaft extends away from a first side of the filter and does not extend away from a second side of the filter opposite the first side, or extends away from the second side of the filter by less than 2 mm.

116. The method according to claim 114,
wherein bunching up the at least a portion of the filter comprises bunching up the at least a portion of the filter such that an entirety of a perimeter of the filter extends distally away from the distal portion of the filter shaft, and
wherein inserting the bunched-up portion of the filter into the tube comprises inserting, in a distal direction, the bunched-up portion of the filter into the tube while the entirety of the perimeter of the filter extends distally away from the distal portion of the filter shaft.

117. The method according to claim 114, wherein the distal portion of the filter shaft is indirectly coupled to the central portion of the filter.

118. The method according to claim 114, wherein the distal portion of the filter shaft is directly coupled to the central portion of the filter.

119. The method according to claim 118, wherein a distal plate is fixed to a distal end of the distal portion of the filter shaft such that the central portion of the filter is between the distal end and the distal plate, so as to directly couple the filter to the filter shaft.

120. The method according to claim 114,
wherein the filter shaft is disposed partially within a collection vial that is shaped so as to define a vial opening and a shaft-passage hole at an end of the collection vial opposite the vial opening,
wherein the filter shaft includes a proximal portion that is slidably disposed passing through the shaft-passage hole such that the distal portion of the filter shaft passes through the vial opening, and
wherein bunching up the at least a portion of the filter comprises pulling the filter at least partially into the collection vial via the vial opening, by proximally moving the filter shaft with respect to the collection vial.

121. The method according to claim 120, wherein the collection vial includes a seal that inhibits fluid leakage between the proximal portion of the filter shaft and the shaft-passage hole.

122. The method according to claim 120, wherein inserting the bunched-up portion of the filter into the tube comprises inserting the collection vial at least partially into the tube and distally advancing the collection vial within the tube, such that the tube prevents the collection vial from reaching a distal end of the tube, such that the collection vial slides up a portion of the filter shaft as the filter is exposed from the collection vial and is positioned near the distal end of the tube.

123. The method according to claim 120,
wherein testing the portion of the liquid comprises squeezing the bunched-up portion of the filter to squeeze the portion of the liquid from the filter, by inserting the bunched-up portion of the filter into a channel in fluid communication with a sample pad of a lateral flow test strip disposed partially within a housing that defines the channel, and
wherein the channel and the collection vial are configured such that upon insertion of the collection vial at least partially into the channel and upon distal advancement of the collection vial within the channel, the channel prevents the collection vial from reaching a distal end of the channel, such that the collection vial slides up a portion of the filter shaft as the filter is exposed from the collection vial and is positioned near the distal end of the channel.

124. The method according to claim 114,
wherein passing the at least a portion of the liquid specimen sample through the filter comprises using a filtration assembly while the filter is removably disposed in the filtration assembly and the filter shaft is removably disposed partially within the filtration assembly, and
wherein bunching up the at least a portion of the filter comprises withdrawing the filter shaft out of the filtration assembly such that the withdrawing removes the filter shaft and the filter from the filtration assembly and bunches up at least a portion of the filter.

125. The method according to claim 124, wherein bunching up the at least a portion of the filter comprises withdrawing the filter shaft out of the filtration assembly such that the withdrawing of the filter shaft out of the filtration assembly removes the filter shaft and the filter from the filtration assembly and bunches up the at least a portion of the filter into a flower-like arrangement.

126. The method according to claim 124, wherein bunching up the at least a portion of the filter comprises withdrawing the filter shaft out of the filtration assembly such that the withdrawing of the filter shaft out of the filtration assembly removes the filter shaft and the filter from the filtration assembly and bunches up the at least a portion of the filter such that an entirety of a perimeter of the filter extends distally away from the distal portion of the filter shaft.

127. The method according to claim 124, wherein bunching up the at least a portion of the filter comprises withdrawing the filter shaft out of the filtration assembly such that the withdrawing of the filter shaft out of the filtration assembly removes the filter shaft and the filter from the filtration assembly and bunches up the at least a portion of the filter such that an entirety of a perimeter of the filter points distally.

128. The method according to claim 124, wherein withdrawing the filter shaft out of the filtration assembly pulls the central portion of the filter into a filter receptacle via a distal receptacle opening, thereby causing the remainder of the filter to become bunched up and be disposed at least partially outside the filter receptacle, wherein the filter receptacle is coupled to a distal portion of the filter shaft.

129. The method according to claim 128, wherein the filter receptacle is slidably coupled to the distal portion of the filter shaft.

130. The method according to claim 124, wherein passing the at least a portion of the liquid specimen sample through the filter comprises:
placing the liquid sample in a tubular container of the filtration assembly, the tubular container shaped so as to define an inner wall and a proximal container opening for receiving the liquid specimen sample;
while the filter is removably disposed on a support surface of a filter support of the filtration assembly and the filter shaft is removably disposed partially within the filtration assembly, inserting a plunger into the tubular container via the proximal container opening, such that a lateral surface of a plunger head of the plunger forms a fluid-tight movable seal with the inner wall, wherein the filter support is shaped so as to define a plurality of filtrate-passage openings through the filter support; and
pushing at least a portion of the liquid specimen sample through the filter by moving the plunger head within the tubular container.

131. The method according to claim 130, wherein inserting the plunger into the tubular container comprises inserting the plunger into the tubular container while the filter is removably disposed in a flat shape on the support surface of the filter support.

132. The method according to claim 130, wherein pushing the at least a portion of the liquid specimen sample through the filter comprises pushing the at least a portion of the liquid specimen sample through the filter in a distal-to-proximal direction, thereby trapping, on a distal surface of the filter, a portion of biological particulate present in the liquid specimen.

133. The method according to claim 130, wherein withdrawing the filter shaft out of the filtration assembly comprises removing the filter from the tubular container while the plunger head, including the filter support thereof, remains within the tubular container.

134. The method according to claim 130, further comprising advancing the filter into a collection vial while the collection vial is disengageably coupled to the filtration assembly.

135. The method according to claim 134, further comprising decoupling the collection vial from the filtration assembly while the plunger head remains within the tubular container.

136. The method according to claim 135,
wherein advancing the filter into the collection vial comprises advancing the filter into the collection vial via a vial opening of the collection vial while the collection vial is disengageably coupled to the filtration assembly,
wherein the filter shaft is disposed partially within the collection vial within the filtration assembly, and includes a proximal portion that is slidably disposed passing through a shaft-passage hole at an end of the collection vial opposite the vial opening, such that the distal portion of the filter shaft passes through the vial opening, and
wherein withdrawing the filter shaft, while the plunger head remains within the tubular container, pulls the filter into the collection vial via the vial opening.

137. The method according to claim 136, wherein further proximal withdrawing of the filter shaft out of the filtration assembly, while the plunger head remains within the tubular container, pulls the collection vial out of the filtration assembly.

138. The method according to claim 137, further comprising, after pulling the collection vial out of the filtration assembly, inserting the collection vial at least partially into the tube and distally advancing the collection vial within the tube, such that the tube prevents the collection vial from reaching a distal end of the tube, such that the collection vial slides up a portion of the filter shaft as the filter is exposed from the collection vial and positioned near the distal end of the tube.

139. The method according to any one of claims 58-71, wherein the biological particulate is selected from the group consisting of: a virus, a bacterium, a microorganism, a fungus, a spore, a mite, a biological cell, a biological antigen, a protein, a protein antigen, and a carbohydrate antigen.

140. The method according to claim 139, wherein the biological particulate is the biological antigen.

141. A sampling device for concentrating a liquid specimen sample, the sampling device comprising:
(a) a filter;
(b) a filtration assembly, which comprises:
(i) a tubular container, which is shaped so as to define an inner wall and a proximal container opening for receiving the liquid specimen sample;
(ii) a plunger, which (A) comprises a plunger head and (B) is insertable into the tubular container via the proximal container opening, such that a lateral surface of the plunger head forms a fluid-tight movable seal with the inner wall; and
(iii) a filter support, which is shaped so as to define (A) a support surface on which the filter is removably disposed, and (B) a plurality of filtrate-passage openings through the filter support; and
(c) a withdrawer comprising a filter-withdrawal shaft, which is partially inserted in the filtration assembly,
wherein the filtration assembly is configured such that movement of the plunger head within the tubular container, when the liquid specimen sample is contained in the tubular container and the filter is disposed in the tubular container, pushes at least a portion of the liquid specimen sample through the filter, and
wherein the sampling device is configured such that removal of the filter-withdrawal shaft from the filtration assembly, while a distal portion of the filter-withdrawal shaft is coupled to the filter, while the plunger head remains within the tubular container, and while the filter support remains within the filtration assembly, removes the filter from the filter support and from the filtration assembly.

142. The sampling device according to claim 141,
wherein the filter support is disposed within the tubular container, and
wherein the sampling device is configured such that the removal of the filter-withdrawal shaft from the filtration assembly, while the distal portion of the filter-withdrawal shaft is coupled to the filter, while the plunger head remains within the tubular container, and while the filter support remains within the tubular container, removes the filter from the filter support and from the filtration assembly.

143. The sampling device according to claim 141,
wherein the filter support is shaped so as to define a central opening, in addition to the plurality of filtrate-passage openings, and
wherein the sampling device is configured such that removal of the filter-withdrawal shaft from the filtration assembly, while the plunger head remains within the tubular container and the filter support remains within the filtration assembly, pulls the filter through the central opening and removes the filter from the filter support and from the filtration assembly.

144. The sampling device according to claim 141, wherein the filtration assembly further comprises a waste liquid receptacle, and wherein the filter support is shaped so as to define the plurality of filtrate-passage openings through the filter support into the waste liquid receptacle.

145. The sampling device according to claim 141,
wherein the sampling device comprises a filter receptacle that is slidably coupled to a distal end portion of the filter-withdrawal shaft and shaped so as to define a distal receptacle opening, and
wherein the sampling device is configured such that withdrawal of the filter-withdrawal shaft from the filtration assembly pulls a portion of the filter into the filter receptacle via the distal receptacle opening.

146. The sampling device according to any one of claims 141-145,
wherein the plunger comprises a plunger rod, which (a) has a distal end portion to which the plunger head is coupled, and (b) is shaped so as to define an internal plunger space having a plunger-space proximal opening through a proximal end of the plunger rod,
wherein the plunger head is shaped so as to define a plunger-head opening through the plunger head and into the internal plunger space,
wherein the filter-withdrawal shaft is disposed passing through the internal plunger space, and
wherein the sampling device is configured such that proximal withdrawal of the filter-withdrawal shaft out of the internal plunger space, while the plunger head remains within the tubular container, pulls the filter into the internal plunger space via the plunger-head opening and out of the internal plunger space via the plunger-space proximal opening, and removes the filter-withdrawal shaft from the filtration assembly and the filter from the filter support and from the filtration assembly.

147. The sampling device according to claim 146,
wherein the filtration assembly and the withdrawer are shaped so as to define corresponding screw threads, respectively, which (a) removably couple the filter-withdrawal shaft to the plunger rod while the filter-withdrawal shaft is disposed passing through the internal plunger space, and (b) prevent premature proximal withdrawal of the filter-withdrawal shaft out of the internal plunger space, and
wherein the sampling device is configured such that rotation of the filter-withdrawal shaft and the plunger-space proximal opening with respect to each other (a) causes at least an initial portion of the proximal withdrawal of the filter-withdrawal shaft out of the internal plunger space, and (b) decouples the screw threads from each other.

148. The sampling device according to claim 147, wherein the sampling device is configured such that the filter-withdrawal shaft does not rotate during at least an initial portion of the proximal withdrawal of the filter-withdrawal shaft out of the internal plunger space.

149. The sampling device according to claim 148,
wherein the withdrawer further comprises a shaft handle, which is coupled to a proximal portion of the filter-withdrawal shaft such that the shaft handle is free to rotate with respect to the filter-withdrawal shaft,
wherein the shaft handle of the withdrawer is shaped so as to define the screw thread of the withdrawer,
wherein the plunger comprises a plunger rod that comprises a hollow shaft that is shaped so as to define (a) the internal plunger space within the hollow shaft and (b) a non-circular inner surface along at least a longitudinal portion of the hollow shaft,
wherein the filter-withdrawal shaft is shaped so as to define a non-circular outer surface along at least a longitudinal portion of the filter-withdrawal shaft, and
wherein the sampling device is configured such that the non-circular outer surface engages the non-circular inner surface so as to prevent rotation of the filter-withdrawal shaft with respect to the hollow shaft during at least an initial portion of the proximal withdrawal of the filter-withdrawal shaft out of the internal plunger space.

150. The sampling device according to claim 146, wherein the plunger rod is shaped so as to define therewithin a waste liquid receptacle, and wherein the filter support is shaped so as to define the plurality of filtrate-passage openings through the filter support into the waste liquid receptacle.

151. The sampling device according to any one of claims 141-145,
wherein the sampling device further comprises a collection vial, which is disengageably coupled to the filtration assembly, and
wherein the sampling device is configured such that the filter is advanceable into the collection vial while the collection vial is disengageably coupled to the filtration assembly.

152. The sampling device according to claim 151, wherein the sampling device is configured such that the collection vial is decouplable from the filtration assembly while the plunger head remains within the tubular container.

153. The sampling device according to claim 152,
wherein the collection vial is shaped so as to define a vial opening and a shaft-passage hole at an end of the collection vial opposite the vial opening,
wherein the sampling device is configured such that the filter is advanceable into the collection vial via the vial opening while the collection vial is disengageably coupled to the filtration assembly,
wherein the filter-withdrawal shaft is disposed partially within the collection vial within the filtration assembly, and includes a proximal portion that is slidably disposed passing through the shaft-passage hole such that the distal portion of the filter-withdrawal shaft passes through the vial opening, and
wherein the sampling device is configured such that proximal withdrawal of the filter-withdrawal shaft, while the distal portion of the filter-withdrawal shaft is coupled to the filter and while the plunger head remains within the tubular container, pulls the filter into the collection vial via the vial opening.

154. The sampling device according to claim 153, further comprising a seal that inhibits fluid leakage between the proximal portion of the filter-withdrawal shaft and the shaft-passage hole.

155. The sampling device according to claim 153, wherein the sampling device is configured such that further proximal withdrawal of the filter-withdrawal shaft out of the filtration assembly, while the plunger head remains within the tubular container, pulls the collection vial out of the filtration assembly.

156. The sampling device according to claim 155, further comprising a tube selected from the group of tubes consisting of: an extraction tube and a transport tube, wherein the tube and the collection vial are configured such that upon insertion of the collection vial at least partially into the tube and upon distal advancement of the collection vial within the tube, the tube prevents the collection vial from reaching a distal end of the tube, such that the collection vial slides up a portion of the filter-withdrawal shaft as the filter is exposed from the collection vial and positioned near the distal end of the tube.

157. The sampling device according to claim 151,
wherein the plunger comprises a plunger rod, which (a) has a distal end portion to which the plunger head is coupled, and (b) is shaped so as to define an internal plunger space having a plunger-space proximal opening through a proximal end of the plunger rod, and
wherein the collection vial is removably disposed at least partially within the internal plunger space.

158. The sampling device according to claim 157, wherein the sampling device is configured such that the collection vial is decouplable from the filtration assembly while the plunger head remains within the tubular container.

159. The sampling device according to claim 157,
wherein the plunger head is shaped so as to define a plunger-head opening through the plunger head and into the internal plunger space,
wherein the collection vial is shaped so as to define a vial opening and a shaft-passage hole at an end of the collection vial opposite the vial opening, wherein the sampling device is configured such that the filter is advanceable into the collection vial via the plunger-head opening and the vial opening while the collection vial is disengageably coupled to the filtration assembly,
wherein the filter-withdrawal shaft is disposed partially within the collection vial within the internal plunger space, and includes a proximal portion that is slidably disposed passing through the shaft-passage hole such that the distal portion of the filter-withdrawal shaft passes through the vial opening, and
wherein the sampling device is configured such that proximal withdrawal of the filter-withdrawal shaft, while the distal portion of the filter-withdrawal shaft is coupled to the filter and while the plunger head remains within the tubular container, pulls the filter into the collection vial via the plunger-head opening and the vial opening.

160. The sampling device according to claim 159, further comprising a seal that inhibits fluid leakage between the proximal portion of the filter-withdrawal shaft and the shaft-passage hole.

161. The sampling device according to claim 159, wherein the sampling device is configured such that further proximal withdrawal of the filter-withdrawal shaft out of the internal plunger space, while the plunger head remains within the tubular container, pulls the collection vial out of the internal plunger space via the plunger-space proximal opening.

162. The sampling device according to any one of claims 141-145,
wherein the filter-withdrawal shaft is disposed passing through a distal opening defined by a distal bottom surface of the tubular container, and
wherein the sampling device is configured such that removal of the filter-withdrawal shaft from the filtration assembly, while a distal portion of the filter-withdrawal shaft is coupled to the filter, while the plunger head remains within the tubular container, and while the filter support remains within the filtration assembly, removes the filter from the filter support and from the filtration assembly via the distal opening defined by the bottom surface of the tubular container.

163. The sampling device according to claim 162,
wherein the tubular container comprises a hollow shaft that is shaped so as to define an internal shaft space within the hollow shaft,
wherein the filter-withdrawal shaft is disposed passing through (a) the distal opening defined by the distal bottom surface of the tubular container and (b) the internal shaft space, and
wherein the sampling device is configured such that the removal of the filter-withdrawal shaft from the filtration assembly, while the distal portion of the filter-withdrawal shaft is coupled to the filter, while the plunger head remains within the tubular container, and while the filter support remains within the filtration assembly, removes the filter from the filter support and from the filtration assembly via (a) the distal opening defined by the bottom surface of the tubular container and (b) the internal shaft space.

164. A testing kit comprising the sampling device according to any one of claims 141-163, the testing kit further comprising a diagnostic test configured to detect the presence of a biological particulate trapped by the filter.

165. The testing kit according to claim 164, wherein the diagnostic test comprises a lateral flow test strip.

166. The testing kit according to claim 165, further comprising reagents for use with the lateral flow test strip.

167. The testing kit according to claim 164, wherein the diagnostic test comprises a rapid molecular test.

168. The testing kit according to claim 164, wherein the biological particulate is selected from the group consisting of: a virus, a bacterium, a microorganism, a fungus, a spore, a mite, a biological cell, a biological antigen, a protein, a protein antigen, and a carbohydrate antigen.

169. The sampling device according to any one of claims 141-163, wherein the filter comprises a polyethersulfone (PES) membrane filter.

170. A method for concentrating a liquid specimen sample, the method comprising:
placing the liquid specimen sample in a tubular container of a filtration assembly of a sampling device;
inserting a plunger head of a plunger of the filtration assembly into the tubular container via a proximal container opening of the tubular container, such that a lateral surface of the plunger head forms a fluid-tight movable seal with an inner wall of the tubular container;
distally advancing the plunger head within the tubular container to drive at least a portion of the liquid specimen sample through a filter removably disposed in the tubular container on a support surface of a filter support, wherein the filter support is shaped so as to define a plurality of filtrate-passage openings through the filter support; and
removing the filter from the filter support and from the filtration assembly while the plunger head remains within the tubular container and the filter support remains within the filtration assembly.

171. The method according to claim 170,
wherein the filter support is disposed within the tubular container, and
wherein removing the filter from the filter support and from the filtration assembly comprises removing the filter from the filter support and from the filtration assembly while the plunger head remains within the tubular container and the filter support remains within the tubular container.

172. The method according to claim 170, wherein removing the filter from the filter support and from the filtration assembly comprises removing a filter-withdrawal shaft from being partially inserted in the filtration assembly, while a distal portion of the filter-withdrawal shaft is coupled to the filter, while the plunger head remains within the tubular container, and while the filter support remains within the filtration assembly.

173. The method according to claim 172,
wherein the filter support is shaped so as to define a central opening, in addition to the plurality of filtrate-passage openings, and
wherein removing the filter from the filter support and from the filtration assembly comprises pulling the filter through the central opening by removing the filter-withdrawal shaft from being partially inserted in the filtration assembly, while the distal portion of the filter-withdrawal shaft is coupled to the filter, while the plunger head remains within the tubular container, and while the filter support remains within the filtration assembly.

174. The method according to any one of claims 170-173, further comprising, after the filter has been removed from the tubular container, detecting the presence of a biological particulate trapped by the filter.

175. The method according to claim 174, wherein detecting the presence of the biological particulate trapped by the filter comprising using a lateral flow test strip to detect the presence of the biological particulate trapped by the filter.

176. The method according to claim 174, wherein the biological particulate is selected from the group consisting of: a virus, a bacterium, a microorganism, a fungus, a spore, a mite, a biological cell, a biological antigen, a protein, a protein antigen, and a carbohydrate antigen.

177. The method according to any one of claims 170-173, wherein the liquid specimen sample includes gargled fluid.

178. The method according to any one of claims 170-173, wherein the filter comprises a polyethersulfone (PES) membrane filter.

179. A method comprising:
passing at least a portion of a liquid specimen sample through a filter;
inserting at least a portion of the filter into an extraction tube and bathing the at least a portion of the filter in the extraction tube in a liquid comprising one or more reagents;
thereafter, discarding some of the liquid; and
thereafter, squeezing the filter to squeeze some of the liquid from the filter.

180. The method according to claim 179, wherein discarding some of the liquid comprises removing the filter from the extraction tube while some of the liquid remain in the extraction tube.

181. The method according to claim 179, wherein discarding some of the liquid comprises draining some of the liquid from the extraction tube while the at least a portion of the filter remains in the tube.

182. The method according to claim 179, further comprising, after bathing the at least a portion of the filter in the liquid and before discarding some of the liquid:
squeezing the filter to squeeze some of the liquid from the filter while the at least a portion of the filter is within the extraction tube.

183. The method according to any one of claims 179-182, further comprising, after squeezing the filter, testing for the presence of a biological particulate trapped by the filter.

184. The method according to claim 183, wherein the biological particulate is selected from the group consisting of: a virus, a bacterium, a microorganism, a fungus, a spore, a mite, a biological cell, a biological antigen, a protein, a protein antigen, and a carbohydrate antigen.

185. The method according to claim 183, wherein testing for the presence of the biological particular trapped by the filter comprises testing a portion of the liquid for the presence of a target analyte released into the liquid from the biological particulate trapped by the filter.

186. The method according to claim 183, wherein testing for the presence of a biological particulate trapped by the filter comprises bringing the filter into direct contact with a sample pad of a lateral flow test strip.

187. The method according to claim 186, wherein the lateral flow test strip is contained at least partially within a housing.

188. The method according to claim 187, wherein the housing is selected from the group consisting of: a cartridge and a card.

189. The method according to claim 187, wherein the lateral flow test strip is disposed at least partially within the housing such that a test area of the lateral flow test strip is visible through one or more result windows defined by the housing.

190. The method according to claim 187,
wherein the housing is shaped so as to define a channel in fluid communication with the sample pad, and
wherein bringing the filter into the direct contact with the sample pad comprises inserting the filter into the channel and into the direct contact with the sample pad of the lateral flow test strip.

191. The method according to any one of claims 179-182, further comprising bunching up at least a portion of the filter after passing the at least a portion of the liquid specimen sample through the filter and before inserting the at least a portion of the filter into the extraction tube.

192. The method according to claim 191, wherein bunching up the at least a portion of the filter comprises bunching up the at least a portion of the filter into a flower-like arrangement.

193. The method according to claim 191, wherein passing the at least a portion of the liquid specimen sample through the filter comprises passing the at least a portion of the liquid specimen sample through the filter while the filter is in a flat shape.

194. The method according to claim 191, wherein squeezing the filter comprises squeezing the bunched-up portion of the filter by longitudinally compacting the bunched-up portion of the filter against a distal blocking surface intercepted by a straight central longitudinal axis of the extraction tube.

195. The method according to claim 191, wherein squeezing the filter comprises:
inserting the bunched-up portion of the filter into a second tube separate and distinct from the extraction tube; and
squeezing the bunched-up portion of the filter to squeeze some of the liquid from the filter while the at least a portion of the filter is within the second tube.

196. The method according to claim 195, wherein squeezing the bunched-up portion of the filter by longitudinally compacting the bunched-up portion of the filter against a distal blocking surface intercepted by a straight central longitudinal axis of the second tube.

197. A method comprising:
collecting a liquid specimen sample potentially containing biological particulate; and
testing for the presence of the biological particulate in the liquid specimen sample, by:
passing at least a portion of the liquid specimen sample through a filter;
applying one or more reagents to the filter; and
thereafter, bringing the filter into direct contact with a sample pad of a lateral flow test strip.

198. The method according to claim 197, wherein the biological particulate is selected from the group consisting of: a virus, a bacterium, a microorganism, a fungus, a spore, a mite, a biological cell, a biological antigen, a protein, a protein antigen, and a carbohydrate antigen.

199. The method according to claim 197, further comprising, after applying the one or more reagents to the filter and before bringing the filter into the direct contact with the sample pad, discarding a portion of the one or more reagents.

200. The method according to claim 197, wherein bringing the filter into the direct contact with the sample pad comprises pressing at least a portion of the filter against the sample pad.

201. The method according to claim 197, wherein bringing the filter into the direct contact with the sample pad comprises squeezing the filter against the sample pad of the lateral flow test strip.

202. The method according to claim 201, wherein squeezing the filter against the sample pad comprises squeezing the filter by longitudinally compacting the bunched-up portion of the filter against the sample pad.

203. The method according to claim 197, wherein bringing the filter into the direct contact with the sample pad comprises bringing the filter into the direct contact with the sample pad using a filter shaft that includes a distal portion that is coupled to the filter.

204. The method according to claim 203, wherein the distal portion of the filter shaft is coupled to a central portion of the filter.

205. The method according to claim 203, wherein bringing the filter into the direct contact with the sample pad comprises orienting the filter shaft so that a straight central longitudinal axis of the filter shaft forms an angle of 30-90 degrees with the sample pad.

206. The method according to claim 205, wherein orienting the filter shaft comprises orienting the filter shaft such that the angle is 45-90 degrees.

207. The method according to any one of claims 197-206, further comprising bunching up at least a portion of the filter after passing the at least a portion of the liquid specimen sample through the filter and before bringing the filter into the direct contact with the sample pad.

208. The method according to claim 207, wherein bunching up the at least a portion of the filter comprises bunching up the at least a portion of the filter into a flower-like arrangement.

209. The method according to claim 207, wherein bringing the filter into the direct contact with the sample pad comprises squeezing the bunched-up portion of the filter against the sample pad of the lateral flow test strip.

210. The method according to claim 209, wherein squeezing the bunched-up portion of the filter against the sample pad comprises squeezing the bunched-up portion of the filter by longitudinally compacting the bunched-up portion of the filter against the sample pad.

211. The method according to claim 209, wherein squeezing the bunched-up portion of the filter against the sample pad delivers to the sample pad both (a) some of the one or more reagents contained in or on the filter at locations of the filter that directly contact the sample pad, and (b) some of the one or more reagents contained in or on the filter at locations other than the locations of the filter that directly contact the sample pad.

212. The method according to claim 207, wherein passing the at least a portion of the liquid specimen sample through the filter comprises passing the at least a portion of the liquid specimen sample through the filter while the filter is in a flat shape.

213. The method according to any one of claims 197-206, wherein the lateral flow test strip is contained at least partially within a housing.

214. The method according to claim 213, wherein the housing is selected from the group consisting of: a cartridge and a card.

215. The method according to claim 213, wherein the lateral flow test strip is disposed at least partially within the housing such that a test area of the lateral flow test strip is visible through one or more result windows defined by the housing.

216. The method according to claim 213,
wherein the housing is shaped so as to define a channel in fluid communication with the sample pad, and
wherein bringing the filter into the direct contact with the sample pad comprises inserting the filter into the channel and into the direct contact with the sample pad of the lateral flow test strip.

217. The method according to claim 216,
wherein the method further comprises bunching up at least a portion of the filter after passing the at least a portion of the liquid specimen sample through the filter and before bringing the filter into the direct contact with the sample pad, and
wherein bringing the filter into the direct contact with the sample pad comprises inserting the bunched-up portion of the filter into the channel and advancing a distal portion of the bunched-up portion of the filter through the channel and into the direct contact with the sample pad of the lateral flow test strip.

218. The method according to claim 217, wherein bringing the filter into the direct contact with the sample pad comprises squeezing the bunched-up portion of the filter by longitudinally compacting the bunched-up portion of the filter against the sample pad of the lateral flow test strip.

219. The method according to claim 217,
wherein bunching up the at least a portion of the filter comprises bunching up the at least a portion of the filter such that an entirety of a perimeter of the filter points distally,
wherein inserting the bunched-up portion of the filter into the channel comprises inserting, in a distal direction, the bunched-up portion of the filter into the channel while the entirety of the perimeter of the filter points distally, and
wherein advancing the distal portion of the bunched-up portion of the filter comprises advancing the distal portion of the bunched-up portion of the filter through the channel so that at least a portion of the perimeter makes direct contact with the sample pad of the lateral flow test strip.

220. The method according to claim 213,
wherein the sample pad is disposed at an upstream end portion of the lateral flow test strip, and the lateral flow test strip further includes (a) an absorbent pad disposed at a downstream end portion of the lateral flow test strip, and (b) a membrane disposed longitudinally between the sample pad and the absorbent pad, the membrane comprising a test area, which comprises a test line and a control line, and
wherein the housing is configured such that when the housing is placed on a flat surface, the test area of the membrane is farther from flat surface than the sample pad is from the flat surface.

221. The method according to claim 220, wherein the housing is shaped so as to define a raised portion at least under the test area of the membrane.

222. The method according to claim 220, wherein the lateral flow test strip is oriented obliquely with respect to the flat surface when the housing is placed on the flat surface, such that the downstream end portion of the lateral flow test strip is more elevated from the flat surface than is the upstream end portion of the lateral flow test strip.

223. The method according to claim 222, wherein the lateral flow test strip is oriented at an angle of 1-20 degrees with respect to the flat surface when the housing is placed on the flat surface.

224. A sampling device for concentrating a liquid specimen sample, the sampling device comprising:
a container;
a filter;
a filter support, which is shaped so as to define (a) a support surface on which the filter is removably disposed, and (b) a plurality of filtrate-passage openings through the filter support; and
a filter reinforcement, which is coupled to a surface of the filter so as to cover 1%-50% of a surface area of the surface.

225. The sampling device according to claim 224, wherein the filter reinforcement has a greater tensile strength than the filter.

226. The sampling device according to claim 224, wherein the filter reinforcement is not porous.

227. The sampling device according to claim 224, wherein the filter reinforcement comprises metal.

228. The sampling device according to claim 224, wherein the filter reinforcement comprises a polymer.

229. The sampling device according to claim 224, wherein the filter comprises a polyethersulfone (PES) membrane filter.

230. The sampling device according to claim 224, wherein the filter reinforcement is coupled to the surface of the filter so as to cover 1%-30% of the surface area of the surface.

231. The sampling device according to claim 230, wherein the filter reinforcement is coupled to the surface of the filter so as to cover 5%-25% of the surface area of the surface.

232. The sampling device according to claim 224, wherein the filter reinforcement is shaped so as to define a plurality of thin strips.

233. The sampling device according to claim 224, wherein the filter reinforcement is shaped so as to define a central hub.

234. The sampling device according to claim 233, wherein the central hub is shaped so as to define a central opening therethrough.

235. The sampling device according to claim 224, wherein the filter reinforcement is shaped so as to define a peripheral rim.

236. The sampling device according to claim 224, wherein the filter reinforcement is shaped so as to define a plurality of spokes.

237. The sampling device according to claim 236, wherein the filter reinforcement is shaped so as to define a peripheral rim connected to the spokes.

238. The sampling device according to claim 236, wherein the filter reinforcement is shaped so as to define a central hub connected to the spokes.

239. The sampling device according to claim 238, wherein the filter reinforcement is shaped so as to define a peripheral rim connected to the spokes.

240. The sampling device according to claim 224, wherein the filter is circular when flat.

241. The sampling device according to any one of claims 224-240,
wherein the container is tubular, and is shaped so as to define an inner wall and a proximal container opening for receiving the liquid specimen sample,
wherein the sampling device further comprises a plunger, which (a) comprises a plunger head and (b) is insertable into the tubular container via the proximal container opening, such that a lateral surface of the plunger head forms a fluid-tight movable seal with the inner wall, and
wherein the sampling device is configured such that movement of the plunger head within the tubular container, when the liquid specimen sample is contained in the tubular container and the filter is disposed in the tubular container, pushes at least a portion of the liquid specimen sample through the filter in an upstream-to-downstream direction.

242. The sampling device according to claim 241, wherein the surface to which the filter reinforcement is coupled is an upstream surface of the filter.

243. The sampling device according to any one of claims 224-240, wherein the filter reinforcement is an upstream filter reinforcement, and wherein the sampling device further comprises a downstream filter reinforcement, which is coupled to a downstream surface of the filter.

244. The sampling device according to claim 243, wherein the downstream filter reinforcement is shaped so as to define a central hub.

245. The sampling device according to any one of claims 224-240,
wherein the sampling device further comprises a withdrawer comprising a filter-withdrawal shaft, which is partially inserted in the container,
wherein the sampling device is configured such that removal of the filter-withdrawal shaft from the container, while a distal portion of the filter-withdrawal shaft is coupled to the filter and the filter support remains within the sampling device, removes the filter from the sampling device.

246. The sampling device according to claim 245,
wherein the filter support is shaped so as to define a central opening, in addition to the plurality of filtrate-passage openings, and
wherein the sampling device is configured such that removal of the filter-withdrawal shaft from the sampling device, while the filter support remains within the sampling device, pulls the filter through the central opening and removes the filter from the sampling device.

247. The sampling device according to any one of claims 224-240, wherein the sampling device is configured such that removal of the filter-withdrawal shaft from the container, while the filter support remains within the sampling device, removes the filter from the sampling device and bunches up at least a portion of the filter.

248. The sampling device according to claim 247, wherein the sampling device is configured such that the removal of the filter-withdrawal shaft from the container, while the filter support remains within the sampling device, removes the filter from the sampling device and bunches up the at least a portion of the filter into a flower-like arrangement.

249. The sampling device according to any one of claims 224-240, further comprising a filter shaft having a distal portion coupled to the filter.

250. The sampling device according to claim 249, wherein the distal portion of the filter shaft is indirectly coupled to the filter.

251. The sampling device according to claim 249, wherein the distal portion of the filter shaft is directly coupled to the filter.

252. The sampling device according to claim 251, further comprising a distal plate that is fixed to a distal end of the distal portion of the filter shaft such that a central portion of the filter is between the distal end and the distal plate, so as to directly couple the filter to the filter shaft.

253. A testing kit comprising the sampling device according to any one of claims 224-240, the testing kit further comprising a diagnostic test configured to detect the presence of a biological particulate trapped by the filter.

254. The testing kit according to claim 253, wherein the diagnostic test comprises a lateral flow test strip.

255. The testing kit according to claim 254, further comprising reagents for use with the lateral flow test strip.

256. The testing kit according to claim 253, wherein the diagnostic test comprises a rapid molecular test.

257. The testing kit according to claim 253, wherein the biological particulate is selected from the group consisting of: a virus, a bacterium, a microorganism, a fungus, a spore, a mite, a biological cell, a biological antigen, a protein, a protein antigen, and a carbohydrate antigen.

258. A diagnostic test for use with a flat surface, the diagnostic test comprising:
a housing selected from the group consisting of: a cartridge and a card; and
a lateral flow test strip, which:
comprises (a) a sample pad disposed at an upstream end portion of the lateral flow test strip, (b) an absorbent pad disposed at a downstream end portion of the lateral flow test strip, and (c) a membrane disposed longitudinally between the sample pad and the absorbent pad, the membrane comprising a test area, which comprises a test line and a control line, and
is contained at least partially within the housing,
wherein the housing is configured such that when the housing is placed on the flat surface, the test area of the membrane is farther from flat surface than the sample pad is from the flat surface.

259. The diagnostic test according to claim 258, wherein the housing is shaped so as to define a raised portion at least under the test area of the membrane.

260. The diagnostic test according to claim 258, wherein the lateral flow test strip is oriented obliquely with respect to the flat surface when the housing is placed on the flat surface, such that the downstream end portion of the lateral flow test strip is more elevated from the flat surface than is the upstream end portion of the lateral flow test strip.

261. The diagnostic test according to claim 260, wherein the lateral flow test strip is oriented at an angle of 1-20 degrees with respect to the flat surface when the housing is placed on the flat surface.

262. The diagnostic test according to claim 258, wherein the housing is shaped so as to define a channel in fluid communication with the sample pad.

263. The diagnostic test according to claim 262, wherein the channel has an internal length of 1-5 cm.

264. The diagnostic test according to claim 258, wherein the housing is shaped so as to define one or more result windows, and wherein the lateral flow test strip is disposed at least partially within the housing such that a test area of the lateral flow test strip is visible through the one or more result windows defined by the housing.

265. A sampling device for concentrating a liquid specimen sample, the sampling device comprising a filtration assembly, which comprises:
a tubular container, which is shaped so as to define an inner wall and a proximal container opening for receiving the liquid specimen sample;
a plunger, which (i) comprises a plunger head and (ii) is insertable into the tubular container via the proximal container opening, such that a lateral surface of the plunger head forms a fluid-tight movable seal with the inner wall;
a filter;
a filter support, which is shaped so as to define (A) a support surface on which the filter is removably disposed, and (B) a plurality of filtrate-passage openings through the filter support; and
a clamping surface,
wherein sampling device is transitionable from:
a filter-clamping state, in which the filter is removably disposed on the support surface, clamped axially between the clamping surface and a peripheral portion of the support surface, to
a filter-release state, in which the filter is not clamped axially between the clamping surface and the peripheral portion of the support surface,
wherein the filtration assembly is configured such that movement of the plunger head within the tubular container, when the liquid specimen sample is contained in the tubular container and the sampling device is in the filter-clamping state, pushes at least a portion of the liquid specimen sample through the filter and the filtrate-passage openings, and
wherein the sampling device is configured such that the filter is removable from the tubular container while the plunger head and the filter support remain within the tubular container and the sampling device is in the filter-release state.

266. A method for concentrating a liquid specimen sample, the method comprising:
placing the liquid specimen sample in a tubular container of a filtration assembly of a sampling device;
inserting a plunger head of a plunger of the filtration assembly into the tubular container via a proximal container opening of the tubular container, such that a lateral surface of the plunger head forms a fluid-tight movable seal with an inner wall of the tubular container;
distally advancing the plunger head within the tubular container to drive at least a portion of the liquid specimen sample through a filter, while the sampling device is in a filter-clamping state, in which the filter is removably disposed on a support surface of a filter support, clamped axially between a clamping surface of the filtration assembly and a peripheral portion of the support surface, wherein the filter support is shaped so as to define a plurality of filtrate-passage openings through the filter support; and
removing the filter from the tubular container via the plunger-space proximal opening while the plunger head and the filter support remain within the tubular container and the sampling device is in a filter-release state, in which the filter is not clamped axially between the clamping surface and the peripheral portion of the support surface.

267. A sampling device for concentrating a liquid specimen sample, the sampling device comprising a filtration assembly, which comprises:
a container housing, which is shaped so as to define (a) a cylindrical space within the container housing, and (b) one or more first threads;
a tubular container, which (a) is shaped so as to define an inner wall and a proximal container opening for receiving the liquid specimen sample, and (b) is disposed at least partially within the cylindrical space of the container housing, such that the tubular container is rotatable with respect to the cylindrical space;
a plunger support, which is shaped so as to define one or more second threads, shaped so as to engage the one or more first threads;
a plunger, which (a) comprises a plunger head, (b) is insertable into the tubular container via the proximal container opening, such that a lateral surface of the plunger head forms a fluid-tight movable seal with the inner wall, and (c) is coupled to the plunger support, such that rotation of the plunger support with respect to the container housing, when the one or more second threads are engaged with the one or more first threads, distally advances the plunger support with respect to the container housing and thus the plunger within the tubular container as the tubular container rotates with respect to the container housing; and
a filter,
wherein the filtration assembly is configured such that movement of the plunger head within the tubular container, when the liquid specimen sample is contained in the tubular container and the filter is disposed in the tubular container, pushes at least a portion of the liquid specimen sample through the filter.

268. A method for concentrating a liquid specimen sample, the method comprising:
placing the liquid specimen sample in a tubular container of a filtration assembly of a sampling device, wherein the tubular container is shaped so as to define an inner wall and is disposed at least partially within a cylindrical space within a container housing of the filtration assembly, such that the tubular container is rotatable with respect to the cylindrical space;
inserting a plunger head of a plunger of the filtration assembly into the tubular container via a proximal container opening of the tubular container, such that a lateral surface of the plunger head forms a fluid-tight movable seal with an inner wall of the tubular container, wherein the plunger is coupled to a plunger support of the filtration assembly; and
rotating the plunger support with respect to the container housing while one or more second threads defined by the plunger support are engaged with one or more first threads defined by the container housing, so as to distally advance the plunger support with respect to the container housing and thus the plunger within the tubular container as the tubular container rotates with respect to the container housing, thereby pushing at least a portion of the liquid specimen sample through a filter removably disposed in the tubular container.

269. A sampling device for concentrating a liquid specimen sample, the sampling device comprising a filtration assembly, which comprises:
a tubular container, which is shaped so as to define an inner wall and a proximal container opening for receiving the liquid specimen sample;
a plunger, which (i) comprises a plunger head and a plunger rod, which (a) has a distal end portion to which the plunger head is coupled, and (b) is shaped so as to define a waste liquid receptacle within the plunger rod, and (ii) is insertable into the tubular container via the proximal container opening, such that a lateral surface of the plunger head forms a fluid-tight movable seal with the inner wall;
a filter; and
a mechanical energy storage element,
wherein the plunger head comprises filter support, which is shaped so as to define (a) a support surface on which the filter is removably disposed, and (b) a plurality of filtrate-passage openings through the filter support into the waste liquid receptacle, and
wherein the filtration assembly is configured such that movement of the plunger head within the tubular container, when the liquid specimen sample is contained in the tubular container and the filter is disposed on the support surface:
pushes at least a portion of the liquid specimen sample through the filter and the filtrate-passage openings and into the waste liquid receptacle, and
stores energy in the energy storage element.

270. A method for concentrating a liquid specimen sample, the method comprising:
placing the liquid specimen sample in a tubular container of a filtration assembly of a sampling device;
inserting a plunger head of a plunger of the filtration assembly into the tubular container via a proximal container opening of the tubular container, such that a lateral surface of the plunger head forms a fluid-tight movable seal with an inner wall of the tubular container, wherein the plunger further comprises a plunger rod, which (a) has a distal end portion to which the plunger head is coupled, and (b) is shaped so as to define a waste liquid receptacle within the plunger rod; and
distally advancing the plunger head within the tubular container so as to:
drive at least a portion of the liquid specimen sample through a filter removably disposed in the tubular container on a support surface of a filter support, wherein the filter support is shaped so as to define a plurality of filtrate-passage openings through the filter support into the waste liquid receptacle, and
stores energy in an energy storage element of the filtration assembly.
